(19) United States
(12) Patent Application Publication
(10) Pub. No.: US 2026/0022360 A1
(43) Pub. Date: Jan. 22, 2026

(54) CRISPR SYSTEMS

(71) Applicant: BIOCONSORTIA, INC., DAVIS, CA (US)

(72) Inventors: BETSY ALFORD, DAVIS, CA (US); HONG ZHU, WEST SACRAMENTO, CA (US); STEPHEN BOLARIS

(73) Assignee: BIOCONSORTIA, INC., DAVIS, CA (US)

(21) Appl. No.: 18/866,231

(22) PCT Filed: May 25, 2023
(86) PCT No.: PCT/US2023/067482

§ 371 Date: Nov. 15, 2024

Related U.S. Application Data
(60) Provisional application No. 63/346,322, filed on May 27, 2022.

(51) Int. Cl. C12N 9/22; C12N 9/78; C12N 15/11

(52) U.S. Cl. CPC C12N 9/222; C12N 9/78; C12N 15/111; C12N 2310/20; C12Y 305/04

(57) ABSTRACT

The disclosure relates to novel CRISPR-Cas systems identified in a variety of bacterial species. The compositions identified herein may be used to edit a heterologous polynucleotide, for example in a eukaryotic or prokaryotic cell.

### CROSS-REFERENCE TO RELATED APPLICATIONS

This application is a National Stage Entry of International Patent Application No. PCT/US2023/067482 filed 25 May 2023, which claims the benefit under 35 U.S.C. 119(e) of U.S. Provisional Patent Application Ser. No. 63/346,322 filed 27 May 2022, all of which are herein incorporated by reference in their entireties.

### REFERENCE TO SEQUENCE LISTING SUBMITTED ELECTRONICALLY

The official copy of the sequence listing is submitted electronically as an XML formatted sequence listing with a file named created on 25 Apr. 2023 and having a size of 687,882 bytes and is filed concurrently with the specification. The sequence listing comprised in this XML formatted document is part of the specification and is herein incorporated by reference in its entirety.

### FIELD

The instant disclosure relates generally to the field of biology, specifically genome-editing molecules derived from microbes, and microbial compositions for the improvement of traits in both eukaryotes and prokaryotes.

### BACKGROUND

Nucleotide-editing technology has enabled the modification of genomes across a wide variety of species, for the improvement of different traits of interest. Spanning both eukaryotes and prokaryotes, techniques including TAL Effector Nucleases, Meganucleases, Zinc Finger Nucleases, and CRISPR endonucleases provide ways of targeting and editing within the genomes of cells.

CRISPR-Cas systems are extensively leveraged in the treatment of human and animal diseases, curing cancer and genetic conditions, producing various compositions for industrial and pharmaceutical use, and improving the quality and quantity of food crops.

Cas endonucleases can be precisely developed for specific site modifications. A large number of Cas endonucleases have been described, each with different properties, such as PAM recognition site preferences.

Despite the advances during the past few years, there is a need for a larger variety of Cas endonucleases for target-specific modification of polynucleotides.

### SUMMARY

The microbes provided herein comprise at least one CRISPR-Cas system that is capable of creating a double strand break in, or adjacent to, a target polynucleotide that comprises an appropriate PAM, and to which it is directed by a guide polynucleotide, in any prokaryotic or eukaryotic cell. In some cases, the cell is a plant cell or an animal cell or a fungal cell.

Included are single-effector endonucleases, capable of recognizing, binding to, and optionally nicking (on a single strand of two strands) or cleaving (a single strand or a double strand) a target polynucleotide, for example double-stranded DNA.

### BRIEF DESCRIPTION OF THE SEQUENCE LISTING

The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

Sequence sources are given as <Strain #_Contig/Locus ID>.

SEQID NO:1 is the Strain 10022_03091 Cas endonuclease DNA sequence from Pseudarthrobacter chlorophenolicus.

SEQID NO:2 is the Strain 10078_01326 Cas endonuclease DNA sequence from Microbacterium sp. TPU 3598.

SEQID NO:3 is the Strain 10082_02958 Cas endonuclease DNA sequence from Microbacterium testaceum.

SEQID NO:4 is the Strain 100944_02101 Cas endonuclease DNA sequence from Bacillus sp. X1(2014).

SEQID NO:5 is the Strain 100949_02456 Cas endonuclease DNA sequence from Bacillus sp. X1(2014).

SEQID NO:6 is the Strain 10099_01758 Cas endonuclease DNA sequence from Rathayibacter tritici.

SEQID NO:7 is the Strain 10105_01169 Cas endonuclease DNA sequence from Microbacterium sp. TPU 3598.

SEQID NO:8 is the Strain 10107_02044 Cas endonuclease DNA sequence from Rathayibacter tritici.

SEQID NO:9 is the Strain 101119_04896 Cas endonuclease DNA sequence from Bacillus megaterium.

SEQID NO:10 is the Strain 101168_00758 Cas endonuclease DNA sequence from Bacillus megaterium.

SEQID NO:11 is the Strain 101169_03904 Cas endonuclease DNA sequence from Bacillus megaterium.

SEQID NO:12 is the Strain 101395_04849 Cas endonuclease DNA sequence from Paenibacillus xylanexedens.

SEQID NO:13 is the Strain 101632_00966 Cas endonuclease DNA sequence from Bacillus megaterium.

SEQID NO:14 is the Strain 101632_03946 Cas endonuclease DNA sequence from Bacillus megaterium.

SEQID NO:15 is the Strain 1017_03442 Cas endonuclease DNA sequence from Microbacterium sp. 1.5R.

SEQID NO: 16 is the Strain 1019_01381 Cas endonuclease DNA sequence from Ottowia sp. KADR8-3.

SEQID NO:17 is the Strain 102002_02944 Cas endonuclease DNA sequence from Bacillus simplex.

SEQID NO: 18 is the Strain 102413_04815 Cas endonuclease DNA sequence from Paenibacillus sp. JDR-2.

SEQID NO:19 is the Strain 102958_01637 Cas endonuclease DNA sequence from Bacillus sp. Y-01.

SEQID NO:20 is the Strain 103102_04419 Cas endonuclease DNA sequence from Paenibacillus sp. IHBB 10380.

SEQID NO:21 is the Strain 103112_04355 Cas endonuclease DNA sequence from Paenibacillus sp. IHBB 10380.

SEQID NO:22 is the Strain 103113_04392 Cas endonuclease DNA sequence from Paenibacillus sp. IHBB 10380.

SEQID NO:23 is the Strain 1038_02403 Cas endonuclease DNA sequence from Microbacterium sp. 1.5R.

SEQID NO:24 is the Strain 10387_01168 Cas endonuclease DNA sequence from Microbacterium sp. XT11.

SEQID NO:25 is the Strain 1040_01689 Cas endonuclease DNA sequence from Microbacterium sp. 1.5R.

SEQID NO:26 is the Strain 104168_03005 Cas endonuclease DNA sequence from Paenibacillus crassostreae.

SEQID NO:27 is the Strain 10419_02480 Cas endonuclease DNA sequence from Microbacterium sp. XT11.

SEQID NO:28 is the Strain 10420_00522 Cas endonuclease DNA sequence from Microbacterium sp. XT11.

SEQID NO:29 is the Strain 10426_00468 Cas endonuclease DNA sequence from Microbacterium sp. XT11.

SEQID NO:30 is the Strain 10455_02553 Cas endonuclease DNA sequence from Microbacterium sp. XT11.

SEQID NO:31 is the Strain 104624_02918 Cas endonuclease DNA sequence from Bacillus megaterium.

SEQID NO:32 is the Strain 10466_00895 Cas endonuclease DNA sequence from Pseudarthrobacter chlorophenolicus.

SEQID NO:33 is the Strain 1047_02160 Cas endonuclease DNA sequence from Microbacterium sp. XT11.

SEQID NO:34 is the Strain 10494_01762 Cas endonuclease DNA sequence from Microbacterium sp. XT11.

SEQID NO:35 is the Strain 10504_03293 Cas endonuclease DNA sequence from Microbacterium sp. XT11.

SEQID NO:36 is the Strain 10506_03105 Cas endonuclease DNA sequence from Microbacterium sp. XT11.

SEQID NO:37 is the Strain 10507_00398 Cas endonuclease DNA sequence from Microbacterium sp. XT11.

SEQID NO:38 is the Strain 105094_04656 Cas endonuclease DNA sequence from Bacillus megaterium.

SEQID NO:39 is the Strain 1051_01436 Cas endonuclease DNA sequence from Microbacterium sp. 1.5R.

SEQID NO:40 is the Strain 10511_03526 Cas endonuclease DNA sequence from Microbacterium sp. XT11.

SEQID NO:41 is the Strain 105149_04104 Cas endonuclease DNA sequence from Bacillus sp. X1(2014).

SEQID NO:42 is the Strain 105149_04649 Cas endonuclease DNA sequence from Bacillus sp. X1(2014).

SEQID NO:43 is the Strain 105194_03162 Cas endonuclease DNA sequence from Bacillus sp. X1(2014).

SEQID NO:44 is the Strain 105195_04607 Cas endonuclease DNA sequence from Bacillus sp. X1(2014).

SEQID NO:45 is the Strain 105195_04745 Cas endonuclease DNA sequence from Bacillus sp. X1(2014).

SEQID NO:46 is the Strain 105210_04468 Cas endonuclease DNA sequence from Bacillus sp. X1(2014).

SEQID NO:47 is the Strain 10522_02429 Cas endonuclease DNA sequence from Microbacterium sp. XT11.

SEQID NO:48 is the Strain 10533_01275 Cas endonuclease DNA sequence from Microbacterium sp. XT11.

SEQID NO:49 is the Strain 1063_00998 Cas endonuclease DNA sequence from Microbacterium sp. 1.5R.

SEQID NO:50 is the Strain 10631_01157 Cas endonuclease DNA sequence from Microbacterium sp. XT11.

SEQID NO:51 is the Strain 10634_01684 Cas endonuclease DNA sequence from Microbacterium sp. XT11.

SEQID NO:52 is the Strain 10635_01773 Cas endonuclease DNA sequence from Microbacterium sp. XT11.

SEQID NO:53 is the Strain 10638_03764 Cas endonuclease DNA sequence from Pseudarthrobacter chlorophenolicus.

SEQID NO:54 is the Strain 10647_02458 Cas endonuclease DNA sequence from Microbacterium sp. XT11.

SEQID NO:55 is the Strain 10660_01480 Cas endonuclease DNA sequence from Microbacterium sp. XT11.

SEQID NO:56 is the Strain 10669_01791 Cas endonuclease DNA sequence from Microbacterium sp. XT11.

SEQID NO:57 is the Strain 10671_00807 Cas endonuclease DNA sequence from Microbacterium sp. XT11.

SEQID NO:58 is the Strain 1078_01481 Cas endonuclease DNA sequence from Microbacterium sp. 1.5R.

SEQID NO:59 is the Strain 10785_00266 Cas endonuclease DNA sequence from Microbacterium sp. XT11.

SEQID NO:60 is the Strain 1080_02464 Cas endonuclease DNA sequence from Microbacterium sp. 1.5R.

SEQID NO:61 is the Strain 10805_00375 Cas endonuclease DNA sequence from Microbacterium sp. XT11.

SEQID NO:62 is the Strain 10806_03196 Cas endonuclease DNA sequence from Microbacterium sp. XT11.

SEQID NO:63 is the Strain 10808_01266 Cas endonuclease DNA sequence from Microbacterium sp. XT11.

SEQID NO:64 is the Strain 10817_00887 Cas endonuclease DNA sequence from Unknown.

SEQID NO:65 is the Strain 1082_03198 Cas endonuclease DNA sequence from Microbacterium sp. XT11.

SEQID NO:66 is the Strain 10822_00582 Cas endonuclease DNA sequence from Microbacterium sp. XT11.

SEQID NO:67 is the Strain 1083_01536 Cas endonuclease DNA sequence from Microbacterium sp. 1.5R.

SEQID NO:68 is the Strain 10839_02372 Cas endonuclease DNA sequence from Microbacterium sp. XT11.

SEQID NO:69 is the Strain 10842_01772 Cas endonuclease DNA sequence from Microbacterium sp. XT11.

SEQID NO:70 is the Strain 10844_02570 Cas endonuclease DNA sequence from Microbacterium sp. XT11.

SEQID NO:71 is the Strain 1087_01291 Cas endonuclease DNA sequence from Microbacterium sp. CGR1.

SEQID NO:72 is the Strain 1094_00967 Cas endonuclease DNA sequence from Microbacterium sp. 1.5R.

SEQID NO:73 is the Strain 1098_00553 Cas endonuclease DNA sequence from Microbacterium sp. CGR1.

SEQID NO:74 is the Strain 1109_01133 Cas endonuclease DNA sequence from Microbacterium sp. 1.5R.

SEQID NO:75 is the Strain 1113_01742 Cas endonuclease DNA sequence from Microbacterium sp. TPU 3598.

SEQID NO:76 is the Strain 1115_01841 Cas endonuclease DNA sequence from Microbacterium aurum.

SEQID NO:77 is the Strain 11199_02635 Cas endonuclease DNA sequence from Bacillus sp. X1(2014).

SEQID NO:78 is the Strain 1124_01292 Cas endonuclease DNA sequence from Glutamicibacter arilaitensis.

SEQID NO:79 is the Strain 11345_00502 Cas endonuclease DNA sequence from Microbacterium sp. XT11.

SEQID NO:80 is the Strain 11364_01125 Cas endonuclease DNA sequence from Microbacterium sp. XT11.

SEQID NO:81 is the Strain 11393_00661 Cas endonuclease DNA sequence from Microbacterium sp. XT11.

SEQID NO:82 is the Strain 11425_02393 Cas endonuclease DNA sequence from Microbacterium sp. XT11.

SEQID NO:83 is the Strain 11456_02753 Cas endonuclease DNA sequence from Microbacterium sp. XT11.

SEQID NO:84 is the Strain 1155_02473 Cas endonuclease DNA sequence from Microbacterium sp. CGR1.

SEQID NO:85 is the Strain 1165_01871 Cas endonuclease DNA sequence from Microbacterium sp. CGR1.

SEQID NO:86 is the Strain 11716_05148 Cas endonuclease DNA sequence from Unknown.

SEQID NO:87 is the Strain 11723_04575 Cas endonuclease DNA sequence from Bacillus megaterium.

SEQID NO:88 is the Strain 1176_01531 Cas endonuclease DNA sequence from Microbacterium sp. XT11.

SEQID NO:89 is the Strain 11762_04354 Cas endonuclease DNA sequence from Bacillus megaterium.

SEQID NO:90 is the Strain 11773_01698 Cas endonuclease DNA sequence from Microbacterium sp. XT11.

SEQID NO:91 is the Strain 1178_01705 Cas endonuclease DNA sequence from Microbacterium sp. XT11.

SEQID NO:92 is the Strain 11784_03035 Cas endonuclease DNA sequence from Bacillus megaterium.

SEQID NO:93 is the Strain 11787_03445 Cas endonuclease DNA sequence from Bacillus megaterium.

SEQID NO:94 is the Strain 1198_01200 Cas endonuclease DNA sequence from Pseudarthrobacter chlorophenolicus.

SEQID NO:95 is the Strain 1212_01737 Cas endonuclease DNA sequence from Glutamicibacter arilaitensis.

SEQID NO:96 is the Strain 12148_02440 Cas endonuclease DNA sequence from Paenibacillus naphthalenovorans.

SEQID NO:97 is the Strain 12193_04094 Cas endonuclease DNA sequence from Paenibacillus naphthalenovorans.

SEQID NO:98 is the Strain 12301_00979 Cas endonuclease DNA sequence from Unknown.

SEQID NO:99 is the Strain 1253_02264 Cas endonuclease DNA sequence from Pseudarthrobacter chlorophenolicus.

SEQID NO:100 is the Strain 1271_01237 Cas endonuclease DNA sequence from Pseudarthrobacter chlorophenolicus.

SEQID NO:101 is the Strain 1286_01295 Cas endonuclease DNA sequence from Pseudarthrobacter chlorophenolicus.

SEQID NO:102 is the Strain 12917_02996 Cas endonuclease DNA sequence from Curtobacterium pusillum.

SEQID NO:103 is the Strain 13445_04630 Cas endonuclease DNA sequence from Azospirillum lipoferum.

SEQID NO:104 is the Strain 1396_03114 Cas endonuclease DNA sequence from Paenibacillus sp. CAA11.

SEQID NO:105 is the Strain 14053_00313 Cas endonuclease DNA sequence from Paenibacillus yonginensis.

SEQID NO:106 is the Strain 14166_02703 Cas endonuclease DNA sequence from Arthrobacter sp. QXT-31.

SEQID NO:107 is the Strain 14167_01587 Cas endonuclease DNA sequence from Arthrobacter sp. PGP41.

SEQID NO:108 is the Strain 14171_02048 Cas endonuclease DNA sequence from Pseudarthrobacter phenanthrenivorans.

SEQID NO:109 is the Strain 14186_01112 Cas endonuclease DNA sequence from Arthrobacter sp. FB24.

SEQID NO:110 is the Strain 14193_00438 Cas endonuclease DNA sequence from Arthrobacter sp. PGP41.

SEQID NO:111 is the Strain 14196_00143 Cas endonuclease DNA sequence from Arthrobacter sp. PGP41.

SEQID NO:112 is the Strain 14202_02580 Cas endonuclease DNA sequence from Arthrobacter sp. PGP41.

SEQID NO: 113 is the Strain 14229_00612 Cas endonuclease DNA sequence from Arthrobacter sp. FB24.

SEQID NO:114 is the Strain 1431_00729 Cas endonuclease DNA sequence from Pseudarthrobacter phenanthrenivorans.

SEQID NO:115 is the Strain 1438_01477 Cas endonuclease DNA sequence from Pseudarthrobacter phenanthrenivorans.

SEQID NO:116 is the Strain 14544_01404 Cas endonuclease DNA sequence from Bacillus megaterium.

SEQID NO:117 is the Strain 14596_01694 Cas endonuclease DNA sequence from Bacillus sp. Y-01.

SEQID NO:118 is the Strain 14627_01883 Cas endonuclease DNA sequence from Microbacterium testaceum.

SEQID NO:119 is the Strain 14650_03793 Cas endonuclease DNA sequence from Pseudarthrobacter chlorophenolicus.

SEQID NO:120 is the Strain 14658_03699 Cas endonuclease DNA sequence from Arthrobacter sp. QXT-31.

SEQID NO:121 is the Strain 1471_00680 Cas endonuclease DNA sequence from Pseudarthrobacter phenanthrenivorans.

SEQID NO:122 is the Strain 1472_00433 Cas endonuclease DNA sequence from Pseudarthrobacter phenanthrenivorans.

SEQID NO:123 is the Strain 14727_02881 Cas endonuclease DNA sequence from Pseudarthrobacter chlorophenolicus.

SEQID NO:124 is the Strain 14743_03592 Cas endonuclease DNA sequence from Leifsonia xyli.

SEQID NO:125 is the Strain 14779_01378 Cas endonuclease DNA sequence from Pseudarthrobacter chlorophenolicus.

SEQID NO:126 is the Strain 14808_02364 Cas endonuclease DNA sequence from Paenibacillus xylanexedens.

SEQID NO:127 is the Strain 14817_01476 Cas endonuclease DNA sequence from Paenibacillus xylanexedens.

SEQID NO:128 is the Strain 14824_01629 Cas endonuclease DNA sequence from Paenibacillus xylanexedens.

SEQID NO:129 is the Strain 14881_01920 Cas endonuclease DNA sequence from Microbacterium sp. XT11.

SEQID NO:130 is the Strain 14945_00031 Cas endonuclease DNA sequence from Arthrobacter sp. ATCC 21022.

SEQID NO:131 is the Strain 14968_01911 Cas endonuclease DNA sequence from Arthrobacter sp. ZXY-2.

SEQID NO: 132 is the Strain 14970_00612 Cas endonuclease DNA sequence from Arthrobacter sp. FB24.

SEQID NO:133 is the Strain 14977_00931 Cas endonuclease DNA sequence from Pseudarthrobacter chlorophenolicus.

SEQID NO:134 is the Strain 15010_02299 Cas endonuclease DNA sequence from Arthrobacter sp. PGP41.

SEQID NO:135 is the Strain 15062_00931 Cas endonuclease DNA sequence from Pseudarthrobacter chlorophenolicus.

SEQID NO:136 is the Strain 15158_01222 Cas endonuclease DNA sequence from Pseudarthrobacter phenanthrenivorans.

SEQID NO:137 is the Strain 15306_00078 Cas endonuclease DNA sequence from Paenibacillus yonginensis.

SEQID NO:138 is the Strain 15309_00706 Cas endonuclease DNA sequence from Paenibacillus yonginensis.

SEQID NO:139 is the Strain 15353_01923 Cas endonuclease DNA sequence from Paenibacillus yonginensis.

SEQID NO:140 is the Strain 15393_03167 Cas endonuclease DNA sequence from Paenibacillus sp. Y412MC10.

SEQID NO:141 is the Strain 15407_04378 Cas endonuclease DNA sequence from Paenibacillus yonginensis.

SEQID NO:142 is the Strain 15448_04948 Cas endonuclease DNA sequence from Sinorhizobium meliloti.

SEQID NO:143 is the Strain 15469_04140 Cas endonuclease DNA sequence from Mitsuaria sp. 7.

SEQID NO:144 is the Strain 15531_01961 Cas endonuclease DNA sequence from Mitsuaria sp. 7.

SEQID NO:145 is the Strain 15546_00078 Cas endonuclease DNA sequence from Pseudarthrobacter chlorophenolicus.

SEQID NO:146 is the Strain 15832_01937 Cas endonuclease DNA sequence from Arthrobacter sp. PGP41.

SEQID NO:147 is the Strain 15859_02500 Cas endonuclease DNA sequence from Pseudarthrobacter chlorophenolicus.

SEQID NO:148 is the Strain 15875_01814 Cas endonuclease DNA sequence from Pseudarthrobacter chlorophenolicus.

SEQID NO:149 is the Strain 15939_01790 Cas endonuclease DNA sequence from Arthrobacter sp. ZXY-2.

SEQID NO:150 is the Strain 15940_03638 Cas endonuclease DNA sequence from Arthrobacter sp. FB24.

SEQID NO:151 is the Strain 16_02923 Cas endonuclease DNA sequence from Pseudarthrobacter chlorophenolicus.

SEQID NO:152 is the Strain 16023_03049 Cas endonuclease DNA sequence from Pseudarthrobacter chlorophenolicus.

SEQID NO:153 is the Strain 16060_02050 Cas endonuclease DNA sequence from Pseudarthrobacter phenanthrenivorans.

SEQID NO:154 is the Strain 16064_03513 Cas endonuclease DNA sequence from Methylorubrum extorquens.

SEQID NO:155 is the Strain 16095_00628 Cas endonuclease DNA sequence from Sinorhizobium fredii.

SEQID NO:156 is the Strain 16107_02971 Cas endonuclease DNA sequence from Arthrobacter sp. ATCC 21022.

SEQID NO:157 is the Strain 16130_03182 Cas endonuclease DNA sequence from Arthrobacter sp. U41.

SEQID NO: 158 is the Strain 16135_03880 Cas endonuclease DNA sequence from Bacillus sp. Y-01.

SEQID NO:159 is the Strain 16157_00325 Cas endonuclease DNA sequence from Arthrobacter sp. ATCC 21022.

SEQID NO:160 is the Strain 16158_01725 Cas endonuclease DNA sequence from Arthrobacter sp. QXT-31.

SEQID NO:161 is the Strain 16194_01685 Cas endonuclease DNA sequence from Arthrobacter sp. ATCC 21022.

SEQID NO:162 is the Strain 16216_02576 Cas endonuclease DNA sequence from Arthrobacter sp. ZXY-2.

SEQID NO:163 is the Strain 16233_01732 Cas endonuclease DNA sequence from Pseudarthrobacter chlorophenolicus.

SEQID NO:164 is the Strain 16237_03292 Cas endonuclease DNA sequence from Pseudarthrobacter chlorophenolicus.

SEQID NO:165 is the Strain 16248_01824 Cas endonuclease DNA sequence from Pseudarthrobacter phenanthrenivorans.

SEQID NO:166 is the Strain 1625_02362 Cas endonuclease DNA sequence from Chryseobacterium glaciei.

SEQID NO:167 is the Strain 16274_00773 Cas endonuclease DNA sequence from Pseudarthrobacter phenanthrenivorans.

SEQID NO:168 is the Strain 16288_06493 Cas endonuclease DNA sequence from Azospirillum thiophilum.

SEQID NO:169 is the Strain 16288_06718 Cas endonuclease DNA sequence from Azospirillum thiophilum.

SEQID NO:170 is the Strain 16299_00539 Cas endonuclease DNA sequence from Rathayibacter tritici.

SEQID NO:171 is the Strain 16333_02521 Cas endonuclease DNA sequence from Pseudarthrobacter chlorophenolicus.

SEQID NO:172 is the Strain 16334_02912 Cas endonuclease DNA sequence from Pseudarthrobacter chlorophenolicus.

SEQID NO:173 is the Strain 16349_00705 Cas endonuclease DNA sequence from Arthrobacter sp. ZXY-2.

SEQID NO:174 is the Strain 16351_01257 Cas endonuclease DNA sequence from Pseudarthrobacter phenanthrenivorans.

SEQID NO:175 is the Strain 16369_02319 Cas endonuclease DNA sequence from Arthrobacter sp. ZXY-2.

SEQID NO:176 is the Strain 16370_00277 Cas endonuclease DNA sequence from Arthrobacter sp. PGP41.

SEQID NO: 177 is the Strain 16372_00908 Cas endonuclease DNA sequence from Microbacterium sp. No. 7.

SEQID NO: 178 is the Strain 16396_02202 Cas endonuclease DNA sequence from Arthrobacter crystallopoietes.

SEQID NO:179 is the Strain 16404_01792 Cas endonuclease DNA sequence from Pseudarthrobacter chlorophenolicus.

### DETAILED DESCRIPTION

While the following terms are believed to be well understood by one of ordinary skill in the art, the following are set forth to facilitate explanation of the presently disclosed subject matter.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein the terms “microorganism” or “microbe” should be taken broadly. These terms are used interchangeably and include, but are not limited to, the two prokaryotic domains, Bacteria and Archaea, as well as eukaryotic Fungi and Protists. In some embodiments, the disclosure refers to the “microbes” of Table 1, or the “microbes” of various other tables or paragraphs present in the disclosure. This characterization can refer to not only the identified taxonomic bacterial genera of the tables, but also the identified taxonomic species, as well as the various novel and newly identified bacterial strains of said tables.

As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism encompasses individual cells (e.g., unicellular microorganisms) or more than one cell (e.g., multi-cellular microorganism). A “population of microorganisms” may thus refer to a multiple cells of a single microorganism, in which the cells share common genetic derivation.

As used herein, the term “bacterium” or “bacteria” refers in general to any prokaryotic organism, and may reference an organism from either Kingdom Eubacteria (Bacteria), Kingdom Archaebacteria (Archae), or both. In some cases, bacterial genera or other taxonomic classifications have been reassigned due to various reasons (such as but not limited to the evolving field of whole genome sequencing), and it is understood that such nomenclature reassignments are within the scope of any claimed taxonomy. For example, certain species of the genus Erwinia have been described in the literature as belonging to genus Pantoea (Zhang, Y., Qiu, S. Examining phylogenetic relationships of Erwinia and Pantoea species using whole genome sequence data. Antonie van Leeuwenhoek 108, 1037-1046 (2015).).

The term “16S” refers to the DNA sequence of the 16S ribosomal RNA (rRNA) sequence of a bacterium. 16S rRNA gene sequencing is a well-established method for studying phylogeny and taxonomy of bacteria.

As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. In 1981, the Sydney Congress of the International Mycological Association laid out rules for the naming of fungi according to their status as anamorph, teleomorph, or holomorph (Taylor, J. W. One Fungus=One Name: DNA and fungal nomenclature twenty years after PCR. IMA Fungus 2, 113-120 (2011).). With the development of genomic sequencing, it became evident that taxonomic classification based on molecular phylogenetics did not align with morphological-based nomenclature (Shenoy, B. D., Jeewon, R. and Hyde, K. D. (2007). Impact of DNA sequence-data on the taxonomy of anamorphic fungi. Fungal Diversity 26: 1-54.). As a result, in 2011 the International Botanical Congress adopted a resolution approving the International Code of Nomenclature for Algae, Fungi, and Plants (Melbourne Code) (2012), with the stated outcome of designating “One Fungus=One Name” (Hawksworth, D. L. Managing and coping with names of pleomorphic fungi in a period of transition. IMA Fungus 3, 15-24 (2012)).

The term “Internal Transcribed Spacer” (“ITS”) refers to the spacer DNA (non-coding DNA) situated between the small-subunit ribosomal RNA (rRNA) and large-subunit (LSU) rRNA genes in the chromosome or the corresponding transcribed region in the polycistronic rRNA precursor transcript. ITS gene sequencing is a well-established method for studying phylogeny and taxonomy of fungi. In some cases, the “Large SubUnit” (“LSU”) sequence is used to identify fungi. LSU gene sequencing is a well-established method for studying phylogeny and taxonomy of fungi. Some fungal microbes of the present invention may be described by an ITS sequence and some may be described by an LSU sequence. Both are understood to be equally descriptive and accurate for determining taxonomy.

The term “microbial consortia” or “microbial consortium” refers to a subset of a microbial community of individual microbial species, or strains of a species, which can be described as carrying out a common function, or can be described as participating in, or leading to, or correlating with, a recognizable parameter or plant phenotypic trait. The community may comprise one or more species, or strains of a species, of microbes. In some instances, the microbes coexist within the community symbiotically.

The term “microbial community” means a group of microbes comprising two or more species or strains. Unlike microbial consortia, a microbial community does not have to be carrying out a common function, or does not have to be participating in, or leading to, or correlating with, a recognizable parameter or plant phenotypic trait.

The term “accelerated microbial selection” or “AMS” is used interchangeably with the term “directed microbial selection” or “DMS” and refers to the iterative selection methodology that was utilized, in some embodiments of the disclosure, to derive the claimed microbial species or consortia of said species.

As used herein, “isolate,” “isolated,” “isolated microbe,” and like terms, are intended to mean that the one or more microorganisms has been separated from at least one of the materials with which it is associated in a particular environment (for example soil, water, plant tissue).

Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an agricultural carrier.

In certain aspects of the disclosure, the isolated microbes exist as isolated and biologically pure cultures. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” See, e.g., In re Bergstrom, 427 F.2d 1394, (CCPA 1970)(discussing purified prostaglandins), see also, In re Bergy, 596 F.2d 952 (CCPA 1979)(discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

The term “growth medium” as used herein, is any medium which is suitable to support growth of a plant. By way of example, the media may be natural or artificial including, but not limited to: soil, potting mixes, bark, vermiculite, hydroponic solutions alone and applied to solid plant support systems, and tissue culture gels. It should be appreciated that the media may be used alone or in combination with one or more other media. It may also be used with or without the addition of exogenous nutrients and physical support systems for roots and foliage.

In one embodiment, the growth medium is a naturally occurring medium such as soil, sand, mud, clay, humus, regolith, rock, or water. In another embodiment, the growth medium is artificial. Such an artificial growth medium may be constructed to mimic the conditions of a naturally occurring medium; however, this is not necessary. Artificial growth media can be made from one or more of any number and combination of materials including sand, minerals, glass, rock, water, metals, salts, nutrients, water. In one embodiment, the growth medium is sterile. In another embodiment, the growth medium is not sterile.

The medium may be amended or enriched with additional compounds or components, for example, a component which may assist in the interaction and/or selection of specific groups of microorganisms with the plant and each other. For example, antibiotics (such as penicillin) or sterilants (for example, quaternary ammonium salts and oxidizing agents) could be present and/or the physical conditions (such as salinity, plant nutrients (for example organic and inorganic minerals (such as phosphorus, nitrogenous salts, ammonia, potassium and micronutrients such as cobalt and magnesium), pH, and/or temperature) could be amended.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant part” is synonymous to a “portion” of a plant, and refers to any part of the plant, and can include distinct tissues and/or organs, and may be used interchangeably with the term “tissue” throughout.

“Progeny” comprises any subsequent generation of an organism, produced via sexual or asexual reproduction.

As used herein, the term “plant element” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, the term “cultivar” refers to a variety, strain, or race, of plant that has been produced by horticultural or agronomic techniques and is not normally found in wild populations.

As used herein, “improved” should be taken broadly to encompass improvement of a characteristic of a plant, as compared to a control plant, or as compared to a known average quantity associated with the characteristic in question. For example, “improved” plant biomass associated with application of a beneficial microbe, or consortia, of the disclosure can be demonstrated by comparing the biomass of a plant treated by the microbes taught herein to the biomass of a control plant not treated. Alternatively, one could compare the biomass of a plant treated by the microbes taught herein to the average biomass normally attained by the given plant, as represented in scientific or agricultural publications known to those of skill in the art. In the present disclosure, “improved” does not necessarily demand that the data be statistically significant (e.g., p<0.05); rather, any quantifiable difference demonstrating that one value (e.g., the average treatment value) is different from another (e.g., the average control value) can rise to the level of “improved.”

As used herein, “inhibiting and suppressing” and like terms should not be construed to require complete inhibition or suppression, although this may be desired in some embodiments.

As used herein, the term “genotype” refers to the genetic makeup of an individual cell, cell culture, tissue, organism (e.g., a plant), or group of organisms.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein

“Agronomic trait potential” is intended to mean a capability of a plant element for exhibiting a phenotype, preferably an improved agronomic trait, at some point during its life cycle, or conveying said phenotype to another plant element with which it is associated in the same plant.

As used herein, the term “molecular marker”, “marker”, or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed by the average person skilled in molecular-biological techniques.

As used herein, the term “trait” refers to a characteristic or phenotype. For example, in the context of some embodiments of the present disclosure, yield of a crop relates to the amount of marketable biomass produced by a plant (e.g., fruit, fiber, grain). Desirable traits may also include other plant characteristics, including but not limited to: water use efficiency, nutrient use efficiency, production, mechanical harvestability, fruit maturity, shelf life, pest/disease resistance, early plant maturity, tolerance to stresses, etc. A trait may be inherited in a dominant or recessive manner, or in a partial or incomplete-dominant manner. A trait may be monogenic (i.e., determined by a single locus) or polygenic (i.e., determined by more than one locus) or may also result from the interaction of one or more genes with the environment.

As used herein, the term “phenotype” refers to the observable characteristics of an individual cell, cell culture, organism (e.g., a plant), or group of organisms which results from the interaction between that individual's genetic makeup (i.e., genotype) and the environment.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue”, “homolog”, or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters.

As used herein, the term “nucleotide change” refers to, e.g., nucleotide substitution, deletion, insertion, chemical alteration, or any of the preceeding, as is well understood in the art.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g., at least 2-fold over background). Stringent conditions are sequence dependent and will be different in different circumstances. Longer sequences hybridize specifically at higher temperatures.

Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na+ ion, typically about 0.01 to 1.0 M Na+ ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g., Ausubel et al., 1998 and Sambrook et al., 2001. In some embodiments, stringent conditions are hybridization in 0.25 M Na2HPO4 buffer (pH 7.2) containing 1 mM Na2EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

In some embodiments, the cell or organism has at least one heterologous trait. As used herein, the term “heterologous trait” refers to a phenotype imparted to a cell or organism by an exogenous molecule or other organism (e.g., a microbe), DNA segment, heterologous polynucleotide or heterologous nucleic acid.

Various changes in phenotype are of interest to the present disclosure, including but not limited to modifying the fatty acid composition in a plant, altering the amino acid content of a plant, altering a plant's pathogen defense mechanism, increasing a plant's yield of an economically important trait (e.g., grain yield, forage yield, etc.) and the like. These results can be achieved by providing expression of heterologous products or increased expression of endogenous products in plants using the methods and compositions of the present disclosure

A “synthetic combination” can include a combination of a plant and a microbe of the disclosure. The combination may be achieved, for example, by coating the surface of a seed of a plant, such as an agricultural plant, or host plant tissue (root, stem, leaf, etc.), with a microbe of the disclosure. Further, a “synthetic combination” can include a combination of microbes of various strains or species. Synthetic combinations have at lest one variable that distinguishes the combination from any combination that occurs in nature. That variable may be, inter alia, a concentration of microbe on a seed or plant tissue that does not occur naturally, or a combination of microbe and plant that does not naturally occur, or a combination of microbes or strains that do not occur naturally together. In each of these instances, the synthetic combination demonstrates the hand of man and possesses structural and/or functional attributes that are not present when the individual elements of the combination are considered in isolation.

In some embodiments, a microbe can be “endogenous” to a seed or plant. As used herein, a microbe is considered “endogenous” to a plant or seed, if the microbe is derived from the plant specimen from which it is sourced. That is, if the microbe is naturally found associated with said plant. In embodiments in which an endogenous microbe is applied to a plant, then the endogenous microbe is applied in an amount that differs from the levels found on the plant in nature. Thus, a microbe that is endogenous to a given plant can still form a synthetic combination with the plant, if the microbe is present on said plant at a level that does not occur naturally.

In some embodiments, a composition (such as a microbe) can be “heterologous” (also termed “exogenous”) to another composition (such as a seed or plant), and in some aspects is referred to herein as a “heterologous composition”. As used herein, a microbe is considered “heterologous” to a plant or seed, if the microbe is not derived from the plant specimen from which it is sourced. That is, if the microbe is not naturally found associated with said plant. For example, a microbe that is normally associated with leaf tissue of a maize plant is considered exogenous to a leaf tissue of another maize plant that naturally lacks said microbe. In another example, a microbe that is normally associated with a maize plant is considered exogenous to a wheat plant that naturally lacks said microbe.

A composition is “heterologously disposed” when mechanically or manually applied, artificially inoculated, associated with, or disposed onto or into a plant element, seedling, plant or onto or into a plant growth medium or onto or into a treatment formulation so that the treatment exists on or in the plant element, seedling, plant, plant growth medium, or formulation in a manner not found in nature prior to the application of the treatment, e.g., said combination which is not found in nature in that plant variety, at that stage in plant development, in that plant tissue, in that abundance, or in that growth environment (for example, drought). In some embodiments, such a manner is contemplated to be selected from the group consisting of: the presence of the microbe; presence of the microbe in a different number of cells, concentration, or amount; the presence of the microbe in a different plant element, tissue, cell type, or other physical location in or on the plant; the presence of the microbe at different time period, e.g., developmental phase of the plant or plant element, time of day, time of season, and combinations thereof. In some embodiments, “heterologously disposed” means that the microbe being applied to a different tissue or cell type of the plant element than that in which the microbe is naturally found. In some embodiments, “heterologously disposed” means that the microbe is applied to a developmental stage of the plant element, seedling, or plant in which said microbe is not naturally associated, but may be associated at other stages. For example, if a microbe is normally found at the flowering stage of a plant and no other stage, a microbe applied at the seedling stage may be considered to be heterologously disposed. In some embodiments, a microbe is heterologously disposed the microbe is normally found in the root tissue of a plant element but not in the leaf tissue, and the microbe is applied to the leaf. In another non-limiting example, if a microbe is naturally found in the mesophyll layer of leaf tissue but is being applied to the epithelial layer, the microbe would be considered to be heterologously disposed. In some embodiments, “heterologously disposed” means that the native plant element, seedling, or plant does not contain detectable levels of the microbe in that same plant element, seedling, or plant. In some embodiments, “heterologously disposed” means that the microbe being applied is at a greater concentration, number, or amount of the plant element, seedling, or plant, than that which is naturally found in said plant element, seedling, or plant. For example, a microbe is heterologously disposed when present at a concentration that is at least 1.5 times greater, between 1.5 and 2 times greater, 2 times greater, between 2 and 3 times greater, 3 times greater, between 3 and 5 times greater, 5 times greater, between 5 and 7 times greater, 7 times greater, between 7 and 10 times greater, 10 times greater, or even greater than 10 times higher number, amount, or concentration than the concentration that was present prior to the disposition of said microbe. In another non-limiting example, a microbe that is naturally found in a tissue of a cupressaceous tree would be considered heterologous to tissue of a maize, wheat, cotton, soybean plant. In another example, a microbe that is naturally found in leaf tissue of a maize, spring wheat, cotton, soybean plant is considered heterologous to a leaf tissue of another maize, spring wheat, cotton, soybean plant that naturally lacks said microbe, or comprises the microbe in a different quantity.

Microbes can also be “heterologously disposed” on a given plant tissue. This means that the microbe is placed upon a plant tissue that it is not naturally found upon. For instance, if a given microbe only naturally occurs on the roots of a given plant, then that microbe could be exogenously applied to the above-ground tissue of a plant and would thereby be “heterologously disposed” upon said plant tissue. As such, a microbe is deemed heterologously disposed, when applied on a plant that does not naturally have the microbe present or does not naturally have the microbe present in the number that is being applied.

The compositions and methods herein may provide for a “modulated” “agronomic trait” or “trait of agronomic importance” to a host plant, which may include, but not be limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation. By the term “modulated”, it is intended to refer to a change in an agronomic trait that is changed by virtue of the presence of the microbe(s), exudate, broth, metabolite, etc. In aspects, the modulation provides for the imparting of a beneficial trait.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327: 167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus, and include adaptation molecules as well as interference molecules. An interference molecule of a bacterial adaptive immunity complex includes endonucleases. A Cas endonuclease described herein comprises one or more nuclease domains. A Cas endonuclease includes but is not limited to: a Cas9 protein, a Cpfl (Casl2) protein, a C2cl protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A “functional fragment”, “fragment that is functionally equivalent”, and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double strand break in) the target site is retained. The portion or subsequence of the Cas endonuclease can comprise a complete or partial (functional) peptide of any one of its domains such as for example, but not limiting to a complete of functional part of a Cas3 HD domain, a complete of functional part of a Cas3 Helicase domain, complete of functional part of a protein (such as but not limiting to a Cas5, Cas5d, Cas7 and Cas8bl).

The terms “functional variant”, “variant that is functionally equivalent”, and “functionally equivalent variant” of a Cas endonuclease or Cas effector protein are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “cascade” and “cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC”, and “crCascade system” are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%,

99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example E1S20150059010A1, published 26 Feb. 2015), or any combination thereof.

As used herein, the terms “guide polynucleotide/Cas endonuclease complex”, “guide polynucleotide/Cas endonuclease system”,“guide polynucleotide/Cas complex”, “guide polynucleotide/Cas system” and “guided Cas system” “Polynucleotide-guided endonuclease”, are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the CRISPR systems known in the art or described herein.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The terms “target site”, “target sequence”, “target site sequence, “target DNA”, “target locus”, “genomic target site”, “genomic target sequence”, and “genomic target locus” are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave The target site can be an endogenous site in the genome of a cell, or alternatively, the target site can be heterologous to the cell and thereby not be naturally occurring in the genome of the cell, or the target site can be found in a heterologous genomic location compared to where it occurs in nature. In some aspects, the target site is simply referred to as “target” when discussed with respect to a polynucleotide sequence. A “target cell” or “target organism” is one that comprises a target polynucleotide.

A “protospacer adjacent motif (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “alterations”, and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, and/or (v) any combination of the preceding.

A “modified nucleotide” or “edited nucleotide” refers to a nucleotide sequence of interest that comprises at least one alteration when compared to its non-modified nucleotide sequence.

As used herein, “donor polynucleotide” is a polynucleotide construct (e.g., DNA) that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

### Microbes and Microorganisms

As used herein the term “microorganism” should be taken broadly. It includes, but is not limited to, prokaryotic Bacteria and Archaea, as well as eukaryotic Fungi and Protists.

By way of example, the microorganisms may include: Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium, Variovorax and Halomonas), Firmicutes (such as Bacillus, Paenibacillus, Lactobacillus, Mycoplasma, and Acetobacterium), Actinobacteria (such as Brevibacterium, Janibacter, Streptomyces, Rhodococcus, Microbacterium, Curtobacterium, Cellulomonas, and Nocardioides), and the fungi Ascomycota (such as Trichoderma, Ampelomyces, Coniothyrium, Paecoelomyces, Penicillium, Cladosporium, Hypocrea, Beauveria, Metarhizium, Verticullium, Cordyceps, Pichea, and Candida), Basidiomycota (such as Coprinus, Corticium, and Agaricus) and Oomycota (such as Pythium), and Mucoromycota (such as Mucor, and Mortierella); as well as Orbilia/Arthrobotrys, Lysinibacillus, Microbacterium, Talaromyces, Arthrobacter, Kosakonia, Masillia, Novosphingobium, and Tumebacillus.

In a particular embodiment, the microorganism is an endophyte, or an epiphyte, or a microorganism inhabiting the plant rhizosphere or rhizosheath. That is, the microorganism may be found present in the soil material adhered to the roots of a plant or in the area immediately adjacent a plant's roots.

In one embodiment, the microorganism is an endophyte. Endophytes may benefit host plants by preventing pathogenic organisms from colonizing them. Extensive colonization of the plant tissue by endophytes creates a “barrier effect,” where the local endophytes outcompete and prevent pathogenic organisms from taking hold. Endophytes may also produce chemicals which inhibit the growth of competitors, including pathogenic organisms.

In certain embodiments, the microorganism is unculturable. This should be taken to mean that the microorganism is not known to be culturable or is difficult to culture using methods known to one skilled in the art.

Microorganisms of the present disclosure may be collected or obtained from any source or contained within and/or associated with material collected from any source.

In an embodiment, the microorganisms are obtained from any general terrestrial environment, including its soils, plants, fungi, animals (including invertebrates) and other biota, including the sediments, water and biota of lakes and rivers; from the marine environment, its biota and sediments (for example sea water, marine muds, marine plants, marine invertebrates (for example sponges), marine vertebrates (for example, fish)); the terrestrial and marine geosphere (regolith and rock, for example crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, road surfaces).

In another embodiment the microorganisms are collected from a source likely to favor the selection of appropriate microorganisms. By way of example, the source may be a particular environment in which it is desirable for other plants to grow, or which is thought to be associated with terroir. In another example, the source may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the microorganisms may be collected from commercial crops grown in such environments, or more specifically from individual crop plants best displaying a trait of interest amongst a crop grown in any specific environment, for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste, or smell. The microorganisms may be collected from a plant of interest or any material occurring in the environment of interest, including fungi and other animal and plant biota, soil, water, sediments, and other elements of the environment as referred to previously. In certain embodiments, the microorganisms are individual isolates separated from different environments.

In one embodiment, a microorganism or a combination of microorganisms, of use in the methods of the disclosure may be selected from a pre-existing collection of individual microbial species or strains based on some knowledge of their likely or predicted benefit to a plant. For example, the microorganism may be predicted to: improve nitrogen fixation; release phosphate from the soil organic matter; release phosphate from the inorganic forms of phosphate (e.g., rock phosphate); “fix carbon” in the root microsphere; live in the rhizosphere of the plant thereby assisting the plant in absorbing nutrients from the surrounding soil and then providing these more readily to the plant; increase the number of nodules on the plant roots and thereby increase the number of symbiotic nitrogen fixing bacteria (e.g., Rhizobium species) per plant and the amount of nitrogen fixed by the plant; elicit plant defensive responses such as ISR (induced systemic resistance) or SAR (systemic acquired resistance) which help the plant resist the invasion and spread of pathogenic microorganisms; compete with microorganisms deleterious to plant growth or health by antagonism, or competitive utilization of resources such as nutrients or space; change the color of one or more part of the plant, or change the chemical profile of the plant, its smell, taste or one or more other quality.

In one embodiment a microorganism or combination of microorganisms is selected from a pre-existing collection of individual microbial species or strains that provides no knowledge of their likely or predicted benefit to a plant. For example, a collection of unidentified microorganisms isolated from plant tissues without any knowledge of their ability to improve plant growth or health, or a collection of microorganisms collected to explore their potential for producing compounds that could lead to the development of pharmaceutical drugs.

In one embodiment, the microorganisms are acquired from the source material (for example, soil, rock, water, air, dust, plant or other organism) in which they naturally reside. The microorganisms may be provided in any appropriate form, having regard to its intended use in the methods of the disclosure. However, by way of example only, the microorganisms may be provided as an aqueous suspension, gel, homogenate, granule, powder, slurry, live organism or dried material.

The microorganisms of the disclosure may be isolated in substantially pure or mixed cultures. They may be concentrated, diluted, or provided in the natural concentrations in which they are found in the source material. For example, microorganisms from saline sediments may be isolated for use in this disclosure by suspending the sediment in fresh water and allowing the sediment to fall to the bottom. The water containing the bulk of the microorganisms may be removed by decantation after a suitable period of settling and either applied directly to the plant growth medium, or concentrated by filtering or centrifugation, diluted to an appropriate concentration and applied to the plant growth medium with the bulk of the salt removed. By way of further example, microorganisms from mineralized or toxic sources may be similarly treated to recover the microbes for application to the plant growth material to minimize the potential for damage to the plant.

In another embodiment, the microorganisms are used in a crude form, in which they are not isolated from the source material in which they naturally reside. For example, the microorganisms are provided in combination with the source material in which they reside; for example, as soil, or the roots, seed or foliage of a plant. In this embodiment, the source material may include one or more species of microorganisms.

In some embodiments, a mixed population of microorganisms is used in the methods of the disclosure.

In embodiments of the disclosure where the microorganisms are isolated from a source material (for example, the material in which they naturally reside), any one or a combination of a number of standard techniques which will be readily known to skilled persons may be used.

However, by way of example, these in general employ processes by which a solid or liquid culture of a single microorganism can be obtained in a substantially pure form, usually by physical separation on the surface of a solid microbial growth medium or by volumetric dilutive isolation into a liquid microbial growth medium. These processes may include isolation from dry material, liquid suspension, slurries or homogenates in which the material is spread in a thin layer over an appropriate solid gel growth medium, or serial dilutions of the material made into a sterile medium and inoculated into liquid or solid culture media.

Whilst not essential, in one embodiment, the material containing the microorganisms may be pre-treated prior to the isolation process in order to either multiply all microorganisms in the material, or select portions of the microbial population, either by enriching the material with microbial nutrients (for example, by pasteurizing the sample to select for microorganisms resistant to heat exposure (for example, bacilli), or by exposing the sample to low concentrations of an organic solvent or sterilant (for example, household bleach) to enhance the survival of spore-forming or solvent-resistant microorganisms). Microorganisms can then be isolated from the enriched materials or materials treated for selective survival, as above.

In an embodiment of the disclosure, endophytic or epiphytic microorganisms are isolated from plant material. Any number of standard techniques known in the art may be used and the microorganisms may be isolated from any appropriate tissue in the plant, including for example root, stem and leaves, and plant reproductive tissues. By way of example, conventional methods for isolation from plants typically include the sterile excision of the plant material of interest (e.g., root or stem lengths, leaves), surface sterilization with an appropriate solution (e.g., 2% sodium hypochlorite), after which the plant material is placed on nutrient medium for microbial growth (See, for example, Strobel G and Daisy B (2003) Microbiology and Molecular Biology Reviews 67 (4): 491-502; Zinniel D K et al. (2002) Applied and Environmental Microbiology 68 (5): 2198-2208).

In one embodiment of the disclosure, the microorganisms are isolated from root tissue. Further methodology for isolating microorganisms from plant material are detailed hereinafter.

In one embodiment, the microbial population is exposed (prior to the method or at any stage of the method) to a selective pressure. For example, exposure of the microorganisms to pasteurization before their addition to a plant growth medium (preferably sterile) is likely to enhance the probability that the plants selected for a desired trait will be associated with spore-forming microbes that can more easily survive in adverse conditions, in commercial storage, or if applied to seed as a coating, in an adverse environment.

In certain embodiments, as mentioned herein before, the microorganism(s) may be used in crude form and need not be isolated from a plant or a media. For example, plant material or growth media which includes the microorganisms identified to be of benefit to a selected plant may be obtained and used as a crude source of microorganisms for the next round of the method or as a crude source of microorganisms at the conclusion of the method. For example, whole plant material could be obtained and optionally processed, such as mulched or crushed.

Alternatively, individual tissues or parts of selected plants (such as leaves, stems, roots, and seeds) may be separated from the plant and optionally processed, such as mulched or crushed. In certain embodiments, one or more part of a plant which is associated with the second set of one or more microorganisms may be removed from one or more selected plants and, where any successive repeat of the method is to be conducted, grafted on to one or more plant used in any step of the plant breeding methods.

### Exemplary Microbes

In aspects, the present disclosure provides isolated microbes, including novel strains of identified microbial species, presented in Table 1.

In other aspects, the present disclosure provides isolated whole microbial cultures of the species and strains identified in Table 1. These cultures may comprise microbes at various concentrations.

In aspects, the disclosure provides for utilizing a microbe selected from Table 1 in agriculture.

In some embodiments, a microbe from the genus Bacillus is utilized in agriculture to impart one or more beneficial properties to a plant species.

Furthermore, the disclosure relates to microbes having characteristics substantially similar to that of a microbe identified in Table 1.

The isolated microbial species, and novel strains of said species, identified in the present disclosure, are able to impart beneficial properties or traits, such as a trait of agronomic importance, to target plant species.

For instance, the isolated microbes described in Table 1, or consortia of said microbes, are able to improve plant health and vitality. The improved plant health and vitality can be quantitatively measured, for example, by measuring the effect that said microbial application has upon a plant phenotypic or genotypic trait.

### Sourcing of Microbes

The microbes of the present disclosure were obtained, among other places, at various locales in New Zealand and the United States

### Isolation and Culturing of Microbes

The microbes of Table 1 were identified by utilizing standard techniques to characterize the microbes' phenotype, which was then utilized to identify the microbe to a taxonomically recognized species. Alternatively, the microbes of Table 1 were sequenced (16S and/or Whole Genome Sequencing, according to methods known in the art) to determine taxonomy.

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described hereinabove (e.g., Gram positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

For example, for isolated bacteria of the disclosure, biologically pure isolates can be obtained through repeated subculture of biological samples, each subculture followed by streaking onto solid media to obtain individual colonies. Methods of preparing, thawing, and growing lyophilized bacteria are commonly known, for example, Gherna, R. L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze-dried liquid formulations and cultures stored long term at −70° C. in solutions containing glycerol are contemplated for use in providing formulations of the present inventions.

The bacteria of the disclosure can be propagated in a liquid medium under aerobic conditions. Medium for growing the bacterial strains of the present disclosure includes a carbon source, a nitrogen source, and inorganic salts, as well as specially required substances such as vitamins, amino acids, nucleic acids and the like. Examples of suitable carbon sources which can be used for growing the bacterial strains include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram(s) per liter of medium. Preferably, glucose, starch, and/or peptone is contained in the medium as a major carbon source, at a concentration of 0.1-5% (W/V). Examples of suitable nitrogen sources which can be used for growing the bacterial strains of the present invention include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according to the type of nitrogen source, typically between 0.1 to 30 gram per liter of medium. The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram per liter of medium. Examples of specially required substances include, but are not limited to, vitamins, nucleic acids, yeast extract, peptone, meat extract, malt extract, dried yeast and combinations thereof. Cultivation can be effected at a temperature, which allows the growth of the bacterial strains, essentially, between 20° C. and 46° C. In some aspects, a temperature range is 30° C.-37° C. For optimal growth, in some embodiments, the medium can be adjusted to pH 7.0-7.4. It will be appreciated that commercially available media may also be used to culture the bacterial strains, such as Nutrient Broth or Nutrient Agar available from Difco, Detroit, MI. It will be appreciated that cultivation time may differ depending on the type of culture medium used and the concentration of sugar as a major carbon source.

In aspects, cultivation lasts between 24-96 hours. Bacterial cells thus obtained are isolated using methods, which are well known in the art. Examples include, but are not limited to, membrane filtration and centrifugal separation. The pH may be adjusted using sodium hydroxide and the like and the culture may be dried using a freeze dryer, until the water content becomes equal to 4% or less. Microbial co-cultures may be obtained by propagating each strain as described hereinabove. It will be appreciated that the microbial strains may be cultured together when compatible culture conditions can be employed.

### Identification of Microbes

Microbes can be distinguished into a genus based on polyphasic taxonomy, which incorporates all available phenotypic and genotypic data into a consensus classification (Vandamme et al. 1996. Polyphasic taxonomy, a consensus approach to bacterial systematics. Microbiol Rev 1996, 60:407-438). One accepted genotypic method for defining species is based on overall genomic relatedness, such that strains which share approximately 70% or more relatedness using DNA-DNA hybridization, with 5° C. or less ΔTm (the difference in the melting temperature between homologous and heterologous hybrids), under standard conditions, are considered to be members of the same species. Thus, populations that share greater than the aforementioned 70% threshold can be considered to be variants of the same species.

For bacterial microbes, the 16S rRNA sequences are often used for determining taxonomy and making distinctions between species, in that if a 16S rRNA sequence shares less than a specified % sequence identity from a reference sequence, then the two organisms from which the sequences were obtained are said to be of different species.

Thus, one could consider microbes to be of the same species, if they share at least 80%, 85%, 90%, 95%, 97%, 98%, or 99% sequence identity across the 16S or 16S rRNA or rDNA sequence. In some aspects, a microbe could be considered to be the same species only if it shares at least 95% identity.

Further, one could define microbial strains of a species, as those that share at least 80%, 85%, 90%, 95%, 97%, 98%, or 99% sequence identity across the 16S rRNA sequence.

Comparisons may also be made with 23S rRNA sequences against reference sequences. In some aspects, a microbe could be considered to be the same strain only if it shares at least 95% identity. In some embodiments, “substantially similar genetic characteristics” means a microbe sharing at least 95% identity.

For fungal microbes, the ITS (Internal Transcriber Sequence) is often used for identification of taxonomy. Among the regions of the ribosomal cistron, the internal transcribed spacer (ITS) region has the highest probability of successful identification for the broadest range of fungi, with the most clearly defined barcode gap between inter- and intraspecific variation, and has been proposed as the formal fungal identification sequence (Schoch et al., PNAS Apr. 17, 2012 109 (16) 6241-6246).

In one embodiment, microbial strains of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-179.

In one embodiment, microbes of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-179.

In one embodiment, microbial consortia of the present disclosure include two or more microbes that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-179.

In one embodiment, microbial consortia of the present disclosure include two or more microbial strains, wherein at least one of those comprises a polynucleotide sequences that shares at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-179.

In one embodiment, microbial consortia of the present disclosure include two or more microbial strains, wherein at least one of those comprises a polynucleotide sequences that shares at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-179, and wherein at least one of the microbes is optionally selected from Table 1.

Unculturable microbes often cannot be assigned to a definite species in the absence of a phenotype determination, the microbes can be given a candidatus designation within a genus provided their 16S rRNA sequences subscribes to the principles of identity with known species.

One approach is to observe the distribution of a large number of strains of closely related species in sequence space and to identify clusters of strains that are well resolved from other clusters. This approach has been developed by using the concatenated sequences of multiple core (house-keeping) genes to assess clustering patterns, and has been called multilocus sequence analysis (MLSA) or multilocus sequence phylogenetic analysis. MLSA has been used successfully to explore clustering patterns among large numbers of strains assigned to very closely related species by current taxonomic methods, to look at the relationships between small numbers of strains within a genus, or within a broader taxonomic grouping, and to address specific taxonomic questions. More generally, the method can be used to ask whether bacterial species exist—that is, to observe whether large populations of similar strains invariably fall into well-resolved clusters, or whether in some cases there is a genetic continuum in which clear separation into clusters is not observed.

In order to more accurately make a determination of genera, a determination of phenotypic traits, such as morphological, biochemical, and physiological characteristics are made for comparison with a reference genus archetype. The colony morphology can include color, shape, pigmentation, production of slime, etc. Features of the cell are described as to shape, size, Gram reaction, extracellular material, presence of endospores, flagella presence and location, motility, and inclusion bodies. Biochemical and physiological features describe growth of the organism at different ranges of temperature, pH, salinity and atmospheric conditions, growth in presence of different sole carbon and nitrogen sources. One of ordinary skill in the art would be reasonably apprised as to the phenotypic traits that define the genera of the present disclosure. For instance, colony color, form, and texture on a particular agar (e.g., YMA) was used to identify species of Rhizobium.

In one embodiment, bacterial microbes taught herein were identified utilizing 16S rRNA gene sequences. It is known in the art that 16S rRNA contains hypervariable regions that can provide species/strain-specific signature sequences useful for bacterial identification. In the present disclosure, many of the microbes were identified via partial (500-1200 bp) 16S rRNA sequence signatures. In aspects, each strain represents a pure colony isolate that was selected from an agar plate. Selections were made to represent the diversity of organisms present based on any defining morphological characteristics of colonies on agar medium. The medium used, in embodiments, was R2A, PDA, Nitrogen-free semi-solid medium, or MRS agar. Colony descriptions of each of the ‘picked’ isolates were made after 24-hour growth and then entered into our database. Sequence data was subsequently obtained for each of the isolates.

Phylogenetic analysis using the 16S rRNA gene was used to define “substantially similar” species belonging to common genera and also to define “substantially similar” strains of a given taxonomic species. Further, we recorded physiological and/or biochemical properties of the isolates that can be utilized to highlight both minor and significant differences between strains that could lead to advantageous behavior on plants.

### CRISPR-Cas Systems

CRISPR (Clustered Regularly Interspaced Short Palindromic Repeats) endonucleases were first identified in bacteria as example of an “adaptive immunity” system, by which certain bacteria were able to survive viral infection utilizing an immune-type response. Short repeats of an infective virus' DNA were found in an array pattern within the target bacterium, with the repeats interspersed with spacer sequences unique to that bacterium. It was determined that future infections in that bacterium by the same virus were able to be combatted, as the bacterium produced an endonuclease (Cas endonuclease) that was able to specifically target that virus, using the repeat sequences stored in the array.

A CRISPR-Cas system comprises, at a minimum, a CRISPR RNA (crRNA) molecule and at least one CRISPR-associated (Cas) protein to form aa crRNA ribonucleoprotein (crRNP) effector complex. CRISPR-Cas loci comprise an array of identical repeats interspersed with DNA-targeting spacers that encode the crRNA components and an operon-like unit of cas genes encoding the Cas protein components. The resulting ribonucleoprotein complex recognizes a polynucleotide in a sequence-specific manner (Jore et al., Nature Structural & Molecular Biology 18, 529-536 (2011)). The crRNA serves as a guide RNA for sequence specific binding of the effector (protein or complex) to double strand DNA sequences, by forming base pairs with the complementary DNA strand while displacing the noncomplementary strand to form a so called R-loop.

CRISPR-Cas systems have been classified according to sequence and structural analysis of genomic loci and the associated encoded protein(s). Multiple CRISPR/Cas systems have been described including Class 1 systems, with multi-subunit effector complexes (comprising type I, type III, and type IV), and Class 2 systems, with single protein effectors (comprising type II, type V, and type VI) (Makarova et al. 2015, Nature Reviews Microbiology Vol. 13: 1-15; Zetsche el a/., 2015, Cell 163, 1-13; Shmakov et al., 2015, Molecular Cell 60, 1-13; Haft et al., 2005, Computational Biology, PLoS Comput Biol 1(6):e60; and Koonin et al. 2017, Curr Opinion Microbiology 37:67-78).

Methods are now known in the art for identifying and classifying Cas proteins, including Cas endonucleases, in bacteria.

### CRISPR System Taxonomy

Many CRISPR systems have been identified in the literature and described, based upon the native bacterial genomic locus architecture, composition, and the domain structures of the effector protein(s).

### Class I CRISPR-Cas Systems

Class I CRISPR-Cas systems comprise Types I, III, and IV. A characteristic feature of Class I systems is the presence of an effector endonuclease complex instead of a single protein. A Cascade complex comprises a RNA recognition motif (RRM) and a nucleic acid-binding domain that is the core fold of the diverse RAMP (Repeat-Associated Mysterious Proteins) protein superfamily (Makarova et al. 2013, Biochem Soc Trans 41, 1392-1400; Makarova et al. 2015, Nature Reviews Microbiology Vol. 13:1-15). RAMP protein subunits include Cas5 and Cas7 (which comprise the skeleton of the crRNA-effector complex), wherein the Cas5 subunit binds the 5′ handle of the crRNA and interacts with the large subunit, and often includes Cas6 which is loosely associated with the effector complex and typically functions as the repeat-specific RNase in the pre-crRNA processing (Charpentier et al., FEMS Microbiol Rev 2015, 39:428-441; Niewoehner et al., RNA 2016, 22:318-329).

Type I CRISPR-Cas systems comprise a complex of effector proteins, termed Cascade (CRISPR-associated complex for antiviral defense) comprising at a minimum Cas5 and Cas7. The effector complex functions together with a single CRISPR RNA (crRNA) and Cas3 to defend against invading viral DNA (Brouns, S. J. J. et al. Science 321:960-964; Makarova et al. 2015, Nature Reviews Microbiology Vol. 13:1-15). Type I CRISPR-Cas loci comprise the signature gene cas3 (or a variant cas3′ or cas3”), which encodes a metal-dependent nuclease that possesses a single-stranded DNA (ssDNA)-stimulated superfamily 2 helicase with a demonstrated capacity to unwind double stranded DNA (dsDNA) and RNA-DNA duplexes (Makarova et al. 2015, Nature Reviews; Microbiology Vol. 13:1-15). Following target recognition, the Cas3 endonuclease is recruited to the Cascade-crRNA-target DNA complex to cleave and degrade the DNA target (Westra, E. R. et al. (2012) Molecular Cell 46:595-605, Sinkunas, T. et al. (2011) EMBO J. 30:1335-1342, and Sinkunas, T. et al. (2013) EMBO J. 32:385-394). In some type I systems, Cas6 can be the active endonuclease that is responsible for crRNA processing, and Cas5 and Cas7 function as non-catalytic RNA-binding proteins; although in type I-C systems, crRNA processing can be catalyzed by Cas5 (Makarova et al. 2015, Nature Reviews Microbiology Vol. 13:1-15). Type I systems are divided into seven subtypes (Makarova et al. 2011, Nat Rev Microbiol. 2011 9(6):467-477; Koonin et al. 2017, Curr Opinion Microbiology 37:67-78). A modified type I CRISPR-associated complex for adaptive antiviral defense (Cascade) comprising at least the protein subunits Cas7, Cas5 and Cas6, wherein one of these subunits is synthetically fused to a Cas3 endonuclease or a modified restriction endonuclease, FokI, have been described (WO2013098244 published 4 Jul. 4, 2013).

Type III CRISPR-Cas systems, comprising a plurality of cas7 genes, target either ssRNA or ssDNA, and function as either an RNase as well as a target RNA-activated DNA nuclease (Tamulaitis et al., Trends in Microbiology 25(10)49-61, 2017). Csm (Type III-A) and Cmr (Type III-B) complexes function as RNA-activated single-stranded (ss) DNases that couple the target RNA binding/cleavage with ssDNA degradation. Upon foreign DNA infection, the CRISPR RNA (crRNA)-guided binding of the Csm or Cmr complex to the emerging transcript recruits Cas10 DNase to the actively transcribed phage DNA, resulting in degradation of both the transcript and phage DNA, but not the host DNA. The Cas10 HD-domain is responsible for the ssDNase activity, and Csm3/Cmr4 subunits are responsible for the endoribonuclease activity of the Csm/Cmr complex. The 3′-flanking sequence of the target RNA is critical for the ssDNase activity of Csm/Cmr: the basepairing with the 5′-handle of crRNA protects host DNA from degradation.

Type IV systems, although comprising typical type I cas5 and cas7 domains in addition to a cas8-like domain, may lack the CRISPR array that is characteristic of most other CRISPR-Cas systems.

### Class II CRISPR-Cas Systems

Class II CRISPR-Cas systems comprise Types II, V, and VI. A characteristic feature of Class II systems is the presence of a single Cas effector protein instead of an effector complex.

Types II and V Cas proteins comprise an RuvC endonuclease domain that adopts the RNase H fold.

Type II CRISPR/Cas systems employ a crRNA and tracrRNA (trans-activating CRISPR RNA) to guide the Cas endonuclease to its DNA target. The crRNA comprises a spacer region complementary to one strand of the double strand DNA target and a region that base pairs with the tracrRNA (trans-activating CRISPR RNA) forming a RNA duplex that directs the Cas endonuclease to cleave the DNA target, leaving a blunt end. Spacers are acquired through a not fully understood process involving Cas1 and Cas2 proteins. Type II CRISPR/Cas loci typically comprise cast and cast genes in addition to the cas9 gene (Chylinski et al., 2013, RNA Biology 10:726-737; Makarova et al. 2015, Nature Reviews Microbiology Vol. 13:1-15). Type II CRISR-Cas loci can encode a tracrRNA, which is partially complementary to the repeats within the respective CRISPR array, and can comprise other proteins such as Csn1 and Csn2. The presence of cas9 in the vicinity of cas1 and cas2 genes is the hallmark of type II loci (Makarova et al. 2015, Nature Reviews Microbiology Vol. 13:1-15).

Type V CRISPR/Cas systems comprise a single Cas endonuclease, including Cpf1 (Casl2) (Koonin et al., Curr Opinion Microbiology 37:67-78, 2017), that is an active RNA-guided endonuclease that does not necessarily require the additional trans-activating CRISPR (tracr) RNA for target cleavage, unlike Cas9.

Type VI CRISPR-Cas systems comprise a cas13 gene that encodes a nuclease with two HEPN (Higher Eukaryotes and Prokaryotes Nucleotide-binding) domains but no HNH or RuvC domains, and are not dependent upon tracrRNA activity. The majority of HEPN domains comprise conserved motifs that constitute a metal-independent endoRNase active site (Anantharam et al., Biol Direct 8:15, 2013). Because of this feature, it is thought that type VI systems may act on RNA targets instead of the DNA targets that are common to other CRISPR-Cas systems.

### Cas Proteins

The proteins encoded in a Cas locus within a bacterial genome include endonucleases that are responsible for effecting cleavage of a nucleotide target (e.g., double-stranded DNA cleavage, DNA nicking, ssDNA cleavage, RNA cleavage), and are variously referred to as “Cas Endonucleases”, “Effector Proteins”, and “Effector Complexes. Other genes within the locus encode proteins of other functions that may be required for complete activity, as discussed below.

### Cas Endonucleases, Effector Proteins, and Effector Complexes

Endonucleases are enzymes that cleave the phosphodiester bond within a polynucleotide chain, and include restriction endonucleases that cleave DNA at specific sites without damaging the bases. Examples of endonucleases include restriction endonucleases, meganucleases, TAL effector nucleases (TALENs), zinc finger nucleases, and Cas (CRISPR-associated) effector endonucleases.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases may occur as individual effectors (Class 2 CRISPR systems) or as part of larger effector complexes (Class I CRISPR systems).

Cas endonucleases that have been described include, but are not limited to, for example: Cas3 (a feature of Class 1 type I systems), Cas9 (a feature of Class 2 type II systems) and Cas12 (Cpf1) (a feature of Class 2 type V systems).

Cas3 (and its variants Cas3′ and Cas3”) functions as a single-stranded DNA nuclease (HD domain) and an ATP-dependent helicase. A variant of the Cas3 endonuclease can be obtained by disabling the functional activity of one or both domains of the Cas3 endonuclease poly peptide. Disabling the ATPase dependent helicase activity (by deletion, knockout of the Cas3-helicase domain, or through mutagenesis of critical residues or by assembling the reaction in the absence of ATP as described previously (Sinkunas, T. et al., 2013, EMBO J. 32:385-394) can convert the cleavage ready Cascade comprising the modified Cas3 endonuclease into a nickase (as the HD domain is still functional). Disabling the HD endonuclease activity can be accomplished by any method known in the art, such as but not limited to, mutagenesis of critical residues of the HD domain, can convert the cleavage ready Cascade comprising the modified Cas3 endonuclease into a helicase. Disabling the both the Cas helicase and Cas3 HD endonuclease activity can be accomplished by any method known in the art, such as but not limited to, mutagenesis of critical residues of both the helicase and HD domains, can convert the cleavage ready Cascade comprising the modified Cas3 endonuclease into a binder protein that binds to a target sequence.

Cas9 (formerly referred to as Cas5, Csn1, or Csx12) is a Cas endonuclease that forms a complex with a crNucleotide and a tracrNucleotide, or with a single guide polynucleotide, for specifically recognizing and cleaving all or part of a DNA target sequence. Cas9 recognizes a 3′ GC-rich PAM sequence on the target dsDNA. A Cas9 protein comprises a RuvC nuclease with an HNH (H—N—H) nuclease adjacent to the RuvC-II domain. The RuvC nuclease and HNH nuclease each can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al., 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

Cas12 (formerly referred to as Cpf1, and variants c2c1, c2c3, CasX, and CasY) comprise an RuvC nuclease domain and produced staggered, 5′ overhangs on the dsDNA target. Some variants do not require a tracrRNA, unlike the functionality of Cas9. Cas12 and its variants recognize a 5′ AT-rich PAM sequence on the target dsDNA. An insert domain, called Nuc, of the Casl2a protein has been demonstrated to be responsible for target strand cleavage (Yamano et al., Cell 2016, 165:949-962). Additional mutation studies in other Cas12 proteins demonstrated the Nuc domain contributes to guide and target binding, with the RuvC domain responsible for cleavage (Swarts et al., Mol Cell 2017, 66:221-233 e224).

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10:957-963).

To effect cleavage, the Cas endonuclease (or effector complex) forms a complex with a guide polynucleotide, and recognizes a sequence on a target sequence, called a Protospacer Adjacent Motif (PAM). A “protospacer adjacent motif” (PAM) herein refers to a short nucleotide sequence adjacent to a target sequence (protospacer) that can be recognized (targeted) by a guide polynucleotide/Cas endonuclease system. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A guide polynucleotide/Cas endonuclease complex described herein is capable of recognizing, binding to, and optionally nicking, unwinding, or cleaving all or part of a target sequence.

A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein (e.g., a Cas protein disclosed herein), or a variant thereof retaining some or all activity in each endonuclease domain of the Cas protein, is a suitable example of a Cas endonuclease that can cleave both strands of a DNA target sequence.

A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are disclosed in US20140189896 published on 3 Jul. 2014. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end-joining, NHEJ (prone to imperfect repair leading to mutations) or homologous recombination, HR. Each nick in these embodiments can be at least about 5, between 5 and 10, at least 10, between 10 and 15, at least 15, between 15 and 20, at least 20, between 20 and 30, at least 30, between 30 and 40, at least 40, between 40 and 50, at least 50, between 50 and 60, at least 60, between 60 and 70, at least 70, between 70 and 80, at least 80, between 80 and 90, at least 90, between 90 and 100, or 100 or greater (or any integer between 5 and 100) bases apart from each other, for example. One or two Cas nickase proteins herein can be used in a Cas nickase pair. For example, a Cas9 nickase with a mutant RuvC domain, but functioning HNH domain (i.e., Cas9 HNH+/RuvC−), can be used (e.g., Streptococcus pyogenes Cas9 HNH+/RuvC−). Each Cas9 nickase (e.g., Cas9 HNH+/RuvC−) can be directed to specific DNA sites nearby each other (up to 100 base pairs apart) by using suitable RNA components herein with guide RNA sequences targeting each nickase to each specific DNA site.

A guide polynucleotide/Cas endonuclease complex in certain embodiments can bind to a DNA target site sequence, but does not cleave any strand at the target site sequence. Such a complex may comprise a Cas protein in which all of its nuclease domains are mutant, dysfunctional. For example, a Cas9 protein that can bind to a DNA target site sequence, but does not cleave any strand at the target site sequence, may comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator).

Other proteins involved in adaptation, expression and interference are encoded by other cas genes located in the genomic neighborhood of the CRISPR arrays. Some core proteins are found to be universally present across most Types and Classes of CRISPR systems, while others are specific to a particular Type or Class. See, for example, Zhang et al., Nucleic Acids Research, Volume 42, Issue 4, 1 Feb. 2014, Pages 2448-2459; and Hille et al., Cell, Volume 172, Issue 6, 8 Mar. 2018, Pages 1239-1259; Makarova and Koonin, Methods Mol Biol. 2015; 1311: 47-75.

### Recombinant Constructs and Cell Transformation

The disclosed guide polynucleotides, Cas endonucleases, polynucleotide modification templates, donor DNAs, guide polynucleotide/Cas endonuclease systems disclosed herein, and any one combination thereof, optionally further comprising one or more polynucleotide(s) of interest, can be introduced into a cell. Cells include, but are not limited to, human, non-human, animal, bacterial, fungal, insect, yeast, non-conventional yeast, and plant cells, as well as progeny and/or derivatives of the cells produced by the methods described herein.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, N.Y. (1989). Transformation methods are well known to those skilled in the art.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

The disclosure further provides expression constructs for expressing in a prokaryotic or eukaryotic cell/organism a guide RNA/Cas system that is capable of recognizing, binding to, and optionally nicking, unwinding, or cleaving all or part of a target sequence.

In one embodiment, the expression constructs of the disclosure comprise a promoter operably linked to a nucleotide sequence encoding a Cas gene (or optimized gene) and a promoter operably linked to a guide RNA of the present disclosure. The promoter is capable of driving expression of an operably linked nucleotide sequence in a prokaryotic or eukaryotic cell/organism.

In one embodiment, a Cas endonuclease provided herein is introduced to a target polynucleotide (e.g., in vitro or in a cell) to effect a single-strand nick or a double-strand break into the target polynucleotide. The nick or break may be leveraged to introduce an edit into the polynucleotide, for example but not limited to the insertion of a heterologous polynucleotide e.g., a polynucleotide of interest), the deletion of a particular sequence, or the introduction of one or more modified nucleotides.

Other uses are contemplated and within the scope of the disclosure, for example but not limited to gene targeting, and use of the endonuclease as a targeting molecule without leveraging its capability of nicking or cleaving.

Modification of a target sequence may be in the form of a nucleotide insertion, a nucleotide deletion, a nucleotide substitution, the addition of an atom molecule to an existing nucleotide, a nucleotide modification, or the binding of a heterologous polynucleotide or polypeptide to said target sequence. The insertion of one or more nucleotides may be accomplished by the inclusion of a donor polynucleotide in the reaction mixture: said donor polynucleotide is inserted into a double-strand break created by said Cas-alpha ortholog polypeptide. The insertion may be via non-homologous end joining or via homologous recombination.

### Uses in Microbiology, Agriculture, Pharmaceuticals, and Medical Research

The presently disclosed polynucleotides and polypeptides can be introduced into a cell. Cells include, but are not limited to, human, non-human, animal, mammalian, bacterial, fungal, insect, yeast, non-conventional yeast, and plant cells as well as plants and seeds produced by the methods described herein. Any plant can be used with the compositions and methods described herein, including monocot and dicot plants, and plant elements.

The presently disclosed polynucleotides and polypeptides can be introduced into an animal cell. Animal cells can include, but are not limited to: an organism of a phylum including chordates, arthropods, mollusks, annelids, cnidarians, or echinoderms; or an organism of a class including mammals, insects, birds, amphibians, reptiles, or fishes. In some aspects, the animal is human, mouse, C. elegans, rat, fruit fly (Drosophila spp.), zebrafish, chicken, dog, cat, guinea pig, hamster, chicken, Japanese ricefish, sea lamprey, pufferfish, tree frog (e.g., Xenopus spp.), monkey, or chimpanzee. Particular cell types that are contemplated include haploid cells, diploid cells, reproductive cells, neurons, muscle cells, endocrine or exocrine cells, epithelial cells, muscle cells, tumor cells, embryonic cells, hematopoietic cells, bone cells, germ cells, somatic cells, stem cells, pluripotent stem cells, induced pluripotent stem cells, progenitor cells, meiotic cells, and mitotic cells. In some aspects, a plurality of cells from an organism may be used.

Genome modification via a Cas endonuclease described herein may be used to effect a genotypic and/or phenotypic change on the target organism. Such a change is preferably related to an improved phenotype of interest or a physiologically-important characteristic, the correction of an endogenous defect, or the expression of some type of expression marker. In some aspects, the phenotype of interest or physiologically-important characteristic is related to the overall health, fitness, or fertility of the organism, the ecological fitness of the organism, or the relationship or interaction of the organism with other organisms or abiotic factors in its environment.

While the invention has been particularly shown and described with reference to a preferred embodiment and various alternate embodiments, it will be understood by persons skilled in the relevant art that various changes in form and details can be made therein without departing from the spirit and scope of the invention. Various alterations, modifications, and improvements of the present disclosure that readily occur to those skilled in the art, including certain alterations, modifications, substitutions, and improvements are also part of this disclosure. For instance, while the particular examples below may illustrate the methods and embodiments described herein using a specific plant, the principles in these examples may be applied to any plant. Therefore, it will be appreciated that the scope of this invention is encompassed by the embodiments recited herein rather than solely by the specific examples that are exemplified below.

All cited patents and publications referred to in this application are herein incorporated by reference in their entirety, for all purposes, to the same extent as if each were individually and specifically incorporated by reference.

### EXAMPLES

The cas genes provided herein may be used to improve the phenotype of an organism via polynucleotide modification.

### Example 1: Identification and Characterization of CRISPR Proteins in Bacterial Systems

A selection of microbes from the collection were sequenced according to methods known in the art, and assigned taxonomy based on either 16S sequence similarity or whole genome sequencing BLAST.

Briefly, bacterial genomes were annotated using Prokka (Seemann, Bioinformatics, Volume 30, Issue 14, 15 Jul. 2014, Pages 2068-2069). CRISPR-associated genes were identified in each genome and assigned nomenclature based on percent identity matches to closest-relative sequences in public databases.

Isolates of interest were grown to mid-log phase in R2D media. DNA was extracted with the Qiagen Powersoil DNA extraction kit and sequencing libraries were constructed with the iGenomix RipTide kit as per manufacturer instructions. Sequencing was performed on an Illumina HiSeq with PE150. Raw Illumina reads were trimmed to Q15 with Trimmomatic v38 (Bolger A M, Lohse M, and Usadel B. (2014). Trimmomatic: A flexible trimmer for Illumina Sequence Data. Bioinformatics, btu170) and assembled with SPAdes (Prjibelski A, Antipov D, Meleshko D, Lapidus A, and Korobeynikov A. (2020) Using SPAdes de novo assembler. Curr. Protoc. Bioinform. 70, e102) using default parameters. Assembled contigs were analyzed with BinSantity 0.5.4. (Graham E D, Heidelberg J F, and Tully B J. (2017) BinSanity: unsupervised clustering of environmental microbial assemblies using coverage and affinity propagation. PeerJ 5:e3035) for purity with a contamination cutoff of <5%. The largest bin was extracted and annotated with Prokka 1.8 (Seemann T. (2014) Prokka: rapid prokaryotic genome annotation. Bioinformatics 30(14):2068-9). Taxonomy was assigned using GTDB-tk. All genomes containing the cas9 annotation were parsed and the cas9 sequence was exported.

Each microbe may have a single or multiple different types of CRISPR systems. In one example, a particular microbe may have a Class II Type V endonuclease system (such as Cas9) and a Class 1 Type I endonuclease system.

Table 1 lists selected microbes comprising identified cas genes, including those encoding Cas endonucleases. Each microbe is listed by a reference ID, unique Strain number, and taxonomy.

Table 2 shows the identity of each particular cas and CRISPR-system related gene discovered in the microbes of Table 1, listed by reference ID. The key for the genes listed by number as Table 2 column headings is given in Table 3.

In some cases, a Cas endonuclease identified herein is used to edit a target polynucleotide. In some cases, a different cas gene or Cas protein identified herein is used to effect an improvement to a system, target polynucleotide, or cell comprising such.

### Example 2: Use of Novel Cas Endonucleases for Polynucleotide Editing

PAM preferences for each of the endonucleases discovered in Example 1 may be determined by methods known in the art (e.g., Karvelis et al., Methods, Volumes 121-122, 15 May 2017, Pages 3-8). Alternatively, a Cas endonuclease may be engineered to accommodate different PAM sequences than that which it naturally prefers (e.g., Leenay and Beisel, J Mol Bio, Volume 429, Issue 2, 20 Jan. 2017, Pages 177-191).

A Cas endonuclease (or effector complex) is identified from its source bacterium, such as those disclosed in Table 1, and either expressed and isolated or synthesized de novo from its corresponding DNA sequence. The compositions disclosed herein may be utilized outside of a typical cellular environment for in vitro modification of one or more target polynucleotides. In some aspects, the target polynucleotide is isolated and purified from a genomic source. In some aspects, the target polynucleotide is on a circularized or linearized plasmid. In some aspects, the target polynucleotide is a PCR product. In some aspects, the target polynucleotide is a synthesized oligonucleotide. In some aspects, said modification includes binding to, nicking, and/or or cleaving a target polynucleotide.

Creation of a guide polynucleotide-endonuclease complex is achieved by methods known in the art. Delivery may be accomplished to an isolated target polynucleotide in vivo, or to a target polynucleotide within a cell (see, e.g., Wilbie et al., Acc. Chem. Res. 2019, 52, 6, 1555-1564).

The target polynucleotide is selected for its capability to accommodate and bind to a particular Cas endonuclease or effector complex. Alternatively, a Cas endonuclease or effector complex is selected based on its capability to bind to a particular target polynucleotide.

Genome modification of the target polynucleotide includes recognition of the target by the endonuclease complex, with or without subsequent nicking or cleaving. When a nick or a double strand break (DSB) is created, it is repaired either by simple re-annealing, or using mechanisms in the cell (NHEJ or HR).

In some examples, a donor polynucleotide is introduced and inserted into the double strand break.

In some examples, a polynucleotide modification template is provided to the break site, whereby the break is repaired according to the template.

Regardless of the mechanism of repair or the provision of additional heterologous polynucleotides, the resulting repair of the nick or break results in perfect repair (no edit observed), the insertion of at least one nucleotide, the deletion of at least one nucleotide, the substitution of at least one nucleotide, and/or the molecular alteration of at least one nucleotide.

The edit of the target polynucleotide creates a beneficial phenotype to the organism that contains the target, or to an organism to which the edited polynucleotide is introduced.

Examples of CRISPR proteins found in bacterial strains described in Table 1 are presented in Table 2, with the edit type descriptions of the Table 2 columns defined in Table 3.

TABLE 1
Microbial strains analyzed for the
presence of CRISPR system proteins

| ID | Strain # | Taxonomy |
|---|---|---|
| 1 | 18772 | Agrobacterium vitis |
| 2 | 16396 | Arthrobacter crystallopoietes |
| 3 | 2032 | Arthrobacter crystallopoietes |
| 4 | 14945 | Arthrobacter sp. ATCC 21022 |
| 5 | 16107 | Arthrobacter sp. ATCC 21022 |
| 6 | 16157 | Arthrobacter sp. ATCC 21022 |
| 7 | 16194 | Arthrobacter sp. ATCC 21022 |
| 8 | 7649 | Arthrobacter sp. ATCC 21022 |
| 9 | 8689 | Arthrobacter sp. ATCC 21022 |
| 10 | 14186 | Arthrobacter sp. FB24 |
| 11 | 14229 | Arthrobacter sp. FB24 |
| 12 | 14970 | Arthrobacter sp. FB24 |
| 13 | 15940 | Arthrobacter sp. FB24 |
| 14 | 2224 | Arthrobacter sp. FB24 |
| 15 | 8522 | Arthrobacter sp. FB24 |
| 16 | 14167 | Arthrobacter sp. PGP41 |
| 17 | 14193 | Arthrobacter sp. PGP41 |
| 18 | 14196 | Arthrobacter sp. PGP41 |
| 19 | 14202 | Arthrobacter sp. PGP41 |
| 20 | 15010 | Arthrobacter sp. PGP41 |
| 21 | 15832 | Arthrobacter sp. PGP41 |
| 22 | 16370 | Arthrobacter sp. PGP41 |
| 23 | 1707 | Arthrobacter sp. PGP41 |
| 24 | 1960 | Arthrobacter sp. PGP41 |
| 25 | 2023 | Arthrobacter sp. PGP41 |
| 26 | 2033 | Arthrobacter sp. PGP41 |
| 27 | 2880 | Arthrobacter sp. PGP41 |
| 28 | 3912 | Arthrobacter sp. PGP41 |
| 29 | 3980 | Arthrobacter sp. PGP41 |
| 30 | 4010 | Arthrobacter sp. PGP41 |
| 31 | 4085 | Arthrobacter sp. PGP41 |
| 32 | 4177 | Arthrobacter sp. PGP41 |
| 33 | 4585 | Arthrobacter sp. PGP41 |
| 34 | 8029 | Arthrobacter sp. PGP41 |
| 35 | 8043 | Arthrobacter sp. PGP41 |
| 36 | 8066 | Arthrobacter sp. PGP41 |
| 37 | 14166 | Arthrobacter sp. QXT-31 |
| 38 | 14658 | Arthrobacter sp. QXT-31 |
| 39 | 16158 | Arthrobacter sp. QXT-31 |
| 40 | 18984 | Arthrobacter sp. QXT-31 |
| 41 | 7301 | Arthrobacter sp. QXT-31 |
| 42 | 7684 | Arthrobacter sp. QXT-31 |
| 43 | 16130 | Arthrobacter sp. U41 |
| 44 | 18588 | Arthrobacter sp. U41 |
| 45 | 17630 | Arthrobacter sp. YC-RL1 |
| 46 | 17635 | Arthrobacter sp. YC-RL1 |
| 47 | 17707 | Arthrobacter sp. YC-RL1 |
| 48 | 17708 | Arthrobacter sp. YC-RL1 |
| 49 | 17725 | Arthrobacter sp. YC-RL1 |
| 50 | 14968 | Arthrobacter sp. ZXY-2 |
| 51 | 15939 | Arthrobacter sp. ZXY-2 |
| 52 | 16216 | Arthrobacter sp. ZXY-2 |
| 53 | 16349 | Arthrobacter sp. ZXY-2 |
| 54 | 16369 | Arthrobacter sp. ZXY-2 |
| 55 | 19177 | Arthrobacter sp. ZXY-2 |
| 56 | 7359 | Arthrobacter sp. ZXY-2 |
| 57 | 7975 | Arthrobacter sp. ZXY-2 |
| 58 | 8017 | Arthrobacter sp. ZXY-2 |
| 59 | 8038 | Arthrobacter sp. ZXY-2 |
| 60 | Azo | Azospirillum |
| 61 | 3797-U6 | Azospirillum brasilense |
| 62 | 941 | Azospirillum brasilense |
| 63 | 12991 | Azospirillum lipoferum |
| 64 | 13445 | Azospirillum lipoferum |
| 65 | 16385 | Azospirillum lipoferum |
| 66 | 1886 | Azospirillum lipoferum |
| 67 | 3706 | Azospirillum lipoferum |
| 68 | 3717 | Azospirillum lipoferum |
| 69 | 3730 | Azospirillum lipoferum |
| 70 | 3737 | Azospirillum lipoferum |
| 71 | 7149 | Azospirillum lipoferum |
| 72 | 7185 | Azospirillum lipoferum |
| 73 | 7330 | Azospirillum lipoferum |
| 74 | 7665 | Azospirillum lipoferum |
| 75 | 16288 | Azospirillum thiophilum |
| 76 | 53693 | Bacillus cereus |
| 77 | 101119 | Bacillus megaterium |
| 78 | 101168 | Bacillus megaterium |
| 79 | 101169 | Bacillus megaterium |
| 80 | 101632 | Bacillus megaterium |
| 81 | 104624 | Bacillus megaterium |
| 82 | 105094 | Bacillus megaterium |
| 83 | 11723 | Bacillus megaterium |
| 84 | 11762 | Bacillus megaterium |
| 85 | 11784 | Bacillus megaterium |
| 86 | 11787 | Bacillus megaterium |
| 87 | 14544 | Bacillus megaterium |
| 88 | 16711 | Bacillus megaterium |
| 89 | 16731 | Bacillus megaterium |
| 90 | 18308 | Bacillus megaterium |
| 91 | 18321 | Bacillus megaterium |
| 92 | 18357 | Bacillus megaterium |
| 93 | 2151 | Bacillus megaterium |
| 94 | 251 | Bacillus megaterium |
| 95 | 255 | Bacillus megaterium |
| 96 | 262 | Bacillus megaterium |
| 97 | 2627 | Bacillus megaterium |
| 98 | 2633 | Bacillus megaterium |
| 99 | 2645 | Bacillus megaterium |
| 100 | 2652 | Bacillus megaterium |
| 101 | 3112 | Bacillus megaterium |
| 102 | 3148 | Bacillus megaterium |
| 103 | 3380 | Bacillus megaterium |
| 104 | 3402 | Bacillus megaterium |
| 105 | 3475 | Bacillus megaterium |
| 106 | 3491 | Bacillus megaterium |
| 107 | 3511 | Bacillus megaterium |
| 108 | 3817 | Bacillus megaterium |
| 109 | 4007 | Bacillus megaterium |
| 110 | 4089 | Bacillus megaterium |
| 111 | 4169 | Bacillus megaterium |
| 112 | 4890 | Bacillus megaterium |
| 113 | 5736 | Bacillus megaterium |
| 114 | 5805 | Bacillus megaterium |
| 115 | 6565 | Bacillus megaterium |
| 116 | 6602 | Bacillus megaterium |
| 117 | 6604 | Bacillus megaterium |
| 118 | 6608 | Bacillus megaterium |
| 119 | 7233 | Bacillus megaterium |
| 120 | 8336 | Bacillus megaterium |
| 121 | 8342 | Bacillus megaterium |
| 122 | 8946 | Bacillus megaterium |
| 123 | 38202 | Bacillus megaterium |
| 124 | 70485 | Bacillus megaterium |
| 125 | 72231 | Bacillus megaterium |
| 126 | 77769 | Bacillus megaterium |
| 127 | 77964 | Bacillus megaterium |
| 128 | 78001 | Bacillus megaterium |
| 129 | 78395 | Bacillus megaterium |
| 130 | 14833 | Bacillus mycoides |
| 131 | 33712 | Bacillus mycoides |
| 132 | 58160 | Bacillus mycoides |
| 133 | 2104 | Bacillus oceanisediminis |
| 134 | 15765 | Bacillus pumilus |
| 135 | 102970 | Bacillus safensis |
| 136 | 102002 | Bacillus simplex |
| 137 | 4631 | Bacillus simplex |
| 138 | 5821 | Bacillus simplex |
| 139 | 66518 | Bacillus simplex |
| 140 | 79680 | Bacillus simplex |
| 141 | 2833 | Bacillus sp. 1NLA3E |
| 142 | 38434 | Bacillus sp. FDAARGOS_235 |
| 143 | 46414 | Bacillus sp. WP8 |
| 144 | 100944 | Bacillus sp. X1(2014) |
| 145 | 100949 | Bacillus sp. X1(2014) |
| 146 | 101665 | Bacillus sp. X1(2014) |
| 147 | 10422 | Bacillus sp. X1(2014) |
| 148 | 104628 | Bacillus sp. X1(2014) |
| 149 | 104651 | Bacillus sp. X1(2014) |
| 150 | 105149 | Bacillus sp. X1(2014) |
| 151 | 105194 | Bacillus sp. X1(2014) |
| 152 | 105195 | Bacillus sp. X1(2014) |
| 153 | 105209 | Bacillus sp. X1(2014) |
| 154 | 105210 | Bacillus sp. X1(2014) |
| 155 | 11199 | Bacillus sp. X1(2014) |
| 156 | 11317 | Bacillus sp. X1(2014) |
| 157 | 13819 | Bacillus sp. X1(2014) |
| 158 | 4309 | Bacillus sp. X1(2014) |
| 159 | 4316 | Bacillus sp. X1(2014) |
| 160 | 5804 | Bacillus sp. X1(2014) |
| 161 | 6600 | Bacillus sp. X1(2014) |
| 162 | 78053 | Bacillus sp. X1(2014) |
| 163 | 102958 | Bacillus sp. Y-01 |
| 164 | 14596 | Bacillus sp. Y-01 |
| 165 | 16135 | Bacillus sp. Y-01 |
| 166 | 4473 | Bacillus sp. Y-01 |
| 167 | 5884 | Bacillus sp. Y-01 |
| 168 | 5875 | B. thermoamylovorans |
| 169 | 52761 | Bacillus thuringiensis |
| 170 | 52785 | Bacillus thuringiensis |
| 171 | 52889 | Bacillus thuringiensis |
| 172 | 52903 | Bacillus thuringiensis |
| 173 | 52916 | Bacillus thuringiensis |
| 174 | 52922 | Bacillus thuringiensis |
| 175 | 52928 | Bacillus thuringiensis |
| 176 | 52939 | Bacillus thuringiensis |
| 177 | 53050 | Bacillus thuringiensis |
| 178 | 53330 | Bacillus thuringiensis |
| 179 | 53474 | Bacillus thuringiensis |
| 180 | 54812 | Bacillus thuringiensis |
| 181 | 55214 | Bacillus thuringiensis |
| 182 | 56132 | Bacillus thuringiensis |
| 183 | 62847 | Bacillus thuringiensis |
| 184 | 14803 | Bacillus toyonensis |
| 185 | 14805 | Bacillus toyonensis |
| 186 | 14826 | Bacillus toyonensis |
| 187 | 721 | Bacillus toyonensis |
| 188 | 2103 | Bacillus weihaiensis |
| 189 | 5881 | Brevibacillus brevis |
| 190 | 7976 | B. laterosporus |
| 191 | 1625 | Chryseobacterium glaciei |
| 192 | 6586 | Chryseobacterium sp. IHB B 17019 |
| 193 | 79645 | Chryseobacterium sp. IHB B 17019 |
| 194 | 79655 | Chryseobacterium sp. IHB B 17019 |
| 195 | 12917 | Curtobacterium pusillum |
| 196 | 7125 | Enterobacteriaceae bacterium ENNIH2 |
| 197 | 7168 | Enterobacteriaceae bacterium ENNIH2 |
| 198 | 7176 | Enterobacteriaceae bacterium ENNIH2 |
| 199 | 7179 | Enterobacteriaceae bacterium ENNIH2 |
| 200 | 18175 | Enterobacteriaceae bacterium ENNIH3 |
| 201 | IM56 | Escherichia coli |
| 202 | 4251 | Fictibacillus arsenicus |
| 203 | 1124 | Glutamicibacter arilaitensis |
| 204 | 1212 | Glutamicibacter arilaitensis |
| 205 | 16826 | Glutamicibacter arilaitensis |
| 206 | 17658 | Glutamicibacter arilaitensis |
| 207 | 182 | Glutamicibacter arilaitensis |
| 208 | 18333 | Glutamicibacter arilaitensis |
| 209 | 18391 | Glutamicibacter arilaitensis |
| 210 | 18667 | Glutamicibacter arilaitensis |
| 211 | 18722 | Glutamicibacter arilaitensis |
| 212 | 18810 | Glutamicibacter arilaitensis |
| 213 | 3439 | Glutamicibacter arilaitensis |
| 214 | 4394 | Glutamicibacter arilaitensis |
| 215 | 452 | Glutamicibacter arilaitensis |
| 216 | 507 | Glutamicibacter arilaitensis |
| 217 | 6300 | Glutamicibacter arilaitensis |
| 218 | 6580 | Glutamicibacter arilaitensis |
| 219 | 700 | Glutamicibacter arilaitensis |
| 220 | 7372 | Glutamicibacter arilaitensis |
| 221 | 7437 | Glutamicibacter arilaitensis |
| 222 | 797 | Glutamicibacter arilaitensis |
| 223 | 11146 | Klebsiella pneumoniae |
| 224 | 11434 | Klebsiella pneumoniae |
| 225 | 11687 | Klebsiella pneumoniae |
| 226 | 11815 | Klebsiella pneumoniae |
| 227 | 85 | Klebsiella sp. M5al |
| 228 | 196-U9 | Klebsiella variicola |
| 229 | 196 | Klebsiella variicola |
| 230 | 7148 | Kluyvera intermedia |
| 231 | 7154 | Kluyvera intermedia |
| 232 | 14743 | Leifsonia xyli |
| 233 | 6913 | Leifsonia xyli |
| 234 | 105097 | Lysinibacillus sphaericus |
| 235 | 5806 | Lysinibacillus sphaericus |
| 236 | 101676 | Lysinibacillus varians |
| 237 | 7337 | Mesorhizobium oceanicum |
| 238 | 16064 | Methylorubrum extorquens |
| 239 | 1115 | Microbacterium aurum |
| 240 | 9983 | Microbacterium aurum |
| 241 | 15455 | Microbacterium hominis |
| 242 | 1017 | Microbacterium sp. 1.5R |
| 243 | 1038 | Microbacterium sp. 1.5R |
| 244 | 1040 | Microbacterium sp. 1.5R |
| 245 | 1051 | Microbacterium sp. 1.5R |
| 246 | 1063 | Microbacterium sp. 1.5R |
| 247 | 1078 | Microbacterium sp. 1.5R |
| 248 | 1080 | Microbacterium sp. 1.5R |
| 249 | 1083 | Microbacterium sp. 1.5R |
| 250 | 1094 | Microbacterium sp. 1.5R |
| 251 | 1109 | Microbacterium sp. 1.5R |
| 252 | 1087 | Microbacterium sp. CGR1 |
| 253 | 1098 | Microbacterium sp. CGR1 |
| 254 | 1155 | Microbacterium sp. CGR1 |
| 255 | 1165 | Microbacterium sp. CGR1 |
| 256 | 16372 | Microbacterium sp. No. 7 |
| 257 | 10078 | Microbacterium sp. TPU 3598 |
| 258 | 10105 | Microbacterium sp. TPU 3598 |
| 259 | 1113 | Microbacterium sp. TPU 3598 |
| 260 | 10387 | Microbacterium sp. XT11 |
| 261 | 10419 | Microbacterium sp. XT11 |
| 262 | 10420 | Microbacterium sp. XT11 |
| 263 | 10426 | Microbacterium sp. XT11 |
| 264 | 10455 | Microbacterium sp. XT11 |
| 265 | 1047 | Microbacterium sp. XT11 |
| 266 | 10494 | Microbacterium sp. XT11 |
| 267 | 10504 | Microbacterium sp. XT11 |
| 268 | 10506 | Microbacterium sp. XT11 |
| 269 | 10507 | Microbacterium sp. XT11 |
| 270 | 10511 | Microbacterium sp. XT11 |
| 271 | 10522 | Microbacterium sp. XT11 |
| 272 | 10533 | Microbacterium sp. XT11 |
| 273 | 10631 | Microbacterium sp. XT11 |
| 274 | 10634 | Microbacterium sp. XT11 |
| 275 | 10635 | Microbacterium sp. XT11 |
| 276 | 10647 | Microbacterium sp. XT11 |
| 277 | 10660 | Microbacterium sp. XT11 |
| 278 | 10669 | Microbacterium sp. XT11 |
| 279 | 10671 | Microbacterium sp. XT11 |
| 280 | 10785 | Microbacterium sp. XT11 |
| 281 | 10805 | Microbacterium sp. XT11 |
| 282 | 10806 | Microbacterium sp. XT11 |
| 283 | 10808 | Microbacterium sp. XT11 |
| 284 | 1082 | Microbacterium sp. XT11 |
| 285 | 10822 | Microbacterium sp. XT11 |
| 286 | 10839 | Microbacterium sp. XT11 |
| 287 | 10842 | Microbacterium sp. XT11 |
| 288 | 10844 | Microbacterium sp. XT11 |
| 289 | 11345 | Microbacterium sp. XT11 |
| 290 | 11364 | Microbacterium sp. XT11 |
| 291 | 11393 | Microbacterium sp. XT11 |
| 292 | 11425 | Microbacterium sp. XT11 |
| 293 | 11456 | Microbacterium sp. XT11 |
| 294 | 1176 | Microbacterium sp. XT11 |
| 295 | 11773 | Microbacterium sp. XT11 |
| 296 | 1178 | Microbacterium sp. XT11 |
| 297 | 14881 | Microbacterium sp. XT11 |
| 298 | 5954 | Microbacterium sp. XT11 |
| 299 | 5998 | Microbacterium sp. XT11 |
| 300 | 6136 | Microbacterium sp. XT11 |
| 301 | 10082 | Microbacterium testaceum |
| 302 | 14627 | Microbacterium testaceum |
| 303 | 5773 | Microbacterium testaceum |
| 304 | 5776 | Microbacterium testaceum |
| 305 | 5797 | Microbacterium testaceum |
| 306 | 7752 | Microcella alkaliphila |
| 307 | 15469 | Mitsuaria sp. 7 |
| 308 | 15531 | Mitsuaria sp. 7 |
| 309 | 18791 | Niveispirillum cyanobacteriorum |
| 310 | 1019 | Ottowia sp. KADR8-3 |
| 311 | 12592 | Paenibacillus beijingensis |
| 312 | 13751 | Paenibacillus beijingensis |
| 313 | 9649 | Paenibacillus beijingensis |
| 314 | 9655 | Paenibacillus beijingensis |
| 315 | 9677 | Paenibacillus beijingensis |
| 316 | 56051 | Paenibacillus beijingensis |
| 317 | 104080 | Paenibacillus borealis |
| 318 | 104081 | Paenibacillus borealis |
| 319 | 7032 | Paenibacillus borealis |
| 320 | 7037 | Paenibacillus borealis |
| 321 | 7039 | Paenibacillus borealis |
| 322 | 7040 | Paenibacillus borealis |
| 323 | 7087 | Paenibacillus borealis |
| 324 | 104168 | Paenibacillus crassostreae |
| 325 | 5937 | Paenibacillus crassostreae |
| 326 | 7033 | Paenibacillus crassostreae |
| 327 | 104107 | Paenibacillus donghaensis |
| 328 | 104182 | Paenibacillus donghaensis |
| 329 | 2651 | Paenibacillus donghaensis |
| 330 | 4296 | Paenibacillus donghaensis |
| 331 | 18002 | Paenibacillus durus |
| 332 | 102551 | Paenibacillus graminis |
| 333 | 102579 | Paenibacillus graminis |
| 334 | 102589 | Paenibacillus graminis |
| 335 | 103406 | Paenibacillus graminis |
| 336 | 11225 | Paenibacillus ihbetae |
| 337 | 11503 | Paenibacillus ihbetae |
| 338 | 11527 | Paenibacillus ihbetae |
| 339 | 12579 | Paenibacillus ihbetae |
| 340 | 12583 | Paenibacillus ihbetae |
| 341 | 14471 | Paenibacillus ihbetae |
| 342 | 15368 | Paenibacillus ihbetae |
| 343 | 15414 | Paenibacillus ihbetae |
| 344 | 15766 | Paenibacillus ihbetae |
| 345 | 15794 | Paenibacillus ihbetae |
| 346 | 15795 | Paenibacillus ihbetae |
| 347 | 6248 | Paenibacillus ihbetae |
| 348 | 8267 | Paenibacillus larvae |
| 349 | 9553 | Paenibacillus larvae |
| 350 | 67482 | Paenibacillus larvae |
| 351 | 67537 | Paenibacillus larvae |
| 352 | 100960 | Paenibacillus mucilaginosus |
| 353 | 101209 | Paenibacillus mucilaginosus |
| 354 | 12127 | Paenibacillus mucilaginosus |
| 355 | 12147 | Paenibacillus mucilaginosus |
| 356 | 12149 | Paenibacillus mucilaginosus |
| 357 | 12150 | Paenibacillus mucilaginosus |
| 358 | 12178 | Paenibacillus mucilaginosus |
| 359 | 12199 | Paenibacillus mucilaginosus |
| 360 | 12331 | Paenibacillus mucilaginosus |
| 361 | 12398 | Paenibacillus mucilaginosus |
| 362 | 12515 | Paenibacillus mucilaginosus |
| 363 | 13193 | Paenibacillus mucilaginosus |
| 364 | 13221 | Paenibacillus mucilaginosus |
| 365 | 13640 | Paenibacillus mucilaginosus |
| 366 | 13763 | Paenibacillus mucilaginosus |
| 367 | 13774 | Paenibacillus mucilaginosus |
| 368 | 13790 | Paenibacillus mucilaginosus |
| 369 | 13946 | Paenibacillus mucilaginosus |
| 370 | 13996 | Paenibacillus mucilaginosus |
| 371 | 14051 | Paenibacillus mucilaginosus |
| 372 | 14090 | Paenibacillus mucilaginosus |
| 373 | 14508 | Paenibacillus mucilaginosus |
| 374 | 14552 | Paenibacillus mucilaginosus |
| 375 | 14869 | Paenibacillus mucilaginosus |
| 376 | 17889 | Paenibacillus mucilaginosus |
| 377 | 18001 | Paenibacillus mucilaginosus |
| 378 | 18003 | Paenibacillus mucilaginosus |
| 379 | 18025 | Paenibacillus mucilaginosus |
| 380 | 2129 | Paenibacillus mucilaginosus |
| 381 | 7474 | Paenibacillus mucilaginosus |
| 382 | 8397 | Paenibacillus mucilaginosus |
| 383 | 9640 | Paenibacillus mucilaginosus |
| 384 | 9641 | Paenibacillus mucilaginosus |
| 385 | 9667 | Paenibacillus mucilaginosus |
| 386 | 9706 | Paenibacillus mucilaginosus |
| 387 | 100748 | Paenibacillus naphthalenovorans |
| 388 | 100951 | Paenibacillus naphthalenovorans |
| 389 | 101039 | Paenibacillus naphthalenovorans |
| 390 | 101051 | Paenibacillus naphthalenovorans |
| 391 | 105153 | Paenibacillus naphthalenovorans |
| 392 | 105160 | Paenibacillus naphthalenovorans |
| 393 | 12148 | Paenibacillus naphthalenovorans |
| 394 | 12179 | Paenibacillus naphthalenovorans |
| 395 | 12193 | Paenibacillus naphthalenovorans |
| 396 | 12222 | Paenibacillus naphthalenovorans |
| 397 | 12240 | Paenibacillus naphthalenovorans |
| 398 | 12397 | Paenibacillus naphthalenovorans |
| 399 | 12408 | Paenibacillus naphthalenovorans |
| 400 | 12409 | Paenibacillus naphthalenovorans |
| 401 | 12645 | Paenibacillus naphthalenovorans |
| 402 | 13218 | Paenibacillus naphthalenovorans |
| 403 | 13375 | Paenibacillus naphthalenovorans |
| 404 | 13614 | Paenibacillus naphthalenovorans |
| 405 | 13631 | Paenibacillus naphthalenovorans |
| 406 | 13783 | Paenibacillus naphthalenovorans |
| 407 | 13825 | Paenibacillus naphthalenovorans |
| 408 | 13843 | Paenibacillus naphthalenovorans |
| 409 | 13844 | Paenibacillus naphthalenovorans |
| 410 | 13864 | Paenibacillus naphthalenovorans |
| 411 | 13880 | Paenibacillus naphthalenovorans |
| 412 | 13917 | Paenibacillus naphthalenovorans |
| 413 | 13944 | Paenibacillus naphthalenovorans |
| 414 | 13970 | Paenibacillus naphthalenovorans |
| 415 | 13979 | Paenibacillus naphthalenovorans |
| 416 | 14072 | Paenibacillus naphthalenovorans |
| 417 | 14130 | Paenibacillus naphthalenovorans |
| 418 | 14131 | Paenibacillus naphthalenovorans |
| 419 | 14133 | Paenibacillus naphthalenovorans |
| 420 | 14136 | Paenibacillus naphthalenovorans |
| 421 | 14441 | Paenibacillus naphthalenovorans |
| 422 | 14575 | Paenibacillus naphthalenovorans |
| 423 | 15381 | Paenibacillus naphthalenovorans |
| 424 | 15764 | Paenibacillus naphthalenovorans |
| 425 | 15768 | Paenibacillus naphthalenovorans |
| 426 | 15771 | Paenibacillus naphthalenovorans |
| 427 | 15793 | Paenibacillus naphthalenovorans |
| 428 | 16949 | Paenibacillus naphthalenovorans |
| 429 | 17882 | Paenibacillus naphthalenovorans |
| 430 | 17883 | Paenibacillus naphthalenovorans |
| 431 | 17892 | Paenibacillus naphthalenovorans |
| 432 | 17978 | Paenibacillus naphthalenovorans |
| 433 | 415 | Paenibacillus naphthalenovorans |
| 434 | 9643 | Paenibacillus naphthalenovorans |
| 435 | 9644 | Paenibacillus naphthalenovorans |
| 436 | 9659 | Paenibacillus naphthalenovorans |
| 437 | 9666 | Paenibacillus naphthalenovorans |
| 438 | 9672 | Paenibacillus naphthalenovorans |
| 439 | 14814 | Paenibacillus odorifer |
| 440 | 15772 | Paenibacillus odorifer |
| 441 | 17881 | Paenibacillus odorifer |
| 442 | 7239 | Paenibacillus odorifer |
| 443 | 102488 | Paenibacillus polymyxa |
| 444 | 102952 | Paenibacillus polymyxa |
| 445 | 104492 | Paenibacillus polymyxa |
| 446 | 104495 | Paenibacillus polymyxa |
| 447 | 1118 | Paenibacillus polymyxa |
| 448 | 6645 | Paenibacillus polymyxa |
| 449 | 53072 | Paenibacillus polymyxa |
| 450 | 53107 | Paenibacillus polymyxa |
| 451 | 53144 | Paenibacillus polymyxa |
| 452 | 53145 | Paenibacillus polymyxa |
| 453 | 53146 | Paenibacillus polymyxa |
| 454 | 53150 | Paenibacillus polymyxa |
| 455 | 53378 | Paenibacillus polymyxa |
| 456 | 54701 | Paenibacillus polymyxa |
| 457 | 54805 | Paenibacillus polymyxa |
| 458 | 54911 | Paenibacillus polymyxa |
| 459 | 54997 | Paenibacillus polymyxa |
| 460 | 55083 | Paenibacillus polymyxa |
| 461 | 55115 | Paenibacillus polymyxa |
| 462 | 55136 | Paenibacillus polymyxa |
| 463 | 55146 | Paenibacillus polymyxa |
| 464 | 55470 | Paenibacillus polymyxa |
| 465 | 55965 | Paenibacillus polymyxa |
| 466 | 56089 | Paenibacillus polymyxa |
| 467 | 57529 | Paenibacillus polymyxa |
| 468 | 60721 | Paenibacillus polymyxa |
| 469 | 62529 | Paenibacillus polymyxa |
| 470 | 63764 | Paenibacillus polymyxa |
| 471 | 66545 | Paenibacillus polymyxa |
| 472 | 68890 | Paenibacillus polymyxa |
| 473 | 68892 | Paenibacillus polymyxa |
| 474 | 70947 | Paenibacillus polymyxa |
| 475 | 70952 | Paenibacillus polymyxa |
| 476 | 70957 | Paenibacillus polymyxa |
| 477 | 70971 | Paenibacillus polymyxa |
| 478 | 70995 | Paenibacillus polymyxa |
| 479 | 71001 | Paenibacillus polymyxa |
| 480 | 71004 | Paenibacillus polymyxa |
| 481 | 71343 | Paenibacillus polymyxa |
| 482 | 71347 | Paenibacillus polymyxa |
| 483 | 71348 | Paenibacillus polymyxa |
| 484 | 72994 | Paenibacillus polymyxa |
| 485 | 77155 | Paenibacillus polymyxa |
| 486 | 77357 | Paenibacillus polymyxa |
| 487 | 77359 | Paenibacillus polymyxa |
| 488 | 77370 | Paenibacillus polymyxa |
| 489 | 77457 | Paenibacillus polymyxa |
| 490 | 77559 | Paenibacillus polymyxa |
| 491 | 18006 | Paenibacillus sp. 32O-W |
| 492 | 18015 | Paenibacillus sp. 32O-W |
| 493 | 3487 | Paenibacillus sp. 32O-W |
| 494 | 4352 | Paenibacillus sp. 32O-W |
| 495 | 4356 | Paenibacillus sp. 32O-W |
| 496 | 4382 | Paenibacillus sp. 32O-W |
| 497 | 9658 | Paenibacillus sp. 32O-W |
| 498 | 12606 | Paenibacillus sp. CAA11 |
| 499 | 12632 | Paenibacillus sp. CAA11 |
| 500 | 13878 | Paenibacillus sp. CAA11 |
| 501 | 1396 | Paenibacillus sp. CAA11 |
| 502 | 4302 | Paenibacillus sp. CAA11 |
| 503 | 4362 | Paenibacillus sp. CAA11 |
| 504 | 6276 | Paenibacillus sp. CAA11 |
| 505 | 6630 | Paenibacillus sp. CAA11 |
| 506 | 6640 | Paenibacillus sp. CAA11 |
| 507 | 8646 | Paenibacillus sp. CAA11 |
| 508 | 100433 | Paenibacillus sp. FSL H7-0357 |
| 509 | 100790 | Paenibacillus sp. FSL H7-0357 |
| 510 | 100796 | Paenibacillus sp. FSL H7-0357 |
| 511 | 101021 | Paenibacillus sp. FSL H7-0357 |
| 512 | 101030 | Paenibacillus sp. FSL H7-0357 |
| 513 | 101035 | Paenibacillus sp. FSL H7-0357 |
| 514 | 17897 | Paenibacillus sp. FSL H7-0357 |
| 515 | 17912 | Paenibacillus sp. FSL H7-0357 |
| 516 | 18047 | Paenibacillus sp. FSL H7-0357 |
| 517 | 3392 | Paenibacillus sp. FSL H7-0357 |
| 518 | 4328 | Paenibacillus sp. FSL H7-0357 |
| 519 | 15763 | Paenibacillus sp. FSL H7-0737 |
| 520 | 9651 | Paenibacillus sp. FSL H7-0737 |
| 521 | 18041 | Paenibacillus sp. FSL P4-0081 |
| 522 | 2026 | Paenibacillus sp. FSL P4-0081 |
| 523 | 2849 | Paenibacillus sp. FSL P4-0081 |
| 524 | 17947 | Paenibacillus sp. FSL R7-0273 |
| 525 | 17948 | Paenibacillus sp. FSL R7-0273 |
| 526 | 102895 | Paenibacillus sp. IHBB 10380 |
| 527 | 103069 | Paenibacillus sp. IHBB 10380 |
| 528 | 103070 | Paenibacillus sp. IHBB 10380 |
| 529 | 103102 | Paenibacillus sp. IHBB 10380 |
| 530 | 103112 | Paenibacillus sp. IHBB 10380 |
| 531 | 103113 | Paenibacillus sp. IHBB 10380 |
| 532 | 103122 | Paenibacillus sp. IHBB 10380 |
| 533 | 103535 | Paenibacillus sp. IHBB 10380 |
| 534 | 104114 | Paenibacillus sp. IHBB 10380 |
| 535 | 104115 | Paenibacillus sp. IHBB 10380 |
| 536 | 104175 | Paenibacillus sp. IHBB 10380 |
| 537 | 104176 | Paenibacillus sp. IHBB 10380 |
| 538 | 104208 | Paenibacillus sp. IHBB 10380 |
| 539 | 104218 | Paenibacillus sp. IHBB 10380 |
| 540 | 104219 | Paenibacillus sp. IHBB 10380 |
| 541 | 104596 | Paenibacillus sp. IHBB 10380 |
| 542 | 6135 | Paenibacillus sp. IHBB 10380 |
| 543 | 100033 | Paenibacillus sp. JDR-2 |
| 544 | 101044 | Paenibacillus sp. JDR-2 |
| 545 | 101559 | Paenibacillus sp. JDR-2 |
| 546 | 1022 | Paenibacillus sp. JDR-2 |
| 547 | 102413 | Paenibacillus sp. JDR-2 |
| 548 | 105159 | Paenibacillus sp. JDR-2 |
| 549 | 10630 | Paenibacillus sp. JDR-2 |
| 550 | 10970 | Paenibacillus sp. JDR-2 |
| 551 | 12130 | Paenibacillus sp. JDR-2 |
| 552 | 12603 | Paenibacillus sp. JDR-2 |
| 553 | 12604 | Paenibacillus sp. JDR-2 |
| 554 | 12615 | Paenibacillus sp. JDR-2 |
| 555 | 12642 | Paenibacillus sp. JDR-2 |
| 556 | 13195 | Paenibacillus sp. JDR-2 |
| 557 | 13203 | Paenibacillus sp. JDR-2 |
| 558 | 13215 | Paenibacillus sp. JDR-2 |
| 559 | 13269 | Paenibacillus sp. JDR-2 |
| 560 | 13408 | Paenibacillus sp. JDR-2 |
| 561 | 13489 | Paenibacillus sp. JDR-2 |
| 562 | 13752 | Paenibacillus sp. JDR-2 |
| 563 | 13779 | Paenibacillus sp. JDR-2 |
| 564 | 14830 | Paenibacillus sp. JDR-2 |
| 565 | 15330 | Paenibacillus sp. JDR-2 |
| 566 | 18445 | Paenibacillus sp. JDR-2 |
| 567 | 19062 | Paenibacillus sp. JDR-2 |
| 568 | 19333 | Paenibacillus sp. JDR-2 |
| 569 | 19334 | Paenibacillus sp. JDR-2 |
| 570 | 3983 | Paenibacillus sp. JDR-2 |
| 571 | 418 | Paenibacillus sp. JDR-2 |
| 572 | 4397 | Paenibacillus sp. JDR-2 |
| 573 | 5948 | Paenibacillus sp. JDR-2 |
| 574 | 6230 | Paenibacillus sp. JDR-2 |
| 575 | 6396 | Paenibacillus sp. JDR-2 |
| 576 | 6984 | Paenibacillus sp. JDR-2 |
| 577 | 7572 | Paenibacillus sp. JDR-2 |
| 578 | 7595 | Paenibacillus sp. JDR-2 |
| 579 | 8579 | Paenibacillus sp. JDR-2 |
| 580 | 8623 | Paenibacillus sp. JDR-2 |
| 581 | 8627 | Paenibacillus sp. JDR-2 |
| 582 | 8639 | Paenibacillus sp. JDR-2 |
| 583 | 9110 | Paenibacillus sp. JDR-2 |
| 584 | 56708 | Paenibacillus sp. JDR-2 |
| 585 | 104199 | Paenibacillus sp. Y412MC10 |
| 586 | 10962 | Paenibacillus sp. Y412MC10 |
| 587 | 11195 | Paenibacillus sp. Y412MC10 |
| 588 | 14841 | Paenibacillus sp. Y412MC10 |
| 589 | 15393 | Paenibacillus sp. Y412MC10 |
| 590 | 15408 | Paenibacillus sp. Y412MC10 |
| 591 | 15802 | Paenibacillus sp. Y412MC10 |
| 592 | 4307 | Paenibacillus sp. Y412MC10 |
| 593 | 8595 | Paenibacillus sp. Y412MC10 |
| 594 | 8614 | Paenibacillus sp. Y412MC10 |
| 595 | 8632 | Paenibacillus sp. Y412MC10 |
| 596 | 8642 | Paenibacillus sp. Y412MC10 |
| 597 | 11494 | Paenibacillus terrae |
| 598 | 9763 | Paenibacillus terrae |
| 599 | 9775 | Paenibacillus terrae |
| 600 | 101395 | Paenibacillus xylanexedens |
| 601 | 102032 | Paenibacillus xylanexedens |
| 602 | 102641 | Paenibacillus xylanexedens |
| 603 | 102642 | Paenibacillus xylanexedens |
| 604 | 102653 | Paenibacillus xylanexedens |
| 605 | 103115 | Paenibacillus xylanexedens |
| 606 | 103120 | Paenibacillus xylanexedens |
| 607 | 103121 | Paenibacillus xylanexedens |
| 608 | 105436 | Paenibacillus xylanexedens |
| 609 | 105451 | Paenibacillus xylanexedens |
| 610 | 10994 | Paenibacillus xylanexedens |
| 611 | 12160 | Paenibacillus xylanexedens |
| 612 | 12187 | Paenibacillus xylanexedens |
| 613 | 12188 | Paenibacillus xylanexedens |
| 614 | 12306 | Paenibacillus xylanexedens |
| 615 | 12324 | Paenibacillus xylanexedens |
| 616 | 12365 | Paenibacillus xylanexedens |
| 617 | 12366 | Paenibacillus xylanexedens |
| 618 | 13208 | Paenibacillus xylanexedens |
| 619 | 14808 | Paenibacillus xylanexedens |
| 620 | 14809 | Paenibacillus xylanexedens |
| 621 | 14817 | Paenibacillus xylanexedens |
| 622 | 14824 | Paenibacillus xylanexedens |
| 623 | 14828 | Paenibacillus xylanexedens |
| 624 | 17374 | Paenibacillus xylanexedens |
| 625 | 17874 | Paenibacillus xylanexedens |
| 626 | 2167 | Paenibacillus xylanexedens |
| 627 | 3371 | Paenibacillus xylanexedens |
| 628 | 4292 | Paenibacillus xylanexedens |
| 629 | 4301 | Paenibacillus xylanexedens |
| 630 | 102590 | Paenibacillus yonginensis |
| 631 | 10954 | Paenibacillus yonginensis |
| 632 | 12582 | Paenibacillus yonginensis |
| 633 | 12621 | Paenibacillus yonginensis |
| 634 | 14053 | Paenibacillus yonginensis |
| 635 | 15306 | Paenibacillus yonginensis |
| 636 | 15309 | Paenibacillus yonginensis |
| 637 | 15313 | Paenibacillus yonginensis |
| 638 | 15323 | Paenibacillus yonginensis |
| 639 | 15353 | Paenibacillus yonginensis |
| 640 | 15362 | Paenibacillus yonginensis |
| 641 | 15407 | Paenibacillus yonginensis |
| 642 | 15760 | Paenibacillus yonginensis |
| 643 | 15784 | Paenibacillus yonginensis |
| 644 | 5802 | Paenibacillus yonginensis |
| 645 | 5824 | Paenibacillus yonginensis |
| 646 | 5833 | Paenibacillus yonginensis |
| 647 | 6013 | Paenibacillus yonginensis |
| 648 | 6240 | Paenibacillus yonginensis |
| 649 | 6277 | Paenibacillus yonginensis |
| 650 | 6278 | Paenibacillus yonginensis |
| 651 | 6284 | Paenibacillus yonginensis |
| 652 | 6360 | Paenibacillus yonginensis |
| 653 | 6366 | Paenibacillus yonginensis |
| 654 | 8432 | Paenibacillus yonginensis |
| 655 | 8601 | Paenibacillus yonginensis |
| 656 | 8602 | Paenibacillus yonginensis |
| 657 | 8628 | Paenibacillus yonginensis |
| 658 | 1355 | Pantoea agglomerans |
| 659 | 1570 | Pantoea agglomerans |
| 660 | 1640 | Pantoea agglomerans |
| 661 | 55529 | Pantoea agglomerans |
| 662 | 77666 | Pantoea vagans |
| 663 | Pl | Plarvae |
| 664 | 10022 | Pseudarthrobacter chlorophenolicus |
| 665 | 10466 | Pseudarthrobacter chlorophenolicus |
| 666 | 10638 | Pseudarthrobacter chlorophenolicus |
| 667 | 1198 | Pseudarthrobacter chlorophenolicus |
| 668 | 1253 | Pseudarthrobacter chlorophenolicus |
| 669 | 1271 | Pseudarthrobacter chlorophenolicus |
| 670 | 1286 | Pseudarthrobacter chlorophenolicus |
| 671 | 14650 | Pseudarthrobacter chlorophenolicus |
| 672 | 14727 | Pseudarthrobacter chlorophenolicus |
| 673 | 14779 | Pseudarthrobacter chlorophenolicus |
| 674 | 14977 | Pseudarthrobacter chlorophenolicus |
| 675 | 15062 | Pseudarthrobacter chlorophenolicus |
| 676 | 15546 | Pseudarthrobacter chlorophenolicus |
| 677 | 15859 | Pseudarthrobacter chlorophenolicus |
| 678 | 15875 | Pseudarthrobacter chlorophenolicus |
| 679 | 16 | Pseudarthrobacter chlorophenolicus |
| 680 | 16023 | Pseudarthrobacter chlorophenolicus |
| 681 | 16233 | Pseudarthrobacter chlorophenolicus |
| 682 | 16237 | Pseudarthrobacter chlorophenolicus |
| 683 | 16333 | Pseudarthrobacter chlorophenolicus |
| 684 | 16334 | Pseudarthrobacter chlorophenolicus |
| 685 | 16404 | Pseudarthrobacter chlorophenolicus |
| 686 | 17719 | Pseudarthrobacter chlorophenolicus |
| 687 | 17730 | Pseudarthrobacter chlorophenolicus |
| 688 | 19 | Pseudarthrobacter chlorophenolicus |
| 689 | 1916 | Pseudarthrobacter chlorophenolicus |
| 690 | 2085 | Pseudarthrobacter chlorophenolicus |
| 691 | 2146 | Pseudarthrobacter chlorophenolicus |
| 692 | 2148 | Pseudarthrobacter chlorophenolicus |
| 693 | 24 | Pseudarthrobacter chlorophenolicus |
| 694 | 3 | Pseudarthrobacter chlorophenolicus |
| 695 | 3008 | Pseudarthrobacter chlorophenolicus |
| 696 | 3042 | Pseudarthrobacter chlorophenolicus |
| 697 | 3433 | Pseudarthrobacter chlorophenolicus |
| 698 | 3451 | Pseudarthrobacter chlorophenolicus |
| 699 | 3832 | Pseudarthrobacter chlorophenolicus |
| 700 | 3833 | Pseudarthrobacter chlorophenolicus |
| 701 | 3834 | Pseudarthrobacter chlorophenolicus |
| 702 | 3878 | Pseudarthrobacter chlorophenolicus |
| 703 | 4055 | Pseudarthrobacter chlorophenolicus |
| 704 | 4128 | Pseudarthrobacter chlorophenolicus |
| 705 | 4432 | Pseudarthrobacter chlorophenolicus |
| 706 | 4445 | Pseudarthrobacter chlorophenolicus |
| 707 | 4447 | Pseudarthrobacter chlorophenolicus |
| 708 | 4482 | Pseudarthrobacter chlorophenolicus |
| 709 | 4487 | Pseudarthrobacter chlorophenolicus |
| 710 | 4495 | Pseudarthrobacter chlorophenolicus |
| 711 | 4528 | Pseudarthrobacter chlorophenolicus |
| 712 | 4534 | Pseudarthrobacter chlorophenolicus |
| 713 | 4549 | Pseudarthrobacter chlorophenolicus |
| 714 | 4560 | Pseudarthrobacter chlorophenolicus |
| 715 | 4686 | Pseudarthrobacter chlorophenolicus |
| 716 | 5025 | Pseudarthrobacter chlorophenolicus |
| 717 | 5767 | Pseudarthrobacter chlorophenolicus |
| 718 | 5965 | Pseudarthrobacter chlorophenolicus |
| 719 | 5972 | Pseudarthrobacter chlorophenolicus |
| 720 | 60 | Pseudarthrobacter chlorophenolicus |
| 721 | 6024 | Pseudarthrobacter chlorophenolicus |
| 722 | 62 | Pseudarthrobacter chlorophenolicus |
| 723 | 7246 | Pseudarthrobacter chlorophenolicus |
| 724 | 14171 | Pseudarthrobacter phenanthrenivorans |
| 725 | 1431 | Pseudarthrobacter phenanthrenivorans |
| 726 | 1438 | Pseudarthrobacter phenanthrenivorans |
| 727 | 1471 | Pseudarthrobacter phenanthrenivorans |
| 728 | 1472 | Pseudarthrobacter phenanthrenivorans |
| 729 | 15158 | Pseudarthrobacter phenanthrenivorans |
| 730 | 16060 | Pseudarthrobacter phenanthrenivorans |
| 731 | 16248 | Pseudarthrobacter phenanthrenivorans |
| 732 | 16274 | Pseudarthrobacter phenanthrenivorans |
| 733 | 16351 | Pseudarthrobacter phenanthrenivorans |
| 734 | 16410 | Pseudarthrobacter phenanthrenivorans |
| 735 | 1713 | Pseudarthrobacter phenanthrenivorans |
| 736 | 1730 | Pseudarthrobacter phenanthrenivorans |
| 737 | 17759 | Pseudarthrobacter phenanthrenivorans |
| 738 | 1779 | Pseudarthrobacter phenanthrenivorans |
| 739 | 1783 | Pseudarthrobacter phenanthrenivorans |
| 740 | 18723 | Pseudarthrobacter phenanthrenivorans |
| 741 | 19108 | Pseudarthrobacter phenanthrenivorans |
| 742 | 2025 | Pseudarthrobacter phenanthrenivorans |
| 743 | 2035 | Pseudarthrobacter phenanthrenivorans |
| 744 | 2036 | Pseudarthrobacter phenanthrenivorans |
| 745 | 2155 | Pseudarthrobacter phenanthrenivorans |
| 746 | 2156 | Pseudarthrobacter phenanthrenivorans |
| 747 | 3030 | Pseudarthrobacter phenanthrenivorans |
| 748 | 3219 | Pseudarthrobacter phenanthrenivorans |
| 749 | 3899 | Pseudarthrobacter phenanthrenivorans |
| 750 | 4464 | Pseudarthrobacter phenanthrenivorans |
| 751 | 4479 | Pseudarthrobacter phenanthrenivorans |
| 752 | 4546 | Pseudarthrobacter phenanthrenivorans |
| 753 | 4579 | Pseudarthrobacter phenanthrenivorans |
| 754 | 4592 | Pseudarthrobacter phenanthrenivorans |
| 755 | 4713 | Pseudarthrobacter phenanthrenivorans |
| 756 | 7631 | Pseudarthrobacter phenanthrenivorans |
| 757 | 7639 | Pseudarthrobacter phenanthrenivorans |
| 758 | 2289 | Pseudarthrobacter sulfonivorans |
| 759 | 682 | Pseudarthrobacter sulfonivorans |
| 760 | 5771 | Pseudomonas citronellolis |
| 761 | 961 | Pseudomonas citronellolis |
| 762 | 172 | Pseudomonas fulva |
| 763 | 5777 | Pseudomonas stutzeri |
| 764 | 9351 | Pseudomonas stutzeri |
| 765 | 9870 | Pseudomonas stutzeri |
| 766 | 3298 | Rahnella sp. Y9602 |
| 767 | 16925 | Raoultella ornithinolytica |
| 768 | 10099 | Rathayibacter tritici |
| 769 | 10107 | Rathayibacter tritici |
| 770 | 16299 | Rathayibacter tritici |
| 771 | 7648 | Rathayibacter tritici |
| 772 | 7685 | Rathayibacter tritici |
| 773 | 8983 | Rathayibacter tritici |
| 774 | 7181 | Rhizobium sp. ACO-34A |
| 775 | 7498 | Runella sp. HYN0085 |
| 776 | 7534 | Runella sp. HYN0085 |
| 777 | 366 | Serratia marcescens |
| 778 | 18758 | Shinella sp. HZN7 |
| 779 | 6047 | Shinella sp. HZN7 |
| 780 | 9177 | Shinella sp. HZN7 |
| 781 | 16095 | Sinorhizobium fredii |
| 782 | 15448 | Sinorhizobium meliloti |
| 783 | 7559 | Spirosoma aerolatum |
| 784 | 7915 | Spirosoma radiotolerans |
| 785 | 15172 | Streptomyces reticuli |
| 786 | 8945 | Variovorax paradoxus |
| 787 | 918 | Variovorax paradoxus |
| 788 | 8266 | Virgibacillus pantothenticus |
| 789 | 9962 | undetermined |
| 790 | ASM976v2 | undetermined |
| 791 | 101813 | undetermined |
| 792 | 10817 | undetermined |
| 793 | 11716 | undetermined |
| 794 | 12301 | undetermined |
| 795 | 18330 | undetermined |
| 796 | 18389 | undetermined |
| 797 | 4150 | undetermined |
| 798 | 55076 | undetermined |
| 799 | 56123 | undetermined |

TABLE 2
CRISPR Proteins (column headers) Identified in Microbial Strains (Rows)
(See Table 1 for Microbe ID designations and descriptions, and Table 3 for Protein Descriptions)

| ID | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
|---|---|---|---|---|---|---|---|---|---|---|---|---|---|---|---|---|---|---|---|---|---|---|---|---|---|---|
| 1 | | | | | | | | | | | X | | | | | X | | | | | | | | X | | X |
| 2 | | | | | | | | | | | | | | | | | | | | | | | | | | |
| 3 | | | | | | | | | | | | | | | | | | | | | | | | | | |
| 4 | | | | | | | | | | | | | | | | | | | | | | | | | | |
| 5 | | | | | | | | | | | | | | | | | | | | | | | | | | |
| 6 | | | | | | | | | | | | | | | | | | | | | | | | | | |
| 7 | | | | | | | | | | | | | | | | | | | | | | | | | | |
| 8 | | | | | | | | | | | | | | | | | | | | | | | | | | |
| 9 | | | | | | | | | | | | | | | | | | | | | | | | | | |
| 10 | | | | | | | | | | | | | | | | | | | | | | | | | | |
| 11 | | | | | | | | | | | | | | | | | | | | | | | | | | |
| 12 | | | | | | | | | | | | | | | | | | | | | | | | | | |
| 13 | | | | | | | | | | | | | | | | | | | | | | | | | | |
| 14 | | | | | | | | | | | | | | | | | | | | | | | | | | |
| 15 | | | | | | | | | | | | | | | | | | | | | | | | | | |
| 16 | | | | | | | | | | | | | | | | | | | | | | | | | | |
| 17 | | | | | | | | | | | | | | | | | | | | | | | | | | |
| 18 | | | | | | | | | | | | | | | | | | | | | | | | | | |
| 19 | | | | | | | | | | | | | | | | | | | | | | | | | | |
| 20 | | | | | | | | | | | | | | | | | | | | | | | | | | |
| 21 | | | | | | | | | | | | | | | | | | | | | | | | | | |
| 22 | | | | | | | | | | | | | | | | | | | | | | | | | | |
| 23 | | | | | | | | | | | | | | | | | | | | | | | | | | |
| 24 | | | | | | | | | | | | | | | | | | | | | | | | | | |
| 25 | | | | | | | | | | | | | | | | | | | | | | | | | | |
| 26 | | | | | | | | | | | | | | | | | | | | | | | | | | |
| 27 | | | | | | | | | | | | | | | | | | | | | | | | | | |
| 28 | | | | | | | | | | | | | | | | | | | | | | | | | | |
| 29 | | | | | | | | | | | | | | | | | | | | | | | | | | |
| 30 | | | | | | | | | | | | | | | | | | | | | | | | | | |
| 31 | | | | | | | | | | | | | | | | | | | | | | | | | | |
| 32 | | | | | | | | | | | | | | | | | | | | | | | | | | |
| 33 | | | | | | | | | | | | | | | | | | | | | | | | | | |
| 34 | | | | | | | | | | | | | | | | | | | | | | | | | | |
| 35 | | | | | | | | | | | | | | | | | | | | | | | | | | |
| 36 | | | | | | | | | | | | | | | | | | | | | | | | | | |
| 37 | | | | | | | | | | | | | | | | | | | | | | | | | | |
| 38 | | | | | | | | | | | | | | | | | | | | | | | | | | |
| 39 | | | | | | | | | | | | | | | | | | | | | | | | | | |
| 40 | | | | | | | | | | | | | | | | | | | | | | | | | | |
| 41 | | | | | | | | | | | | | | | | | | | | | | | | | | |
| 42 | | | | | | | | | | | | | | | | | | | | | | | | | | |
| 43 | | | | | | | | | | | | | | | | | | | | | | | | | | |
| 44 | | | | | | | | | | | | | | | | | | | | | | | | | | |
| 45 | | | | | | | | | | | | | | | | | | | | | | | | | | |
| 46 | | | | | | | | | | | | | | | | | | | | | | | | | | |
| 47 | | | | | | | | | | | | | | | | | | | | | | | | | | |
| 48 | | | | | | | | | | | | | | | | | | | | | | | | | | |
| 49 | | | | | | | | | | | | | | | | | | | | | | | | | | |
| 50 | | | | | | | | | | | | | | | | | | | | | | | | | | |
| 51 | | | | | | | | | | | | | | | | | | | | | | | | | | |
| 52 | | | | | | | | | | | | | | | | | | | | | | | | | | |
| 53 | | | | | | | | | | | | | | | | | | | | | | | | | | |
| 54 | | | | | | | | | | | | | | | | | | | | | | | | | | |
| 55 | | | | | | | | | | | | | | | | | | | | | | | | | | |
| 56 | | | | | | | | | | | | | | | | | | | | | | | | | | |
| 57 | | | | | | | | | | | | | | | | | | | | | | | | | | |
| 58 | | | | | | | | | | | | | | | | | | | | | | | | | | |
| 59 | | | | | | | | | | | | | | | | | | | | | | | | | | |
| 60 | | | | | | | | | | | X | | | | | X | | | | | | | | X | | X |
| 61 | | | | | | | | | | | X | X | X | | | X | | | | | | | | X | | |
| 62 | | | | | | | | | | | X | | | | | X | | | | | | | | | | |
| 63 | | | | | | | | | | | | | | | | | | | | | | | | | | |
| 64 | | | | | | | | | | | | | | X | | | | | X | | | | | | | |
| 65 | | | | | | | | | | | | | | | | | | | | | | | | | | |
| 66 | | | | | | | | | | | | | | | | | | | | | | | | X | | X |
| 67 | | | | | | | | | | | | | | | | | | | | | | | | | | |
| 68 | | | | | | | | | | | X | X | X | | | X | | | | | | | | X | | |
| 69 | | | | | | | | | | | X | | | | | X | | | | | | | | X | | X |
| 70 | | | | | | | | | | | X | | | | | X | | | | | | | | X | | X |
| 71 | | | | | | | | | | | X | | | X | | X | | | X | | | | | X | | X |
| 72 | | | | | | | | | | | X | | | X | | X | | | X | | | | | X | | X |
| 73 | | | | | | | | | | | | | | X | | | | | X | | | | | | | |
| 74 | | | | | | | | | | | | | | X | | | | | X | | | | | | | |
| 75 | | | | | | | | | | | | | | | | | | | | | | | | | | |
| 76 | | | | | | | | | | | X | | | | | X | | | | | | | | | | |
| 77 | | | | | | | | | | | | | | | | | | | | | | | | | | |
| 78 | | | | | | | | | | | | | | | | | | | | | | | | | | |
| 79 | | | | | | | | | | | | | | | | | | | | | | | | | | |
| 80 | | | | | | | | | | | | | | | | | | | | | | | | | | |
| 81 | | | | | | | | | | | | | | | | | | | | | | | | | | |
| 82 | | | | | | | | | | | | | | | | | | | | | | | | | | |
| 83 | | | | | | | | | | | | | | | | | | | | | | | | | | |
| 84 | | | | | | | | | | | | | | | | | | | | | | | | | | |
| 85 | | | | | | | | | | | | | | | | | | | | | | | | | | |
| 86 | | | | | | | | | | | | | | | | | | | | | | | | | | |
| 87 | | | | | | | | | | | | | | | | | | | | | | | | | | |
| 88 | | | | | | | | | | | | | | | | | | | | | | | | | | |
| 89 | | | | | | | | | | | | | | | | | | | | | | | | | | |
| 90 | | | | | | | | | | | | | | | | | | | | | | | | | | |
| 91 | | | | | | | | | | | | | | | | | | | | | | | | | | |
| 92 | | | | | | | | | | | | | | | | | | | | | | | | | | |
| 93 | | | | | | | | | | | | | | | | | | | | | | | | | | |
| 94 | | | | | | | | | | | | | | | | | | | | | | | | | | |
| 95 | | | | | | | | | | | | | | | | | | | | | | | | | | |
| 96 | | | | | | | | | | | | | | | | | | | | | | | | | | |
| 97 | | | | | | | | | | | | | | | | | | | | | | | | | | |
| 98 | | | | | | | | | | | | | | | | | | | | | | | | | | |
| 99 | | | | | | | | | | | | | | | | | | | | | | | | | | |
| 100 | | | | | | | | | | | | | | | | | | | | | | | | | | |
| 101 | | | | | | | | | | | | | | | | | | | | | | | | | | |
| 102 | | | | | | | | | | | | | | | | | | | | | | | | | | |
| 103 | | | | | | | | | | | | | | | | | | | | | | | | | | |
| 104 | | | | | | | | | | | | | | | | | | | | | | | | | | |
| 105 | | | | | | | | | | | | | | | | | | | | | | | | | | |
| 106 | | | | | | | | | | | | | | | | | | | | | | | | | | |
| 107 | | | | | | | | | | | | | | | | | | | | | | | | | | |
| 108 | | | | | | | | | | | | | | | | | | | | | | | | | | |
| 109 | | | | | | | | | | | | | | | | | | | | | | | | | | |
| 110 | | | | | | | | | | | | | | | | | | | | | | | | | | |
| 111 | | | | | | | | | | | | | | | | | | | | | | | | | | |
| 112 | | | | | | | | | | | | | | | | | | | | | | | | | | |
| 113 | | | | | | | | | | | | | | | | | | | | | | | | | | |
| 114 | | | | | | | | | | | | | | | | | | | | | | | | | | |
| 115 | | | | | | | | | | | | | | | | | | | | | | | | | | |
| 116 | | | | | | | | | | | | | | | | | | | | | | | | | | |
| 117 | | | | | | | | | | | | | | | | | | | | | | | | | | |
| 118 | | | | | | | | | | | | | | | | | | | | | | | | | | |
| 119 | | | | | | | | | | | | | | | | | | | | | | | | | | |
| 120 | | | | | | | | | | | | | | | | | | | | | | | | | | |
| 121 | | | | | | | | | | | | | | | | | | | | | | | | | | |
| 122 | | | | | | | | | | | | | | | | | | | | | | | | | | |
| 123 | | | | | | | | | | | | | | | | | | | | | | | | | | |
| 124 | | | | | | | | | | | | | | | | | | | | | | | | | | |
| 125 | | | | | | | | | | | | | | | | | | | | | | | | | | |
| 126 | | | | | | | | | | | | | | | | | | | | | | | | | | |
| 127 | | | | | | | | | | | | | | | | | | | | | | | | | | |
| 128 | | | | | | | | | | | | | | | | | | | | | | | | | | |
| 129 | | | | | | | | | | | | | | | | | | | | | | | | | | |
| 130 | | | | | | | | | | | X | | | | | X | | | | | | | | | | |
| 131 | | | | | | | | | | | X | | | | | X | | | | | | | | | | |
| 132 | | | | | | | | | | | X | | | | | X | | | | | | | | | | |
| 133 | | | | | | | | | | | | | | | | | | | | | | | | | | |
| 134 | | | | | | | | | X | X | | | | | | | | | | X | | | | | | |
| 135 | | | | | | | | | X | X | | | | | | | | | | X | | | | | | |
| 136 | | | | | | | | | | | | | | | | | | | | | | | | | | |
| 137 | | | | X | | | | | | | | | | | | | | | | | | | | | | |
| 138 | | | | | | | | | | | | | | | | | | | | | | | | | | |
| 139 | | | | | | | | | | | | | | | | | | | | | | | | | | |
| 140 | | | | | | | | | | | | | | | | | | | | | | | | | | |
| 141 | | | | | | | | | | | | | | | | | | | | | | | | | | |
| 142 | | | | | | | | | | | | | | | | | | | | | | | | | | |
| 143 | | | | | | | | | X | X | | | | | | | | | | X | | | | | | |
| 144 | | | | | | | | | | | | | | | | | | | | | | | | | | |
| 145 | | | | | | | | | | | | | | | | | | | | | | | | | | |
| 146 | | | | | | | | | | | X | | | | | X | | | | | | | | X | X | |
| 147 | | | | | | | | | | | X | | | | | X | | | | | | | | X | X | |
| 148 | | | | | | | | | | | X | | | | | X | | | | | | | | X | X | |
| 149 | | | | | | | | | | | X | | | | | X | | | | | | | | X | X | |
| 150 | | | | | | | | | | | | | | | | | | | | | | | | | | |
| 151 | | | | | | | | | | | X | | | | | X | | | | | | | | X | X | |
| 152 | | | | | | | | | | | | | | | | | | | | | | | | | | |
| 153 | | | | | | | | | | | X | | | | | X | | | | | | | | X | X | |
| 154 | | | | | | | | | | | | | | | | | | | | | | | | | | |
| 155 | | | | | | | | | | | X | | | | | X | | | | | | | | X | X | |
| 156 | | | | | | | | | | | X | | | | | X | | | | | | | | X | X | |
| 157 | | | | | | | | | | | X | | | | | X | | | | | | | | X | X | |
| 158 | | | | | | | | | | | | | | | | | | | | | | | | | | |
| 159 | | | | | | | X | | | | | | | | | | | | | | | | | | | |
| 160 | | | | | | | | | | | | | | | | | | | | | | | | | | |
| 161 | | | | | | | | | | | | | | | | | | | | | | | | | | |
| 162 | | | | | | | | | | | | | | | | | | | | | | | | | | |
| 163 | | | | | | | | | | | | | | | | | | | | | | | | | | |
| 164 | | | | | | | | | | | | | | | | | | | | | | | | | | |
| 165 | | | | | | | | | | | | | | | | | | | | | | | | | | |
| 166 | | | | | | | | | | | | | | | | | | | | | | | | | | |
| 167 | | | | | | | | | | | | | | | | X | | | | | | | | | | |
| 168 | | | | | | | | | | | | | | | | | | | | | | | | | | |
| 169 | | | | | | | | | | | X | | | | | X | | | | | | | | | | |
| 170 | | | | | | | | | | | X | | | | | X | | | | | | | | | | |
| 171 | | | | | | | | | | | X | | | | | X | | | | | | | | | | |
| 172 | | | | | | | | | | | X | | | | | X | | | | | | | | | | |
| 173 | | | | | | | | | | | X | | | | | X | | | | | | | | | | |
| 174 | | | | | | | | | | | X | | | | | X | | | | | | | | | | |
| 175 | | | | | | | | | | | X | | | | | | | | | | | | | | | |
| 176 | | | | | | | | | | | X | | | | | X | | | | | | | | | | |
| 177 | | | | | | | | X | | | X | | | | | X | | | | | | | | | | |
| 178 | | | | | | | | | | | X | | | | | X | | | | | | | | | | |
| 179 | | | | | | | | | | | X | | | | | X | | | | | | | | | | |
| 180 | | | | | | | | | | | X | | | | | X | | | | | | | | | | |
| 181 | | X | | | X | | | X | | | | | | | | | | | | | X | | | | | |
| 182 | | | | | | | | | | | | | | | | | | | | | | | | | | |
| 183 | | | | | | | | | | | X | | | | | X | | | | | | | | | | |
| 184 | | | | | | | | | | | X | | | | | X | | | | | | | | | | |
| 185 | | | | | | | | | | | X | | | | | X | | | | | | | | | | |
| 186 | | | | | | | | | | | X | | | | | X | | | | | | | | | | |
| 187 | | | | | | | | | | | | | | | | | | | | | | | | | | |

CRISPR Proteins (column headers) Identified in Microbial Strains (Rows)
(See Table 1 for Microbe ID designations and descriptions, and Table 3 for Protein Descriptions)

| ID | 27 | 28 | 29 | 30 | 31 | 32 | 33 | 34 | 35 | 36 | 37 | 38 | 39 | 40 | 41 | 42 | 43 | 44 | 45 | 46 | 47 | 48 | 49 | 50 | 51 |
|---|---|---|---|---|---|---|---|---|---|---|---|---|---|---|---|---|---|---|---|---|---|---|---|---|---|
| 1 | | | | | | X | | | | | | | | | | | | | | | | | | | |
| 2 | | | | | | | | | | X | | | | | | | | | | | | | | | |
| 3 | | | | | | | | | | X | | | | | | | | | | | | | | | |
| 4 | | | | | | | | | | X | | | | | | | | | | | | | | | |
| 5 | | | | | | | | | | X | | | | | | | | | | | | | | | |
| 6 | | | | | | | | | | X | | | | | | | | | | | | | | | |
| 7 | | | | | | | | | | X | | | | | | | | | | | | | | | |
| 8 | | | | | | | | | | X | | | | | | | | | | | | | | | |
| 9 | | | | | | | | | | X | | | | | | | | | | | | | | | |
| 10 | | | | | | | | | | X | | | | | | | | | | | | | | | |
| 11 | | | | | | | | | | X | | | | | | | | | | | | | | | |
| 12 | | | | | | | | | | X | | | | | | | | | | | | | | | |
| 13 | | | | | | | | | | X | | | | | | | | | | | | | | | |
| 14 | | | | | | | | | | X | | | | | | | | | | | | | | | |
| 15 | | | | | | | | | | X | | | | | | | | | | | | | | | |
| 16 | | | | | | | | | | X | | | | | | | | | | | | | | | |
| 17 | | | | | | | | | | X | | | | | | | | | | | | | | | |
| 18 | | | | | | | | | | X | | | | | | | | | | | | | | | |
| 19 | | | | | | | | | | X | | | | | | | | | | | | | | | |
| 20 | | | | | | | | | | X | | | | | | | | | | | | | | | |
| 21 | | | | | | | | | | X | | | | | | | | | | | | | | | |
| 22 | | | | | | | | | | X | | | | | | | | | | | | | | | |
| 23 | | | | | | | | | | X | | | | | | | | | | | | | | | |
| 24 | | | | | | | | | | X | | | | | | | | | | | | | | | |
| 25 | | | | | | | | | | X | | | | | | | | | | | | | | | |
| 26 | | | | | | | | | | X | | | | | | | | | | | | | | | |
| 27 | | | | | | | | | | X | | | | | | | | | | | | | | | |
| 28 | | | | | | | | | | X | | | | | | | | | | | | | | | |
| 29 | | | | | | | | | | X | | | | | | | | | | | | | | | |
| 30 | | | | | | | | | | X | | | | | | | | | | | | | | | |
| 31 | | | | | | | | | | X | | | | | | | | | | | | | | | |
| 32 | | | | | | | | | | X | | | | | | | | | | | | | | | |
| 33 | | | | | | | | | | X | | | | | | | | | | | | | | | |
| 34 | | | | | | | | | | X | | | | | | | | | | | | | | | |
| 35 | | | | | | | | | | X | | | | | | | | | | | | | | | |
| 36 | | | | | | | | | | X | | | | | | | | | | | | | | | |
| 37 | | | | | | | | | | X | | | | | | | | | | | | | | | |
| 38 | | | | | | | | | | X | | | | | | | | | | | | | | | |
| 39 | | | | | | | | | | X | | | | | | | | | | | | | | | |
| 40 | | | | | | | | | | X | | | | | | | | | | | | | | | |
| 41 | | | | | | | | | | X | | | | | | | | | | | | | | | |
| 42 | | | | | | | | | | X | | | | | | | | | | | | | | | |
| 43 | | | | | | | | | | X | | | | | | | | | | | | | | | |
| 44 | | | | | | | | | | X | | | | | | | | | | | | | | | |
| 45 | | | | | | | | | | X | | | | | | | | | | | | | | | |
| 46 | | | | | | | | | | X | | | | | | | | | | | | | | | |
| 47 | | | | | | | | | | X | | | | | | | | | | | | | | | |
| 48 | | | | | | | | | | X | | | | | | | | | | | | | | | |
| 49 | | | | | | | | | | X | | | | | | | | | | | | | | | |
| 50 | | | | | | | | | | X | | | | | | | | | | | | | | | |
| 51 | | | | | | | | | | X | | | | | | | | | | | | | | | |
| 52 | | | | | | | | | | X | | | | | | | | | | | | | | | |
| 53 | | | | | | | | | | X | | | | | | | | | | | | | | | |
| 54 | | | | | | | | | | X | | | | | | | | | | | | | | | |
| 55 | | | | | | | | | | X | | | | | | | | | | | | | | | |
| 56 | | | | | | | | | | X | | | | | | | | | | | | | | | |
| 57 | | | | | | | | | | X | | | | | | | | | | | | | | | |
| 58 | | | | | | | | | | X | | | | | | | | | | | | | | | |
| 59 | | | | | | | | | | X | | | | | | | | | | | | | | | |
| 60 | | | | X | | X | | | | X | | | | | | | | | | | | | | | |
| 61 | | | X | | | X | | | | X | | | | | | | | | | X | | | | | |
| 62 | | | | | | X | | | | X | | | | | | | | | | | | | | | |
| 63 | | | | | | | | | | X | | | | | | | | | | | | | | | |
| 64 | | | | | | | | | | X | | | | | | | | | | | | | | | |
| 65 | | | | | | | | | | X | | | | | | | | | | | | | | | |
| 66 | | | | X | | | | | | X | | | | | | | | | | | | | | | |
| 67 | | | | | | X | | | | X | | | | | | | | | | | | | | | |
| 68 | | | X | | | | | | | X | | | | | | | | | | X | | | | | |
| 69 | | | | | X | X | | | | X | | | | | | | | | | | | | | | |
| 70 | | | | | X | X | | | | X | | | | | | | | | | | | | | | |
| 71 | | | | X | | X | | | | X | | | | | | | | | | | | | | | |
| 72 | | | | X | | X | | | | X | | | | | | | | | | | | | | | |
| 73 | | | | | | | | | | X | | | | | | | | | | | | | | | |
| 74 | | | | | | | | | | X | | | | | | | | | | | | | | | |
| 75 | | | | | | | | | | X | | | | | | | | | | | | | | | |
| 76 | | | | | | | | | | | | | | | | | | | | | | | | | |
| 77 | | | | | | | | | | X | | | | | | | | | | | | | | | |
| 78 | | | | | | | | | | X | | | | | | | | | | | | | | | |
| 79 | | | | | | | | | | X | | | | | | | | | | | | | | | |
| 80 | | | | | | | | | | X | | | | | | | | | | | | | | | |
| 81 | | | | | | | | | | X | | | | | | | | | | | | | | | |
| 82 | | | | | | | | | | X | | | | | | | | | | | | | | | |
| 83 | | | | | | | | | | X | | | | | | | | | | | | | | | |
| 84 | | | | | | | | | | X | | | | | | | | | | | | | | | |
| 85 | | | | | | | | | | X | | | | | | | | | | | | | | | |
| 86 | | | | | | | | | | X | | | | | | | | | | | | | | | |
| 87 | | | | | | | | | | X | | | | | | | | | | | | | | | |
| 88 | | | | | | | | | | X | | | | | | | | | | | | | | | |
| 89 | | | | | | | | | | X | | | | | | | | | | | | | | | |
| 90 | | | | | | | | | | X | | | | | | | | | | | | | | | |
| 91 | | | | | | | | | | X | | | | | | | | | | | | | | | |
| 92 | | | | | | | | | | X | | | | | | | | | | | | | | | |
| 93 | | | | | | | | | | X | | | | | | | | | | | | | | | |
| 94 | | | | | | | | | | X | | | | | | | | | | | | | | | |
| 95 | | | | | | | | | | X | | | | | | | | | | | | | | | |
| 96 | | | | | | | | | | X | | | | | | | | | | | | | | | |
| 97 | | | | | | | | | | X | | | | | | | | | | | | | | | |
| 98 | | | | | | | | | | X | | | | | | | | | | | | | | | |
| 99 | | | | | | | | | | X | | | | | | | | | | | | | | | |
| 100 | | | | | | | | | | X | | | | | | | | | | | | | | | |
| 101 | | | | | | | | | | X | | | | | | | | | | | | | | | |
| 102 | | | | | | | | | | X | | | | | | | | | | | | | | | |
| 103 | | | | | | | | | | X | | | | | | | | | | | | | | | |
| 104 | | | | | | | | | | X | | | | | | | | | | | | | | | |
| 105 | | | | | | | | | | X | | | | | | | | | | | | | | | |
| 106 | | | | | | | | | | X | | | | | | | | | | | | | | | |
| 107 | | | | | | | | | | X | | | | | | | | | | | | | | | |
| 108 | | | | | | | | | | X | | | | | | | | | | | | | | | |
| 109 | | | | | | | | | | X | | | | | | | | | | | | | | | |
| 110 | | | | | | | | | | X | | | | | | | | | | | | | | | |
| 111 | | | | | | | | | | X | | | | | | | | | | | | | | | |
| 112 | | | | | | | | | | X | | | | | | | | | | | | | | | |
| 113 | | | | | | | | | | X | | | | | | | | | | | | | | | |
| 114 | | | | | | | | | | X | | | | | | | | | | | | | | | |
| 115 | | | | | | | | | | X | | | | | | | | | | | | | | | |
| 116 | | | | | | | | | | X | | | | | | | | | | | | | | | |
| 117 | | | | | | | | | | X | | | | | | | | | | | | | | | |
| 118 | | | | | | | | | | X | | | | | | | | | | | | | | | |
| 119 | | | | | | | | | | X | | | | | | | | | | | | | | | |
| 120 | | | | | | | | | | X | | | | | | | | | | | | | | | |
| 121 | | | | | | | | | | X | | | | | | | | | | | | | | | |
| 122 | | | | | | | | | | X | | | | | | | | | | | | | | | |
| 123 | | | | | | | | | | X | | | | | | | | | | | | | | | |
| 124 | | | | | | | | | | X | | | | | | | | | | | | | | | |
| 125 | | | | | | | | | | X | | | | | | | | | | | | | | | |
| 126 | | | | | | | | | | X | | | | | | | | | | | | | | | |
| 127 | | | | | | | | | | X | | | | | | | | | | | | | | | |
| 128 | | | | | | | | | | X | | | | | | | | | | | | | | | |
| 129 | | | | | | | | | | X | | | | | | | | | | | | | | | |
| 130 | | | | | | X | | | | | | | | | | | | | | | | | | | |
| 131 | | | | | | X | | | | | | | | | | | | | | | | | | | |
| 132 | | | | | | X | | | | | | | | | | | | | | | | | | | |
| 133 | | | | | | | | | | X | | | | | | | | | | | | | | | |
| 134 | | | | | | | | | | | | | | | | | | | | | | | | | |
| 135 | | | | | | | | | | | | | | | | | | | | | | | | | |
| 136 | | | | | | | | | | X | | | | | | | | | | | | | | | |
| 137 | | | | | | | | | | X | | | | | | | | | | | | | | | |
| 138 | | | | | | | | | | X | | | | | | | | | | | | | | | |
| 139 | | | | | | | | | | X | | | | | | | | | | | | | | | |
| 140 | | | | | | | | | | X | | | | | | | | | | | | | | | |
| 141 | | | | | | | | | | X | | | | | | | | | | | | | | | |
| 142 | | | | | | | | | | X | | | | | | | | | | | | | | | |
| 143 | | | | | | | | | | | | | | | | | | | | | | | | | |
| 144 | | | | | | | | | | X | | | | | | | | | | | | | | | |
| 145 | | | | | | | | | | X | | | | | | | | | | | | | | | |
| 146 | | X | | | | X | | | | | | | | | | | | | | | | | | | |
| 147 | | X | | | | X | | | | | | | | | | | | | | | | | | | |
| 148 | | X | | | | X | | | | | | | | | | | | | | | | | | | |
| 149 | | X | | | | X | | | | | | | | | | | | | | | | | | | |
| 150 | | | | | | | | | | X | | | | | | | | | | | | | | | |
| 151 | | X | | | | X | | | | X | | | | | | | | | | | | | | | |
| 152 | | | | | | | | | | X | | | | | | | | | | | | | | | |
| 153 | | X | | | | X | | | | | | | | | | | | | | | | | | | |
| 154 | | | | | | | | | | X | | | | | | | | | | | | | | | |
| 155 | | | | | | X | | | | X | | | | | | | | | | | | | | | |
| 156 | | X | | | | X | | | | | | | | | | | | | | | | | | | |
| 157 | | X | | | | X | | | | | | | | | | | | | | | | | | | |
| 158 | | | | | | | | | | X | | | | | | | | | | | | | | | |
| 159 | | | | | | | | | | | | | | | | | | | | | | | | | |
| 160 | | | | | | | | | | X | | | | | | | | | | | | | | | |
| 161 | | | | | | | | | | X | | | | | | | | | | | | | | | |
| 162 | | | | | | | | | | X | | | | | | | | | | | | | | | |
| 163 | | | | | | | | | | X | | | | | | | | | | | | | | | |
| 164 | | | | | | | | | | X | | | | | | | | | | | | | | | |
| 165 | | | | | | | | | | X | | | | | | | | | | | | | | | |
| 166 | | | | | | | | | | X | | | | | | | | | | | | | | | |
| 167 | | | | | | | | | | X | | | | | | | | | | | | | | | |
| 168 | | | | | | | | | | X | | | | | | | | | | | | | | | |
| 169 | | X | | | | X | | | | | | | | | | | | | | | | | | | |
| 170 | | | | | | X | | | | | | | | | | | | | | | | | | | |
| 171 | | X | | | | X | | | | | | | | | | | | | | | | | | | |
| 172 | | X | | | | X | | | | | | | | | | | | | | | | | | | |
| 173 | | X | | | | X | | | | | | | | | | | | | | | | | | | |
| 174 | | X | | | | X | | | | | | | | | | | | | | | | | | | |
| 175 | | | | | | X | | | | | | | | | | | | | | | | | | | |
| 176 | | X | | | | X | | | | | | | | | | | | | | | | | | | |
| 177 | | | | | | | | | | | | | | | | | | | | | | | | | |
| 178 | | X | | | | X | | | | | | | | | | | | | | | | | | | |
| 179 | | X | | | | X | | | | | | | | | | | | | | | | | | | |
| 180 | | X | | | | X | | | | | | | | | | | | | | | | | | | |
| 181 | | | | | | | | | | | | | | | | | | | | | | | | | |
| 182 | | | | | | | | | | X | | | | | | | | | | | | | | | |
| 183 | | X | | | | X | | | | | | | | | | | | | | | | | | | |
| 184 | | X | | | | X | | | | | | | | | | | | | | | | | | | |
| 185 | | X | | | | X | | | | | | | | | | | | | | | | | | | |
| 186 | | X | | | | X | | | | | | | | | | | | | | | | | | | |
| 187 | | | | | | | | | | X | | | | | | | | | | | | | | | |

| ID | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
|---|---|---|---|---|---|---|---|---|---|---|---|---|---|---|---|---|---|---|---|---|---|---|---|---|---|---|
| 188 | | | | | | | | | | | | | | | | | | | | | | | | | | |
| 189 | | | | | | | | | | | | | | | | | | | | | X | | | | | X |
| 190 | | | | | | | | | | | | | | | | | | | | | | | | X | | X |
| 191 | | | | | | | X | | | | | | | | | | | | | | | | | X | | X |
| 192 | | | | | | | | | | | | | | | | | | | | | | | | X | | X |
| 193 | | | | | | | | | | | | | | | | | | | | | | | | X | | X |
| 194 | | | | | | | | | | | | | | | | | | | | | | | | X | | X |
| 195 | | | | | | | | | | | | | | | | | | | | | X | X | | X | | |
| 196 | | | | | | | | | | | | | | | | | | | | | | | | X | | |
| 197 | | | | | | | | | | | | | | | | | | | | | | | | | | |
| 198 | | | | | | | | | | | | | | | | | | | | | | | | X | | |
| 199 | | | | | | | | | | | | | | | | | | | | | | | | X | | |
| 200 | | | | | | | | | | | | | | | | | | X | | | X | X | | X | | |
| 201 | | | | | | | | | | | | | | | | | | | | | | X | | X | | |
| 202 | | | | | | | | | | | | | | | | | | | | | | | | | | |
| 203 | | | | | | | | | | | | | | | | | | | | | | | | | | |
| 204 | | | | | | | | | | | | | | | | | | | | | | | | | | |
| 205 | | | | | | | | | | | | | | | | | | | | | | | | | | |
| 206 | | | | | | | | | | | | | | | | | | | | | | | | | | |
| 207 | | | | | | | | | | | | | | | | | | | | | | | | | | |
| 208 | | | | | | | | | | | | | | | | | | | | | | | | | | |
| 209 | | | | | | | | | | | | | | | | | | | | | | | | | | |
| 210 | | | | | | | | | | | | | | | | | | | | | | | | | | |
| 211 | | | | | | | | | | | | | | | | | | | | | | | | | | |
| 212 | | | | | | | | | | | | | | | | | | | | | | | | | | |
| 213 | | | | | | | | | | | | | | | | | | | | | | | | | | |
| 214 | | | | | | | | | | | | | | | | | | | | | | | | | | |
| 215 | | | | | | | | | | | | | | | | | | | | | | | | | | |
| 216 | | | | | | | | | | | | | | | | | | | | | | | | | | |
| 217 | | | | | | | | | | | | | | | | | | | | | | | | | | |
| 218 | | | | | | | | | | | | | | | | | | | | | | | | | | |
| 219 | | | | | | | | | | | | | | | | | | | | | | | | | | |
| 220 | | | | | | | | | | | | | | | | | | | | | | | | | | |
| 221 | | | | | | | | | | | | | | | | | | | | | | | | | | |
| 222 | | | | | | | | | | | | | | | | | | | | | | | | | | |
| 223 | | | | | | | | | | | | | | | | | | X | | | X | X | | | | |
| 224 | | | | | | | | | | | | | | | | | | X | | | X | X | | | | |
| 225 | | | | | | | | | | | | | | | | | | X | | | X | X | | | | |
| 226 | | | | | | | | | | | | | | | | | | X | | | X | X | | | | |
| 227 | | | | | | | | | | | | | | | | | | X | | | X | X | | | | |
| 228 | | | X | | | | | | | | | | | | | | | | | | | | | | | |
| 229 | | | X | | | | | | | | | | | | | | | | | | | | | | | |
| 230 | | | | | | | | | | | | | | | | | | | | | | | | X | | |
| 231 | | | | | | | | | | | | | | | | | | | | | | | | X | | |
| 232 | | | | | | | | | | | | | | | | | | | | | | | | | | |
| 233 | | | | | | | | | | | | | | | | | | | | | | | | | | |
| 234 | | | | | | | | | | | X | | | | X | | | | | | | | | X | X | |
| 235 | | | | | | | | | | | | | | | | | | | | | | | | X | | X |
| 236 | | | | | | | | | | | X | | | | X | | | | | | | | | X | X | |
| 237 | | | | | | | | | | | | | | | | | | | | | | | | | | |
| 238 | | | | | | | | | | | | | | | | | | | | | | | | | | |
| 239 | | | | | | | | | | | | | | | | | | | | | | | | | | |
| 240 | | | | | | | | | | | | | | | | | | | | | | | | | | |
| 241 | | | | | | | | | | | | | | | | | | | | | | | | | | |
| 242 | | | | | | | | | | | | | | | | | | | | | | | | | | |
| 243 | | | | | | | | | | | | | | | | | | | | | | | | | | |
| 244 | | | | | | | | | | | | | | | | | | | | | | | | | | |
| 245 | | | | | | | | | | | | | | | | | | | | | | | | | | |
| 246 | | | | | | | | | | | | | | | | | | | | | | | | | | |
| 247 | | | | | | | | | | | | | | | | | | | | | | | | | | |
| 248 | | | | | | | | | | | | | | | | | | | | | | | | | | |
| 249 | | | | | | | | | | | | | | | | | | | | | | | | | | |
| 250 | | | | | | | | | | | | | | | | | | | | | | | | | | |
| 251 | | | | | | | | | | | | | | | | | | | | | | | | | | |
| 252 | | | | | | | | | | | | | | | | | | | | | | | | | | |
| 253 | | | | | | | | | | | | | | | | | | | | | | | | | | |
| 254 | | | | | | | | | | | | | | | | | | | | | | | | | | |
| 255 | | | | | | | | | | | | | | | | | | | | | | | | | | |
| 256 | | | | | | | | | | | | | | | | | | | | | | | | | | |
| 257 | | | | | | | | | | | | | | | | | | | | | | | | | | |
| 258 | | | | | | | | | | | | | | | | | | | | | | | | | | |
| 259 | | | | | | | | | | | | | | | | | | | | | | | | | | |
| 260 | | | | | | | | | | | | | | | | | | | | | | | | | | |
| 261 | | | | | | | | | | | | | | | | | | | | | | | | | | |
| 262 | | | | | | | | | | | | | | | | | | | | | | | | | | |
| 263 | | | | | | | | | | | | | | | | | | | | | | | | | | |
| 264 | | | | | | | | | | | | | | | | | | | | | | | | | | |
| 265 | | | | | | | | | | | | | | | | | | | | | | | | | | |
| 266 | | | | | | | | | | | | | | | | | | | | | | | | | | |
| 267 | | | | | | | | | | | | | | | | | | | | | | | | | | |
| 268 | | | | | | | | | | | | | | | | | | | | | | | | | | |
| 269 | | | | | | | | | | | | | | | | | | | | | | | | | | |
| 270 | | | | | | | | | | | | | | | | | | | | | | | | | | |
| 271 | | | | | | | | | | | | | | | | | | | | | | | | | | |
| 272 | | | | | | | | | | | | | | | | | | | | | | | | | | |
| 273 | | | | | | | | | | | | | | | | | | | | | | | | | | |
| 274 | | | | | | | | | | | | | | | | | | | | | | | | | | |
| 275 | | | | | | | | | | | | | | | | | | | | | | | | | | |
| 276 | | | | | | | | | | | | | | | | | | | | | | | | | | |
| 277 | | | | | | | | | | | | | | | | | | | | | | | | | | |
| 278 | | | | | | | | | | | | | | | | | | | | | | | | | | |
| 279 | | | | | | | | | | | | | | | | | | | | | | | | | | |
| 280 | | | | | | | | | | | | | | | | | | | | | | | | | | |
| 281 | | | | | | | | | | | | | | | | | | | | | | | | | | |
| 282 | | | | | | | | | | | | | | | | | | | | | | | | | | |
| 283 | | | | | | | | | | | | | | | | | | | | | | | | | | |
| 284 | | | | | | | | | | | | | | | | | | | | | | | | | | |
| 285 | | | | | | | | | | | | | | | | | | | | | | | | | | |
| 286 | | | | | | | | | | | | | | | | | | | | | | | | | | |
| 287 | | | | | | | | | | | | | | | | | | | | | | | | | | |
| 288 | | | | | | | | | | | | | | | | | | | | | | | | | | |
| 289 | | | | | | | | | | | | | | | | | | | | | | | | | | |
| 290 | | | | | | | | | | | | | | | | | | | | | | | | | | |
| 291 | | | | | | | | | | | | | | | | | | | | | | | | | | |
| 292 | | | | | | | | | | | | | | | | | | | | | | | | | | |
| 293 | | | | | | | | | | | | | | | | | | | | | | | | | | |
| 294 | | | | | | | | | | | | | | | | | | | | | | | | | | |
| 295 | | | | | | | | | | | | | | | | | | | | | | | | | | |
| 296 | | | | | | | | | | | | | | | | | | | | | | | | | | |
| 297 | | | | | | | | | | | | | | | | | | | | | | | | | | |
| 298 | | | | | | | | | | | | | | | | | | | | | | | | | | |
| 299 | | | | | | | | | | | | | | | | | | | | | | | | | | |
| 300 | | | | | | | | | | | | | | | | | | | | | | | | | | |
| 301 | | | | | | | | | | | | | | | | | | | | | | | | | | |
| 302 | | | | | | | | | | | | | | | | | | | | | | | | | | |
| 303 | | | | | | | | | | | | | | | | | | | | | | | | | | |
| 304 | | | | | | | | | | | | | | | | | | | | | | | | | | |
| 305 | | | | | | | | | | | | | | | | | | | | | | | | | | |
| 306 | | | | | | | | | | | | | | | | | | | | | | | | | | |
| 307 | | | | | | | | | | | | | | | | | | | | | | | | | | |
| 308 | | | | | | | | | | | | | | | | | | | | | | | | | | |
| 309 | | | | | | | | | | | | | | X | | | | | X | | | | | X | | X |
| 310 | | | | | | | | | | | | | | | | | | | | | | | | X | | X |
| 311 | | | | | | | | | | | X | | | | | X | | | | | | | | | | X |
| 312 | | | | X | X | | | | | | X | | | | | X | | | | | | | | X | | |
| 313 | | | | | | | | | | | X | | | | | X | | | | | | | | X | X | |
| 314 | | | | | | | | | X | X | X | | | | | X | | | | X | | | | | | X |
| 315 | | | | | | | | | | | X | | | | | X | | | | | | | | | | |
| 316 | | | | | | | | | | | X | | | | | X | | | | | | | | X | X | |
| 317 | | | | X | | | | | | | | | | | | | | | | | | | | | | |
| 318 | | | | X | | | | | | | | | | | | | | | | | | | | | | |
| 319 | | | | | | | | | | | X | | | | | X | | | | | | | | X | X | |
| 320 | | | | | | | | | | | X | | | | | X | | | | | | | | X | X | |
| 321 | | | | | | | | | | | X | | | | | X | | | | | | | | X | X | |
| 322 | | | | | | | | | | | X | | | | | X | | | | | | | | X | X | |
| 323 | | | | | | | | | | | X | | | | | X | | | | | | | | X | X | |
| 324 | | | | | | | | | | | X | | | | | X | | | | | | | | X | X | |
| 325 | | | | | | | | | | | X | | | | | X | | | | | | | | X | X | |
| 326 | | | | | | | | | | | X | | | | | X | | | | | | | | X | X | |
| 327 | | | | | | | | | X | | X | | | | | X | | | | X | | | | X | X | |
| 328 | | | | | | | | | | | X | | | | | X | | | | | | | | X | X | |
| 329 | | | | | | | | | | | X | | | | | X | | | | | | | | X | X | |
| 330 | | | | | | | | | | | X | | | | | X | | | | | | | | X | X | |
| 331 | | | | | | | | | | | X | | | | | X | | | | | | | | | | |
| 332 | | | | | | | | | | | X | | | | | X | | | | | | | | X | X | |
| 333 | | | | | | | | | | | X | | | | | X | | | | | | | | X | X | |
| 334 | | | | | | | | | | | X | | | | | X | | | | | | | | X | X | |
| 335 | | | | | | | | | | | X | | | | | X | | | | | | | | X | X | |
| 336 | | | | | | | | | | | X | | | | | X | | | | | | | | X | X | |
| 337 | | | | | | | | | | | X | | | | | X | | | | | | | | X | X | |
| 338 | | | | | | | | | | | X | | | | | X | | | | | | | | X | X | |
| 339 | | | | | | | | | | | X | | | | | X | | | | | | | | | | |
| 340 | | | | | | | | | | | X | | | | | X | | | | | | | | | | |
| 341 | | | | | | | | | | | X | | | | | X | | | | | | | | X | X | |
| 342 | | | | | | | | | | | X | | | | | X | | | | | | | | X | X | |
| 343 | | | | | | | | | | | X | | | | | X | | | | | | | | X | X | |
| 344 | | | | | | | | | | | X | | | | | X | | | | | | | | X | X | |
| 345 | | | | | | | | | | | X | | | | | X | | | | | | | | X | X | |
| 346 | | | | | | | | | | | X | | | | | X | | | | | | | | X | X | |
| 347 | | | | | | | | | | | X | | | | | X | | | | | | | | X | X | |
| 348 | | X | | | | | | X | | | | | | | X | X | | | | | | | | X | | X |
| 349 | | | | | | | | | | | X | | | | | X | | | | | | | | X | X | |
| 350 | | | | | | | | | | | X | | | | | X | | | | | | | | X | X | X |
| 351 | | | | | | | | | | | X | | | | | X | | | | | | | | X | X | |
| 352 | | | | | | | | | | | X | | | | | X | | | | | | | | X | X | |
| 353 | | | | | | | | | | | X | | | | | X | | | | | | | | X | X | |
| 354 | | | | | | | | | | | X | | | | | X | | | | | | | | | | |
| 355 | | | | | | | | | | | X | | | | | X | | | | | | | | X | X | |
| 356 | | | | | | | | | | | X | | | | | X | | | | | | | | X | X | |
| 357 | | | | | | | | | | | X | | | | | X | | | | | | | | X | X | |
| 358 | | | | X | | | | | | | X | | | | | X | | | | | | | | X | X | |
| 359 | | | | | | | | | | | X | | | | | X | | | | | | | | X | X | |
| 360 | | | | X | | | | | X | | X | | | | | X | | | X | | | | | | | |
| 361 | | | | | | | | | | | X | | | | | X | | | | | | | | X | X | |
| 362 | | | | | | X | | | | | X | | | | | X | | | | | | | | X | X | |
| 363 | | | | | | | | | | | X | | | | | X | | | | | | | | X | X | |
| 364 | | | | | | | | | | | X | | | | | X | | | | | | | | X | X | |
| 365 | | | | X | X | | | | | | | | | | | | | | | | | | | | | |
| 366 | | | | | | | | | | | X | | | | | X | | | | | | | | X | X | |
| 367 | | | | | | | | | | | X | | | | | X | | | | | | | | X | X | |
| 368 | | | | | | | | | | | X | | | | | X | | | | | | | | X | X | |
| 369 | | | | X | | | | | | | X | | | | | X | | | | | | | | X | | |
| 370 | | | | | | | | | | | X | | | | | X | | | | | | | | X | | |
| 371 | | | | X | | | | | | | X | | | | | X | | | | | | | | X | | |
| 372 | | | | | | | | | | | X | | | | | X | | | | | | | | X | | |
| 373 | | | | | | | | | | | X | | | | | X | | | | | | | | X | X | |
| 374 | | | | | | | | | | | X | | | | | X | | | | | | | | X | | |
| 375 | | | | | | | | | | | X | | | | | X | | | | | | | | | X | |
| 376 | | | | | | | | | | | X | | | | | X | | | | | | | | | | |
| 377 | | | | | | | | | | | X | | | | | X | | | | | | | | | | |

| ID | 27 | 28 | 29 | 30 | 31 | 32 | 33 | 34 | 35 | 36 | 37 | 38 | 39 | 40 | 41 | 42 | 43 | 44 | 45 | 46 | 47 | 48 | 49 | 50 | 51 |
|---|---|---|---|---|---|---|---|---|---|---|---|---|---|---|---|---|---|---|---|---|---|---|---|---|---|
| 188 | | | | | | | | | | X | | | | | | | | | | | | | | | |
| 189 | | | | X | | X | | | | | | | | | | | | | | | X | X | X | | |
| 190 | | | | | | | | | | X | | | | | | | | | | | | | | | |
| 191 | | | | | | | | | | X | | | | | | | | | | | | | | | |
| 192 | | | | | | | | | | X | | | | | | | | | | | | | | | |
| 193 | | | | | | | | | | X | | | | | | | | | | | | | | | |
| 194 | | | | | | | | | | X | | | | | | | | | | | | | | | |
| 195 | | | | | | | | | | X | | | | | | | | | | | | | | | |
| 196 | | | X | | | | | X | | | | | | | | | | X | X | X | | | | | |
| 197 | | | X | | | | | X | | | | | | | | | | X | X | X | | | | | |
| 198 | | | X | | | | | X | | | | | | | | | | X | X | X | | | | | |
| 199 | | | X | | | | | X | | | | | | | | | | X | X | X | | | | | |
| 200 | | | | | | | | | | | X | | | | | | | | | | | | | | |
| 201 | | | | | | | | | | | X | X | | | | | | | | | | | | | |
| 202 | | | | | | | | | | X | | | | | | | | | | | | | | | |
| 203 | | | | | | | | | | X | | | | | | | | | | | | | | | |
| 204 | | | | | | | | | | X | | | | | | | | | | | | | | | |
| 205 | | | | | | | | | | X | | | | | | | | | | | | | | | |
| 206 | | | | | | | | | | X | | | | | | | | | | | | | | | |
| 207 | | | | | | | | | | X | | | | | | | | | | | | | | | |
| 208 | | | | | | | | | | X | | | | | | | | | | | | | | | |
| 209 | | | | | | | | | | X | | | | | | | | | | | | | | | |
| 210 | | | | | | | | | | X | | | | | | | | | | | | | | | |
| 211 | | | | | | | | | | X | | | | | | | | | | | | | | | |
| 212 | | | | | | | | | | X | | | | | | | | | | | | | | | |
| 213 | | | | | | | | | | X | | | | | | | | | | | | | | | |
| 214 | | | | | | | | | | X | | | | | | | | | | | | | | | |
| 215 | | | | | | | | | | X | | | | | | | | | | | | | | | |
| 216 | | | | | | | | | | X | | | | | | | | | | | | | | | |
| 217 | | | | | | | | | | X | | | | | | | | | | | | | | | |
| 218 | | | | | | | | | | X | | | | | | | | | | | | | | | |
| 219 | | | | | | | | | | X | | | | | | | | | | | | | | | |
| 220 | | | | | | | | | | X | | | | | | | | | | | | | | | |
| 221 | | | | | | | | | | | | | | | | | | | | | | | | | |
| 222 | | | | | | | | | | X | | | | | | | | | | | | | | | |
| 223 | | | | | | | | | | | | | | | X | | | | | | | | | X | |
| 224 | | | | | | | | | | | X | | X | | | | | | | | | | | | |
| 225 | | | | | | | | | | | | | | | X | | | | | | | | | X | |
| 226 | | | | | | | | | | | | | | | | | | | | | | | | X | |
| 227 | | | | | | | | | | | X | | | | | | | | | | | | | | |
| 228 | | | | | | | | | | | | | | | | | | | | | | | | | |
| 229 | | | | | | | | | | | | | | | | | | | | | | | | | |
| 230 | | | X | | | | | X | | | | | | | | | | X | X | X | | | | | |
| 231 | | | X | | | | | X | | | | | | | | | | X | X | X | | | | | |
| 232 | | | | | | | | | | X | | | | | | | | | | | | | | | |
| 233 | | | | | | | | | | X | | | | | | | | | | | | | | | |
| 234 | | X | | | | X | | | | | | | | | | | | | | | | | | | |
| 235 | | | | | | | | | | X | | | | | | | | | | | | | | | |
| 236 | | X | | | | X | | | | | | | | | | | | | | | | | | | |
| 237 | | | | | | | | | | X | | | | | | | | | | | | | | | |
| 238 | | | | | | | | | | X | | | | | | | | | | | | | | | |
| 239 | | | | | | | | | | X | | | | | | | | | | | | | | | |
| 240 | | | | | | | | | | X | | | | | | | | | | | | | | | |
| 241 | | | | | | | | | | X | | | | | | | | | | | | | | | |
| 242 | | | | | | | | | | X | | | | | | | | | | | | | | | |
| 243 | | | | | | | | | | X | | | | | | | | | | | | | | | |
| 244 | | | | | | | | | | X | | | | | | | | | | | | | | | |
| 245 | | | | | | | | | | X | | | | | | | | | | | | | | | |
| 246 | | | | | | | | | | X | | | | | | | | | | | | | | | |
| 247 | | | | | | | | | | X | | | | | | | | | | | | | | | |
| 248 | | | | | | | | | | X | | | | | | | | | | | | | | | |
| 249 | | | | | | | | | | X | | | | | | | | | | | | | | | |
| 250 | | | | | | | | | | X | | | | | | | | | | | | | | | |
| 251 | | | | | | | | | | X | | | | | | | | | | | | | | | |
| 252 | | | | | | | | | | X | | | | | | | | | | | | | | | |
| 253 | | | | | | | | | | X | | | | | | | | | | | | | | | |
| 254 | | | | | | | | | | X | | | | | | | | | | | | | | | |
| 255 | | | | | | | | | | X | | | | | | | | | | | | | | | |
| 256 | | | | | | | | | | X | | | | | | | | | | | | | | | |
| 257 | | | | | | | | | | X | | | | | | | | | | | | | | | |
| 258 | | | | | | | | | | X | | | | | | | | | | | | | | | |
| 259 | | | | | | | | | | X | | | | | | | | | | | | | | | |
| 260 | | | | | | | | | | X | | | | | | | | | | | | | | | |
| 261 | | | | | | | | | | X | | | | | | | | | | | | | | | |
| 262 | | | | | | | | | | X | | | | | | | | | | | | | | | |
| 263 | | | | | | | | | | X | | | | | | | | | | | | | | | |
| 264 | | | | | | | | | | X | | | | | | | | | | | | | | | |
| 265 | | | | | | | | | | X | | | | | | | | | | | | | | | |
| 266 | | | | | | | | | | X | | | | | | | | | | | | | | | |
| 267 | | | | | | | | | | X | | | | | | | | | | | | | | | |
| 268 | | | | | | | | | | X | | | | | | | | | | | | | | | |
| 269 | | | | | | | | | | X | | | | | | | | | | | | | | | |
| 270 | | | | | | | | | | X | | | | | | | | | | | | | | | |
| 271 | | | | | | | | | | X | | | | | | | | | | | | | | | |
| 272 | | | | | | | | | | X | | | | | | | | | | | | | | | |
| 273 | | | | | | | | | | X | | | | | | | | | | | | | | | |
| 274 | | | | | | | | | | X | | | | | | | | | | | | | | | |
| 275 | | | | | | | | | | X | | | | | | | | | | | | | | | |
| 276 | | | | | | | | | | X | | | | | | | | | | | | | | | |
| 277 | | | | | | | | | | X | | | | | | | | | | | | | | | |
| 278 | | | | | | | | | | X | | | | | | | | | | | | | | | |
| 279 | | | | | | | | | | X | | | | | | | | | | | | | | | |
| 280 | | | | | | | | | | X | | | | | | | | | | | | | | | |
| 281 | | | | | | | | | | X | | | | | | | | | | | | | | | |
| 282 | | | | | | | | | | X | | | | | | | | | | | | | | | |
| 283 | | | | | | | | | | X | | | | | | | | | | | | | | | |
| 284 | | | | | | | | | | X | | | | | | | | | | | | | | | |
| 285 | | | | | | | | | | X | | | | | | | | | | | | | | | |
| 286 | | | | | | | | | | X | | | | | | | | | | | | | | | |
| 287 | | | | | | | | | | X | | | | | | | | | | | | | | | |
| 288 | | | | | | | | | | X | | | | | | | | | | | | | | | |
| 289 | | | | | | | | | | X | | | | | | | | | | | | | | | |
| 290 | | | | | | | | | | X | | | | | | | | | | | | | | | |
| 291 | | | | | | | | | | X | | | | | | | | | | | | | | | |
| 292 | | | | | | | | | | X | | | | | | | | | | | | | | | |
| 293 | | | | | | | | | | X | | | | | | | | | | | | | | | |
| 294 | | | | | | | | | | X | | | | | | | | | | | | | | | |
| 295 | | | | | | | | | | X | | | | | | | | | | | | | | | |
| 296 | | | | | | | | | | X | | | | | | | | | | | | | | | |
| 297 | | | | | | | | | | X | | | | | | | | | | | | | | | |
| 298 | | | | | | | | | | X | | | | | | | | | | | | | | | |
| 299 | | | | | | | | | | X | | | | | | | | | | | | | | | |
| 300 | | | | | | | | | | X | | | | | | | | | | | | | | | |
| 301 | | | | | | | | | | X | | | | | | | | | | | | | | | |
| 302 | | | | | | | | | | X | | | | | | | | | | | | | | | |
| 303 | | | | | | | | | | X | | | | | | | | | | | | | | | |
| 304 | | | | | | | | | | X | | | | | | | | | | | | | | | |
| 305 | | | | | | | | | | X | | | | | | | | | | | | | | | |
| 306 | | | | | | | | | | X | | | | | | | | | | | | | | | |
| 307 | | | | | | | | | | X | | | | | | | | | | | | | | | |
| 308 | | | | | | | | | | X | | | | | | | | | | | | | | | |
| 309 | | | | X | | | | | | X | | | | | | | | | | | | | | | |
| 310 | | | | | | | | | | X | | | | | | | | | | | | | | | |
| 311 | | | | X | | X | | | | | | | | | | | | | | | | | | | |
| 312 | | | | | | X | | | | | | | | | | | | | | | | | | | |
| 313 | | X | | | | X | | | | | | | | | | | | | | | | | | | |
| 314 | | | | X | | X | | | | | | | | | | | | | | | | | | | |
| 315 | | | | | | X | | | | | | | | | | | | | | | | | | | |
| 316 | | X | | | | X | | | | X | | | | | | | | | | | | | | | |
| 317 | | | | | | | | | | | | | | | | | | | | | | | | | |
| 318 | | | | | | | | | | | | | | | | | | | | | | | | | |
| 319 | | | | | | X | | | | | | | | | | | | | | | | | | | |
| 320 | | | | | | X | | | | | | | | | | | | | | | | | | | |
| 321 | | | | | | X | | | | | | | | | | | | | | | | | | | |
| 322 | | | | | | X | | | | | | | | | | | | | | | | | | | |
| 323 | | | | | | X | | | | | | | | | | | | | | | | | | | |
| 324 | | | | | | X | | | | X | | | | | | | | | | | | | | | |
| 325 | | | | | | X | | | | | | | | | | | | | | | | | | | |
| 326 | | | | | | X | | | | | | | | | | | | | | | | | | | |
| 327 | | | | | | X | | | | | | | | X | | | | | | | | | | | |
| 328 | | | | | | X | | | | | | | | | | | | | | | | | | | |
| 329 | | X | | | | X | | | | | | | | | | | | | | | | | | | |
| 330 | | X | | | | X | | | | | | | | | | | | | | | | | | | |
| 331 | | X | | | | X | | | | | | | | | | | | | | | | | | | |
| 332 | | | | | | X | | | | | | | | | | | | | | | | | | | |
| 333 | | | | | | X | | | | | | | | | | | | | | | | | | | |
| 334 | | | | | | X | | | | | | | | | | | | | | | | | | | |
| 335 | | X | | | | X | | | | | | | | | | | | | | | | | | | |
| 336 | | X | | | | X | | | | | | | | | | | | | | | | | | | |
| 337 | | | | | | X | | | | | | | | | | | | | | | | | | | |
| 338 | | X | | | | X | | | | | | | | | | | | | | | | | | | |
| 339 | | | | | | X | | | | | | | | | | | | | | | | | | | |
| 340 | | | | | | X | | | | | | | | | | | | | | | | | | | |
| 341 | | | | | | X | | | | | | | | | | | | | | | | | | | |
| 342 | | X | | | | X | | | | | | | | | | | | | | | | | | | |
| 343 | | X | | | | X | | | | | | | | | | | | | | | | | | | |
| 344 | | | | | | X | | | | | | | | | | | | | | | | | | | |
| 345 | | | | | | X | | | | | | | | | | | | | | | | | | | |
| 346 | | X | | | | X | | | | | | | | | | | | | | | | | | | |
| 347 | | | | | | X | | | | | | | | | | | | | | | | | | | |
| 348 | | | | | | | | | | | | | | | | | | | | | | | | | |
| 349 | | X | | | | X | | | | | | | | | | | | | | | | | | | |
| 350 | | | | | | X | | | | | | | | | | | | | | | | | | | |
| 351 | | | | | | X | | | | | | | | | | | | | | | | | | | |
| 352 | | X | | | | X | | | | | | | | | | | | | | | | | | | |
| 353 | | X | | | | X | | | | | | | | | | | | | | | | | | | |
| 354 | | | | | | X | | | | | | | | | | | | | | | | | | | |
| 355 | | X | | | | X | | | | | | | | | | | | | | | | | | | |
| 356 | | X | | | | X | | | | | | | | | | | | | | | | | | | |
| 357 | | X | | | | X | | | | | | | | | | | | | | | | | | | |
| 358 | | X | | | | X | | | | | | | | | | | | | | | | | | | |
| 359 | | X | | | | X | | | | | | | | | | | | | | | | | | | |
| 360 | | | | | | X | | | | | | | | X | | | | | | | | | | | |
| 361 | | X | | | | X | | | | | | | | | | | | | | | | | | | |
| 362 | | X | | | | X | | | | | | | | | | | | | | | | | | | |
| 363 | | X | | | | X | | | | | | | | | | | | | | | | | | | |
| 364 | | X | | | | X | | | | | | | | | | | | | | | | | | | |
| 365 | | | | | | | | | | | | | | | | | | | | | | | | | |
| 366 | | | | | | X | | | | | | | | | | | | | | | | | | | |
| 367 | | X | | | | X | | | | | | | | | | | | | | | | | | | |
| 368 | | X | | | | X | | | | | | | | | | | | | | | | | | | |
| 369 | | X | | | | X | | | | | | | | | | | | | | | | | | | |
| 370 | | X | | | | X | | | | | | | | | | | | | | | | | | | |
| 371 | | X | | | | X | | | | | | | | | | | | | | | | | | | |
| 372 | | X | | | | X | | | | | | | | | | | | | | | | | | | |
| 373 | | X | | | | X | | | | | | | | | | | | | | | | | | | |
| 374 | | X | | | | X | | | | | | | | | | | | | | | | | | | |
| 375 | | X | | X | | X | | | | | | | | | | | | | | | | | | | |
| 376 | | | | | | X | | | | | | | | | | | | | | | | | | | |
| 377 | | X | | | | X | | | | | | | | | | | | | | | | | | | |

| ID | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
|---|---|---|---|---|---|---|---|---|---|---|---|---|---|---|---|---|---|---|---|---|---|---|---|---|---|---|
| 378 | | | | | | | | | | | X | | | | | X | | | | | | | | | | |
| 379 | | | | | | | | | | | X | | | | | X | | | | | | | | X | X | |
| 380 | | | | X | X | | | | | | | | | | | | | | | | | | X | | | X |
| 381 | | | | | | | | | | | X | | | | | X | | | | | | | | X | X | |
| 382 | | | | | | | | | | | X | | | | | X | | | | | | | | X | | |
| 383 | | | | | | | | | | | X | | | | | X | | | | | | | | | | |
| 384 | | | | | | | | | | | X | | | | | X | | | | | | | | | | |
| 385 | | | | | | | | | | | X | | | | | X | | | | | | | | X | X | |
| 386 | | | | | | | | | | | X | | | | | X | | | | | | | | X | X | |
| 387 | | | | | | | | | | | X | | | | | X | | | | | | | | X | X | |
| 388 | | | | | | | | | | | X | | | | | X | | | | | | | | X | X | |
| 389 | | | | | | | | | | | | | | | | X | | | | | | | | | | |
| 390 | | | | | | | | | | | X | | | | | X | | | | | | | | X | X | |
| 391 | | | | | | | | | | | X | | | | | X | | | | | | | | X | X | |
| 392 | | | | | | | | | | | X | | | | | X | | | | | | | | X | X | |
| 393 | | | | | | | | | | | X | | | | | X | | | | | | | | X | X | |
| 394 | | | | X | | | | | | | X | | | | | X | | | | | | | | X | X | |
| 395 | | | | X | | | | | | | X | | | | | X | | | | | | | | X | X | |
| 396 | | | | X | | | | | | | X | | | | | X | | | | | | | | X | X | |
| 397 | | | | X | | | | | | | X | | | | | X | | | | | | | | X | X | |
| 398 | | | | | | | | | | | X | | | | | X | | | | | | | | X | X | |
| 399 | | | | X | | | | | | | X | | | | | X | | | | | | | | X | X | |
| 400 | | | | X | | | | | | | X | | | | | X | | | | | | | | X | X | |
| 401 | | | | | | | | | | | X | | | | | X | | | | | | | | X | X | |
| 402 | | | | | | | | | | | X | | | | | X | | | | | | | | X | | |
| 403 | | | | | | | | | | | X | | | | | X | | | | | | | | X | X | |
| 404 | | | | | | | | | | | X | | | | | X | | | | | | | | X | X | |
| 405 | | | | | | | | | | | X | | | | | X | | | | | | | | X | X | |
| 406 | | | | | | | | | | | X | | | | | X | | | | | | | | X | X | |
| 407 | | | | | | | | | | | X | | | | | X | | | | | | | | X | X | |
| 408 | | | | | | | | | | | X | | | | | X | | | | | | | | X | X | |
| 409 | | | | | | | | | X | X | X | | | | | X | | | | X | | | | X | X | |
| 410 | | | | | | | | | | | X | | | | | X | | | | | | | | X | X | |
| 411 | | | | X | | X | | | | | | | | | | X | | | | | | | | X | X | |
| 412 | | | | | | | | | X | X | X | | | | | X | | | | X | | | | | | |
| 413 | | | | | | | | | | | X | | | | | X | | | | | | | | X | X | |
| 414 | | | | | | | | | | | X | | | | | X | | | | | | | | X | X | |
| 415 | | | | | | | | | | | X | | | | | X | | | | | | | | X | X | |
| 416 | | | | | | | | | | | X | | | | | X | | | | | | | | X | X | |
| 417 | | | | | | | | | | | X | | | | | | | | | | X | | | X | X | X |
| 418 | | | | | | | | | | | X | | | | | X | | | | | X | | | X | X | X |
| 419 | | | | | | | | | | | X | | | | | X | | | | | | | | X | X | |
| 420 | | | | | | | | | | | X | | | | | X | | | | | X | | | X | X | X |
| 421 | | | | | | | | | | | X | | | | | X | | | | | | | | X | X | |
| 422 | | | | | | | | | | | X | | | | | X | | | | | | | | X | | |
| 423 | | | | | | | | | | | X | | | | | X | | | | | | | | X | | |
| 424 | | | | | | | | | | | X | | | | | X | | | | | | | | X | X | |
| 425 | | | | | | | | | | | X | | | | | X | | | | | | | | X | X | |
| 426 | | | | | | | | | | | X | | | | | X | | | | | | | | X | X | |
| 427 | | | | | | | | | | | X | | | | | X | | | | | | | | X | X | |
| 428 | | | | | | | | | | | X | | | | | X | | | | | | | | X | X | |
| 429 | | | | | | | | | | | X | | | | | X | | | | | | | | X | X | |
| 430 | | | | | | | | | | | X | | | | | X | | | | | | | | | | |
| 431 | | | | | | | | | | | X | | | | | X | | | | | | | | X | X | |
| 432 | | | | X | | | | | | | X | | | | | X | | | | | | | | X | X | |
| 433 | | | | | | | | | | | X | | | | | X | | | | | | | | X | X | |
| 434 | | | | | | | | | | | X | | | | | X | | | | | | | | X | X | |
| 435 | | | | | | | | | | | X | | | | | X | | | | | | | | | | |
| 436 | | | | | | | | | X | X | X | | | | | X | | | | X | | | | X | X | |
| 437 | | | | | | | | | X | X | X | | | | | X | | | | X | | | | X | X | |
| 438 | | | | | | | | | X | X | X | | | | | X | | | | X | | | | X | X | |
| 439 | | | | | | | | | | | | | | | | | | | | | | | | | | |
| 440 | | | | | | | | | | | X | | | | | X | | | | | | | | X | X | |
| 441 | | | | | | | | | | | X | | | | | X | | | | | | | | | | |
| 442 | | | | | | | | | | | | | | | | | | | | | | | X | | | X |
| 443 | | | | | | | | | | | | | | | | X | | | | | | | | X | | X |
| 444 | | | | | | | | | | | | | | | | X | | | | | | | | X | | X |
| 445 | | | | | | | | | X | X | | | | | | | | | | | | | | | | |
| 446 | | | | | | | | | X | X | | | | | | | | | | | | | | | | |
| 447 | | | | | | | | | | | X | | | | | X | | | | | | | | X | X | |
| 448 | | | | X | | | | | X | X | X | | | | | X | | | | X | | | | X | X | |
| 449 | | | | | | | | | | | X | | | | | X | | | | | | | | X | X | |
| 450 | | | | | | | | | | | | | | | | | | | | | | | | X | X | |
| 451 | | | | | | | | | | | X | | | | | X | | | | | | | | X | X | |
| 452 | | | | | | | | | | | X | | | | | X | | | | | | | | X | X | |
| 453 | | | | | | | | | | | X | | | | | X | | | | | | | | X | X | |
| 454 | | | | | | | | | | | X | | | | | X | | | | | | | | X | X | |
| 455 | | | | | | | | | | | X | | | | | X | | | | | | | | X | X | |
| 456 | | | | | | | | | | | X | | | | | X | | | | | | | | X | X | |
| 457 | | | | | | | | | | | X | | | | | X | | | | | | | | X | X | |
| 458 | | | | | | | | | | | | | | | | | | | | | | | | X | X | |
| 459 | | | | | | | | | | | | | | | | | | | | | | | | X | X | |
| 460 | | | | | | | | | | | X | | | | | X | | | | | | | | X | X | |
| 461 | | | | | | | | | | | X | | | | | X | | | | | | | | X | X | |
| 462 | | | | | | | | | | | | | | | | X | | | | | | | | X | | X |
| 463 | | | | | | | | | | | X | | | | | X | | | | | | | | X | X | |
| 464 | | | | | | | | | | | | | | | | X | | | | | | | | X | | X |
| 465 | | | | | | | | | | | X | | | | | X | | | | | | | | X | X | |
| 466 | | | | | | | | | | | X | | | | | X | | | | | | | | X | X | |
| 467 | | | | | | | | | | | X | | | | | X | | | | | | | | X | X | |
| 468 | | | | | | | | | X | X | X | | | | | X | | | | X | | | | X | | |
| 469 | | | | | | | | | | | | | | | | X | | | | | | | | X | | X |
| 470 | | | | | | | | | | | X | | | | | X | | | | | | | | X | X | |
| 471 | | | | | | | | | | | X | | | | | X | | | | | | | | X | X | |
| 472 | | | | | | | | | | | X | | | | | X | | | | | | | | X | X | |
| 473 | | | | | | | | | | | X | | | | | X | | | | | | | | X | X | |
| 474 | | | | | | | | | | | X | | | | | X | | | | | | | | X | X | |
| 475 | | | | | | | | | | | X | | | | | X | | | | | | | | X | X | |
| 476 | | | | | | | | | | | X | | | | | X | | | | | | | | X | X | |
| 477 | | | | | | | | | | | X | | | | | X | | | | | | | | X | X | |
| 478 | | | | | | | | | | | X | | | | | X | | | | | | | | X | X | |
| 479 | | | | | | | | X | | | X | | | | | X | | | | | | | | X | X | |
| 480 | | | | | | | | | | | X | | | | | X | | | | | | | | X | X | |
| 481 | | | | | | | | | | | X | | | | | X | | | | | | | | X | X | |
| 482 | | | | | | | | | | | X | | | | | X | | | | | | | | X | X | |
| 483 | | | | | | | | | | | X | | | | | X | | | | | | | | X | X | |
| 484 | | | | | | | | | | | X | | | | | X | | | | | | | | X | X | |
| 485 | | | | | | | | | | | | | | | | X | | | | | | | | X | | X |
| 486 | | | | | | | | | | | | | | | | X | | | | | | | | X | | X |
| 487 | | | | | | | | | | | | | | | | X | | | | | | | | X | | X |
| 488 | | | | | | | | | | | X | | | | | X | | | | | | | | X | X | |
| 489 | | | | | | | | | | | X | | | | | X | | | | | | | | X | X | |
| 490 | | | | | | | | | | | X | | | | | X | | | | | | | | X | X | |
| 491 | | | | | | | | | | | X | | | | | X | | | | | | | | | | |
| 492 | | | | | | | | | | | X | | | | | X | | | | | | | | | | X |
| 493 | | | | | | | | | | | X | | | | | X | | | | | | | X | | | X |
| 494 | | | | X | | | | | | | X | | | | | X | | | | | | | | X | X | |
| 495 | | | | X | | | | | | | X | | | | | X | | | | | | | | X | X | |
| 496 | | | | X | | | | | | | X | | | | | X | | | | | | | | X | X | |
| 497 | | | | X | | | | | | | X | | | | | X | | | | | | | | X | X | |
| 498 | | | | | | | | | | | X | | | | | X | | | | | | | | X | X | |
| 499 | | | | | | | | | | | X | | | | | X | | | | | | | | X | X | |
| 500 | | | | | | | | | | | X | | | | | X | | | | | | | | | X | |
| 501 | | | | | | | | | | | | | | | | | | | | | | | | | | |
| 502 | | | | X | | | | | | | | | | | | | | | | | | | | | | |
| 503 | | | | X | | | | | | | X | | | | | X | | | | | | | | X | X | |
| 504 | | | | | | | | | | | | | | | | | | | | | | | | | | |
| 505 | | | | X | | | | | | | X | | | | | X | | | | | | | | X | X | |
| 506 | | | | X | | | | | | | X | | | | | X | | | | | | | | X | X | |
| 507 | | | | | | | | | | | X | | | | | X | | | | | | | | X | X | |
| 508 | | | | | | | | | | | X | | | | | X | | | | | | | | X | | X |
| 509 | | | | | | | | | | | X | | | | | X | | | | | | | | X | | X |
| 510 | | | | | | | | | | | X | | | | | X | | | | | | | | X | | X |
| 511 | | | | X | | | | | | | X | | | | | X | | | | | | | | X | | X |
| 512 | | | | X | | | | | | | X | | | | | X | | | | | | | | X | | X |
| 513 | | | | X | | | | | | | X | | | | | X | | | | | | | | X | | X |
| 514 | | | | | | | | | | | X | | | | | X | | | | | | | | X | X | |
| 515 | | | | | | | | | | | X | | | | | X | | | | | | | | X | X | |
| 516 | | | | | | | | | | | X | | | | | X | | | | | | | | X | X | |
| 517 | | | | | X | | | | | | | | | | | | | | | | | | | | | |
| 518 | | | | X | | | | | | | | | | | | | | | | | | | | | | |
| 519 | | | | | | | | | | | X | | | | | X | | | | | | | | X | X | |
| 520 | | | | | | | | | | | | | | | | | | | | | | | | | | |
| 521 | | | | | | | | | | | X | | | | | X | | | | | | | | X | X | |
| 522 | | | | | | | | | | | X | | | | | X | | | | | | | | X | X | |
| 523 | | | | | | | | | | | X | | | | | X | | | | | | | | X | X | |
| 524 | | | | | | | | | | | X | | | | | X | | | | | | | | X | X | |
| 525 | | | | | | | | | | | X | | | | | X | | | | | | | | X | X | |
| 526 | | | | | | | | | | | X | | | | | X | | | | | | | | X | X | X |
| 527 | | | | | | | | | | | X | | | | | X | | | | | | | | X | | |
| 528 | | | | | | | | | | | X | | | | | X | | | | | | | | X | | |
| 529 | | | | | | | | | | | | | | | | | | | | | | | | | | |
| 530 | | | | | | | | | | | | | | | | | | | | | | | | | | |
| 531 | | | | | | | | | | | | | | | | | | | | | | | | | | |
| 532 | | | | | | | | | X | X | X | | | | | X | | | | X | | | | X | | |
| 533 | | | | | | | | | X | X | X | | | | | X | | | | X | | | | X | | |
| 534 | | | | | | | | | X | X | X | | | | | X | | | | X | | | | X | X | |
| 535 | | | | | | | | | | | X | | | | | X | | | | | | | | X | X | |
| 536 | | | | | | | | | | | X | | | | | X | | | | | | | | X | | |
| 537 | | | | | | | | | | | X | | | | | X | | | | | | | | X | | |
| 538 | | | | | | | | | | | X | | | | | X | | | | | | | | X | X | |
| 539 | | | | | | | | | | | X | | | | | X | | | | | | | | X | X | |
| 540 | | | | | | | | | | | X | | | | | X | | | | | | | | X | X | |
| 541 | | | | | | | | | X | X | X | | | | | X | | | | X | | | | X | | |
| 542 | | | | | | | | | | | X | | | | | X | | | | | | | | X | | |
| 543 | | | | | | | | | X | | X | | | | | X | | | | X | | | | X | X | |
| 544 | | | | X | | | | | | | | | | | | | | | | | | | | | | |
| 545 | | | | | | | | | | | X | | | | | X | | | | | | | | X | | |
| 546 | | | | | | | | | | | X | | | | | X | | | | | | | | X | X | |
| 547 | | | | | | | | | | | | | | | | | | | | | | | | | | |
| 548 | | | | | | | | | | | X | | | | | X | | | | | | | | X | X | |
| 549 | | | | | | | | | | | X | | | | | X | | | | | | | | X | X | |
| 550 | | | | | | | | | | | X | | | | | X | | | | | | | | X | X | |
| 551 | | | | | | | | | | | X | | | | | X | | | | | | | | | | |
| 552 | | | | | | | | | | | X | | | | | X | | | | | | | | X | X | |
| 553 | | | | | | | | | | | X | | | | | X | | | | | | | | X | | |
| 554 | | | | | | | | | | | X | | | | | X | | | | | | | | | | |
| 555 | | | | | | | | | | | X | | | | | X | | | | | | | | | | |
| 556 | | | | | | | | | | | X | | | | | X | | | | | | | | X | | |
| 557 | | | | | | | | | | | X | | | | | X | | | | | | | | X | X | |
| 558 | | | | | | | | | | | X | | | | | X | | | | | | | | X | X | |
| 559 | | | | | | | | | | | X | | | | | X | | | | | | | | | X | |
| 560 | | | | | | | | | | | X | | | | | X | | | | | | | | | | |
| 561 | | | | | | | | | | | X | | | | | X | | | | | | | | X | X | |
| 562 | | | | | | | | | | | X | | | | | X | | | | | | | | | X | |
| 563 | | | | | | | | | | | X | | | | | X | | | | | | | | | | |
| 564 | | | | | | | | | | | X | | | | | X | | | | | | | | X | X | |
| 565 | | | | | | | | | | | X | | | | | X | | | | | | | | X | | |
| 566 | | | | X | | | | | | | | | | | | | | | | | | | | | | |
| 567 | | | | | | | | | | | X | | | | | X | | | | | | | | X | X | |
| 568 | | | | | | | | | | | X | | | | | X | | | | | | | | | | |
| 569 | | | | | | | | | | | X | | | | | X | | | | | | | | | | |
| 570 | | | | | | | | | | | X | | | | | X | | | | | | | | X | X | |
| 571 | | | | | | | | | | | X | | | | | X | | | | | | | | | | |
| 572 | | | | | | | | | | | X | | | | | X | | | | | | | | X | X | |
| 573 | | | | X | X | | | | | | | | | | | | | | | | | | X | | | X |
| 574 | | | | | | | | | | | X | | | | | X | | | | | | | | X | X | |
| 575 | | | | | | | | | | | X | | | | | X | | | | | | | | | X | |
| 576 | | | | | | | | | | | | | | | | | | | | | | | X | | | X |
| 577 | | | | | | | | X | | | X | | | | | X | | | | | | | | X | X | |
| 578 | | | | | | | | X | | | X | | | | | X | | | | | | | | X | X | |
| 579 | | | | | | | | | | | X | | | | | X | | | | | | | | X | X | |
| 580 | | | | | | | | | | | X | | | | | X | | | | | | | | X | X | |
| 581 | | | | | | | | | | | X | | | | | X | | | | | | | | X | X | |
| 582 | | | | | | | | | | | X | | | | | X | | | | | | | | X | X | X |
| 583 | | | | | | | | | | | X | | | | | X | | | | | | | | X | X | |
| 584 | | | | X | | | | | | | | | | | | | | | | | | | | | | |
| 585 | | | | | | | | | | | X | | | | | X | | | | | | | | X | X | |
| 586 | | | | | | | | | | | X | | | | | X | | | | | | | | X | | |
| 587 | | | | | | | | | | | X | | | | | X | | | | | | | | X | X | |
| 588 | | | | | | | | | | | X | | | | | X | | | | | | | | X | X | |
| 589 | | | | | | | | | | | | | | | | | | | | | | | | | | |
| 590 | | | | | | | | | | | X | | | | | X | | | | | | | | X | X | |
| 591 | | | | | | | | | | | X | | | | | X | | | | | | | | X | X | |
| 592 | | | | X | | | | | | | X | | | | | X | | | | | | | | X | X | |
| 593 | | | | | | | | | | | | | | | | | | | | | | | | | | |
| 594 | | | | | | | | | | | | | | | | | | | | | | | | | | |
| 595 | | | | | | | | | | | | | | | | | | | | | | | | | | |
| 596 | | | | | | | | | | | | | | | | | | | | | | | | | | |
| 597 | | | | X | | | | | | | X | | | | | X | | | | | | | | X | X | |
| 598 | | | | X | | | | | | | X | | | | | X | | | | | | | | X | X | |
| 599 | | | | X | | | | | | | X | | | | | X | | | | | | | | X | X | |
| 600 | | | | | | | | | | | | | | | | | | | | | | | | | | |
| 601 | | | | | | | | | | | X | | | | | X | | | | | | | | X | X | |
| 602 | | | | | | | | | | | X | | | | | X | | | | | | | | X | X | |
| 603 | | | | | | | | | | | X | | | | | X | | | | | | | | X | X | |
| 604 | | | | | | | | | | | X | | | | | X | | | | | | | | X | X | |
| 605 | | | | X | | | | | | | X | | | | | X | | | | | | | | X | X | |

| ID | 27 | 28 | 29 | 30 | 31 | 32 | 33 | 34 | 35 | 36 | 37 | 38 | 39 | 40 | 41 | 42 | 43 | 44 | 45 | 46 | 47 | 48 | 49 | 50 | 51 |
|---|---|---|---|---|---|---|---|---|---|---|---|---|---|---|---|---|---|---|---|---|---|---|---|---|---|
| 378 | | X | | | | X | | | | | | | | | | | | | | | | | | | |
| 379 | | X | | | | X | | | | | | | | | | | | | | | | | | | |
| 380 | | | | | X | | | | | | | | | | | | | | | | | | | | |
| 381 | | X | | | | X | | | | | | | | | | | | | | | | | | | |
| 382 | | | | | | X | | | | | | | | | | | | | | | | | | | |
| 383 | | | | | | X | | | | | | | | | | | | | | | | | | | |
| 384 | | | | | | X | | | | | | | | | | | | | | | | | | | |
| 385 | | | | | | X | | | | | | | | | | | | | | | | | | | |
| 386 | | X | | | | X | | | | | | | | | | | | | | | | | | | |
| 387 | | X | | | | X | | | | | | | | | | | | | | | | | | | |
| 388 | | X | | | | X | | | | | | | | | | | | | | | | | | | |
| 389 | | | | | | X | | | | | | | | | | | | | | | | | | | |
| 390 | | X | | | | X | | | | | | | | | | | | | | | | | | | |
| 391 | | X | | | | X | | | | | | | | | | | | | | | | | | | |
| 392 | | X | | | | X | | | | | | | | | | | | | | | | | | | |
| 393 | | | | | | X | | | | X | | | | | | | | | | | | | | | |
| 394 | | X | | | | X | | | | | | | | | | | | | | | | | | | |
| 395 | | X | | | | X | | | | X | | | | | | | | | | | | | | | |
| 396 | | X | | | | X | | | | | | | | | | | | | | | | | | | |
| 397 | | | | | | X | | | | | | | | | | | | | | | | | | | |
| 398 | | X | | | | X | | | | | | | | | | | | | | | | | | | |
| 399 | | X | | | | X | | | | | | | | | | | | | | | | | | | |
| 400 | | X | | | | X | | | | | | | | | | | | | | | | | | | |
| 401 | | X | | | | X | | | | | | | | | | | | | | | | | | | |
| 402 | | X | | | | X | | | | | | | | | | | | | | | | | | | |
| 403 | | X | | | | X | | | | | | | | | | | | | | | | | | | |
| 404 | | X | | | | X | | | | | | | | | | | | | | | | | | | |
| 405 | | X | | | | X | | | | | | | | | | | | | | | | | | | |
| 406 | | X | | | | X | | | | | | | | | | | | | | | | | | | |
| 407 | | X | | | | X | | | | | | | | | | | | | | | | | | | |
| 408 | | X | | | | X | | | | | | | | | | | | | | | | | | | |
| 409 | | | | | | X | | | | | | | | | | | | | | | | | | | |
| 410 | | X | | | | X | | | | | | | | | | | | | | | | | | | |
| 411 | | | | | | X | | | | | | | | | | | | | | | | | | | |
| 412 | | | | | | X | | | | | | | | | | | | | | | | | | | |
| 413 | | X | | | | X | | | | | | | | | | | | | | | | | | | |
| 414 | | X | | | | X | | | | | | | | | | | | | | | | | | | |
| 415 | | | | | | X | | | | | | | | | | | | | | | | | | | |
| 416 | | | | | | | X | | | | | | | | | | | | | | | | | | |
| 417 | | | | X | | X | X | | | | | | | | | | | | | | X | X | X | | |
| 418 | | | | X | | X | X | | | | | | | | | | | | | | X | X | X | | |
| 419 | | | | | | X | | | | | | | | | | | | | | | | | | | |
| 420 | | | | X | | X | X | | | | | | | | | | | | | | X | X | X | | |
| 421 | | X | | | | X | | | | | | | | | | | | | | | | | | | |
| 422 | | X | | | | X | | | | | | | | | | | | | | | | | | | |
| 423 | | X | | | | X | | | | | | | | | | | | | | | | | | | |
| 424 | | X | | | | X | | | | | | | | | | | | | | | | | | | |
| 425 | | X | | | | X | | | | | | | | | | | | | | | | | | | |
| 426 | | X | | | | X | | | | | | | | | | | | | | | | | | | |
| 427 | | X | | | | X | | | | | | | | | | | | | | | | | | | |
| 428 | | X | | | | X | | | | | | | | | | | | | | | | | | | |
| 429 | | | | | | X | | | | | | | | | | | | | | | | | | | |
| 430 | | | | | | X | | | | | | | | | | | | | | | | | | | |
| 431 | | | | | | X | | | | | | | | | | | | | | | | | | | |
| 432 | | X | | | | X | | | | | | | | | | | | | | | | | | | |
| 433 | | X | | | | X | | | | | | | | | | | | | | | | | | | |
| 434 | | X | | | | X | | | | | | | | | | | | | | | | | | | |
| 435 | | X | | | | X | | | | | | | | | | | | | | | | | | | |
| 436 | | X | | | | X | | | | | | | | | | | | | | | | | | | |
| 437 | | X | | | | X | | | | | | | | | | | | | | | | | | | |
| 438 | | | | | | X | | | | | | | | | | | | | | | | | | | |
| 439 | | X | | | | | | | | | | | | | | | | | | | | | | | |
| 440 | | X | | | | X | | | | | | | | | | | | | | | | | | | |
| 441 | | | | | | X | | | | | | | | | | | | | | | | | | | |
| 442 | | | | | X | | | | | | | | | | | | | | | | | | | | |
| 443 | | | | | | | | | | | | | | | | | | | | | | | | | |
| 444 | | | | | | | | | | | | | | | | | | | | | | | | | |
| 445 | | | | | | | | | | | | | | | | | | X | | | | | | | |
| 446 | | | | | | | | | | | | | | | | | | X | | | | | | | |
| 447 | | | | | | X | | | | | | | | | | | | | | | | | | | |
| 448 | | X | | | | X | | | | | | | | | | | | X | | | | | | | |
| 449 | | | | | | X | | | | | | | | | | | | | | | | | | | |
| 450 | | | | | | | | | | | | | | | | | | | | | | | | | |
| 451 | | | | | | X | | | | | | | | | | | | | | | | | | | |
| 452 | | | | | | X | | | | | | | | | | | | | | | | | | | |
| 453 | | | | | | X | | | | | | | | | | | | | | | | | | | |
| 454 | | | | | | X | | | | | | | | | | | | | | | | | | | |
| 455 | | X | | | | X | | | | | | | | | | | | | | | | | | | |
| 456 | | | | | | X | | | | | | | | | | | | | | | | | | | |
| 457 | | | | | | X | | | | | | | | | | | | | | | | | | | |
| 458 | | | | | | | | | | | | | | | | | | | | | | | | | |
| 459 | | | | | | | | | | | | | | | | | | | | | | | | | |
| 460 | | X | | | | X | | | | | | | | | | | | | | | | | | | |
| 461 | | | | | | X | | | | | | | | | | | | | | | | | | | |
| 462 | | X | | | | | | | | X | | | | | | | | | | | | | | | |
| 463 | | | | | | X | | | | | | | | | | | | | | | | | | | |
| 464 | | X | | | | | | | | X | | | | | | | | | | | | | | | |
| 465 | | X | | | | X | | | | | | | | | | | | | | | | | | | |
| 466 | | | | | | X | | | | | | | | | | | | | | | | | | | |
| 467 | | X | | | | X | | | | | | | | | | | | | | | | | | | |
| 468 | | | | | | X | | | | | | | | | | | | | | | | | | | |
| 469 | | X | | | | | | | | X | | | | | | | | | | | | | | | |
| 470 | | X | | | | X | | | | | | | | | | | | | | | | | | | |
| 471 | | X | | | | X | | | | | | | | | | | | | | | | | | | |
| 472 | | X | | | | X | | | | | | | | | | | | | | | | | | | |
| 473 | | X | | | | X | | | | | | | | | | | | | | | | | | | |
| 474 | | X | | | | X | | | | | | | | | | | | | | | | | | | |
| 475 | | X | | | | X | | | | | | | | | | | | | | | | | | | |
| 476 | | | | | | X | | | | | | | | | | | | | | | | | | | |
| 477 | | | | X | | X | | | | | | | | | | | | | | | | | | | |
| 478 | | X | | | | X | | | | | | | | | | | | | | | | | | | |
| 479 | | X | | | | | | | | | | | | | | | | | | | | | | | |
| 480 | | | | | | X | | | | | | | | | | | | | | | | | | | |
| 481 | | | | | | X | | | | | | | | | | | | | | | | | | | |
| 482 | | | | | | X | | | | | | | | | | | | | | | | | | | |
| 483 | | | | | | X | | | | | | | | | | | | | | | | | | | |
| 484 | | X | | | | X | | | | | | | | | | | | | | | | | | | |
| 485 | | X | | | | | | | | | | | | | | | | | | | | | | | |
| 486 | | X | | | | | | | | | | | | | | | | | | | | | | | |
| 487 | | X | | | | | | | | | | | | | | | | | | | | | | | |
| 488 | | X | | | | X | | | | | | | | | | | | | | | | | | | |
| 489 | | X | | | | X | | | | | | | | | | | | | | | | | | | |
| 490 | | X | | | | X | | | | | | | | | | | | | | | | | | | |
| 491 | | | | | | X | | | | | | | | | | | | | | | | | | | |
| 492 | | | | X | | X | | | | | | | | | | | | | | | | | | | |
| 493 | | | | | | X | | | | | | | | | | | | | | | | | | | |
| 494 | | X | | | | X | | | | | | | | | | | | | | | | | | | |
| 495 | | | | | | X | | | | | | | | | | | | | | | | | | | |
| 496 | | | | | | X | | | | | | | | | | | | | | | | | | | |
| 497 | | | | | | X | | | | | | | | | | | | | | | | | | | |
| 498 | | X | | | | X | | | | | | | | | | | | | | | | | | | |
| 499 | | X | | | | X | | | | | | | | | | | | | | | | | | | |
| 500 | | X | | | | X | | | | | | | | | | | | | | | | | | | |
| 501 | | | | | | | | | | X | | | | | | | | | | | | | | | |
| 502 | | | | | | | | | | | | | | | | | | | | | | | | | |
| 503 | | | | | | | | | | | | | | | | | | | | | | | | | |
| 504 | | | | | | | | | | X | | | | | | | | | | | | | | | |
| 505 | | X | | | | X | | | | | | | | | | | | | | | | | | | |
| 506 | | X | | | | X | | | | | | | | | | | | | | | | | | | |
| 507 | | X | | | | X | | | | | | | | | | | | | | | | | | | |
| 508 | | | | | | X | | | | | | | | | | | | | | | | | | | |
| 509 | | | | | | X | | | | | | | | | | | | | | | | | | | |
| 510 | | | | | | X | | | | | | | | | | | | | | | | | | | |
| 511 | | | | | | X | | | | | | | | | | | | | | | | | | | |
| 512 | | | | | | X | | | | | | | | | | | | | | | | | | | |
| 513 | | | | | | X | | | | | | | | | | | | | | | | | | | |
| 514 | | | | | | X | | | | | | | | | | | | | | | | | | | |
| 515 | | | | | | X | | | | | | | | | | | | | | | | | | | |
| 516 | | | | | | X | | | | | | | | | | | | | | | | | | | |
| 517 | | | | | | | | | | | | | | | | | | | | | | | | | |
| 518 | | | | | | | | | | X | | | | | | | | | | | | | | | |
| 519 | | | | | | X | | | | | | | | | | | | | | | | | | | |
| 520 | | | | | | | | | | X | | | | | | | | | | | | | | | |
| 521 | | | | | | X | | | | | | | | | | | | | | | | | | | |
| 522 | | | | | | X | | | | | | | | | | | | | | | | | | | |
| 523 | | | | | | X | | | | | | | | | | | | | | | | | | | |
| 524 | | | | | | X | | | | | | | | | | | | | | | | | | | |
| 525 | | | | | | X | | | | | | | | | | | | | | | | | | | |
| 526 | | X | | | | X | | | | | | | | | | | | | | | | | | | |
| 527 | | | | | | X | | | | | | | | | | | | | | | | | | | |
| 528 | | | | | | X | | | | | | | | | | | | | | | | | | | |
| 529 | | | | | | | | | | X | | | | | | | | | | | | | | | |
| 530 | | | | | | | | | | X | | | | | | | | | | | | | | | |
| 531 | | | | | | | | | | X | | | | | | | | | | | | | | | |
| 532 | | | | | | X | | | | | | | | | | | | | | | | | | | |
| 533 | | | | | | X | | | | | | | | | | | | | | | | | | | |
| 534 | | X | | X | | X | | | | | | | | | | | | | | | | | | | |
| 535 | | X | | | | X | | | | | | | | | | | | | | | | | | | |
| 536 | | | | | | X | | | | | | | | | | | | | | | | | | | |
| 537 | | | | | | X | | | | | | | | | | | | | | | | | | | |
| 538 | | X | | | | X | | | | | | | | | | | | | | | | | | | |
| 539 | | X | | | | X | | | | | | | | | | | | | | | | | | | |
| 540 | | X | | | | X | | | | | | | | | | | | | | | | | | | |
| 541 | | | | | | X | | | | | | | | | | | | | | | | | | | |
| 542 | | | | | | X | | | | | | | | | | | | | | | | | | | |
| 543 | | X | | | | X | | | | | | | | X | | | | | | | | | | | |
| 544 | | | | | | | | | | | | | | | | | | | | | | | | | |
| 545 | | X | | | | X | | | | | | | | | | | | | | | | | | | |
| 546 | | X | | | | X | | | | | | | | | | | | | | | | | | | |
| 547 | | | | | | | | | | X | | | | | | | | | | | | | | | |
| 548 | | | | | | X | | | | | | | | | | | | | | | | | | | |
| 549 | | | | | | X | | | | | | | | | | | | | | | | | | | |
| 550 | | | | | | X | | | | | | | | | | | | | | | | | | | |
| 551 | | | | | | X | | | | | | | | | | | | | | | | | | | |
| 552 | | | | | | X | | | | | | | | | | | | | | | | | | | |
| 553 | | X | | | | X | | | | | | | | | | | | | | | | | | | |
| 554 | | X | | | | X | | | | | | | | | | | | | | | | | | | |
| 555 | | | | | | X | | | | | | | | | | | | | | | | | | | |
| 556 | | X | | | | X | | | | | | | | | | | | | | | | | | | |
| 557 | | | | | | X | | | | | | | | | | | | | | | | | | | |
| 558 | | | | | | X | | | | | | | | | | | | | | | | | | | |
| 559 | | | | | | X | | | | | | | | | | | | | | | | | | | |
| 560 | | | | | | X | | | | | | | | | | | | | | | | | | | |
| 561 | | X | | | | X | | | | | | | | | | | | | | | | | | | |
| 562 | | X | | | | X | | | | | | | | | | | | | | | | | | | |
| 563 | | | | | | X | | | | | | | | | | | | | | | | | | | |
| 564 | | X | | | | X | | | | | | | | | | | | | | | | | | | |
| 565 | | X | | | | X | | | | | | | | | | | | | | | | | | | |
| 566 | | | | | | | | | | | | | | | | | | | | | | | | | |
| 567 | | | | | | X | | | | | | | | | | | | | | | | | | | |
| 568 | | X | | | | X | | | | | | | | | | | | | | | | | | | |
| 569 | | | | | | X | | | | | | | | | | | | | | | | | | | |
| 570 | | X | | | | X | | | | | | | | | | | | | | | | | | | |
| 571 | | | | | | X | | | | | | | | | | | | | | | | | | | |
| 572 | | | | | | X | | | | | | | | | | | | | | | | | | | |
| 573 | | | | | X | | | | | | | | | | | | | | | | | | | | |
| 574 | | | | | | X | | | | | | | | | | | | | | | | | | | |
| 575 | | X | | X | | X | | | | | | | | | | | | | | | | | | | |
| 576 | | | | | X | | | | | | | | | | | | | | | | | | | | |
| 577 | | | | | | | | | | | | | | | | | | | | | | | | | |
| 578 | | | | | | | | | | | | | | | | | | | | | | | | | |
| 579 | | X | | | | X | | | | | | | | | | | | | | | | | | | |
| 580 | | | | | | X | | | | | | | | | | | | | | | | | | | |
| 581 | | X | | | | X | | | | | | | | | | | | | | | | | | | |
| 582 | | X | | | | X | | | | X | | | | | | | | | | | | | | | |
| 583 | | | | | | X | | | | | | | | | | | | | | | | | | | |
| 584 | | | | | | | | | | | | | | | | | | | | | | | | | |
| 585 | | | | | | X | | | | | | | | | | | | | | | | | | | |
| 586 | | | | | | X | | | | | | | | | | | | | | | | | | | |
| 587 | | | | | | X | | | | | | | | | | | | | | | | | | | |
| 588 | | | | | | X | | | | | | | | | | | | | | | | | | | |
| 589 | | | | | | | | | | X | | | | | | | | | | | | | | | |
| 590 | | | | | | X | | | | | | | | | | | | | | | | | | | |
| 591 | | | | | | X | | | | | | | | | | | | | | | | | | | |
| 592 | | X | | | | X | | | | | | | | | | | | | | | | | | | |
| 593 | | | | | | | | | | X | | | | | | | | | | | | | | | |
| 594 | | | | | | | | | | X | | | | | | | | | | | | | | | |
| 595 | | | | | | | | | | X | | | | | | | | | | | | | | | |
| 596 | | | | | | | | | | X | | | | | | | | | | | | | | | |
| 597 | | | | | | X | | | | | | | | | | | | | | | | | | | |
| 598 | | X | | | | X | | | | X | | | | | | | | | | | | | | | |
| 599 | | X | | | | X | | | | X | | | | | | | | | | | | | | | |
| 600 | | | | | | | | | | X | | | | | | | | | | | | | | | |
| 601 | | | | | | X | | | | | | | | | | | | | | | | | | | |
| 602 | | | | | | X | | | | | | | | | | | | | | | | | | | |
| 603 | | | | | | X | | | | | | | | | | | | | | | | | | | |
| 604 | | | | | | X | | | | | | | | | | | | | | | | | | | |
| 605 | | | | | | X | | | | | | | | | | | | | | | | | | | |

| ID | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
|---|---|---|---|---|---|---|---|---|---|---|---|---|---|---|---|---|---|---|---|---|---|---|---|---|---|---|
| 606 | | | | X | | | | | | | X | | | | | X | | | | | | | | X | X | |
| 607 | | | | X | | | | | | | X | | | | | X | | | | | | | | X | X | |
| 608 | | | | | | | | | | | X | | | | | X | | | | | | | | X | X | |
| 609 | | | | | | | | | | | X | | | | | X | | | | | | | | X | X | |
| 610 | | | | X | | | | | X | X | X | | | | | X | | | | | | | | X | X | |
| 611 | | | | | | | | | | | X | | | | | X | | | | | | | | X | X | |
| 612 | | | | | | | | | | | X | | | | | X | | | | | | | | | X | |
| 613 | | | | | | | | | | | X | | | | | X | | | | | | | | X | X | |
| 614 | | | | | | | | | | | X | | | | | X | | | | | | | | X | X | |
| 615 | | | | | | | | | | | X | | | | | X | | | | | | | | X | X | |
| 616 | | | | X | | | | | | | | | | | | | | | | | | | | | | |
| 617 | | | | X | | | | | | | | | | | | | | | | | | | | | | |
| 618 | | | | | | | | | | | X | | | | | X | | | | | | | | X | X | |
| 619 | | | | | | | | | | | | | | | | | | | | | | | | | | |
| 620 | | | | | | | | | | | X | | | | | X | | | | | | | | X | X | |
| 621 | | | | | | | | | | | | | | | | | | | | | | | | | | |
| 622 | | | | | | | | | | | | | | | | | | | | | | | | | | |
| 623 | | | | X | | | | | | | | | | | | | | | | | | | | | | |
| 624 | | | | | | | | | | | | | | | | | | | | | | | | | | |
| 625 | | | | | | | | | | | X | | | | | X | | | | | | | | X | X | |
| 626 | | | | | | | | | | | | | | | | | | | | | | | | X | X | |
| 627 | | | | | | | | | | | | | | | | | | | | | | | | | | |
| 628 | | | | X | | | | | | | | | | | | | | | | | | | | | | |
| 629 | | | | | | | | | | | | | | | | | | | | | | | | | | |
| 630 | | | | | | | | | | | X | | | | | X | | | | | | | | X | X | |
| 631 | | | | | | | | | | | X | | | | | X | | | | | | | | X | X | |
| 632 | | | | | | | | | | | X | | | | | X | | | | | | | | X | X | |
| 633 | | | | | | | | | | | X | | | | | X | | | | | | | | X | X | |
| 634 | | | | | | | | | | | X | | | | | X | | | | | | | | X | X | |
| 635 | | | | X | | | | | | | | | | | | | | | | | | | | | | |
| 636 | | | | X | | | | | | | | | | | | | | | | | | | | | | |
| 637 | | | | | | | | | | | X | | | | | X | | | | | | | | X | X | |
| 638 | | | | | | | | | | | X | | | | | X | | | | | | | | X | X | |
| 639 | | | | X | | | | | | | X | | | | | X | | | | | | | | X | X | |
| 640 | | | | | | | | | | | X | | | | | X | | | | | | | | X | X | |
| 641 | | | | | | | | | | | | | | | | | | | | | | | | | | |
| 642 | | | | | | | | | | | X | | | | | X | | | | | | | | X | X | |
| 643 | | | | | | | | | | | X | | | | | X | | | | | | | | X | X | |
| 644 | | | | | | | | | | | X | | | | | X | | | | | | | | X | X | |
| 645 | | | | | | | | | | | | | | | | | | | | | | | | | | |
| 646 | | | | | | | | | | | | | | | | | | | | | | | | | | |
| 647 | | | | | | | | | | | | | | | | | | | | | | | | | | |
| 648 | | | | X | | | | | | | X | | | | | X | | | | | | | | X | X | |
| 649 | | | | | | | | | | | | | | | | | | | | | | | | | | |
| 650 | | | | | | | | | | | | | | | | | | | | | | | | | | |
| 651 | | | | X | | | | | | | X | | | | | X | | | | | | | | X | X | |
| 652 | | | | | | | | | | | X | | | | | X | | | | | | | | X | X | |
| 653 | | | | | | | | | | | | | | | | | | | | | | | | | | |
| 654 | | | | | | | | | | | | | | | | | | | | | | | | | | |
| 655 | | | | | | | | | | | X | | | | | X | | | | | | | | X | X | |
| 656 | | | | | | | | | | | X | | | | | X | | | | | | | | X | X | |
| 657 | | | | | | | | | | | | | | | | | | | | | | | | | | |
| 658 | X | | | | | | | | | | | | | | | | | | | | | | | | | |
| 659 | | | | | | | | | | | | | | | | | | | | | | | | | | |
| 660 | X | | | | | | | | | | | | | | | | | | | | | | | | | |
| 661 | X | | | | | | | | | | | | | | | | | | | | | | | | | |
| 662 | | | | | | | | | | | | | | | | | | | | | | | | | | |
| 663 | | | | | | | | | X | | | | | | | X | | | | X | | | | X | | X |
| 664 | | | | | | | | | | | | | | | | | | | | | | | | | | |
| 665 | | | | | | | | | | | | | | | | | | | | | | | | | | |
| 666 | | | | | | | | | | | | | | | | | | | | | | | | | | |
| 667 | | | | | | | | | | | | | | | | | | | | | | | | | | |
| 668 | | | | | | | | | | | | | | | | | | | | | | | | | | |
| 669 | | | | | | | | | | | | | | | | | | | | | | | | | | |
| 670 | | | | | | | | | | | | | | | | | | | | | | | | | | |
| 671 | | | | | | | | | | | | | | | | | | | | | | | | | | |
| 672 | | | | | | | | | | | | | | | | | | | | | | | | | | |
| 673 | | | | | | | | | | | | | | | | | | | | | | | | | | |
| 674 | | | | | | | | | | | | | | | | | | | | | | | | | | |
| 675 | | | | | | | | | | | | | | | | | | | | | | | | | | |
| 676 | | | | | | | | | | | | | | | | | | | | | | | | | | |
| 677 | | | | | | | | | | | | | | | | | | | | | | | | | | |
| 678 | | | | | | | | | | | | | | | | | | | | | | | | | | |
| 679 | | | | | | | | | | | | | | | | | | | | | | | | | | |
| 680 | | | | | | | | | | | | | | | | | | | | | | | | | | |
| 681 | | | | | | | | | | | | | | | | | | | | | | | | | | |
| 682 | | | | | | | | | | | | | | | | | | | | | | | | | | |
| 683 | | | | | | | | | | | | | | | | | | | | | | | | | | |
| 684 | | | | | | | | | | | | | | | | | | | | | | | | | | |
| 685 | | | | | | | | | | | | | | | | | | | | | | | | | | |
| 686 | | | | | | | | | | | | | | | | | | | | | | | | | | |
| 687 | | | | | | | | | | | | | | | | | | | | | | | | | | |
| 688 | | | | | | | | | | | | | | | | | | | | | | | | | | |
| 689 | | | | | | | | | | | | | | | | | | | | | | | | | | |
| 690 | | | | | | | | | | | | | | | | | | | | | | | | | | |
| 691 | | | | | | | | | | | | | | | | | | | | | | | | | | |
| 692 | | | | | | | | | | | | | | | | | | | | | | | | | | |
| 693 | | | | | | | | | | | | | | | | | | | | | | | | | | |
| 694 | | | | | | | | | | | | | | | | | | | | | | | | | | |
| 695 | | | | | | | | | | | | | | | | | | | | | | | | | | |
| 696 | | | | | | | | | | | | | | | | | | | | | | | | | | |
| 697 | | | | | | | | | | | | | | | | | | | | | | | | | | |
| 698 | | | | | | | | | | | | | | | | | | | | | | | | | | |
| 699 | | | | | | | | | | | | | | | | | | | | | | | | | | |
| 700 | | | | | | | | | | | | | | | | | | | | | | | | | | |
| 701 | | | | | | | | | | | | | | | | | | | | | | | | | | |
| 702 | | | | | | | | | | | | | | | | | | | | | | | | | | |
| 703 | | | | | | | | | | | | | | | | | | | | | | | | | | |
| 704 | | | | | | | | | | | | | | | | | | | | | | | | | | |
| 705 | | | | | | | | | | | | | | | | | | | | | | | | | | |
| 706 | | | | | | | | | | | | | | | | | | | | | | | | | | |
| 707 | | | | | | | | | | | | | | | | | | | | | | | | | | |
| 708 | | | | | | | | | | | | | | | | | | | | | | | | | | |
| 709 | | | | | | | | | | | | | | | | | | | | | | | | | | |
| 710 | | | | | | | | | | | | | | | | | | | | | | | | | | |
| 711 | | | | | | | | | | | | | | | | | | | | | | | | | | |
| 712 | | | | | | | | | | | | | | | | | | | | | | | | | | |
| 713 | | | | | | | | | | | | | | | | | | | | | | | | | | |
| 714 | | | | | | | | | | | | | | | | | | | | | | | | | | |
| 715 | | | | | | | | | | | | | | | | | | | | | | | | | | |
| 716 | | | | | | | | | | | | | | | | | | | | | | | | | | |
| 717 | | | | | | | | | | | | | | | | | | | | | | | | | | |
| 718 | | | | | | | | | | | | | | | | | | | | | | | | | | |
| 719 | | | | | | | | | | | | | | | | | | | | | | | | | | |
| 720 | | | | | | | | | | | | | | | | | | | | | | | | | | |
| 721 | | | | | | | | | | | | | | | | | | | | | | | | | | |
| 722 | | | | | | | | | | | | | | | | | | | | | | | | | | |
| 723 | | | | | | | | | | | | | | | | | | | | | | | | | | |
| 724 | | | | | | | | | | | | | | | | | | | | | | | | | | |
| 725 | | | | | | | | | | | | | | | | | | | | | | | | | | |
| 726 | | | | | | | | | | | | | | | | | | | | | | | | | | |
| 727 | | | | | | | | | | | | | | | | | | | | | | | | | | |
| 728 | | | | | | | | | | | | | | | | | | | | | | | | | | |
| 729 | | | | | | | | | | | | | | | | | | | | | | | | | | |
| 730 | | | | | | | | | | | | | | | | | | | | | | | | | | |
| 731 | | | | | | | | | | | | | | | | | | | | | | | | | | |
| 732 | | | | | | | | | | | | | | | | | | | | | | | | | | |
| 733 | | | | | | | | | | | | | | | | | | | | | | | | | | |
| 734 | | | | | | | | | | | | | | | | | | | | | | | | | | |
| 735 | | | | | | | | | | | | | | | | | | | | | | | | | | |
| 736 | | | | | | | | | | | | | | | | | | | | | | | | | | |
| 737 | | | | | | | | | | | | | | | | | | | | | | | | | | |
| 738 | | | | | | | | | | | | | | | | | | | | | | | | | | |
| 739 | | | | | | | | | | | | | | | | | | | | | | | | | | |
| 740 | | | | | | | | | | | | | | | | | | | | | | | | | | |
| 741 | | | | | | | | | | | | | | | | | | | | | | | | | | |
| 742 | | | | | | | | | | | | | | | | | | | | | | | | | | |
| 743 | | | | | | | | | | | | | | | | | | | | | | | | | | |
| 744 | | | | | | | | | | | | | | | | | | | | | | | | | | |
| 745 | | | | | | | | | | | | | | | | | | | | | | | | | | |
| 746 | | | | | | | | | | | | | | | | | | | | | | | | | | |
| 747 | | | | | | | | | | | | | | | | | | | | | | | | | | |
| 748 | | | | | | | | | | | | | | | | | | | | | | | | | | |
| 749 | | | | | | | | | | | | | | | | | | | | | | | | | | |
| 750 | | | | | | | | | | | | | | | | | | | | | | | | | | |
| 751 | | | | | | | | | | | | | | | | | | | | | | | | | | |
| 752 | | | | | | | | | | | | | | | | | | | | | | | | | | |
| 753 | | | | | | | | | | | | | | | | | | | | | | | | | | |
| 754 | | | | | | | | | | | | | | | | | | | | | | | | | | |
| 755 | | | | | | | | | | | | | | | | | | | | | | | | | | |
| 756 | | | | | | | | | | | | | | | | | | | | | | | | | | |
| 757 | | | | | | | | | | | | | | | | | | | | | | | | | | |
| 758 | | | | | | | | | | | | | | | | | | | | | | | | | | |
| 759 | | | | | | | | | | | | | | | | | | | | | | | | | | |
| 760 | | | | | | | | | | | | | | | | | | | | | | | | X | | |
| 761 | | | | | | | | | | | | | | | | | | | | | | | | | | |
| 762 | | | | | | | | | | | | | | | | | | | | | | | | X | | |
| 763 | | | | | | | | | | | | | | | | | | | | | | | | | | |
| 764 | | | | | | | | | | | | | | | | | | | | | X | X | | | | |
| 765 | | | | | | | | | | | | | | | | | | | | | X | X | | | | |
| 766 | | | | | | | | | | | | | | | | | | | | | | | | | | |
| 767 | | | | | | | | | | | | | | | | | | X | | | X | X | | | | |
| 768 | | | | | | | | | | | | | | | | | | | | | | | | | | |
| 769 | | | | | | | | | | | | | | | | | | | | | | | | | | |
| 770 | | | | | | | | | | | | | | | | | | | | | | | | | | |
| 771 | | | | | | | | | | | | | | | | | | | | | | | | | | |
| 772 | | | | | | | | | | | | | | | | | | | | | | | | | | |
| 773 | | | | | | | | | | | | | | | | | | | | | | | | | | |
| 774 | | | X | | | | | | | | | | | | | | | X | | | X | X | | | | |
| 775 | | X | | | | | | X | | | | | | | | X | | | | | | | | X | | X |
| 776 | | X | | | | | | X | | | | | | | | X | | | | | | | | X | | X |
| 777 | | | | | | | | | | | | | | | | | | | | | | | | X | | |
| 778 | | | | | | | | | | | X | | | | | X | | | | | | | | X | | X |
| 779 | | | | | | | | | | | X | | | | | X | | | | | | | | X | | X |
| 780 | | | | | | | | | | | | | | | | | | | | | | | | X | | X |
| 781 | | | | | | | | | | | | | | | | | | | | | | | | | | |
| 782 | | | | | | | | | | | | | | | | | | | | | | | | | | |
| 783 | | X | | | | | | | | | | | | | X | X | | | | | | | X | X | X | X |
| 784 | | X | | | | | | | | | | | | | X | X | | | | | | | X | X | X | X |
| 785 | | | | | | | | | | | | | | | | | X | | | | X | X | | | | |
| 786 | | | | | | | | | | | | | | | | | | | | | | | | X | | X |
| 787 | | | | | | | | | | | | | | | | | | | | | | | | X | | X |
| 788 | | | | | | | | | | | X | | | | | X | | | | | | | | X | X | |
| 789 | | | | | | | | | | | | | | | | | | | | | | | | | | |
| 790 | | | | | | | | | | | | | | | | | | | | | X | X | | | | |
| 791 | | | | | | | | | | | X | | | | | X | | | | | | | | X | X | |
| 792 | | | | | | | | | | | | | | | | | | | | | | | | | | |
| 793 | | | | | | | | | | | | | | | | | | | | | | | | | | |
| 794 | | | | | | | | | | | | | | | | | | | | | | | | | | |
| 795 | | | | | | | | | | | | | | | | | | | | | | | | | | |
| 796 | | | | | | | | | | | | | | | | | | | | | | | | | | |
| 797 | | | | | | | | | | | | | X | | | | | | | | | | X | | X | |
| 798 | | | | | | | | | | | | | | | | | | | | | | | | | | |
| 799 | | | | | | | | | | | | X | | | | | X | | | | | | | | X | X |

| ID | 27 | 28 | 29 | 30 | 31 | 32 | 33 | 34 | 35 | 36 | 37 | 38 | 39 | 40 | 41 | 42 | 43 | 44 | 45 | 46 | 47 | 48 | 49 | 50 | 51 |
|---|---|---|---|---|---|---|---|---|---|---|---|---|---|---|---|---|---|---|---|---|---|---|---|---|---|
| 606 | | | | | | X | | | | | | | | | | | | | | | | | | | |
| 607 | | | | | | X | | | | | | | | | | | | | | | | | | | |
| 608 | | | | | | X | | | | | | | | | | | | | | | | | | | |
| 609 | | | | | | X | | | | | | | | | | | | | | | | | | | |
| 610 | | | | | | X | | | | | | | | | | | | | | | | | | | |
| 611 | | | | | | X | | | | | | | | | | | | | | | | | | | |
| 612 | | | | | | X | | | | | | | | | | | | | | | | | | | |
| 613 | | | | | | X | | | | | | | | | | | | | | | | | | | |
| 614 | | X | | | | X | | | | | | | | | | | | | | | | | | | |
| 615 | | X | | | | X | | | | | | | | | | | | | | | | | | | |
| 616 | | | | | | | | | | | | | | | | | | | | | | | | | |
| 617 | | | | | | | | | | | | | | | | | | | | | | | | | |
| 618 | | | | | | X | | | | | | | | | | | | | | | | | | | |
| 619 | | | | | | | | | | X | | | | | | | | | | | | | | | |
| 620 | | | | | | X | | | | | | | | | | | | | | | | | | | |
| 621 | | | | | | | | | | X | | | | | | | | | | | | | | | |
| 622 | | | | | | | | | | X | | | | | | | | | | | | | | | |
| 623 | | | | | | | | | | | | | | | | | | | | | | | | | |
| 624 | | | | | | | | | | X | | | | | | | | | | | | | | | |
| 625 | | X | | | | X | | | | | | | | | | | | | | | | | | | |
| 626 | | | | | | | | | | | | | | | | | | | | | | | | | |
| 627 | | | | | | | | | | X | | | | | | | | | | | | | | | |
| 628 | | | | | | | | | | | | | | | | | | | | | | | | | |
| 629 | | | | | | | | | | X | | | | | | | | | | | | | | | |
| 630 | | | | | | X | | | | | | | | | | | | | | | | | | | |
| 631 | | X | | | | X | | | | | | | | | | | | | | | | | | | |
| 632 | | X | | | | X | | | | | | | | | | | | | | | | | | | |
| 633 | | X | | | | X | | | | | | | | | | | | | | | | | | | |
| 634 | | X | | | | X | | | | X | | | | | | | | | | | | | | | |
| 635 | | | | | | | | | | X | | | | | | | | | | | | | | | |
| 636 | | | | | | | | | | X | | | | | | | | | | | | | | | |
| 637 | | X | | | | X | | | | | | | | | | | | | | | | | | | |
| 638 | | X | | | | X | | | | | | | | | | | | | | | | | | | |
| 639 | | X | | | | X | | | | X | | | | | | | | | | | | | | | |
| 640 | | X | | | | X | | | | | | | | | | | | | | | | | | | |
| 641 | | | | | | | | | | X | | | | | | | | | | | | | | | |
| 642 | | X | | | | X | | | | | | | | | | | | | | | | | | | |
| 643 | | X | | | | X | | | | | | | | | | | | | | | | | | | |
| 644 | | X | | | | X | | | | | | | | | | | | | | | | | | | |
| 645 | | | | | | | | | | X | | | | | | | | | | | | | | | |
| 646 | | | | | | | | | | X | | | | | | | | | | | | | | | |
| 647 | | | | | | | | | | X | | | | | | | | | | | | | | | |
| 648 | | X | | | | X | | | | X | | | | | | | | | | | | | | | |
| 649 | | | | | | | | | | X | | | | | | | | | | | | | | | |
| 650 | | | | | | | | | | X | | | | | | | | | | | | | | | |
| 651 | | X | | | | X | | | | X | | | | | | | | | | | | | | | |
| 652 | | X | | | | X | | | | X | | | | | | | | | | | | | | | |
| 653 | | | | | | | | | | X | | | | | | | | | | | | | | | |
| 654 | | | | | | | | | | X | | | | | | | | | | | | | | | |
| 655 | | | | | | X | | | | | | | | | | | | | | | | | | | |
| 656 | | | | | | X | | | | | | | | | | | | | | | | | | | |
| 657 | | | | | | | | | | X | | | | | | | | | | | | | | | |
| 658 | | | | | | | | | | | | | | | | | | | | | | | | | |
| 659 | | | | | | | | | | | | | | | | | | | | | | | | | X |
| 660 | | | | | | | | | | | | | | | | | | | | | | | | | |
| 661 | | | | | | | | | | | | | | | | | | | | | | | | | |
| 662 | | | | | | | | | | X | | | | | | | | | | | | | | | |
| 663 | | | | | | | | | | | | | | | | | | | | | | | | | |
| 664 | | | | | | | | | | X | | | | | | | | | | | | | | | |
| 665 | | | | | | | | | | X | | | | | | | | | | | | | | | |
| 666 | | | | | | | | | | X | | | | | | | | | | | | | | | |
| 667 | | | | | | | | | | X | | | | | | | | | | | | | | | |
| 668 | | | | | | | | | | X | | | | | | | | | | | | | | | |
| 669 | | | | | | | | | | X | | | | | | | | | | | | | | | |
| 670 | | | | | | | | | | X | | | | | | | | | | | | | | | |
| 671 | | | | | | | | | | X | | | | | | | | | | | | | | | |
| 672 | | | | | | | | | | X | | | | | | | | | | | | | | | |
| 673 | | | | | | | | | | X | | | | | | | | | | | | | | | |
| 674 | | | | | | | | | | X | | | | | | | | | | | | | | | |
| 675 | | | | | | | | | | X | | | | | | | | | | | | | | | |
| 676 | | | | | | | | | | X | | | | | | | | | | | | | | | |
| 677 | | | | | | | | | | X | | | | | | | | | | | | | | | |
| 678 | | | | | | | | | | X | | | | | | | | | | | | | | | |
| 679 | | | | | | | | | | X | | | | | | | | | | | | | | | |
| 680 | | | | | | | | | | X | | | | | | | | | | | | | | | |
| 681 | | | | | | | | | | X | | | | | | | | | | | | | | | |
| 682 | | | | | | | | | | X | | | | | | | | | | | | | | | |
| 683 | | | | | | | | | | X | | | | | | | | | | | | | | | |
| 684 | | | | | | | | | | X | | | | | | | | | | | | | | | |
| 685 | | | | | | | | | | X | | | | | | | | | | | | | | | |
| 686 | | | | | | | | | | X | | | | | | | | | | | | | | | |
| 687 | | | | | | | | | | X | | | | | | | | | | | | | | | |
| 688 | | | | | | | | | | X | | | | | | | | | | | | | | | |
| 689 | | | | | | | | | | X | | | | | | | | | | | | | | | |
| 690 | | | | | | | | | | X | | | | | | | | | | | | | | | |
| 691 | | | | | | | | | | X | | | | | | | | | | | | | | | |
| 692 | | | | | | | | | | X | | | | | | | | | | | | | | | |
| 693 | | | | | | | | | | X | | | | | | | | | | | | | | | |
| 694 | | | | | | | | | | X | | | | | | | | | | | | | | | |
| 695 | | | | | | | | | | X | | | | | | | | | | | | | | | |
| 696 | | | | | | | | | | X | | | | | | | | | | | | | | | |
| 697 | | | | | | | | | | X | | | | | | | | | | | | | | | |
| 698 | | | | | | | | | | X | | | | | | | | | | | | | | | |
| 699 | | | | | | | | | | X | | | | | | | | | | | | | | | |
| 700 | | | | | | | | | | X | | | | | | | | | | | | | | | |
| 701 | | | | | | | | | | X | | | | | | | | | | | | | | | |
| 702 | | | | | | | | | | X | | | | | | | | | | | | | | | |
| 703 | | | | | | | | | | X | | | | | | | | | | | | | | | |
| 704 | | | | | | | | | | X | | | | | | | | | | | | | | | |
| 705 | | | | | | | | | | X | | | | | | | | | | | | | | | |
| 706 | | | | | | | | | | X | | | | | | | | | | | | | | | |
| 707 | | | | | | | | | | X | | | | | | | | | | | | | | | |
| 708 | | | | | | | | | | X | | | | | | | | | | | | | | | |
| 709 | | | | | | | | | | X | | | | | | | | | | | | | | | |
| 710 | | | | | | | | | | X | | | | | | | | | | | | | | | |
| 711 | | | | | | | | | | X | | | | | | | | | | | | | | | |
| 712 | | | | | | | | | | X | | | | | | | | | | | | | | | |
| 713 | | | | | | | | | | X | | | | | | | | | | | | | | | |
| 714 | | | | | | | | | | X | | | | | | | | | | | | | | | |
| 715 | | | | | | | | | | X | | | | | | | | | | | | | | | |
| 716 | | | | | | | | | | X | | | | | | | | | | | | | | | |
| 717 | | | | | | | | | | X | | | | | | | | | | | | | | | |
| 718 | | | | | | | | | | X | | | | | | | | | | | | | | | |
| 719 | | | | | | | | | | X | | | | | | | | | | | | | | | |
| 720 | | | | | | | | | | X | | | | | | | | | | | | | | | |
| 721 | | | | | | | | | | X | | | | | | | | | | | | | | | |
| 722 | | | | | | | | | | X | | | | | | | | | | | | | | | |
| 723 | | | | | | | | | | X | | | | | | | | | | | | | | | |
| 724 | | | | | | | | | | X | | | | | | | | | | | | | | | |
| 725 | | | | | | | | | | X | | | | | | | | | | | | | | | |
| 726 | | | | | | | | | | X | | | | | | | | | | | | | | | |
| 727 | | | | | | | | | | X | | | | | | | | | | | | | | | |
| 728 | | | | | | | | | | X | | | | | | | | | | | | | | | |
| 729 | | | | | | | | | | X | | | | | | | | | | | | | | | |
| 730 | | | | | | | | | | X | | | | | | | | | | | | | | | |
| 731 | | | | | | | | | | X | | | | | | | | | | | | | | | |
| 732 | | | | | | | | | | X | | | | | | | | | | | | | | | |
| 733 | | | | | | | | | | X | | | | | | | | | | | | | | | |
| 734 | | | | | | | | | | X | | | | | | | | | | | | | | | |
| 735 | | | | | | | | | | X | | | | | | | | | | | | | | | |
| 736 | | | | | | | | | | X | | | | | | | | | | | | | | | |
| 737 | | | | | | | | | | X | | | | | | | | | | | | | | | |
| 738 | | | | | | | | | | X | | | | | | | | | | | | | | | |
| 739 | | | | | | | | | | X | | | | | | | | | | | | | | | |
| 740 | | | | | | | | | | X | | | | | | | | | | | | | | | |
| 741 | | | | | | | | | | X | | | | | | | | | | | | | | | |
| 742 | | | | | | | | | | X | | | | | | | | | | | | | | | |
| 743 | | | | | | | | | | X | | | | | | | | | | | | | | | |
| 744 | | | | | | | | | | X | | | | | | | | | | | | | | | |
| 745 | | | | | | | | | | X | | | | | | | | | | | | | | | |
| 746 | | | | | | | | | | X | | | | | | | | | | | | | | | |
| 747 | | | | | | | | | | X | | | | | | | | | | | | | | | |
| 748 | | | | | | | | | | X | | | | | | | | | | | | | | | |
| 749 | | | | | | | | | | X | | | | | | | | | | | | | | | |
| 750 | | | | | | | | | | X | | | | | | | | | | | | | | | |
| 751 | | | | | | | | | | X | | | | | | | | | | | | | | | |
| 752 | | | | | | | | | | X | | | | | | | | | | | | | | | |
| 753 | | | | | | | | | | X | | | | | | | | | | | | | | | |
| 754 | | | | | | | | | | X | | | | | | | | | | | | | | | |
| 755 | | | | | | | | | | X | | | | | | | | | | | | | | | |
| 756 | | | | | | | | | | X | | | | | | | | | | | | | | | |
| 757 | | | | | | | | | | X | | | | | | | | | | | | | | | |
| 758 | | | | | | | | | | X | | | | | | | | | | | | | | | |
| 759 | | | | | | | | | | X | | | | | | | | | | | | | | | |
| 760 | | | X | | | | | X | | | | | | | | | | X | X | X | | | | | |
| 761 | | | X | | | | | X | | | | | | | | | | X | X | X | | | | | |
| 762 | | | X | | | | | X | | | | | | | | | | X | X | X | | | | | |
| 763 | X | | | | | | | | | | | | | | | | | | | | | | | | |
| 764 | | | | | | | | | | | | | | | | X | | | | | | | | | |
| 765 | | | | | | | | | | | | | | | | X | | | | | | | | | |
| 766 | | | X | | | | | | X | | | | | | | | | X | X | X | | | | | |
| 767 | | | | | | | | | | | X | | | | | | | | | | | | | | |
| 768 | | | | | | | | | | X | | | | | | | | | | | | | | | |
| 769 | | | | | | | | | | X | | | | | | | | | | | | | | | |
| 770 | | | | | | | | | | X | | | | | | | | | | | | | | | |
| 771 | | | | | | | | | | X | | | | | | | | | | | | | | | |
| 772 | | | | | | | | | | X | | | | | | | | | | | | | | | |
| 773 | | | | | | | | | | X | | | | | | | | | | | | | | | |
| 774 | | | | | | | | | | | X | | | | | | | | | | | | | X | |
| 775 | | X | | | | | | | | | | | | | | | | | | | | | | | |
| 776 | | X | | | | | | | | | | | | | | | | | | | | | | | |
| 777 | | | X | | | | | | X | | | | | | | | | | X | X | X | | | | |
| 778 | | | | | | X | | | | | | | | | | | | | | | | | | | |
| 779 | | | | | | X | | | | | | | | | | | | | | | | | | | |
| 780 | | | | | | | | | | X | | | | | | | | | | | | | | | |
| 781 | | | | | | | | | | X | | | | | | | | | | | | | | | |
| 782 | | | | | | | | | | X | | | | | | | | | | | | | | | |
| 783 | | X | | | | | | | | | | | | | | | | | | | | | | | |
| 784 | | X | | | | | | | | | | | | | | | | | | | | | | | |
| 785 | | | | | | | | | | | | | | | | | | | | | | | | | |
| 786 | | | | | | | | | | X | | | | | | | | | | | | | | | |
| 787 | | | | | | | | | | X | | | | | | | | | | | | | | | |
| 788 | | | | | | X | | | | | | | | | | | | | | | | | | | |
| 789 | | | | | | | | | | X | | | | | | | | | | | | | | | |
| 790 | | | | | | | | | | | | | | | | | | | | | | | | | |
| 791 | | | | | | X | | | | | | | | | | | | | | | | | | | |
| 792 | | | | | | | | | | X | | | | | | | | | | | | | | | |
| 793 | | | | | | | | | | X | | | | | | | | | | | | | | | |
| 794 | | | | | | | | | | X | | | | | | | | | | | | | | | |
| 795 | | | | | | | | | | X | | | | | | | | | | | | | | | |
| 796 | | | | | | | | | | | X | | | | | | | | | | | | | | |
| 797 | | | | X | | | | | | | | | | | | | X | | | | X | | | | |
| 798 | | | | | | | | | | | X | | | | | | | | | | | | | | |
| 799 | | | X | | | | X | | | | | | | | | | | | | | | | | | |

TABLE 3

| Key | Description |
|---|---|
| 1 | CRISPR associated protein Cas2_CRISPR associated protein Cas2 |
| 2 | CRISPR associated protein Cas6_CRISPR associated protein Cas6 |
| 3 | CRISPR associated protein_CRISPR associated protein |
| 4 | CRISPR-associated endoribonuclease Cas6_CRISPR-associated |
| | endoribonuclease Cas6 |
| 5 | CRISPR-associated negative auto-regulator DevR/Csa2_CRISPR- |
| | associated negative auto-regulator DevR/Csa2 |
| 6 | CRISPR-associated protein (Cas_CRISPR-associated protein |
| | (Cas_APE2256) |
| 7 | CRISPR-associated protein (Cas_CRISPR-associated protein |
| | (Cas_Cas02710) |
| 8 | CRISPR-associated protein (Cas_CRISPR-associated protein |
| | (Cas_Cas5) |
| 9 | CRISPR-associated protein (Cas_CRISPR-associated protein |
| | (Cas_Cmr3) |
| 10 | CRISPR-associated protein (Cas_CRISPR-associated protein |
| | (Cas_Cmr5) |
| 11 | CRISPR-associated protein (Cas_CRISPR-associated protein |
| | (Cas_Csd1) |
| 12 | CRISPR-associated protein (Cas_CRISPR-associated protein |
| | (Cas_Csy2) |
| 13 | CRISPR-associated protein (Cas_CRISPR-associated protein |
| | (Cas_Csy4) |
| 14 | CRISPR-associated protein (Cas_CRISPR-associated protein |
| | (Cas_GSU0053) |
| 15 | CRISPR-associated protein (cas_CRISPR-associated protein |
| | (cas_TM1802) |
| 16 | CRISPR-associated protein Cas7_CRISPR-associated protein Cas7 |
| 17 | CRISPR-associated protein Cse1 (CRISPR_CRISPR-associated |
| | protein Cse1 (CRISPR_cse1) |
| 18 | CRISPR-associated protein Cse2 (CRISPR_CRISPR-associated |
| | protein Cse2 (CRISPR_cse2) |
| 19 | CRISPR-associated protein, family (Cas_CRISPR-associated |
| | protein, family (Cas_GSU0054) |
| 20 | CRISPR-associated protein_CRISPR-associated protein |
| 21 | CRISPR-associated_CRISPR-associated_helicase |
| 22 | CRISPR-associated_CRISPR-associated_protein |
| 23 | cas1-1_CRISPR-associated endonuclease Cas1 1 |
| 24 | cas1_CRISPR-associated endonuclease Cas1 |
| 25 | cas2_CRISPR-associated endonuclease Cas2 |
| 26 | cas2_CRISPR-associated endoribonuclease Cas2 |
| 27 | cas3_CRISPR-associated endonuclease/helicase Cas3 |
| 28 | cas3_CRISPR-associated nuclease/helicase Cas3 |
| 29 | cas3_CRISPR-associated nuclease/helicase Cas3 |
| | subtype I-F/YPEST |
| 30 | cas4-cas1_CRISPR-associated exonuclease |
| | Cas4/endonuclease Cas1 fusion |
| 31 | cas4_CRISPR-associated exonuclease Cas4 |
| 32 | cas5d_CRISPR pre-crRNA endoribonuclease Cas5d |
| 33 | cas6_CRISPR-associated endonuclease Cas6 |
| 34 | cas6f_CRISPR-associated endonuclease Cas6/Csy4 |
| 35 | cas6f_CRISPR-associated endonuclease Cas6f/Csy4 |
| 36 | cas9_CRISPR-associated endonuclease Cas9 |
| 37 | casA_CRISPR system Cascade subunit CasA |
| 38 | casB_CRISPR system Cascade subunit CasB |
| 39 | casE_CRISPR system Cascade subunit CasE |
| 40 | cmr5_CRISPR system Cmr subunit Cmr5 |
| 41 | cse1_CRISPR-associated protein CasA/Cse1 |
| 42 | cse2_CRISPR-associated protein Cse2 |
| 43 | csm3_CRISPR type III-associated RAMP protein Csm3 |
| 44 | csy1_CRISPR-associated protein Csy1 |
| 45 | csy2_CRISPR-associated protein Csy2 |
| 46 | csy3_CRISPR-associated protein Csy3 |
| 47 | devR_CRISPR-associated protein Cas7/Cst2/DevR |
| 48 | devS_CRISPR-associated protein Cas5 |
| 49 | devT_CRISPR-associated protein Cas8a1/Csx13 |
| 50 | ygbF_CRISPR-associated endoribonuclease Cas2 |
| 51 | ygbT_CRISPR-associated endonuclease Cas1 |

It is claimed:

1. A synthetic composition comprising a Cas endonuclease identified from the microbes given in Table 1, and a heterologous polynucleotide.

2. The synthetic composition of claim 1, wherein the Cas endonuclease is encoded by a polynucleotide sharing at least 95% identity with a sequence selected from the group consisting of SEQID NOs: 1-179.

3. The synthetic composition of claim 1, further comprising a target polynucleotide.

4. The synthetic composition of claim 1, further comprising a guide polynucleotide.

5. The synthetic composition of claim 1, wherein the heterologous polynucleotide is an expression element, a transgene, a donor DNA molecule, or a polynucleotide modification template.

6. The synthetic composition of claim 1, further comprising a deaminase.

7. The synthetic composition of claim 1, further comprising a heterologous nuclease.

8. The synthetic composition of claim 1, further comprising a eukaryotic cell.

9. The synthetic composition of claim 1, further comprising a prokaryotic cell.

10. A nucleotide construct comprising the polynucleotide of claim 2.

11. A method of introducing a targeted edit in a target polynucleotide, comprising providing to the target polynucleotide the synthetic composition of claim 1, incubating the target polynucleotide and synthetic composition under conditions suitable for forming a complex, and assessing the target polynucleotide for the presence of the targeted edit, wherein the targeted edit is selected from the group consisting of: the insertion of at least one nucleotide, the deletion of at least one nucleotide, the replacement of at least one nucleotide, the chemical alteration of at least one nucleotide, and any plurality and/or combination of the preceding.

12. A kit comprising the synthetic composition of claim 1.


SEQUENCE LISTING (428 sequences)

SEQ ID NO: 1 - DNA, 501 bp, unidentified
  source 1..501: mol_type = genomic DNA; note = Pseudarthrobacter chlorophenolicus
atgcgcactctcgttctgaatgctggatatgaaccgctggcggttattaccttccgccgggcgctggtgcttgtgcttacaggcaaggccagtgtggtggccgagggggacgaccccgtggtgggccccaccgacgtcctggggcgtccgtccgtgatcctcctcaaccgctacatccgcccccggtacaaccactccacggccgtcagccgtagaggcgtgctccgacgcgacgggcataaatgcgcctactgcgggaaagcggcgcacaccattgaccacgtgcatccgaaatcccggggcggggccgattcgtgggagaacctggtggccgcctgcctgcggtgcaacaacgtcaaaggcgaccacactcctgccgagatgggatggaccttgcggttcgtgccggaacctccgcacggaaccatctggcaaatcaaggagctggagaagcccacgccggcgtgggatcccttcctgcttccggaacgcgctgcttga

SEQ ID NO: 2 - DNA, 498 bp, Microbacterium sp.
  source 1..498: mol_type = genomic DNA
atgcgcacactggtcctgaatgccggatacgagccgctggcgatcgtgtcgttcaaacgagccctggtgctggtgatgaacgacaaggcgaccgtgatcgaacgggtcgaggaagaccccgtctggggcacgcacggcgtctacgatcgcccggcggtcatcatcctggcccgttacgtgcgggtcccgacgagcaggcgtgtgccggtcacccggcgcggtgtgctccgccgcgacaaccaccgctgcggatactgcgggaaggcggcatcgacgatcgatcacgtgctgccgcgctcgcgcgggggagcggactcctgggagaacctcgtcgcgtgctgcctgcgctgcaacaacgtcaagagtgatcgcacgccgcaggagatgcgctggcagctgcgcttcacccctcgaccgccgcacggcaccgcctggaccgtgcgcggaaccgaacgcagcgacccgcgatgggagccgtacctcgctctggccgcctga

SEQ ID NO: 3 - DNA, 498 bp, Microbacterium testaceum
  source 1..498: mol_type = genomic DNA
atgcgcactctggtgctcaatgcgggctacgagccgctcgccgtcgtgtcgttcaagcgggcgatcgtgctcgtgatgaacgagaaggccgtcgtcgtcgagagggtggacgacgatccggtgtggggggcacgcggggcgtatgaacgcccggcggtgatccttctgacgaagtatgtgcgggtgccgggcagccggcacatcccggtcacccggcgcggagtgctgcggcgcgacgctcaccggtgcgggtactgcggcaaggcggcgtccacgatcgatcacatcctgccgcgctcgcgcgggggagcggactcgtgggagaacctcgtcgcgtgctgcctgcgctgcaacaacgtcaagggggaccgcaccccgcaggagatgcactgggagctgcgggtcctgccccggccgccccgcggcgtgcagtggactgtgcgcggcacggagcgcaccgacccgtgctgggagccgtatctcgcgctcgccgcgtga

SEQ ID NO: 4 - DNA, 996 bp, Bacillus sp.
  source 1..996: mol_type = genomic DNA
atgagccataaattacaggtgggagaaatgaaagcatcctatttaacagataaggaaatatgggcacattttaatttcatcttttcgccgaaatcaaagaattcaactacatacaagtttgtcttaattaattccattctagaaaacttatataatgtcaatgacaagctggaattaccatattcaactcttttttcaagttttgccaagatatactggaaccttgttattcatcataacttaaatcaaattaatatgattgataagaaggccgaggttcaaaaaatcctccttgatgtccaggcaaaatatcagatcccgaaaacgctggtttttgataagctatcttatgatttacagatagagattattacaaaggtaatgaagagatgcaagatcaatgttatgggagccatttacggtgacactggctgcaccatttatgattttgataatgggaaggaacagctaaaacttaatagcactttttactcgttcatgcagcgatttcaacggtttttaaattatttaacgaattatcatctggcgttatttttagaaaaatttaatgaaaagggagataccactaatctgctattaaaggtagaaaatgtatctaaaagatcctcgcttgatcagttttatcaggtactctcatcattttataatgggaagtgtttttactgtgggaagaccatcaagaaagaaaacgcacatgtcgatcactttattccctggagttttgtccaagcggatcaactgtggaatttggtgattgcttgtagcgcatgtaatttgtctaaaagtgataagttggccaatgaaccgttcttagagacgatgattgatcgaaatgaatccttaatttccatcccagcacttagaattcgagaagacatgcaagtctattcaagtaaaaaattgaaagatctctatcatttttctgtagagaatgggtttacggatttttgggtgccaaagaaaatgattttatag

SEQ ID NO: 5 - DNA, 996 bp, Bacillus sp.; identical to SEQ ID NO: 4
  source 1..996: mol_type = genomic DNA

SEQ ID NO: 6 - DNA, 498 bp, Rathayibacter tritici
  source 1..498: mol_type = genomic DNA
atgcgcactctggttctcaacgccggctacgagccgctcgcggtggtgtcgttcaagcgggcactggtcctgctgatgacggagaaggcgaccgtgatcgccgcggacgagggcaatccggtgcacagcgcgcacggtgaccacgtgcggccctcggtgatcgtgctgacacgctacgtgcgcatcccgagcggccggagcgtgccggtgtcgcgccggggcgtgctccgccgggacaaccaccgctgcggctactgcggtcagcacgcggcgacgatcgaccacatccagcccaagtcgcgcgggggccgggacacgtgggagaacctcgtcgcctgctgccagcgctgcaacaacgtcaagagcgaccgcaccccgcacgagatgggatggcgtctgcgcaccgtcccccggatgccgcgcgccggctcctggctcgtgcgcggcgcggagcggacgcagcccgactgggacgactacctggctcccgccgcctga

SEQ ID NO: 7 - DNA, 498 bp, Microbacterium sp.
  source 1..498: mol_type = genomic DNA
atgcgcacactggtcctgaatgccggatacgagccgctggcgatcgtgtcattcaaacgagccctggtgctggtgatgaacgacaaggcgaccgtgatcgaacgggtcgaggaagaccccgtctggggcacgcacggcgtctacgatcgcccggcggtcatcatcctggcccgttacgtgcgggtcccgacgagcaggcgtgtgccggtcacccggcgcggtgtgctccgccgcgacaaccaccgctgcggatactgcgggaaggcggcatcgacgatcgatcacgtgctgccgcgctcgcgcgggggagcggactcctgggagaacctcgtcgcgtgctgcctgcgctgcaacaacgtcaagagtgatcgcacgccgcaggagatgcgctggcagctgcgcttcacccctcgaccgccgcacggcaccgcctggaccgtgcgcggaaccgaacgaagcgacccgcgatgggagccgtacctcgctctggccgcctga

SEQ ID NO: 8 - DNA, 501 bp, Rathayibacter tritici
  source 1..501: mol_type = genomic DNA
atgcgcacactcgtcctcaacgccggctacgagccgctcgccgtggtgtcgttcaagcgggcgctggtgctgctgatgagcgagaaggcgatgatcatcgtcgccgatgaggagcacccggtgcacggcgcgaacggcgactacgtgcggccctcggtgatcgtgctgacgcgctacgtccgcatcccgagctcgcgcagcgtgccggtctcgcgccgcggagtgctgcggcgcgacaatcaccgctgcggctactgcgggcagcacgccaacacgatcgaccacatccagccgaagtcgcggggcggccgcgactcctgggagaacctggtcgcctgctgccaccgctgcaacaacctcaagagcgaccggacgccgctcgagatgggctggcagctgcgctcggtgccgcggatgccgcgcgccggctcctggctcgtccgcggcgccgagcgcacccagcccgagtgggacgactacctgggcgcctccgccgcctga

SEQ ID NO: 9 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattttttgctaatggtcactttaccacaacgtataaatatggattaatgaaagctttaattgaaaacctatataacgttgacaatcggttagtactcacgtttgatcaagtatatttttcatttgctaaaatatattggaatttggttattcatcatgatttaaatcaattaaatacacccaatagacaatctggaattcaaaaagagttaaaggaatttcagttaatgcatggtgtccccaataaagtagtatttgatcgtttaccaagtaattttcaattgcagttagtagaaagaactaaaaaagtgggggcaaggtatgtagtaggggcgttatacggagatatggaagcttctatttatgagtttgacaaaagaacagaatacataaagtttaatagcagtatctatatttttttacaaaaatatagacaaattgttactcatttaaccaattatcatttggcgaaatttttagaaaagcataatgacaaaaataagttagaagatgtcttgttgaaagtagaaaatatttcaaaacgcgaatcgctgcatgcattctataacatactttggaaatatgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgagtatgctggaatgaatggatacgtagaaaactggtctccatag

SEQ ID NO: 10 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattttttgctaatggtcactttaccacaacgtataaatatggattaatgaaagctttaattgaaaacctatataacgttgacaatcggttagtactcacgtttgatcaagtatatttttcatttgctaaaatatattggaatttggttattcatcatgatttaaatcaattaaatacacccaatagacaatctggaattcaaaaagagttaaaggaatttcagttaatgcatggtgtccccaataaagtagtatttgatcgtttaccaagtaattttcaattgcagttagtagaaagaactaaaaaagtgggggcaaggtatgtagtaggggcgttatacggagatatggaagcttctatttatgagtttgacaaaagaacagaatacataaagtttaatagcagtatctatatttttttacaaaaatatagacaaattgttactcatttaaccaattatcatttggcgaaatttttagaaaagcataatgacaaaaataagttagaagatgtcttgttgaaagtagaaaatatttcaaaacgcgaatcgctgcatgcattctataacatactttggaaatatgaaacaaaagaatgtttttattgtgggaaagttctttctgataaacgtatcgctactcatgtagatcattttattccatggagttatatccaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgattatgctggaatgaatggatacgtagaaaactggtctccatag

SEQ ID NO: 11 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattctttgctaatggtcactttaccacaacgtataaatatggattaatgaaagctttaattgaaaacctatataacgttgacaatcggttagtactcacgtttgatcaaatatatttttcatttgctaaaatatattggaatttggttattcatcatgatttaaatcaattaaatacacccaatagacaatctggaattcaaaaagagttaaaggaatttcagttaatgcatggtgtccccaataaagtagtatttgatcgtttaccaagtaattttcaattgcagttagtagaaagaactaaaaaagtgggggcaaggtatgtagtaggggcgttatacggagatatggaagcttctatttatgagtttgacaaaagaacagaatacataaagtttaatagcagtatctatatttttttacaaaaatatagacaaattgttactcatttaaccaattatcatttggcgaaatttttagaaaagcataatgacaaaaataagttagaagatgtcttgttgaaagtagaaaatatttcaaaacgcgaatcgctgcatgcattctataacatactttggaaatatgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgagtatgctggaatgaatggatacgtagaaaactggtctccatag

SEQ ID NO: 12 - DNA, 1380 bp, Paenibacillus xylanexedens
  source 1..1380: mol_type = genomic DNA
gtgaacaagtacggcgaagtagcggttcgtgcaatccattccatatttgaagaaacccatccactctctgcttggaagaaagcagcgtcaatttctttcggagaggggacatgggggcagcgtaaaggttgtcctaagaatgtgtttcttggattatgtgagaagggtttagtcaatggtatacctcatggtaattactctgcaagaagtggatcagagaagaataaaaattacgctatccatgcgattgatgtcttgaaaagcaaacctgaattatcaaaggacaagctaggtttgtggagagaaatgatgaatgggaaacaaatgtctcataactcacagatggatgttgtattagcattgtggaataaccatatgatagatcggagtaaaagagtgaacttcttagaaaaatggctacatatcattcggaattgttcttttgataacacttataaaatggcttgggctaaagccattactgaaattgcagtagagaaagaagaagcagttccaacagaagaaataattctagttgaggttccattaagagatattgcagaaatggtacttcgctattattttgagcaaaccatattctttgctcttcagcaaagttcaaatcctgtgaagccgcctgttatggtgacgattgcaaagcagctgataactgcttatcagtctcgtacaggctcgatgaaaccagtaaaatggtttaggtctaatatagagaccgtatgcaatgtagagtatgagaaggctgtatcttctacagtcaaagcgctaaaatcggatgtaagttaccgcttcttacgtgtagaaggaaaagaagtggaaggtgtttatcaatatcagcaaaaagcggatagtctgttcatgcaaagaggggatttacttacccttaagagccattgtctcattgtgtttgatgccattaattatcgatggacgcagatgttagaaaactttaatcactcgcccagattgtcaaagaaagtacgaatcattgacgaagttcaggtgcgccggaaaccccttggtaaatttacggagtttattgaagctgagaacccagagtgtcgttgctttttatgcggtgaaataatagaagctgaaacacctgcgattgatcatgtgatcccatggtcgtatctgtattcggatgatctctggaatttagtgttcgcgcatcaatcttgtaacagtagtaaatccaatgtgataccgagtgaggacatgattcttcggttagaagcaagaaaccaagcacttcatgattatttaaaagagaaaacgaagctgaataagcatgtctcggatctggaactggctatagatagtaatcttgtacggaagttctggattgcttgccagggttaa

SEQ ID NO: 13 - DNA, 975 bp, Bacillus megaterium
  source 1..975: mol_type = genomic DNA
atgaatgactatattaaacgatggcatagtttaattttaaattgcaactttgataacacatataaaatggtttggagcaaagcattagtagaattgtcggttaatgttagtgacgaagttgataaagatataataatttttgattttgacgatattgcaaaactttgcttaaaatattattggaatcaaactatttactttgatttaatacaaagtcctaacttaaagaaacctccagaaatacttaatcatattaaatccttaattacccatttctttgatgcgaaaacttctgttcatccagttagatttgaaaagatagattttaacagacttcaattaatagataagtacgaagaaaccattaaaagaatagtaacaacgttaaagaaagatgtttgctggcgatttaaaattcttaattctaacacatacgatatatatgaattagaccgtaaaaaaggtaaggtgtattttaaaagacaagacatattaacgcttaaacaaaatagtgacttactatttacctttataaattacagatggacacaaatgttagaggggttcaattatagtccacggatttcaagaaaagtacgtgcgatagatgaaaacaatattaagcgtatgccgttaaaaaagtttcataagtatcttgatttaatgtttgaagacagtaaacgtaaatgtttttattgtggtgaagaaatagatgatagcaatctgtcggttgaccatgttataccttggtcttacatgttttcagatgatttatggaatttagtttactgtcataaagatgaaaactccgaaaaatctaatcatctcccattggaagaagatatattaagattagaaaataggaatatagctttactagaacgtatggagaacgcaaccaaagactacgaccaattacaaatagcccttgagaaaaattatgttaaacagttttggatttcatttaaagggtaa

SEQ ID NO: 14 - DNA, 975 bp, Bacillus megaterium; identical to SEQ ID NO: 9
  source 1..975: mol_type = genomic DNA

SEQ ID NO: 15 - DNA, 498 bp, Microbacterium sp.
  source 1..498: mol_type = genomic DNA
atgcgcacacttgtactgaacgcaggatacgaaccgctcgcgatcgtgtcgttcaagagagccctggtcctggtcatgaacgacaaggccaccgtgatcgaacgcgtggaagacgaccccgtctggggcagccacggcgtctacgaccgccccgccgtcatcgtcctctcgcgttacgtgcgcattcccgcaagcagacgggtgccggtgacccggcggggagtcctgcgccgcgacaatcaccgctgcggctactgcggcaaggcggcgtcgaccatcgaccacgtgatgccgcgctcgcggggcggagccgattcatgggagaacctcgtcgcctgctgcctgcgctgcaacaacaccaagagcgaccgcactccgcaggagatgcgctgggagctgcggttcgcgccgcgtcctcctcacggcacggcgtggacggtgcgtggcaccgagcggagcgatccgaggtgggagccgtacctcgctctcgcggcgtga

SEQ ID NO: 16 - DNA, 3081 bp, Ottowia sp.
  source 1..3081: mol_type = genomic DNA
atgaaattcaggctggcaattgatctggggtctacttcgttgggctgggccatgttccgtctcaatggctctggtgaacccacggcgatcgtcaggtcgggggttcgcatctttagcgatggccgcaacccgaaggatggctcttcgcttgctgtcactcgccgagccgcgcgtgccatgcgccggcgacgtgaccgcttgctcaggcgcaaggaacgcatgctgagcaagctgatcgagcatggcttctttccgcaggacgaagcggcgcgaaaagctcttgagttggcaaatccgtatgagctaagagccaaggggctttacgaagtcctgacaccaggtgagtttgccagagcgctatttcatatcaaccaacggcgcggcttcaagagcaatcgcagagcagaccgaagtgacaacgacagcggagcgctcaagtccgctatcaaggccatgagagctacgctgactgaaagcggagcccggaccgtgggcgaactgctgtggcggcgcttacagaacgggctgggagcgcgtgcccgctatcgcgaaacaccttacaccaccgaagagggcaagaagcgaatcgacaaaagctacgacttttatgtagacaggcaaatggtggctgacgagttcgatgcgatctgggaggcgcaggccactcgaaacccaacattgttcaccgaatcagctcaggctgacttacgagatacgctgctgtttcagcgtgatctacgccccgtgaagcccggacgctgcactctgattccagccgaaccgcgcgcgccgctggctttgcccagcacgcaacgctttcgtattcttcaggaggtgaatcatttgcgcatcctggatgcaggcttgaatgaaacgctccttacgctggcacaacgagatgccatcgtggcgttgttggaagcaaaagccaaggtgagtttcaaggagatgcgcaagcaactcaagctgggtgatgcagcgaagttcaatctagaggatgaaaagcgcactgagctgaaaggcaacgccacggccgctgctttgtcgaagaaagagttgtttggatctgtctggaaagattttaatgaatcaatgcaagatgagatcgtctggcgtttggtcacggagcagagcgaggaggtgctggcgagttggttgtgcgaaaccactggcgtggatgagggacgggcgctagcagtcactggtgcaggtttgccaaaggggtacggtagtttgagcaggcaggcgctcgcacagatcgtgccggcgttgcgcgcgggggtcatcacatatgacaaggccgtacaggaggccggcttcgagcaccatagcgcactggggtttgactatgaatccgatgacgtcgagaactggattcatccagaaacaggcgagatcaaagctgtattcaagcagttgccctactacgggaaagcgctgcagcgtcatgtagccttcggcagtggagaccctgaagattcggaagaaaagcggtacggaaaaatcgccaacccgacggtgcacatcggactgaatcaggtacgtactgtagtaaacgccctcattcgtcgctacggtcgccccacggaggtgatcgtggagctggcacgtgatctgaaacaaagtcgcgagcagaaacaagaggctaccaagaagcaagctgaaaatcagcgccgcaacgagcgtatccgaaaggcatttgccgcggtgaggggatgctctgaggagcaggtgaaaaccacggatatccagaagtggatcctatgggaggaactgagttttgacgttgctgatcggcattgcccatacagcggtgtgcaaatcagtgccgccatgctggtgagcgacgaggtggagatcgagcacatcttgccgttctcgcaaacattggacgacacactgaacaaccgcaccgtggctatgaggcaggccaaccgcatcaagcgcaaccgtactccgtggcaggcacgcgcagacttcgaggcacaagggtggtcctatgagcacattttgcaacgtgctgagcgcatgccgctccgcaagcgttatcgcttcgcggcagatggttacgagcgctggctgggcgaagataaaggatttctggcgcgtgccttgagcgacacgcgctatctctcacgcgtggccaagtcatacctcacgctgatctgtccgttgggtgtgcgagtgatcccggggcagatgacggccatgctgcgcgcaaaatttgggctaaacgacgtattgggcttgaacggcgagaaaaacaggaatgaccaccgacatcatgccgtcgacgcatgcgtgatcggagttaccgatcagggcctgctacagcggtttgcctcggcaagtgcgagcgcacacgaacggggcttgacgcgtttggtcgagagcatgccattgccatggccgacctatcgggagcatgtggagcgagccgtcgggaatatatgggttagccacaagcctgaccacggttacgagggcgccatgatggaggagacgagctatggcatccgaagagatggcagtatcaagcagaggcgcaaggcggacggcagcgccggccgcgagatcagcaatttgatccgtattgccgaacccgagcaggtggtgcgtcacggcgtggatgacggcggccatccgttgccatacaaaggctatgtgggcggcagcaactactgcatcgagatcactgtgaacgacaagggcaaatgggaaggccgggttatttccacgttcgacgcatatcgaatcgtgcggcggggcggtatgcgtcaattgcggcatccgacgctagggcaggatggaaggccattggcgatgcggttgatgattggggatagtgtgcggttggaggtggacggggtactgaagacaacgcgagtcgttaagttgttcgcaacgggccaagttttcatggcacctgtgcatgaggccaacgttgatgttcgcaacagtgacaaaatggacgcgtttacctacacttcgaaatatgcgggctctttgcagaaagcgaatggtcggatggtaataatttcggccattggtgagcttcgtgatcccggtgttgaagtctag

SEQ ID NO: 17 - DNA, 972 bp, Bacillus simplex
  source 1..972: mol_type = genomic DNA
atggacatttacattaaacaatggaacgaccttattttgaattgcagctttgataatacatataaaatggcttggtctaaggcattagttgaattaaccgttaataccaacgaacctatagaagaaattattatatttgatttaaatgatattgcaaaactttgtttaaagtattactggaatcaaaccatttattttgatttaatacaaagtccaaacataaaaaaacctcctgaaatcatttcttctacaaaggatttaataactttattctttaataaaaggcaatccgtacaaccaataagatttgaaaagataaactttaactcgattggcttagcccatgaatttgaactaacggtaaaaaacattgtacgaactttaaagcaagacgtttgttggcgttttaaaaacctaaaccgtaaaactcacgatgtatatgatttagaccgtaatgagggtaaggtattctttaataaaaacgatgtaataatgcttaaactgtatagtgattttttgtttactgttattaattacagatggacacaaatgttagaaggcttcaactatagtccacgtatatcaagaaaggtaagagcaatagatgaaaataacattaaacgtgcatcactgaaaaagtttcataagcaccttgatttaatgtttgaggacggtaaaagaaaatgtttttattgcggtgatgttatagctgaaaacgaattatccgttgaccatgctataccgtggtcttatatgttttcggatgatttatggaacttagtttactgccataaaggggaaaactccgaaaagtcaaaccgcttaccttcagaagaagatatagctcgtttagaagagcgaaataaacagttgttagaccgtatgcaaaataatactaaggactatgatcagttacaaattgccgctgaaaaggattacgttaaaaaattttggatttcattcaagggatag

SEQ ID NO: 18 - DNA, 996 bp, Paenibacillus sp.
  source 1..996: mol_type = genomic DNA
ttgaatagtaggcaatttgatgagaaggactattggcgagctgtaatcctctatggattgaacacagctacctataagattgctcttggacaagctcttatcaacttctcaacaatggacaaggattatgtgtctatggatatgttagcagaatttttttttgatttatatcatgaacgcttgaagaacaatatgcctcagatgagtattcaaggcagaagaactgctatggagcgtattattgacttatataatttaggcaaactagatagagatgaagctatcactaaggttaaactggaagcattcaatgatgtcataccacgatttcatactgttaacggagtagatttaccgatttcattctatgaagctggtaaagacgggttgataattaaagactctctatttgaagtgttcaagggcaaagagagtgagccactaacagaggaattacaatctagatgggacttattagaatcagcttttgaaatgagacattcagaggcgagtatgagcaatgatattagacgtatctacctagagagtggatatgatagaaagagcatcacacacacgattcctgtgcttaatggctatcagaatggtgtatgtttttattgtggtgaaaagatagaagggaaaattcatgtagaccatgtgataccaagacaagtggtctatcacgatgagatttggaacttagtgttgtctcatgagttttgcaatcttcaaaagagtgattacttgccaagtatggtttatctggagaaactaattttaaggaatgaacactttatttcaagtaaacatcctatttctaacaagcttgttgaaaagcttggtaagacaccgatagcgaggagaaagtatatattgaaagtttatgaagatgtaaaggttgttctaggtggagcgacatgggaagggattagaggatataatccgatgacagacaccttctataaaacatttattaggagcttgaaactatga

SEQ ID NO: 19 - DNA, 975 bp, Bacillus sp.
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcatttgctaaaatatattggaatttggttattcatcatgatttaaatcaattaaatacacacaatagacaagctggaattcaaaaagagttaaaggaatttcagttaatgcatggtgtccccaataaagtagtatttgatcgtttaccaagtaatcttcaattgcagttagtagaaagaactaaaaaagtgggggcgaggtatgtagtaggggtgttatacggagatatggaaggttctatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagatgatgtcttgttgaaagtggaaaatatttcaaaacacgaatcgctgcatgcattctataacatactttggaaatacgaaacaaaagaatgtttttattgtggaaaacgtctttctgataaacgcatcgctactcatgtagatcattttattccacggagttatatccaatcagataatatgtggaatttagtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 20 - DNA, 984 bp, Paenibacillus sp.
  source 1..984: mol_type = genomic DNA
atgaattttatagacaaatggatgcacatcatcaggaattgtagctttgacaatacatacaaaatggcttgggctaagggtattacggaaatagctgtggagtctgataatttatttttcacaggatcagcagacatggttgaaatccagctaggggaaatagcgaagaaagtacttcgttattattttgaacagacaatgttctttgatcttcagcaaagttcgaatccaataaagcctccagtaattgtaacaaccgtaaagaatctgatagtttcttatcaggcacagtcaggctcagtaaagccagtgaagtggttcagatcgaatatcgaatcagtatgcaatgcggagtatgataaagctctaaacgctactgtgaaagctttaaaggctgatgttagttatcgctttctacgagtagaaggcagagaagtagatggagtctaccagtacgagcaaaaagcagatagcttaataattcgagcagatagcttgctagcacttaaagagaacaggatgattgttttcgatgcaatcaattatcgatggacacaaatgctggagaatttcaaccactcaccaagactttcgaagaaggtgaggatcattgatgaagatcaagtgcgacggaaaccattgaataagtttactccatatcttgaggcagagaatcctgaatttaattgcttcatatgtggtactaggattgttaacgagactcctgcaatagaccatgtaattccttggtcatatctctattcagatgacgtgtggaacctagtatttgcccaccagtcatgcaacagtagtaagtctaatgtgataccaagtgaagaaatgatacagcggcttgaagaaaggaatagggcgctgcttcaactgatgatgaaaaaggagaaacttgataagcacgtttctgaacttagattggctaacgaaggtaacttagtgcgtaagttttggattgcatgtcaaggctaa

SEQ ID NO: 21 - DNA, 981 bp, Paenibacillus sp.
  source 1..981: mol_type = genomic DNA
atgaattttatagataaatggatgcacatcatcaggaattgtagctttgacaatacatacaaaatggcttgggctaagggtatcacggaaatagctgtggagtctgataaattattttccacaggatcagcagacatggttgaaatccagctaggggaaatagctaagaaagtacttcgttattattttgaacagacaatgttctttgaccttcagcaaagttcgaatccaataaaacctccagtaattgtaacaaccgtaaagaatctgatagcttcttatcaggcacagtcagggtcagtgaagccagtaaagtggttcagatcgaatatcgaatcagtatgcagtgcggagtatgataaagctgtcaacgccactgtaaaagctttaaaggctgatgttagttatcgctttctacgagtagaaggcagagaagtagatggagtttaccagtacgaacaaaaagcagatagcttattcattcgagtagatagtgtgctagcacttaaagagaacaggttgattgttttcgatgccatcaattatcggtggactcaaatgttggagaatttcaaccactcaccaagactatcgaagaaggtgaggatcattgatgaagatcaggtgcggcggaaaccacttggaaaattctcaacgtatttggaagccgagaacccagaatgtcattgcttcttatgtggtttaatgatagaggaggcacctgccatagatcatgtcattccttggtcatacctatattcggatgatgtgtggaatctagtatttgcccaccagtcctgtaacagcagtaagtctaatatgatacctagcgagaaaatgataaatcgattagaaaaaagaaatataagaatgcttcaaacattgaatgatcaaggtaagaaagataaacatatctcagaacttaaaatggcaatagaacataatttagtgcgcaagttttggatatcatgtcatggttaa

SEQ ID NO: 22 - DNA, 981 bp, Paenibacillus sp.; identical to SEQ ID NO: 21
  source 1..981: mol_type = genomic DNA

SEQ ID NO: 23 - DNA, 498 bp, Microbacterium sp.
  source 1..498: mol_type = genomic DNA
atgcgcacactggtactgaacgcaggatacgagcctctggcgatcgtgtctttcaagagagcccttgtcctggtcatgaacgacaaggccacggtgatcgaacgcgtggaagacgatcccgtctggggcagtcacggtgtctacgacagaccggccgtcatcatcctgtcgcggtatgtgcgcatccccgtgagcagacgggttcccgtgacccggcggggagtgctgcgacgagacgatcatcggtgcggctactgcggcaaggccgcttcgaccatcgaccatgtgcttcctcgatcgcgtgggggcgcggactcgtgggagaacctcgtcgcctgctgcctccgctgcaacaacgtcaagagcgaccgcacgccgcaggagatgcggtgggctctgcggttcaccccgcgcccaccgcacggcaccgcgtggacggttcgtgggaccgagcgcagcgacccgcggtgggagccctacctggcgctcgccgcgtga

SEQ ID NO: 24 - DNA, 498 bp, Microbacterium sp.
  source 1..498: mol_type = genomic DNA
atgcgcacactggtcctgaacgccggatacgaacccctcaccgtggtctcgttcaagagagccatggtgctcgtgatgaacgacaaggccagcgtgatcgagcacgtggagggcgatcccgtctgggcgggacgcaacgcatacgaccggccggtcgtgatcctgctcacccgctatgtgcgcgtgccgtcatcgaggcgcgttcctgtcacccggcggggcgtgctccgccgcgacgaccaccgctgcggctactgcggacggagtgcgtcgacgatcgaccacatcctgccgcgctcgcgcggaggggcggacagctgggagaacctcgtcgcctgctgcctgaagtgcaacaacatcaagagcgaccgcaccccgcaggagatgggctgggaactgcggatgacgccgcgaccgccgcacggcacggcgtggacggtgcgcgggggcgagcgcggtgacccgcgctgggagccgtacctcgcgctcgcggcgtag

SEQ ID NO: 25 - DNA, 498 bp, Microbacterium sp.; identical to SEQ ID NO: 23
  source 1..498: mol_type = genomic DNA

SEQ ID NO: 26 - DNA, 996 bp, unidentified
  source 1..996: mol_type = genomic DNA; note = Paenibacillus crassostreae
ttgagtagtagacaatttgatgaaaaagactattggagagctgtcattctctatggacttaatacagctacatataaaattgctctagggcaagctctaatcaacttctcaacaatagacaaggattttgtgactatggatatgttagcagaatccttttttgatttatatcatgaacgattgaagaacaatatgccccaaatgagtattaaaggcagaagaacagctatggagcgtattgttgacttatataacttaggtagactagatagagaagaagctatcaacaaagttaagctagaagcttttaatgatgtcatcccaaggtttcatactgttaatggcattgatctgccgatttcattctatgagtcgggtaaagatgggttgataattaaagactctctatttgaggtgttcaagggcaaagagagtgattcactaacagaggaattacaatcaaggtgggacttgttagaatcagcttttgagatgagacattcagaggctagtatgagcaatgatataagacgtatctacctagagagtggctatgatagaaagagcatcacacatacgattcctgtacttaatggctatcagagcggtgtgtgtttctattgtggagaacaaattgaaggtaagattcatgtcgatcatgtgataccaaggcaagttatctttcatgatgagatttggaatctagtgttgtctcatgaattttgcaatctccaaaagagtgattacttgccaagcatggtttatgtggagaaactgattgcaagaaacgaacactttatttcaagcaaacatcccatatcaaacaagctagttgagaagctcggtaagacaccaatagcaaggaaaaggtacatattgaaagtatatgaggatgtaaagactgttcttggtggtgctacatgggaaggtattcgtggctataatccaatgacagatacattctataaaacatttataaggagcttaaagctatga

SEQ ID NO: 27 - DNA, 498 bp, Microbacterium sp.
  source 1..498: mol_type = genomic DNA
atgcgcacactggtcctgaacgccggatacgaacccctcaccgtggtctcgttcaagagagccatggtgctcgtgatgaacgacaaggccagcgtgatcgagcacgtggagggcgatcccgtctgggcgggacgcaacgcatacgaccggccggtcgtgatcctgctcgcccgctatgtgcgcgtgccgtcatcgaggcgcgttcctgtcacccggcggggcgtgctccgccgcgacgaccaccgctgcggctactgcggacggagtgcgtcgacgatcgaccacatcctgccgcgctcgcgcggaggggcggacagctgggagaacctcgtcgcctgctgcctgaagtgcaacaacatcaagagcgaccgcaccccgcaggagatgggctgggaactgcggatgacgccgcgaccgccgcacggcacggcgtggacggtgcgcgggggcgagcgcggtgacccgcgctgggagccgtacctcgcgctcgcggcgtag

SEQ ID NO: 28 - DNA, 498 bp, Microbacterium sp.
  source 1..498: mol_type = genomic DNA
atgcgcacactggtcctgaacgccggatacgaacccctcaccgtggtctcgttcaagagagccatggtgctcgtgatgaacgacaaggccagcgtgatcgagcacgtggagggcgatcccgtctgggcggggcgcaacgcgtacgaccggccggtcgtgatcctgctcacccgctacgtgcgcgtgccgtcgtcgaggcgggttcctgtcacccggcggggcgtgctgcgccgggacgaccaccgctgcggctactgcgggcggagcgcgtcgacgatcgaccacatcctgccgcgctcgcgcggaggggcggacagctgggagaacctcgtcgcctgctgcctgaagtgcaacaacatcaagagcgaccgcaccccgcaggagatgggctgggagctgcggatgacgccgcgtccgcctcacggcacggcctggaccgtgcgcggcggtgagcgcggcgacccgcgctgggagccgtacctcgcgctcgcggcgtag

SEQ ID NO: 29 - DNA, 498 bp, Microbacterium sp.
  source 1..498: mol_type = genomic DNA
atgcgcacactggtcctgaacgccggatacgaacccctcaccgtggtctcgttcaagagagccatggtgctcgtgatgaacgacaaggccagcgtgatcgagcacgtggagggcgatcccgtctgggcgggacgcaacgcatacgaccggccggtcgtgatcctgctcacccgctatgtgcgcgtgccgtcatcgaggcgcgttccggtcacccggcggggcgtgctccgccgggacgaccaccgctgcggctactgcggacggagtgcgtcgacgatcgaccacatcctgccgcgctcgcggggaggggcggacagctgggagaacctcgtcgcctgctgcctgaagtgcaacaacatcaagagcgaccgcaccccgcaggagatgggctgggaactgcggatgacgccgcgaccgccgcacggcacggcgtggacggtgcgcgggggcgagcgcggtgacccgcgctgggagccgtacctcgcgctcgcggcgtag

SEQ ID NO: 30 - DNA, 498 bp, Microbacterium sp.; identical to SEQ ID NO: 29
  source 1..498: mol_type = genomic DNA

SEQ ID NO: 31 - DNA, 975 bp, Bacillus megaterium
  source 1..975: mol_type = genomic DNA
gtgagttttaatttaaaagtaggagaaataaaaaaaacttatttaacagaacaagaaatttggaagatcattaatcaatttttcgctaatggtcactttaccacaacatataaatatggattgatgaaagctttaattgaaaacctatataatgttgacaatcggttagtcctcacttttgatcaagtgtatttttcatttgctaaaatatattggaacttggttattcaccatgatttaaatcaattaaatacgtccaacagacaagctggaattcaaaaagagttaaaggaatttcagttaatgcatggtgtccccaataaagtagtatttgatcatttaccaagtaatcttcaattgcaattagtagaaagaactaaaaaagtgggggcaaggtatgtagtaggggcgttatatggagatatggaaggttctgtttatgagtttgataaaagaacagaatacataaagtttaatagcagtatatatatttttttacaaaaatatagacaaattgttactcatttaaccaattatcatttggcgaaatttttagaaaagcacaatgacaaaaataagttggaagatgtcttgttgaaagtggaaaatatatcaaaacgcgaatcgctgcatgcattctataacatcctttggaaatatgaaacaaaagaatgtttttattgtgggaaagttctttctgataaacgcatcgctactcatgtagatcattttattccatggagttatatccaatcagataatatgtggaatttggtgctaagctgtcaaagctgcaacacaaaaaagagcaataaacttgctaagagcttatatcttgagaagatgtttgttcgtaatgagaatcttaaattaacagcagttcacaatgattttaactattattcggagcgtaagttagaagctctttatgagtatgctggaatgaatggatacgtagaaaactggtctccttaa

SEQ ID NO: 32 - DNA, 501 bp, unidentified
  source 1..501: mol_type = genomic DNA; note = Pseudarthrobacter chlorophenolicus
atgcgcactctcgttctgaatgctggatatgaaccgctggcggttattactttccgccgggcgctggtgcttgtgcttacaggcaaggccagtgtggtggccgaaggggacgaccccgtggtgggccccaccgacgtcctggggcgcccgtccgtgatcctcctcaaccgctacatccgcccccggtacaaccactccacggccgtcagccgcaggggagtgctccgacgcgacgggcataaatgtgcctactgcgggaaagcggcgcacaccattgaccatgtgcacccgaaatcccggggcggggctgattcgtgggagaacctggtggccgcctgcctgcggtgcaacaacgtcaagggcgatcacactcccgccgagatgggatggaccctgcgcttcgtcccggaacctccgcacgggaccatctggcagatcaaggagctggagaagcccaccccggcctgggatccgttcctgcttccggaacgggctgcctga

SEQ ID NO: 33 - DNA, 498 bp, Microbacterium sp.
  source 1..498: mol_type = genomic DNA
atgcgcacactggtcctgaacgccggatacgaaccactcaccgtggtctcgttcaagagagccatggtgctcgtgatgaacgacaaggcgagcgtgatcgagcacgtggagggcgatcccgtctgggcgggccgcaacgcatacgaccggccggtcgtgatcctgctcacccgttacgtgcgcgtgccctcgtcgaggcgagttcctgtcacccggcgcggggtgctccgccgggacgaccaccgctgcggctactgcggaaggagcgcgtcgaccatcgaccacatcctgccgcgctcgcgcggaggggccgacagctgggagaacctggtcgcctgctgcctgaagtgcaacaacatcaagagcgaccgcaccccgcaggagatgggctgggaactgcggatgacgccgcgtccgccgcacggcacggcgtggaccgtccgcggcggtgagcgcggtgacccgcgctgggagccgtacctcgcgctcgcggcgtag

SEQ ID NO: 34 - DNA, 498 bp, Microbacterium sp.
  source 1..498: mol_type = genomic DNA
atgcgcacactggtcctgaacgccggatacgaaccactcactgtggtctcgttcaagagagccatggtgctcgtgatgaacgacaaggccagcgtcatcgagcacgtcgagggcgatcccgtctgggcggggcgcaacgcgtacgaccggccggtcgtgatcctgctcacccgctacgtgcgcgtgccgtcgtcgaggcgcgtgccggtcacgaggcgcggcgtgctgcgccgggacgaccaccgctgcggctactgcggtcgcagcgcatcgacgatcgaccacatcctgccgcgttcgcgcggaggcgcggacagctgggagaacctggtcgcctgctgcctgaagtgcaacaacatcaagagcgaccgcacgccgcaggagatgggctgggcgctgcgcatgacgccgcggccgccgcacggcacagcctggacggtgcgcggcggtgagcgcggcgatccgcggtgggagccgtacctggcgctcgcggcatag

SEQ ID NO: 35 - DNA, 498 bp, Microbacterium sp.; identical to SEQ ID NO: 24
  source 1..498: mol_type = genomic DNA

SEQ ID NO: 36 - DNA, 498 bp, Microbacterium sp.
  source 1..498: mol_type = genomic DNA
atgcgcacactggtcctgaacgccggatacgaacccctcaccgtggtctcgttcaagagagccatggtgctcgtgatgaacgacaaggccagcgtgatcgagcacgttgagggcgatcccgtctgggcgggacgcaacgcatacgaccggccggtcgtgatcctgctcacccgctatgtgcgcgtgccgtcatcgaggcgcgttccggtcacccggcggggcgtgctccgccgcgacgaccagcgctgcggctactgcggacggagtgcgtcgacgatcgaccacatcctgccgcgctcgcgcggaggggcggacagctgggagaacctcgtcgcctgctgcctgaagtgcaacaacatcaagagcgaccgcaccccgcaggagatgggctgggaactgcggatgacgccgcgtccgccgcacggcacggcgtggacggtgcgcgggggcgagcgcggtgacccgcgctgggagccgtacctcgcgctcgcggcgtag

SEQ ID NO: 37 - DNA, 498 bp, Microbacterium sp.; identical to SEQ ID NO: 29
  source 1..498: mol_type = genomic DNA

SEQ ID NO: 38 - DNA, 975 bp, Bacillus megaterium; identical to SEQ ID NO: 31
  source 1..975: mol_type = genomic DNA

SEQ ID NO: 39 - DNA, 498 bp, Microbacterium sp.; identical to SEQ ID NO: 23
  source 1..498: mol_type = genomic DNA

SEQ ID NO: 40 - DNA, 498 bp, Microbacterium sp.
  source 1..498: mol_type = genomic DNA
atgcgcacactggtcctgaacgccggatacgagcccctcaccgtggtctcgttcaagagagccatggtgctcgtgatgaacgacaaagccagcgtgatcgagcacgtggagggcgatcccgtctgggcgggacgcaacgcatacgaccggccggtcgtgatcctgctcacccgctatgtgcgcgtgccgtcatcgaggcgcgttcctgtaacccggcggggcgtgctccgccgggacgaccaccgctgcggctactgcggacggagtgcgtcgacgatcgaccacatccttccgcgctcgcggggaggggcggacagctgggagaacctcgtcgcctgctgcctgaagtgcaacaacatcaagagcgaccgcaccccgcaggagatgggctgggaactgcggatgacgccgcgaccgccgcacggcacggcgtggacggtgcgcggcggtgagcgcggtgacccgcgctgggagccgtacctcgcgctcgcggcgtag

SEQ ID NO: 41 - DNA, 996 bp, Bacillus sp.
  source 1..996: mol_type = genomic DNA
atgagccataaattacaagtaggagaaatgcaagcaggctatctaacagataaagaaatatgggctcattttaattatattttttcttcaaaatcaaaaaattcaactacttacaagtttgtcttgattaaatccattttagaaaatctatacaatgtgaacgagaagctggaactatcgtattcaaccttattttcaagctttgccaagatctactggaatctggttatccatcataatttgaaccaaataaatatgacagataagaaggcagaggtacaaaagatactcctagatgtccaagcaaaatatcaaattccgaaaacacttgtttttgataagctatctcatgaattacagattgagatcattatgaaagtgaagaagaaatgcaagatcaatgtcatgggtgccctatatggggacacaggcagcaccatttatgattttaataatggaaaggaacagctaaaacttaatagcaccttttactcattcatgcagcgatttcaacgggttttaaattatttaaccaattatcatctcgcattatttttagaaaaatttaacgaaaagggagataccactaacttgcttttaaaagtggaaaatgtctctaagagatcctcgctcgaccagttttataaggtactctcgtcattttataatggaaagtgtttttattgtggtaaaactattaagaaagaaaacgcccatgttgatcactttataccatggagctttgtccaagctgaccaattgtggaacttggtgattgcttgcagcgcttgtaatttgtcaaaaagtgataaattagctaacgtacactttttaaatacgatgattgaccgaaatgaaagcttactttctattcaggagttcagtgctagggaagacatgaaggtgtataccagttataaattgaaggatctatatcattactcaatggaaaatggatttacagaattttggacgccaaagaaattgatattgtga

SEQ ID NO: 42 - DNA, 342 bp, Bacillus sp.
  source 1..342: mol_type = genomic DNA
ttgtcattttataatggtaagtgtttttactgtgggaaaaccataaagaaagaaaacgcacatgtcgatcactttattccctggagcttggtccaagcggatcaactgtggaatttggtgattgcttgcagcacttgtaatttgtctaaaagtgataagttggccaatgaactatttttagagacagtgattgatcgcaatgaaacactactttcaataaaggaacttaaagtgagggaagacatgaaggtgtattccagtaataagctgaaagatctgtatcattactcagtagaaaatgggtttacagagttttggacaccgaagaaaaggatagtgtga

SEQ ID NO: 43 - DNA, 996 bp, Bacillus sp.
  source 1..996: mol_type = genomic DNA
atgagccataaattacaagtaggagaaatgaaaacagcctatctaacagataaagaaatatgggctcattttaattacattttttcttcaaaatcaaaaaattcaactacctacaagtttgtcttgattaaatcaattctagaaaacttatataatgtaaatgacaagttggaactatcatactccaccttattttcgagctttgccaagatttactggaatctagttattcatcataatttgaaccaaattaatatgacagaaaagaaggcagaggtacaaaaaatcctaatcggcgtccaggaaaagtatcaaattccgaaaacacttgtttttgataagctatctcatgaattacagattgagatcattacgaaagtgaagaaaaaatgtaagctcaatgtcatgggtgccctatatggagacaccggcagtactatttatgattttgataatggaaaagagcagctaatacttaatagcactttttactcattcatgcaacggtttcaaagggtattaaattatataaccaattatcatctcgcgttatttttagaaaaatttaacgaaaagggagataccactaatctgcttttaaaagtggaaaatgtctcaaagagatcatcgctcgaccagttttataaggtactctcgtctttttataacggaaagtgtttttactgtggtaaaacgattaaaaaaggtaacgctcatgttgatcactttattccctggagctttgtccaagctgaccaactgtggaatttggtgattgcttgcagcgcttgtaatttatcaaaaagtgataaattagctaatgaacgattcttggtttcgatgatggatcgaaatgaatcgttactttccattcaggaattaaggaaaagggaagatatgaaagtgtatacgagtaataaattggaggatttatatcattattcagtggaaaatggttttaccgaattttggactccgaagaaaatcattttataa

SEQ ID NO: 44 - DNA, 996 bp, Bacillus sp.; identical to SEQ ID NO: 41
  source 1..996: mol_type = genomic DNA

SEQ ID NO: 45 - DNA, 342 bp, Bacillus sp.; identical to SEQ ID NO: 42
  source 1..342: mol_type = genomic DNA

SEQ ID NO: 46 - DNA, 741 bp, Bacillus sp.
  source 1..741: mol_type = genomic DNA
atgacagataagaaggcagaggtccaaaaaatcctcatcggtgtccaggaaaagtatcaaatcccgaagacacttgtatttgataagttgtcgcttgaattacagattgagatcattgataaagttaagaaaaaatgcaagatcaatgttatgggtgccttatacggagacaccggcagcactatttatgattttgataatgggaaagagcagctaaaacttaatcgtacattttactcatttatgcagagatttcaacgggttttaaattttttaaccaattatcatctcgcgttatttttagaaaaatttaacgaaaagggagatacaactaacctccttttaaaagtagagaatgtctctaagagatcatcgctcgaccagttttatcaggtactctcgtttttttataatggaaagtgtttttattgcggtaaaactattaagaaagaaaacgctcatgtggatcactttataccatggagctttgtccaagctgaccaactgtggaatttggtgattgcttgcagcgcttgtaatttatctaaaagtgataaattagccaatggaccattcttagatacaatgattgatagaaatgaaaccctacttactatagaggaacttggtctgagggaagatatgagggtgtattccagtcataagctgaaagatctatatcattactctgtagaaaatgggtatacggaattttgggtaccaaggaaaataatattgtga

SEQ ID NO: 47 - DNA, 498 bp, Microbacterium sp.; identical to SEQ ID NO: 24
  source 1..498: mol_type = genomic DNA

SEQ ID NO: 48 - DNA, 498 bp, Microbacterium sp.; identical to SEQ ID NO: 29
  source 1..498: mol_type = genomic DNA

SEQ ID NO: 49 - DNA, 498 bp, Microbacterium sp.
  source 1..498: mol_type = genomic DNA
atgcgcacacttgtactgaacgcaggatacgaaccgctcgcgatcgtgtcgttcaagagagccctggtcctggtcatgaacgacaaggcgacggtcatcgaacgtgtcgaggacgaccccgtctggggcagtcacggcgtctacgaccgtcctgccgtcatcgtcctctcgcggtatgtgcgcattccgatcagcagacgcgtcccggtcacacggcggggggtcctccgccgtgacaaccaccgctgcggctactgcggcaaggccgcctcgaccatcgaccacgtgatgccgcgttcgcgcggaggggctgattcgtgggaaaacctcgtcgcgtgctgcctgcgctgcaacaacaccaagagcgaccgcacgccgcaggagatgcgctgggaactgaggttcgcgcctcgtcctccgcacgggacggcgtggacggtccgcggcacggagcggagcgacccgcgatgggagccgtacctggccctcgccgcgtga

SEQ ID NO: 50 - DNA, 498 bp, Microbacterium sp.; identical to SEQ ID NO: 29
  source 1..498: mol_type = genomic DNA

SEQ ID NO: 51 - DNA, 498 bp, Microbacterium sp.
  source 1..498: mol_type = genomic DNA
atgcgcacactggtcctgaacgccggatacgaacccctcaccgtggtctcgttcaagagagccatggtgctcgtgatgaacgacaaggccagcgtgatcgagcacgtggagggcgatcccgtctgggcgggacgcaacgcatacgaccggccggtcgtgatcctgctcacccgctatgtgcgcgtgccgtcatcgaggcgtgttccggtcacccggcggggcgtgctccgccgggacgaccaccgctgcggctactgcggacggagtgcgtcgacgatcgaccacatcctgccgcgctcgcggggaggggcggacagctgggagaacctcgtcgcctgctgcctgaagtgcaacaacatcaagagcgaccgcaccccgcaggagatgggctgggaactgcggatgacgccgcgaccgccgcacggcacggcgtggacggtgcgcgggggcgagcgcggtgacccgcgctgggagccgtacctcgcgctcgcggcgtag

SEQ ID NO: 52 - DNA, 498 bp, Microbacterium sp.; identical to SEQ ID NO: 36
  source 1..498: mol_type = genomic DNA

SEQ ID NO: 53 - DNA, 501 bp, unidentified
  source 1..501: mol_type = genomic DNA; note = Pseudarthrobacter chlorophenolicus
atgcgcactctcgttctgaatgctggatatgaaccgctggcggttatcactttccgccgggcgctggtgcttgtgctcacgggcaaggcaagcgtggtggccgaaggggatgacccggtggtagggcccaccgacgtcctgggccgcccttccgtgatcctcctcaaccgctacatccggccccggtacaaccactcgacggcagtcagccgcaggggtgtgctccggcgcgacggacataaatgtgcctactgcggcaaggcggcgcacacaatcgaccacgttcacccgaagtcccggggcggtgccgattcatgggagaaccttgtggccgcctgcctgcggtgcaacaacgtcaagggcgaccacactcccgcggaaatgggctggacccttcggttcgtgcccgagccgccgcacggaacgatctggcagatcaaggaactggagaagccgacgccggcgtgggatccgttcctgctcccggagcgcgccgcctga

SEQ ID NO: 54 - DNA, 498 bp, Microbacterium sp.; identical to SEQ ID NO: 36
  source 1..498: mol_type = genomic DNA

SEQ ID NO: 55 - DNA, 498 bp, Microbacterium sp.
  source 1..498: mol_type = genomic DNA
atgcgcacactggtcctgaacgccggatacgagcccctcaccgtggtctcgttcaagagagccatggtgctcgtgatgaacgacaaagccagcgtgatcgagcacgtggagggcgatcccgtctgggcgggacgcaacgcatacgaccggccagtcgtgatcctgctcacccgctatgtgcgcgtgccgtcatcgaggcgcgttcctgtcacccggcggggcgtgctccgccgcgacgaccaccgctgcggctactgcggacggagtgcgtcgacgatcgaccacatccttccgcgctcgcggggaggggcggacagctgggagaacctcgtcgcctgctgcctgaagtgcaacaacatcaagagcgaccgcaccccgcaggagatgggctgggaactgcggatgacgccgcgaccgccgcacggcacggcgtggacggtgcgcggcggtgagcgcggtgacccgcgctgggagccgtacctcgcgctcgcggcgtag

SEQ ID NO: 56 - DNA, 498 bp, Microbacterium sp.; identical to SEQ ID NO: 36
  source 1..498: mol_type = genomic DNA

SEQ ID NO: 57 - DNA, 498 bp, Microbacterium sp.; identical to SEQ ID NO: 29
  source 1..498: mol_type = genomic DNA

SEQ ID NO: 58 - DNA, 498 bp, Microbacterium sp.; identical to SEQ ID NO: 23
  source 1..498: mol_type = genomic DNA

SEQ ID NO: 59 - DNA, 498 bp, Microbacterium sp.; identical to SEQ ID NO: 24
  source 1..498: mol_type = genomic DNA

SEQ ID NO: 60 - DNA, 498 bp, Microbacterium sp.; identical to SEQ ID NO: 23
  source 1..498: mol_type = genomic DNA

SEQ ID NO: 61 - DNA, 498 bp, Microbacterium sp.; identical to SEQ ID NO: 36
  source 1..498: mol_type = genomic DNA

SEQ ID NO: 62 - DNA, 498 bp, Microbacterium sp.; identical to SEQ ID NO: 24
  source 1..498: mol_type = genomic DNA

SEQ ID NO: 63 - DNA, 498 bp, Microbacterium sp.; identical to SEQ ID NO: 29
  source 1..498: mol_type = genomic DNA

SEQ ID NO: 64 - DNA, 498 bp, unidentified; identical to SEQ ID NO: 24
  source 1..498: mol_type = genomic DNA

SEQ ID NO: 65 - DNA, 498 bp, Microbacterium sp.; identical to SEQ ID NO: 55
  source 1..498: mol_type = genomic DNA

SEQ ID NO: 66 - DNA, 498 bp, Microbacterium sp.; identical to SEQ ID NO: 29
  source 1..498: mol_type = genomic DNA

SEQ ID NO: 67 - DNA, 498 bp, Microbacterium sp.; identical to SEQ ID NO: 23
  source 1..498: mol_type = genomic DNA

SEQ ID NO: 68 - DNA, 498 bp, Microbacterium sp.
  source 1..498: mol_type = genomic DNA
atgcgcacactggtcctgaatgccggatacgaacccctcaccgtggtctcgttcaagagagccatggtgctcgtgatgaacgacaaggccagcgtgatcgagcacgtggagggcgatcccgtctgggcggggcgcaacgcgtacgaccggccggtcgtgatcctgctcacccgctacgtgcgcgtgccgtcgtcgaggcgggttcctgtcacccggcggggcgtgctgcgccgggacgaccaccgctgcggctactgcgggcggagcgcgtcgacgatcgaccacatcctgccgcgctcgcgcggaggggcggacagctgggagaacctcgtcgcctgctgcctgaagtgcaacaacatcaagagcgaccgcaccccgcaggagatgggctgggagctgcggatgacgccgcgtccgcctcacggcacggcctggaccgtgcgcggcggtgagcgcggcgacccgcgctgggagccgtacctcgcgctcgcggcgtag

SEQ ID NO: 69 - DNA, 498 bp, Microbacterium sp.; identical to SEQ ID NO: 28
  source 1..498: mol_type = genomic DNA

SEQ ID NO: 70 - DNA, 498 bp, Microbacterium sp.; identical to SEQ ID NO: 24
  source 1..498: mol_type = genomic DNA

SEQ ID NO: 71 - DNA, 498 bp, Microbacterium sp.; identical to SEQ ID NO: 49
  source 1..498: mol_type = genomic DNA

SEQ ID NO: 72 - DNA, 498 bp, Microbacterium sp.
  source 1..498: mol_type = genomic DNA
atgcgcacactggtactgaacgcgggatacgagcctctggcgatcgtgtctttcaagagggccctggtccttgtcatgaacgacaaggcgacggtgatcgagcgcgtggaagacgatcccgtctggggcagtcacggtgtctacgacagaccggccgtcatcatcctgtcgcggtatgtgcgcatccccgtgagcagacgggttcccgtgacccggaggggagtgctgcgacgagacgatcatcggtgcggctactgcggcaaggccgcgtcgaccatcgaccatgtgcttcctcgatcgcgtgggggcgcggactcgtgggagaacctcgtcgcctgctgcctccgctgcaacaacgtcaagagcgaccgcacgccgcaggagatgcggtgggccctgcgattcaccccgcgcccaccgcacggcaccgcgtggacggttcgtggcaccgagcgcagcgacccgcggtgggagccctacctggcgctcgccgcgtga

SEQ ID NO: 73 - DNA, 498 bp, Microbacterium sp.
  source 1..498: mol_type = genomic DNA
atgcgcacacttgtactgaacgcaggatacgaaccgctcgcgatcgtgtcgttcaagagagccctggtcctggtcatgaacgacaaggcgacggtcatcgaacgtgtcgaggacgaccccgtctggggcagtcacggcgtctacgaccgtcctgccgtcatcgtcctctcgcggtatgtgcgcattccgatcagcagacgcgtcccggtcacacggcggggcgtcctccgccgtgacaaccaccgctgcggctactgcggcaaggccgcctcgaccatcgaccacgtgatgccgcgttcgcgcggaggggctgattcgtgggagaacctcgtcgcgtgctgcctgcgctgcaacaacaccaagagcgaccgcacgccgcaggagatgcgctgggaactgaggttcgcacctcgtcctccacacgggacggcgtggacggtccgcggcacggagcggagcgacccgcgatgggagccgtacctggcactcgccgcgtga

SEQ ID NO: 74 - DNA, 498 bp, Microbacterium sp.
  source 1..498: mol_type = genomic DNA
atgcgcacacttgtactgaacgcaggatacgaaccgctcgcgatcgtgtcgttcaagagagccctggtcctggtcatgaacgacaaggcgacggtcatcgaacgtgtcgaggacgaccccgtctggggcagtcacggcgtctacgaccgtcctgccgtcatcgtcctctcgcggtatgtgcgcattccgatcagcagacgcgtcccggtcacacggcggggcgtcctccgccgtgacaaccaccgctgcggctactgcggcaaggccgcctcgaccatcgaccacgtgatgccgcgttcgcgcggaggggctgattcgtgggagaacctcgtcgcgtgctgcctgcgctgcaacaacaccaagagcgaccgcacgccgcaggagatgcgctgggaactgaggttcgcgcctcgtcctccgcacgggacggcgtggacggtccgcggcacggagcggagcgacccgcgatgggagccgtacctggcactcgccgcgtga

SEQ ID NO: 75 - DNA, 498 bp, Microbacterium sp.; identical to SEQ ID NO: 55
  source 1..498: mol_type = genomic DNA

SEQ ID NO: 76 - DNA, 498 bp, Microbacterium aurum
  source 1..498: mol_type = genomic DNA
atgcgcactctggtgctgaacgcaggatacgaaccgctggccgtcgtgtcattcaaacgagctgtggtgctcgtgatgaatgccaaagcgaccgtcattgagcgcgtggagggcgatcccgtctggggcgcgagcggcgctgtcgaccggccggcggtgatcctgctgacgcgttatgtgcgggtgccacagtcgcgcagggtgccggtgacgcggcggggtgtgctgcgtcgcgacggcaaccgctgcgcgtactgcggcaagggtgcgaccacgatcgatcatgtgatgccgcgttcgcgcgggggagcggactcgtgggagaacctcgtggcctgctgcctgcggtgcaacaacctgaagggtgaccggaccccggccgagatgcgctgggatctgcggtacatgccgcggcccccgcacggcacgcagtggacggtgcgcggcaccgagcgcgcggacccgcggtgggagccgtacctcgagctcgcggcgtag

SEQ ID NO: 77 - DNA, 591 bp, Bacillus sp.
  source 1..591: mol_type = genomic DNA
atgggggcgatatatggcgataccggctgcaccatctatgattttgataatcaaaaggaacagctaagaatgaatagtacattttactcgttcatgcagcggttccagcgggtcttaaattatttaaccaattatcatcttgctttatttttggaaaaattcaatgaaaaaggcgataccacaaacctgcttttaaaagttgaaaatgtatccaagaggtcatcccttgatcaattttatcaggtgctttcctctttttataacgggaagtgtttttattgtggaaaggctatcaggaaggaaaatgcccacgttgatcactttattccttggagctttgtccaggcagaccagctttggaatttagttatcgcctgtagtgcttgcaatttatctaaaagtgataaattggctaacaagatatttctaaaaacggttattgaccgaaacgaaaccttaatttctattccggaactaagaagaagggaagatatgaaggtatatacaagcaataaacttaaagacctttatcaatattcaattgaaaatggttttacagatatttggcagccaaagaaagtgattctgtaa

SEQ ID NO: 78 - DNA, 495 bp, unidentified
  source 1..495: mol_type = genomic DNA; note = Glutamicibacter arilaitensis
atgcgaacactggtattaaatgcaggatacgagcccttagcagtcgtgacttttcgacgagcactggtaatggtcatggccgataaagcgaccatcgttctccaggatgacgactcgcctgttcgcagctcccacggacagctacccagacccactgtcattgtcttaaaacgctatgtacgacgcccctaccgcgacatgcttcatgtcacgcgtaggggcgttttgcgtcgagacgcgtatacctgcgcgtactgcgggaaatcggcgaatacgatcgaccacgtcattccgcgaagcaggggagggaagaacacgtgggagaacttggtggctgcttgcctgcactgcaacagcaggaaagccgacaagatgctctctcagttgggctggcagctgcggatcacgccgcatcgccctcggatgggcggacatctggtctccggtcttcagcatattgataccgcatgggacgacttccttcaagcaagctga

SEQ ID NO: 79 - DNA, 498 bp, Microbacterium sp.; identical to SEQ ID NO: 29
  source 1..498: mol_type = genomic DNA

SEQ ID NO: 80 - DNA, 498 bp, Microbacterium sp.; identical to SEQ ID NO: 68
  source 1..498: mol_type = genomic DNA

SEQ ID NO: 81 - DNA, 498 bp, Microbacterium sp.; identical to SEQ ID NO: 29
  source 1..498: mol_type = genomic DNA

SEQ ID NO: 82 - DNA, 498 bp, Microbacterium sp.; identical to SEQ ID NO: 24
  source 1..498: mol_type = genomic DNA

SEQ ID NO: 83 - DNA, 498 bp, Microbacterium sp.
  source 1..498: mol_type = genomic DNA
atgcgcacactggtgctgaatgcaggatatgaacccatcacggtggtgtcgttcaagagagcgatggtgctcgtgatgaacgacaaggccagtgtgatcgagcacgtggagggcgacccggtctggtcgggtcgcaacgcctatgaacgcccggtcgtgatcctcctcaccaggtacgtgcgtctgccgtcgtcccgccgggtgccggtcacccgtcgcggggtgctgcgccgcgacgaccaccgctgcgggtactgcggccgcagcgcatcgaccatcgaccacatcctcccccgctcgcgcgggggagcggacagctgggagaacctggtcgcgtgctgcctgaagtgcaacaacgtcaagagcgaccgcacgccgcgggagatgggctgggagctgcggatgacgccccgtcctccgcatggcacggcgtggaccgtccgcggtgccgagcgcggcgacccgcgctgggagccctacctcgcgctggccgcgtag

SEQ ID NO: 84 - DNA, 498 bp, Microbacterium sp.
  source 1..498: mol_type = genomic DNA
atgcgcacacttgtactgaacgccggatacgaaccgctcgcgatcgtgtcgttcaagagagccctggtcctggtcatgaacgacaaggccacggtgatcgagcacgtggaggacgaccccgtctgggggagccacggagtctacgatcgccctgccgtgatcatcctctcgcgctacgtgcgcatccccgtcagcagacgcgtgccggtcacccggcggggagtgctccgccgcgacaaccaccgctgcggatactgcggcaaggccgcgtcgacgatcgaccacgtgatgccgcggtcgcgcgggggagccgattcgtgggagaacctcgtcgcgtgctgcctgcgctgcaacaacatcaagagcgaccgcacgccgcaggagatgcggtgggaactgcgagtgctgccacgcccgccgcacggtaccgcgtggacggtgcgggggaccgaacgcagcgaccctcgatgggagccgtatctcgcgctggccgcgtga

SEQ ID NO: 85 - DNA, 498 bp, Microbacterium sp.
  source 1..498: mol_type = genomic DNA
atgcgcacacttgtactgaacgcaggatacgaaccgctcgcgatcgtgtcgttcaagagagccctggtcctggtcatgaacgacaaggcgacggtcatcgaacgtgtcgaggacgaccccgtctggggcagtcacggcgtttacgaccgtcctgccgtcatcgtcctctcgcggtatgtgcgcattccgatcagcagacgcgtcccggtcacacggcggggggtcctccgccgtgacaaccaccgctgcggctactgcggcaaggccgcctcgaccatcgaccacgtgatgccgcgttcgcgcggaggggctgattcgtgggagaacctcgtcgcgtgctgcctgcgctgcaacaacaccaagagcgaccgcacgccgcaggagatgcgctgggaactgaggttcgcgcctcgtcctccgcacgggacggcgtggacggtccgcggcacggagcggagcgacccgcgatgggagccgtacctggccctcgccgcgtga

SEQ ID NO: 86 - DNA, 975 bp, unidentified
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcatttgctaaaatatattggaatttggttattcatcatgatttaaatcaattaaatacacacaatagacaagctggaattcaaaaagagctaaaggaatttcagttaatgcatggtgtccccaataaagtagtatttgatcgtttaccaagtaatcttcaattgcagttagtagaaagaactaaaaaagtgggggcgaggtatgtagtaggggtgttatacggagatatggaaggttctatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagatgatgtcttgttgaaagtggaaaatatttcaaaacacgaatcgctgcatgcattctataacatactttggaaatacgaaacaaaagaatgtttttattgtggaaaacgtctttctgataaacgcatcgctactcatgtagatcattttattccatggagttatatccaatcagataatatgtggaatttagtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 87 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcatttgctaaaatatattggaatttggctattcatcatgatttaaatcaattaaatacacacaatagacaagctggaattcaaaaagagctaaaggaatttcagttaatgcatggtgtccccaataaagtagtatttgatcgtttaccaagtaatcttcaattgcagttagtagaaagaactaaaaaagtgggggcgaggtatgtagtaggggtgttatacggagatatggaaggttctatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagatgatgtcttgttgaaagtggaaaatatttcaaaacacgaatcgctgcatgcattctataacatactttggaaatacgaaacaaaagaatgtttttattgtggaaaacgtctttctgataaacgcatcgctactcatgtagatcattttattccatggagttatatccaatcagataatatgtggaatttagtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 88 - DNA, 498 bp, Microbacterium sp.; identical to SEQ ID NO: 29
  source 1..498: mol_type = genomic DNA

SEQ ID NO: 89 - DNA, 975 bp, Bacillus megaterium; identical to SEQ ID NO: 86
  source 1..975: mol_type = genomic DNA

SEQ ID NO: 90 - DNA, 498 bp, Microbacterium sp.
  source 1..498: mol_type = genomic DNA
atgcgcacactggtcctgaacgccggatacgaacccctcaccgtggtctcgttcaagagagccatggtgctcgtgatgaacgacaaggccagcgtgatcgagcacgtggagggcgatcccgtatgggcggggcgcaacgcgtacgatcggccggtcgtgatcctgctcacccgctacgtgcgcgtgccgtcgtcgaggcgggttcctgtcacgcggcggggcgtgctgcgccgggacgaccaccgctgcggctactgcgggcggagtgcgtcgacgatcgaccacatcctgccgcgctcgcgcggaggggcggacagctgggagaacctcgtcgcctgctgtctgaagtgcaacaacatcaagagcgaccgcaccccgcaggagatgggctgggagctgcggatgacgccgcgtccgcctcacggcacggcctggaccgtgcgcggcggtgagcgcggcgacccgcgctgggagccgtacctcgcgctcgcggcgtag

SEQ ID NO: 91 - DNA, 498 bp, Microbacterium sp.; identical to SEQ ID NO: 55
  source 1..498: mol_type = genomic DNA

SEQ ID NO: 92 - DNA, 975 bp, Bacillus megaterium; identical to SEQ ID NO: 87
  source 1..975: mol_type = genomic DNA

SEQ ID NO: 93 - DNA, 975 bp, Bacillus megaterium; identical to SEQ ID NO: 87
  source 1..975: mol_type = genomic DNA

SEQ ID NO: 94 - DNA, 501 bp, unidentified
  source 1..501: mol_type = genomic DNA; note = Pseudarthrobacter chlorophenolicus
atgcgcactctcgttctgaatgctggatatgaaccgctggcggttgttacgttccgccgggcgctggtgcttgtgcttacgggcaaggcgagcgtggtggccgaaggggacgaccccgtcgtgggccccacagacgtcctgggccgcccgtccgtgatcctcctcaaccgctacatccgcccccgatacaaccacgccacggccgtcagccgcaggggggtgttgcgccgcgacggacacaaatgcgcctactgcggaaaagcggcgcacaccattgaccatgtccacccgaagtcccgcggcggggcagattcctgggagaacctggtggcggcgtgcctgcgctgcaacaacgtcaagggcgaccacacccccgccgagatgggctggaccctccggtttgttccggaaccgccgcatggaaccatttggcagatcaaggaactggagaagcccacgcctgcctgggaccccttcctgctgcctgaacgcgctgcctga

SEQ ID NO: 95 - DNA, 537 bp, unidentified
  source 1..537: mol_type = genomic DNA; note = Glutamicibacter arilaitensis
atgccagatggtagccatcggttcatgaaggtagtacgagcaatgcgaacattggtattaaacgcaggatacgagccactggcagtagtcacgttccgtcgagcgctagtgctggtcatgaccgagaaagcgagcatcgtcctccaagacgatctcgcacccgttcgcagttctcaaggagatttaccgcggccaactgtcatcgtgctgaaacgctatgtacgacgcccctaccgggacatgcaacatgtcacccgtaggggcgttttgcgtagggactccttcacctgcgcctattgcggcaaagcagccaatacgattgaccacgtgatcccgcgcagcaggggcgggcagaacacttgggagaacctcgtggccgcctgcctgaattgcaacagccgcaaagcggacaagatgctctcgcagttgggatggcaattgcgcattacgccgcatcgtccacgggttgccgggcatctggtttcgggactgcagcatgttgacgcggcgtgggacgaatacctgcatgctagctag

SEQ ID NO: 96 - DNA, 984 bp, Paenibacillus naphthalenovorans
  source 1..984: mol_type = genomic DNA
ttggaaggttggaaattaaatgcaggtaagttaaacaataaaaatttagagcatagtgatatttggaactattttaacatcttattttcaacaaaatcaaaaaaccaatcgagctataaattcggtcttcttagagcgatactggataatctgtataatactgatattgattttcggctaagttatgagcaattatttcagtcgttcactcgaatttattggaaattgattctgagatatgggttgaaacaaactgatagaagccataaacattcagtcatagaaagattgctcttacgagttgtaatggtaaataaaattcctgtagaagcatctttcgatggtctttcagaagacctgcagaagatgatagtccaatcagttaaacgagaaggaaaacgatatgttttgggtgctgtatacggcgacactgatggggtattttataattttgataataggctcgaatacctaaccctaaatccaaaggttgtagtatttttgcaaacctatcagcaagtgctctttagattgattaattatgagtttctgaagtttcttcaacgaaataatgagccttcaaaatgctcgaacctaattgaaataatagagaatctgaccaaaagaacatcgttagaaagatatcgtcaaacacttcttgataactatggcagtcactgtttttactgtgagaaggacttaacaactgagtcatccaccgaatgtgaccattttatcccttggagtttcgttcatagcgatcaattatggaatcttgtattagcctgcagaaaatgtaataactcaaaaagagacaaaatagccgtgcataaatatctagataaacttatacaatgcaatcgaattttattacacgacagtaagataatgcaaagagatatgttgaattacaaagatgaaaagctcttaaacatgtataactttgcattgttaaatggttattcacagagctggactccgtaa

SEQ ID NO: 97 - DNA, 231 bp, Paenibacillus naphthalenovorans
  source 1..231: mol_type = genomic DNA
gtggtcaaccgtcagaccatccagcggctgtttagcaacaaggagttccggcggctgattctggagcgggaccagtatatatgccggttttgcggccaatacggagatacgatcgaccatatgctaccgcgcgccaaaggcgggcataccacgccggacaactgtgtctgcgcctgccacagctgcaatcaatccaaggccgataaggatctggaggagtttattcaataa

SEQ ID NO: 98 - DNA, 975 bp, unidentified; identical to SEQ ID NO: 9
  source 1..975: mol_type = genomic DNA

SEQ ID NO: 99 - DNA, 501 bp, unidentified
  source 1..501: mol_type = genomic DNA; note = Pseudarthrobacter chlorophenolicus
atgcgcactctcgttctgaatgctggatatgaaccgctggcggttattaccttccgccgggcgctggtgcttgtgcttacaggcaaggccagtgtggtggccgagggggacgaccccgtggtgggccccaccgacgtcctcgggcgtccgtccgtgatcctcctcaaccgctacatccgcccccggtacaaccactccacggccgtcagccgtagaggcgtgctccgacgcgacgggcataaatgtgcctactgcgggaaagcggcgcacaccattgaccacgtgcatccgaaatcccggggcggggccgattcgtgggagaacctggtggccgcctgcctgcggtgcaacaacgtcaaaggcgaccacactcccgccgagatgggatggaccttgcggttcgtgccggaacctccgcacggaaccatctggcaaatcaaggagctggagaagcccacgccggcgtgggatcccttcctgcttccggaacgcgctgcttga

SEQ ID NO: 100 - DNA, 501 bp, unidentified
  source 1..501: mol_type = genomic DNA; note = Pseudarthrobacter chlorophenolicus
atgcgcactctcgttctgaatgctggatatgaaccgctggcggttattactttccgccgggcgctggtgcttgtgcttacaggcaaggccagtgtggtggccgaaggggacgaccccgtggtgggccccaccgacgtcctggggcgcccgtccgtgattctcctcaaccgctacatccgcccccggtacaaccactccacggccgtcagccgcaggggagtgctccgacgcgacgggcataaatgtgcctactgcgggaaagcggcgcacaccattgaccatgtgcacccgaaatcccggggcggggctgattcgtgggagaacctggtggccgcctgcctgcggtgcaacaacgtcaagggcgatcacactcccgccgagatgggatggaccctgcgcttcgtcccggaacctccgcacgggaccatctggcagatcaaggagctggagaagcccaccccggcctgggatccgttcctgcttccggaacgggctgcctga

SEQ ID NO: 101 - DNA, 501 bp, unidentified
  source 1..501: mol_type = genomic DNA; note = Pseudarthrobacter chlorophenolicus
atgcgcactctcgttctgaatgctggatatgaaccgctggcggttgttacgttccgccgggcgctggtgcttgtgcttacgggcaaggcgagcgtggtggccgaaggggacgaccccgtcgtgggccccacagacgtcctgggccgcccgtccgtgatcctcctcaaccgctacatccgcccccgatacaaccacgccacggctgtcagccgcaggggggtgctgcgccgcgacggacacaaatgcgcctactgcgggaaagcggcgcacaccatcgaccacgtccacccgaagtcccgcggcggggcagattcctgggagaacctggtggcggcgtgcctgcgctgcaacaacgtcaagggcgaccacacgcccgccgagatgggctggaccctccggtttgttccggagccgccgcatggaaccatctggcagatcaaggaactggagaagccgacgcctgcctgggatcccttcctgttgcctgaacgcgctgcctga

SEQ ID NO: 102 - DNA, 501 bp, Curtobacterium pusillum
  source 1..501: mol_type = genomic DNA
atgcgcactctcgttctgaatgccggttacgagccgcttgctgtcgtctcgttcaagcgagccctcgccctcgtgatgaatcacaaggcgaccatcgtgcagatcgacacaggccacccggtgttcggcatcacggcggagtgggaccgcccgagcgtcatcctgctgacgcgctacgtgcggctgccgcacggccggcaggtgccggtgtcgcgccgcggggtgctgcgtcgtgacgcgcatcgctgcgcctactgcgcgaagccggcctcgacgatcgatcacgtggtgccacgctcgaagggcggccaggactcatgggagaacctggtcgcgtgctgcctgcgctgcaacaacgtcaagggcgaccgctcgctcgcggacatggggtgggagctgcgcttcacgccgcggatgccgcacgagccggcgtggatggtgcgcggctcggagcacctgctgcccgagtgggaggactacctgcagccgcaggctgcatag

SEQ ID NO: 103 - DNA, 564 bp, Azospirillum lipoferum
  source 1..564: mol_type = genomic DNA
ttggctccaccacccgatcactgtccggctctggtgctgaacgcggatttccgcccgctcagttacttccccttgtcgctatggtcctggcaggaagcggtcaaggcggtctttctcgaacgggtcaacatcgtatcgcactatgaccgggtcgtccgatccccaagtttcgaagtccggttgcccagcgtcatctcgctgaaagagttcatcccggccacgcgccgcccggccttcacccgcttcaacgtcttcctgcgcgaccgcttcacctgccagtattgcggccgccccttccccacccacgatttgaccttcgaccatgtgatcccgcggtcgcgcggcggtcggacgacgtgggaaaacgtcatcacatcctgttcggcctgcaatctggcgaagggcgaccgccttccccacgtctgcggcatgatccccctcagtccgcccttccagcccagtgcctacgagctgcaagagaacggacgcgccttcccgccaaacttcctccatgaaagttggcgggattttctttactgggactcggaactggacccgatgtag

SEQ ID NO: 104 - DNA, 978 bp, Paenibacillus sp.
  source 1..978: mol_type = genomic DNA
atgaacttcattgaaaaatggttgcacatcattagaaattgtagttttgataatacttataaaatggcatgggcaaagtcaatcacagaaattgctattgaaaatgatacacctcccaatcccgatataaaaatcgaaattaacctgcgggatattgcaaagaatgtacttcgatattattttgagcaaactatattttttgatcttcaacaaagtgcaaatcctaacaaaccacctgtgatggtttcgatagcgaaaggtctcattgcatcgtatagagccaatacaggctctgtaaagcctgttaagtggtttagatcgaacattgagtcaatctgttcagagaactatgaaaaggcagttagcgcgactgtaagggcgttgaaaacagatgttagctatcgttttttgcgtcttgaaggtaaagaagtggaaggagtatatgaatatgagcaaaaagctgacagtcttttcattcacgccgacaaattatatgctctaaagcaaaataagctgatagtacttgatgcaattaactatcgctggactcagatgctggaaaattttaatcactcgccaaaattatcgaagaaggtaagaattatcgatgaggatcaagtgcaacgtaaatctttagggaaatttgtaaaatatcttgaagtggagaattcagagtctatttgcttcttgtgtggattgaagattgatggcgagacaccggcaattgatcatgtcattccttggtcttacttgtattcagatgatctatggaatttagtcttcacccaccaatcatgcaatagtgttaagtcaaacttgataccaagtgaagagatgatagcccgacttgaaacaaggaacaatgaattgctcaagcgaatgcggaagatgggcaaaaatgacaaacatatttctgaacttgaaatggcaaacgaaagaaatattgtgcgtaaattttggattgcatgtcagggttag

SEQ ID NO: 105 - DNA, 420 bp, unidentified
  source 1..420: mol_type = genomic DNA; note = Paenibacillus yonginensis
atgattacggtactgaatacagatggtcgggaattaaccccttgtaccctggaccgagcttggcgggaggtgaatcgatcgcgtgccgtttggattaatgagcagaccattcagttgttatacaacccttttctgttccggaaatatcggaagatcgccttaaaaagagaccgttacacatgcctatggtgcggacagcctgcaacaacggtagatcatctgatcccctccagcaagggcggttcggatctgccgcgcaaccttattgcctcctgcggcgagtgcaactcaagacgggggaatcgcccggcgtgggagtattttagagagaagatgggtttcattccccgtcccctcaagcttggttttcatattttgatagctttaggctgctcaaagatcaaaccaagatctcattaa

SEQ ID NO: 106 - DNA, 501 bp, Arthrobacter sp.
  source 1..501: mol_type = genomic DNA
atgcgcactctcgttctgaatgctggatatgaaccgctggcggtagtgacattccgccgggcgctggtccttgtgctcaccggaaaggcaagcgtggtggctgaaggcgacgatcctgtcgtcgggccacaggacatcatgggccgtccgtccgtgattctcctgaaccgctacatccgtccccggtacaacatgatcacagcggtgagcagacgcggtgtgcttcgccgcgacggccatcggtgcgcctactgcggcaaggcagcccacaccatagaccatgtccagccgaaatcccggggcggtgctgactcctgggaaaacctggtggcagcatgcctgcgctgcaacaacgtcaaaggtgaccacacgcccagcgagatgggctggaagctccggttcgatccggctccaccggtgggcaccatgtggcagatcaaggaactggagaagcccacaccggcgtgggacccgttcctgcttccggaacgggcagcctga

SEQ ID NO: 107 - DNA, 501 bp, Arthrobacter sp.
  source 1..501: mol_type = genomic DNA
atgcgcactctcgttctgaatgctggatatgaaccgctggcggtcatcacgttccgccgggcactggtgcttgtgctcacgggcaaagccagtgtggttgccgagggtgacgatcccgtggtggggccccaggagattttgggccgcccatccgttatcctcctgaaccgctatatccgccccaggtacaaccaggccaccgctgtcagccgccgcggcgtgttgaggcgtgatggccaccgttgcgcctactgcgggaaagcggcccacaccattgaccatgtccagcccaagtcccgcggcggtgccgattcctgggagaacctggtggcggcctgcctgcgttgcaacaacgtcaagggcgaccacactcctgctgaaatggggtggaagctgaggttcgtcccggagcctccgcacggaaccatctggcagatcaaggaactcgaaaaacccacccctgcctgggatccgttcctgctgcccgagcgcgcagcctga

SEQ ID NO: 108 - DNA, 501 bp, unidentified
  source 1..501: mol_type = genomic DNA; note = Pseudarthrobacter phenanthrenivorans
atgcgcactctcgttctgaatgctggatatgaaccgctggcggttattactttccgccgggcgctggtgcttgtgctgacaggcaaggccagtgtggtggccgaaggggacgaccccgtggtgggccccaccgacgtcctggggcgcccgtccgtgatcctcctcaaccgctacatccgaccccggtacaaccattccactgccgtcagccgcaggggagtgctccggcgcgatgggcataaatgcgcctactgcgggaaggctgcgcacaccattgaccacgtgcatccgaaatccaggggcggggcagattcgtgggagaacctggtggccgcctgcctgaggtgcaacaacgtcaaaggcgaccacactcccgccgagatgggatggaccctgcggttcgtgccggcacctccgcatggaaccatctggcagatcaaggagctggagaaacccacaccggcgtgggatcccttcctgcttccggaacgcgctgcctga

SEQ ID NO: 109 - DNA, 501 bp, Arthrobacter sp.
  source 1..501: mol_type = genomic DNA
atgcgcactctcgttcttaatgctggatatgaaccgctggcggttgtgaccttccgccgggcgctggttcttgtgctgaccggaaaagctagcgtggtggctgagggcgacgatcctgtcgtcgggccgcaggagattctgggccgcccgtccgtgattctcctcaaccgctacatccggccccggtacaacaccattacggctgtgagccgccggggcgtgctccgccgtgacggccaccgctgcgcctattgcggtaaggcagcacacaccattgaccacgtccacccgaaatcgcgagggggcgccgactcgtgggaaaacctcgtggccgcctgcctgcggtgcaacaacgtcaagggggaccacacgccggccgagatgggctggaagctccggtttgtgccggcgcctccgcacggcaccatctggcagatcaaggagctggaaaagcccacgcctgcgtgggacccgtttctcctgcccgaatcggccgcctga

SEQ ID NO: 110 - DNA, 501 bp, Arthrobacter sp.
  source 1..501: mol_type = genomic DNA
atgcgcactctcgttctgaatgctgggtatgagcccctggcggtgatcactttccgccgggcgctggtgcttgtgctcacgggcaaggccagcgtggttgccgaaggtgatgatcccgtggtgggaccgcaggaaatcctgggccgcccgtccgtgatcctcctgaaccgctacatccgtccaaggtacaaccaggccacggcagtcagccgccgtggagtgcttagacgcgacggccatagatgcgcgtactgcggaaaagcggcccacaccatagaccatgtccagcccaagtcccggggtggtgaggactcctgggaaaacctagtggcggcctgcctgcgctgcaacaacgtcaagggcgaccatacgccagctgaaatgggctggaaactgcgtttcgtcccggagccaccgcacggaaccatctggcagatcaaggaactcgaaaagcctactcctgcctgggatccgttcctgctgccggagcgcgccgcctga

SEQ ID NO: 111 - DNA, 501 bp, Arthrobacter sp.; identical to SEQ ID NO: 110
  source 1..501: mol_type = genomic DNA

SEQ ID NO: 112 - DNA, 501 bp, Arthrobacter sp.
  source 1..501: mol_type = genomic DNA
atgcgcactctcgttctgaatgctggatatgaaccgctggcggttattactttccgccgggcgctggtgcttgtgcttacgggcaaggcgagcgtcgtggccgaaggggacgatcctgtcgtgggaccgcaggagattttggggcgcccgtccgtgatcctcctgaaccgctacatccggcccagatacaaccagtccacggcagtcagtagacgcggggtgctccggcgcgacgggcacaagtgcgcctattgtggaaaagctgcgcacactatcgaccacgtccaccccaagtcccgcggcggagcggattcctgggagaacctcgtggctgcatgccttcggtgcaacaacgtcaaaggtgatcacaccccggcggagatgggctggaagctgcggttcgtgcccgagcctccgcacggcaccatctggcagatcaaggaactcgagaagcccacgcctgcgtgggatccgttcctgctgccggagcgggctgcctga

SEQ ID NO: 113 - DNA, 501 bp, Arthrobacter sp.
  source 1..501: mol_type = genomic DNA
atgcgcactctcgttcttaatgctggatatgaaccgctggcggttgtgaccttccgccgggcgctggttcttgtgctgaccggaaaagctagcgtggtggccgaaggcgacgatcctgtcgtcgggccgcaggagatcctgggccgtccgtccgtgatcctcctgaaccgctacatccgcccgcggtacaacaccatcaccgccgtgagccgtcggggcgtgctgcgccgcgacggccaccgctgtgcctattgcggtaaagcagcgcacaccattgaccacgtccatcccaaatcgaggggcggtgccgactcgtgggaaaacctggtggccgcctgcctcagatgcaacaacgtgaagggcgaccacaccccggccgagatgggctggaagctcaggtttgtgccggcgcctccgcacggcaccatctggcagatcaaggagctggaaaagcccactcccgcgtgggatcctttcctgctgcccgaatccgccgcctga

SEQ ID NO: 114 - DNA, 501 bp, unidentified
  source 1..501: mol_type = genomic DNA; note = Pseudarthrobacter phenanthrenivorans
atgcgcactctcgttctgaatgctggatatgaaccgctggcggttatcactttccgccgggcgctggtgcttgtgctcacgggcaaagcaagcgtggtggccgagggtgacgatcccgttgtgggaccgcaggagatcctgggccggccgtccgtgatactccttaaccgctacatccgtcccagatacaaccaggccacggctgtcagccggcggggagtgctgcggcgtgatgggcacaaatgcgcctactgcggaaaagcggcccacaccatcgaccatgtccaccccaagtcccgcggtggtgcggattcgtgggagaacctcgtggcggcctgcctgcgctgcaacaacgccaagggcgaccacacacccgccgaaatggggtggaaactccggttcgttcccgaaccgccgcacggaagcatgtggcagatcaaggaactcgagaagccgacgccggcgtgggacccgttcctgctgccggagcgcgccgcctga

SEQ ID NO: 115 - DNA, 501 bp, unidentified
  source 1..501: mol_type = genomic DNA; note = Pseudarthrobacter phenanthrenivorans
atgcgcactctcgttctgaatgctggatatgaaccgctggcggttatcactttccgccgggcgctggtgcttgtgctcacgggcaaagcaagcgtggtggccgagggtgacgatcccgttgtgggtccgcaggagatcctgggccggccgtccgtgatactccttaaccgctacatccgtcccagatacaaccaggccacggctgtgagccggcggggagtgctgcggcgtgatgggcacaaatgcgcctactgcggaaaagcggcccacaccatcgaccatgtccaccctaagtcccgcggtggtgcggattcgtgggagaacctcgtggcggcctgcctgcgctgcaacaacgccaagggcgaccacacacccgccgaaatgggatggaaactccggttcgttcccgaaccgccgcacggaagcatgtggcagatcaaggaactcgagaagccgacgccggcgtgggacccgttcctgctgccggagcgcgcagcctga

SEQ ID NO: 116 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaatttttcgctaatggtcactttactacaacgtataaatatggattaatgaaagctttaattgaaaacctatataacgttgacaatcggttagtacttacgtttgatcaagtgtatttttcatttgctaaaatatattggaatttggttattcatcatgatttaaatcaattaaatacacacaatagacaagctggaattcaaaaagagttaaaggaatttcagttaatgcatggtgtccccaataaagtagtatttgatcgtttaccaagtaatcttcaattgcagttagtagaaagaactaaaaaagtgggggcaaggtatgtagtaggggcgttatacggagatatggaaggttctatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagatgatgtcttgttgaaagtggaaaatatttcaaaacacgaatcgctgcatgcattctataacatactttggaaatacgaaacaaaagaatgtttttattgtggaaaacgtctttctgataaacgcatcgctactcatgtagatcattttattccacggagttatatccaatcagataatatgtggaatttagtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 117 - DNA, 975 bp, Bacillus sp.; identical to SEQ ID NO: 86
  source 1..975: mol_type = genomic DNA

SEQ ID NO: 118 - DNA, 498 bp, Microbacterium testaceum
  source 1..498: mol_type = genomic DNA
atgcgcacgctggtgttgaacgcgggatacgagccgctcgcggtggtctcgttcaaacgagccctggtgcttgtgatgagcgacaaggccacggtggtggaacacgacgaggagctgcccgtctggggcaccaccgcgatgtacgaccgtccggcggtgatcgtgctcacccgctacgtgcgcgtgcccacctcgcgcagcatgccggtgacccgcaggggagtgctccggcgcgatgcgaaccactgcgcgtactgcgggaaggcggcatcgacgatcgaccacatcctgccgcgttcgcggggaggcgccgactcgtgggagaacctcgcggcctgctgcctgcgctgcaacaacgtcaagggcgatcgcacgccgcaggagatgggctgggaactgctgctgacaccccgtccgccgcacgggacggcatggaccgttcgcggtgtcgagcgggccgatccgcgctgggagccgtacctcgccctcgcggcgtga

SEQ ID NO: 119 - DNA, 501 bp, unidentified
  source 1..501: mol_type = genomic DNA; note = Pseudarthrobacter chlorophenolicus
atgcgcactctcgttctgaatgctggatatgaaccgctggcggttattactttccgccgggcgctggtgcttgtgctcacgggcaaggcgagcgtggtggccgagggggatgaccccgtggtggggcccaccgacgtcctgggccgtccgtccgtgattctcctcaaccggtacatccggccccggtacaaccattccacggctgtcagccgcaggggtgtgctcagacgcgacggccataaatgcgcatactgcggcaaggcagcgcacaccatcgatcatgttcaccccaaatccaggggcggggctgactcgtgggagaacctggtggccgcctgcctccgctgcaacaacgtcaaaggcgatcacaccccggccgagatgggctggacgctgcggttcgtgcccgagcctccgcgcggcactatctggcagatcaaggaactggagaagcccacccccgcgtgggatccgttccttcttcccgaacgcgctgcctga

SEQ ID NO: 120 - DNA, 501 bp, Arthrobacter sp.; identical to SEQ ID NO: 106
  source 1..501: mol_type = genomic DNA

SEQ ID NO: 121 - DNA, 501 bp, unidentified
  source 1..501: mol_type = genomic DNA; note = Pseudarthrobacter phenanthrenivorans
atgcgcactctcgttctgaatgctggatatgaaccgctggcggttatcactttccgccgggcgctggtgcttgtgctcacgggcaaagcaagcgtggtggccgagggtgacgatcccgttgtgggaccgcaggagatcctgggccggccgtccgtgatactccttaaccgctacatccgtcccagatacaaccaggccacggctgtcagccggcggggagtgctgcggcgtgatgggcacaaatgcgcctactgcggaaaagcggcccacaccatcgaccatgtccaccccaagtcccgcggtggtgcggattcgtgggagaacctcgtggcggcctgcctgcgctgcaacaacgccaagggcgaccacacacccgccgaaatgggatggaaactccggttcgttcccgaaccgccgcacggaagcatgtggcagatcaaggaactcgagaagccgacgccggcgtgggacccgttcctgctgccggagcgcgccgcctga

SEQ ID NO: 122 - DNA, 501 bp, unidentified; identical to SEQ ID NO: 115
  source 1..501: mol_type = genomic DNA; note = Pseudarthrobacter phenanthrenivorans

SEQ ID NO: 123 - DNA, 501 bp, unidentified
  source 1..501: mol_type = genomic DNA; note = Pseudarthrobacter chlorophenolicus
atgcgcactctcgttctgaatgctggatatgaaccgctggcggtcattactttccgccgggcgctggtgcttgtgctcacgggcaaggcgagcgtcgtggccgaaggggatgaccctgtcgtgggacccacggacgtcctgggccgcccgtccgtgatcctcctcaaccggtacatccggccccgatacaaccactccacggctgtcagccgcaggggcgtactcagacgcgacggccataaatgcgcatactgcggcaaggcagcgcataccatcgaccatgtgcaccccaagtcccggggcggggctgactcgtgggagaacctggtggcagcctgccttcgctgcaacaacgtcaagggggaccacaccccgaccgagatgggctggagcctgcgctttgtgccggagcctccgcgcggcaccatctggcagatcaaggaactggaaaagcccacgcccgcgtgggatccgttcctcctgcccgaacgcgccgcctga

SEQ ID NO: 124 - DNA, 498 bp, Leifsonia xyli
  source 1..498: mol_type = genomic DNA
atgagaactctggtcctcaacgccggctacgaaccgctcgcggtcgtctccttcaaacgagccctcgtgctggtgatgaatcagaaggccacggttgtggccatcgacccggaccaccccgtgtggggcatcagcgacagctacgagcgaccgtccgtcatcctgctcacccgctacgtgcggctgccccgaggccgcgccgttccggtcagccgccgtggcgtgctgcgccgtgacgcccaccgttgcggatactgcggcaagaccgcgaccacgatcgaccacgtgcagccccggtcccgcggcggcgcggacagctgggagaacctcgtggcctgctgcctccgctgcaacaacgtgaagggtgaccgtacgcccggcgaaatgggctggtcgctgcgtctgaccccgcgagtgccgcacgacgcgtcgtgggtcgtgcgcggagtggagtcggcgcagcccgagtggcaggactacctggcgcctgccgcgtag

SEQ ID NO: 125 - DNA, 501 bp, unidentified
  source 1..501: mol_type = genomic DNA; note = Pseudarthrobacter chlorophenolicus
atgcgcactctcgttctgaatgctggatatgaaccgctggcggtcatcactttccgccgggcgctggtgcttgtgctcacaggcaaggcgagcgtggtggccgagggggatgaccccgtggtaggccccaccgacgtcctgggccgcccgtccgtgattctcctcaaccgctacatccggccccgatacaaccactccacggccgtgagccgcaggggtgtgctccggcgcgacgggcataaatgcgcctactgcggcaaggctgcgcacaccattgaccatgtgcacccgaaatcccggggtggggcggattcgtgggagaacctggtggccgcctgcctgcggtgcaacaacgtcaagggcgaccatactcccgccgaaatgggctggaccctgcggtttgttccggagccgccgcacggaaccatctggcagatcaaggaattggagaagcccacgccggcgtgggatccgttcctgcttccggagcgcgctgcctga

SEQ ID NO: 126 - DNA, 585 bp, Paenibacillus xylanexedens
  source 1..585: mol_type = genomic DNA
atgagaattcatggggagttagtactagcaagaagaaaagaattttcaattgagaataattattcgaactatagggagatattgcttcaagattttcaaaatatatgtggttattgtgggaaagatatacatttagctaaaaaagggtttgaaatagatcatttttgtccagtttcaactgataattctaaagagaatgattataataatcttgtattatcctgttttacgtgtaatagaaaaaaaagtaaaaaatggcctacgaaaaacaaggatttatgtcatgatggtgtagaaggctttgttgacccaacaactcaagaatatgatactcatataggtagagatattaatggtataatagaacattatactgatttaggtaggtatatgcacggtgtgtttaaatttagattgagacctacaggtgaaatttggaaatcaatggagctaatgaaacgaaaaaagtacttatataaaataaaagaatcgtcaaatctatctaaagaacaaaaagatttgtttattgaaattcaagaggagcttgactcacttcttgattatttgcttggaaaaggagaatag

SEQ ID NO: 127 - DNA, 585 bp, Paenibacillus xylanexedens; identical to SEQ ID NO: 126
  source 1..585: mol_type = genomic DNA

SEQ ID NO: 128 - DNA, 585 bp, Paenibacillus xylanexedens; identical to SEQ ID NO: 126
  source 1..585: mol_type = genomic DNA

SEQ ID NO: 129 - DNA, 498 bp, Microbacterium sp.
  source 1..498: mol_type = genomic DNA
atgcgcacactggtcctgaatgccgggtacgagccgctgacggtggtctcgttcaagagggcgatggtcctcgtgatgaacgagaaggccagcgtgatagagcgggtcgagaacgatcccgtcatcgccggctgcagacgctacgaccggccagtggtgatcctgctgacccgctacgtgcgcgtgccttcggctcgtcgccagccggtcaccaggagaggcgtgcttcgtcgcgacgaccaccggtgcgggtactgcggcaaggcggcctcgaccatcgaccacatcatgccgcgatcacgagggggagcggacagctgggagaacctggtcgcctgctgtcttcggtgcaacaacgacaagagcgaccgcaccccccaagagatggggtgggagctgcgtcgcatcccgcgggcgccgcatggcgggacgtgggtggtgcgcggggcggaacggggtgatcccgtctgggagaggtacctggcgctggcggcatag

SEQ ID NO: 130 - DNA, 501 bp, Arthrobacter sp.
  source 1..501: mol_type = genomic DNA
atgcgcacactcgttctgaatgctggatatgaaccgctggcggtagtcaccttccgccgggcgctggtccttgtgctcaccggaaaagcaagcgtagtggccgaagacggcgaacctgtcgtcgggccacaggagatattgggacgtccgtccgtaatactccttaaccgctacatccgcccccggtacaacaggatcacagccgtaagccgccgcggggttctccgccgcgacggccaccgctgtgcctactgcggtaaaacagcccacacaatagaccacgtccaccccaaatccaggggcggcgcggattcctgggaaaacctggttgcggcgtgcttgaaatgcaacaacgccaagagcgaccacacgccggcggagatgggctggaagctccgtttcactcccggcgttcccaccggaaccatgtggcagatcaaggaactcgagaaacctgcaccgggctgggacccgttcctcttgccggaatcagctgcctga

SEQ ID NO: 131 - DNA, 501 bp, Arthrobacter sp.
  source 1..501: mol_type = genomic DNA
atgcgcacactcgttctgaatgctggatatgaaccgctggcggtagttaccttccgccgggcgctggtccttgtgctcaccggaaaagcaagcgtagtggccgaaggcggcgaacctgtcgtcgggccgcaggagattctgggacgcccgtccgtgatcctcctcaatcgctacatccggccgcggtacaacaggatcacggccgtcagccggcgcggagttctccgccgcgacggccaccgctgcgcctactgcggtaagacagcccacaccatagaccacgtccaccccaaatccaggggcggcgcggattcctgggaaaacctggttgcggcgtgcttgaaatgcaataacgccaagagcgaccacacgccggcagagatgggttggaaactccgtttcacacccggcgttccctccggaaccatgtggcagatcaaagaactcgagaagcctgcgccggactgggatccgttcctcttgccggaatccgctgcctga

SEQ ID NO: 132 - DNA, 501 bp, Arthrobacter sp.; identical to SEQ ID NO: 113
  source 1..501: mol_type = genomic DNA

SEQ ID NO: 133 - DNA, 501 bp, unidentified
  source 1..501: mol_type = genomic DNA; note = Pseudarthrobacter chlorophenolicus
atgcgcactctcgttctgaatgctggatatgaaccgctggcggttgttacgttccgccgggcgctggtgcttgtgcttacgggcaaggcgagcgtggtagccgaaggggacgaccccgtcgtgggccccacagacgtcctgggccgcccgtccgtgatcctcctcaaccgctacatccggccccggtacaaccactccacggccgtgagccgccggggggtgctgcgccgggacggacacaaatgcgcctactgcggaaaagcggcgcacaccatcgaccatgtccacccaaagtcccgcggcggtgcagattcctgggaaaacctggtggcggcgtgcctgcgctgcaacaacgtcaagggtgaccacaccccctccgagatgggctggacgctccggtttgtcccggaaccgccgcatggaaccatctggcagatcaaggagctggagaagccgacgccggcctgggatcccttcctgctgcctgaacgtgctgcctga

SEQ ID NO: 134 - DNA, 501 bp, Arthrobacter sp.
  source 1..501: mol_type = genomic DNA
atgcgcactctcgttctgaatgctgggtatgagcccctggcggtgatcactttccgccgggcgctggtgcttgtgctcacgggcaaggccagcgtcgttgccgaaggcgatgatcccgtggtgggaccgcaggaaatcctgggccgtccctccgtgattctcctgaaccgttacatccggccaaggtacaaccaggccactgctgtcagccgccgcggagtgctcaggcgcgacggccacagatgcgcgtattgcggtaaagcggcccacaccatcgaccacgtccaccccaagtcccggggcggcgaggactcctgggagaacctggtggcggcctgcctgcgctgcaacaacgtcaagggcgaccatacgccggcggaaatgggctggaaactgcgtttcgtcccggagccaccgcatggaaccatctggcagatcaaggaacttgagaagcccactccagcgtgggatccgttccttctgccggagcgggccgcctga

SEQ ID NO: 135 - DNA, 501 bp, unidentified; identical to SEQ ID NO: 133
  source 1..501: mol_type = genomic DNA; note = Pseudarthrobacter chlorophenolicus

SEQ ID NO: 136 - DNA, 501 bp, unidentified; identical to SEQ ID NO: 99
  source 1..501: mol_type = genomic DNA; note = Pseudarthrobacter phenanthrenivorans

SEQ ID NO: 137 - DNA, 420 bp, unidentified
  source 1..420: mol_type = genomic DNA; note = Paenibacillus yonginensis
atgattacggtactgaatacagatggtcgggaattaaccccttgtaccctggaccgagcttggcgggaggtgaatcgatcgcgtgccgtttggattaatgagcagaccattcagttgttatacaacccttttctgttccggaaatatcggaagatcgccttaaaaagagaccgttacacatgcctatggtgcggacagcctgcaacaacggtggatcatctgatcccctccagcaagggcggttcggacctgccgcgcaaccttattgcctcctgcggcgagtgcaactcaagacgggggaatcgcccggcgtgggagtattttagagagaagatgggttccattccccgtcccctcaagcttggttttcatattttgatagctttaggctgctcaaagatcaaaccaagatctcattaa

SEQ ID NO: 138 - DNA, 420 bp, unidentified; identical to SEQ ID NO: 137
  source 1..420: mol_type = genomic DNA; note = Paenibacillus yonginensis

SEQ ID NO: 139 - DNA, 420 bp, unidentified; identical to SEQ ID NO: 137
  source 1..420: mol_type = genomic DNA; note = Paenibacillus yonginensis

SEQ ID NO: 140 - DNA, 456 bp, Paenibacillus sp.
  source 1..456: mol_type = genomic DNA
atgaatcatagtcacgatggagaggaaccattgataacggtactcaatatggatgggcaacaactaacaccatgtttaccccatcgggcttggcgggaagttgagcgatcgcgagctgtctggattgatgagcagacgatccaattgctttataatccattcttatttcggaactatcgaaaaactgcactgaaacgagatcatcacacttgtttgtggtgcggacgacacgccacgacagtggaccatattatcccctcaagcaaaggcggctcggatcttccgcaaaacctgatcgcttcatgcagcgaatgcaatacgaaacgaggaaaccgttctgcattgtcctatctcaaagaaaacgcacactctgttccaaattttatgaagctttgctggcgtatctttctagctaaatacagacatcgtcaaatgaaaaaagggacccttgagtaa

SEQ ID NO: 141 - DNA, 420 bp, unidentified; identical to SEQ ID NO: 137
  source 1..420: mol_type = genomic DNA; note = Paenibacillus yonginensis

SEQ ID NO: 142 - DNA, 558 bp, unidentified
  source 1..558: mol_type = genomic DNA; note = Sinorhizobium meliloti
ttgacgattgcagtctcacctcaggccctgccggcgctcgtgttgaacgccgactataggccgctgagttattatcccttgtcgctctggtcctggcaggacgcgatcaaggcggtctttctcgaccgtgtgactatcctcgccgaatacgaacactccgtctcgtcgccgagtttctcgatgcggctgccgagcgtcgtctgtctcaagagctatgtccagccgtcacgccacccggccttcacccggttcaacgtattcctgcgggacaaattcgagtgccagtattgcggctcgccggacgacctgaccttcgaccacgtggtcccgcgtgcgcatggcggccagaccacctgggaaaacgtcgtcgccgcctgttctccctgcaacttgcgcaagggcagcaagctgccgaagcaggccggcatgttcccgcaccagaagccctatcacccgacggttcaggatctgcacaataacggccggcttttcccgcccaaccacctgcatgacagctggatggattatctctactgggatgtcgagctgcagccgtaa

SEQ ID NO: 143 - DNA, 561 bp, Mitsuaria sp.
  source 1..561: mol_type = genomic DNA
gtggacgttctgcagctggacatctcgggccgaccgcaggcctggatctcgacgaaggaagcagcggtgctgtatgccagcgacgcgatcgcgtggacgctgggcgagcccttccacgtgctgcgcgggggctaccaacggctgtccggcctgcaatcccgcatcgagctgcatccgatcgtcgccgtgcgcggcgccattcccagccgcgcctggcgccaggcgccggcgctgtccaacttcaagctgttcgcgcgtgaccggcatgtctgcgcctactgcggccaccgcttccacgccgacctgctgacccgcgagcacatccagccggtctcgcgcaacgggcgggattcgtggatgaactgcatcaccgcctgccgcgcctgcaatggccgcaagggcaaccgcacgcccgaggaggcgcgcatggcgctgctctacctgccctacgtgcccagcctgcacgaagacatgatcctgcgcggccggcgcatcctggcggaccagatggagttcctgctcgcgagcgtgcccaagtcgtcgcgcttgtatgcgtcgtga

SEQ ID NO: 144 - DNA, 561 bp, Mitsuaria sp.; identical to SEQ ID NO: 143
  source 1..561: mol_type = genomic DNA

SEQ ID NO: 145 - DNA, 501 bp, unidentified
  source 1..501: mol_type = genomic DNA; note = Pseudarthrobacter chlorophenolicus
atgcgcactctcgttctgaatgctggatatgaaccgctggcggttgttacgttccgccgggcgctggtgcttgtgcttacgggcaaggcgagcgttgtggccgaaggggacgaccccgtcgtgggccccacagacgtcctgggccgcccgtccgtgatcctcctcaaccgctacatccgcccccgatacaaccacgccaccgccgtcagccgcaggggggtgctgcgccgcgacggacacaaatgcgcctactgcggaaaagcggcgcacaccattgaccatgtccacccgaagtcccgtggcggggcagattcctgggagaacctggtggcggcgtgcctgcgctgcaataacgtcaagggcgaccacacccccgccgagatgggctggaccctccggtttgttccggaaccgccgcatggaaccatctggcagatcaaggaactggagaagcccacgcctgcctgggaccccttcctgctgcctgagcgcgctgcctga

SEQ ID NO: 146 - DNA, 501 bp, Arthrobacter sp.
  source 1..501: mol_type = genomic DNA
atgcgcactctcgttctgaatgctggatatgaaccgctggcggtcatcacgttccgccgggcactggtgcttgtgctcacgggcaaagccagtgtggttgccgagggtgacgatcccgtggtggggccccaggagattttgggccgcccgtccgttatcctcctgaaccgctatatccgccccaggtacaaccaggccaccgctgtcagccgccgcggcgtgctgaggcgcgatggccaccgttgcgcctactgcgggaaagcggcccacaccattgaccatgtccagcccaagtcccgcggtggtgccgattcctgggagaacctggtggccgcctgcctgcgttgcaacaacgtcaagggcgaccacactcccgctgaaatggggtggaagctgaggttcgtcccggagcccccgcacggaaccatctggcagatcaaggaactcgaaaaacccacccctgcctgggacccgttcctgctgcccgagcgcgcagcctga

SEQ ID NO: 147 - DNA, 501 bp, unidentified
  source 1..501: mol_type = genomic DNA; note = Pseudarthrobacter chlorophenolicus
atgcgcactctcgttctgaatgctggatatgaaccgctggcggtcattactttccgccgggcgctggtgcttgtgctcacgggcaaggcgagcgtcgtggccgaaggggatgaccctgtcgtgggacccacggacgtcctgggccgcccgtccgtgatcctcctcaaccggtacatccggccccgatacaaccactccacggctgtcagccgcaggggcgtactcagacgcgacggccataaatgcgcatactgcggcaaggcagcgcataccatcgaccatgtgcaccccaagtcccggggcggggctgactcgtgggagaacctggtggcagcctgccttcgctgcaacaacgtcaagggcgaccacaccccgaccgagatgggctggagcctgcgctttgtgccggagcctccgcgcggcaccatctggcagatcaaggaactggaaaagcccacgcccgcgtgggatccgttcctcctgcccgaacgcgccgcctga

SEQ ID NO: 148 - DNA, 501 bp, unidentified
  source 1..501: mol_type = genomic DNA; note = Pseudarthrobacter chlorophenolicus
atgcgcactctcgttctgaatgctggatatgaaccgctggcggttgttacgtttcgccgggcgctggtgcttgtgcttacgggcaaggcgagcgtggtggccgaaggggacgaccccgtcgtgggccccacagacgtcctgggccgcccgtccgtgatcctcctcaaccgctacatccgcccccgatacaaccacgccacggctgtcagccgcaggggggtgctgcgccgcgacggacacaaatgcgcctactgcgggaaagcggcgcacaccatcgaccacgtccacccgaagtcccgcggcggggcagattcctgggagaacctggtggcggcgtgcctgcgctgcaacaacgtcaagggcgaccacacgcccgccgagatgggctggaccctccggtttgttccggagccgccgcatggaaccatctggcagatcaaggaactggagaagccgacgcctgcctgggatcccttcctgttgcctgaacgcgctgcctga

SEQ ID NO: 149 - DNA, 501 bp, Arthrobacter sp.
  source 1..501: mol_type = genomic DNA
atgcgcacactcgttctgaatgctggatatgaaccgctggcggtagttaccttccgccgggcgctggtccttgtgctcaccggaaaagcaagcgtagtggccgaaggcggcgaacctgtcgtcgggccgcaggagattctgggacgcccgtccgtgatcctcctcaatcgctacatccggccgcggtacaccaggatcacggccgtcagccggcgcggagttctccgccgcgacggccaccgctgcgcctactgcggtaagacagcccacaccatagaccacgtccaccccaaatccaggggcggcgcggattcctgggaaaacctggttgcggcgtgcttgaaatgcaataacgccaagagcgaccacacgccggcagagatgggttggaaactccgtttcacacccggcgttccctccggaaccatgtggcagatcaaagaactcgagaagcctgcgccggactgggatccgttcctcttgccggaatccgctgcctga

SEQ ID NO: 150 - DNA, 501 bp, Arthrobacter sp.
  source 1..501: mol_type = genomic DNA
atgcgcactctcgttctgaatgctggatatgaaccgctggcggttgtgaccttccgccgggcgctggttcttgtgctgaccggaaaagctagcgtggtggccgagggtgacgatcctgtcgtcgggccgcaggagattctgggccgtccgtccgtgattctcctgaaccgctacatccggcccagatacaacaccatcacggctgtgagccgccggggcgtcctgcgccgcgacggccaccgctgcgcctactgcggcaaagcagcacacaccattgaccacgtccacccgaaatcgcggggcggtgcggattcctgggagaacctggtggccgcctgccttcgctgcaacaacgtaaagggtgaccacacgccggcggagatgggctggaagctgcgttttgtgccggcgccaccccacggcaccatctggcagatcaaggaactggaaaagcccacgcccgcgtgggaccccttcctcctgcctgaatctgctgcctga

SEQ ID NO: 151 - DNA, 501 bp, unidentified
  source 1..501: mol_type = genomic DNA; note = Pseudarthrobacter chlorophenolicus
atgcgcactctcgttctgaatgctggatatgaaccgctggcggttatcactttccgccgggcgctggtgcttgtgctcacgggcaaggcaagcgtggtggccgaaggggatgacccggtggtagggcccaccgacgtcctgggccgcccgtccgtgatcctcctcaaccgctacatccggccccggtacaaccactcgacggcagttagccgcaggggtgtgctccggcgcgacggacataaatgtgcctactgcggcaaggcggcgcatacaatcgaccacgttcacccgaagtcccggggcggtgccgatacatgggagaaccttgtggccgcctgcctgcggtgcaacaacgtcaagggcgaccacactcccgcggaaatgggctggacccttcggttcgtgcccgagccgccgcacggaacgatctggcagatcaaggaactggagaagccgacgccggcctgggatccgttcctgctcccggagcgcgccgcctga

SEQ ID NO: 152 - DNA, 501 bp, unidentified; identical to SEQ ID NO: 123
  source 1..501: mol_type = genomic DNA; note = Pseudarthrobacter chlorophenolicus

SEQ ID NO: 153 - DNA, 501 bp, unidentified
  source 1..501: mol_type = genomic DNA; note = Pseudarthrobacter phenanthrenivorans
atgcgcactctcgttctgaatgctggatatgaaccgctggcggtcatcacgttccgccgggcactggtgcttgtgctcacgggcaaagccagtgtggttgccgagggtgacgatcccgtggtggggccccaggagatattgggccgcccgtccgttatcctcctgaaccgctatatccgccccaggtacaaccaggccaccgctgtcagccgccgcggcgtgttgaggcgcgatggccaccgttgcgcctactgcgggaaagcggcccacaccattgaccatgtccagcccaagtcccgcggtggtgccgattcctgggagaacctggtggccgcctgcctgcgttgcaacaacgtcaagggcgaccacactcccgctgaaatggggtggaagctgaggttcgtcccggagcccccgcacggaaccatctggcagatcaaggaactcgaaaaacccacccctgcctgggacccgttcctgctgcccgagcgcgcagcctga

SEQ ID NO: 154 - DNA, 549 bp, unidentified
  source 1..549: mol_type = genomic DNA; note = Methylorubrum extorquens
atgatcgatctgcaaaccctggtgctcaacgcggattacaggccgctttcctacaacccgctctcgttgtggtcctggaaggacgcattcacggcgctgtttctcgatcgcgtcactttggtcgcgaactacgatgtcgaggcgcacagtcccagccggtccctcaaggtgccgagcgtggtcgccctcaagagctacgtcgcgctggcccgcagcccggccttcacccgctacaacatctacttgcgcgacaccttctcctgccagtattgcggcttgcgtctcccctcgggcgggctgaccttcgatcacgtcgtgccccgctcacgcggcggcctctccacctgggagaacgtcgtggcggcctgctcaccctgcaatctgcgcaaggccaatcgcacgccgcacgaggccgagatgcccctgctcaacgagccgcaccggccgacgcggcacgagctgcaccgccgccaaccggaattcgaccaccgccagtatcaccatacctggctcgactacctctactgggacagcgagctcgacagttga

SEQ ID NO: 155 - DNA, 558 bp, unidentified
  source 1..558: mol_type = genomic DNA; note = Sinorhizobium fredii
ttgacgattgcagtctcacctcaggccttgccggcgctcgtcttgaacgccgactaccggccgctgagttattaccccttgtcgctctggtcctggcaggacgcgatcaaggccgtattccttgatcgcgtcacaatccttgctgaatacgagcattcggtgtcgtcaccgagcttttcgatgcggctgccgagcgtcgtctgtctcaagagctacgtgcagccgtcgcgtcatccggcattcacccgattcaacgttttccttcgcgacaaattcgaatgccagtattgcggatcgcctgacgatttgaccttcgaccatgtggtgccccgcgctcacggcggccagacgacgtgggaaaatgtcgtggcggcctgttcgccctgcaatctgcgcaagggcagcaagttgccgaaacaggccaacatgttccctcatcagaagccctatcacccgacggtgcaggacctgcacaacaacggccggctcttcccgccgaaccatctgcatgaaagctggatggactatctctactgggatgtcgaactgcagccgtaa

SEQ ID NO: 156 - DNA, 501 bp, Arthrobacter sp.
  source 1..501: mol_type = genomic DNA
atgcgcacactcgttctgaatgctggatatgaaccgctggcggtagtcacattccgccgggcgctggtccttgtgctcaccggaaaagcaagcgtagtggccgaaggcgacgatccagtcgtcgggccgcgggaggttcttggacgaccgtccgtgatcctccttaaccgctacatccggccgaggtacaaccggatcaccgctgtcacccgccgcggagtgctgcggcgggacgcacaccggtgcgcctactgcggcaaagcagcccacactattgaccacgtcctccccaaatccaggggcggcgcggattcctgggaaaacctggtggcggcgtgcctgcgctgcaacaacgccaagagtgaccacacgcccgccgaaatgggctggacgttgcggttcaaaccggcggctcccaccggaaccatctggcagatcaaggagctcgagaagcctgctcctgcatgggatccgttcctgctgcctgaatcggcagcctga

SEQ ID NO: 157 - DNA, 501 bp, Arthrobacter sp.
  source 1..501: mol_type = genomic DNA
atgcgcactctcgttctgaatgctggatatgaaccgctggcggtagtcaccttccgccgggcgctggtgcttgtactgaccgggaaggccagcgtggtggccgagggggatgaccccgtggtgggtccgcaggatatcctcggccgaccgacggtgatcctgcttaaccggtacatcaggccgcgctacaacaccctgacggccgtgagccgcaggggagtgctccgcagggacaaccaccgttgcgcctactgcggcaaggctgcccacaccatcgaccatgtccaccccaagtcgcggggcggggcggattcctgggagaacctggtggcggcctgcctgcgctgcaacaacgccaagggagaccacacgcccgccgaaatgggctggaagctgggcttcgttcccgggccgccgaagggcacgatctggcagatcaaggaactggagaagccgacgcccgcctgggatccgttcctgctcccagaaaaggcgcactga

SEQ ID NO: 158 - DNA, 975 bp, Bacillus sp.; identical to SEQ ID NO: 116
  source 1..975: mol_type = genomic DNA

SEQ ID NO: 159 - DNA, 501 bp, Arthrobacter sp.
  source 1..501: mol_type = genomic DNA
atgcgcacactcgttctgaatgctggatatgaaccgctggcggtagtcaccttccgccgggcgctggtccttgtgctcaccggaaaagcgagcgtagtggccgaaggcgacgagcctgtcgtcgggccacgggagattctcggacgtccgtccgtgattctcctcaaccgctacatccggccacgctacaacaggatcactgcggtcacccgccgtggcgtcttgcgccgcgacggacaccgttgcgcctattgcggcaaaaccgcccataccattgaccacgtccaccccaaatccaggggcggcgcggattcctgggaaaacctggtggcggcgtgcctgcgctgcaacaacgccaagagtgaccacacgccggccgagatgggctggaagctgcgtttcactccggcggcccccaccgggaccatctggcagatcaaggagcttgagaaacctgctcctgcatgggatcccttcctgttgcctgaatcggctgcctga

SEQ ID NO: 160 - DNA, 501 bp, Arthrobacter sp.
  source 1..501: mol_type = genomic DNA
atgcgcacactcgttctgaatgctggatatgaaccgctggcggtagtgacattccgccgggcgctggtccttgtgctcaccggaaaggcaagcgtggtggccgaaggcgacgatcctgtcgtcgggccgcaggacatcatgggccgtccgtccgtgattctccttaaccgctacatccgtccccggtacaacatgatcaccgcggtgagcaggcgcggtgtgctccgccgcgacggccaccggtgcgcctactgcggcaaggcagcccacaccatagatcatgtccagcccaaatccaggggcggagcggactcgtgggagaacctcgttgcagcctgcctccgctgcaacaacgtcaagggtgaccacacgcccagtgaaatgggctggaagctccggttcgatccggctcctccggtgggcaccatgtggcagatcaaggaactggagaagcccacaccggcgtgggatccgttcctgcttccggaaagagcagcctga

SEQ ID NO: 161 - DNA, 501 bp, Arthrobacter sp.; identical to SEQ ID NO: 149
  source 1..501: mol_type = genomic DNA

SEQ ID NO: 162 - DNA, 501 bp, Arthrobacter sp.
  source 1..501: mol_type = genomic DNA
atgcgcacactcgttctgaatgctggatatgaaccgctggcggtagttaccttccgccgggcgctggtccttgtgctcaccggaaaagcaagcgtagtggccgaaggcggcgaacctgtcgtcgggccgcaggagattctgggacgcccgtccgtgatcctcctcaatcgctacatccggccgcggtacaacaggattacggccgtcagccggcgcggagttctccgccgcgacggccaccgctgcgcctactgcggtaagacagcccacaccatagaccacgtccaccccaaatccaggggcggcgcggattcctgggaaaacctggttgcggcgtgcctgaaatgcaataacgccaagagcgaccacacgccggcagagatgggttggaaactccgtttcacacccggcgttccctccggaaccatgtggcagatcaaagaactcgagaagcctgcgccggactgggatccgttcctcttgccggaatccgctgcctga

SEQ ID NO: 163 - DNA, 501 bp, unidentified
  source 1..501: mol_type = genomic DNA; note = Pseudarthrobacter chlorophenolicus
atgcgcactctcgttctgaatgctggatatgaaccgctggcggttattaccttccgccgggcgctggtgcttgtgcttacaggcaaggccagcgtggtggccgaaggggacgaccccgtggtgggccccaccgacgtcctggggcgcccgtccgtgatcctcctcaaccgctacatccgcccccggtacaaccactccacggccgtcagccgtagaggcgtgctccgacgcgacgggcataaatgcgcctactgcgggaaagcggcgcacaccattgaccacgtgcatccgaaatcccggggcggggccgattcctgggagaacctggtggccgcctgcctgcggtgcaacaacgtcaaaggcgaccacactcccgccgagatgggatggaccttgcggttcgtgccggaacctccgcgcggaaccatctggcaaatcaaggagctggagaagcccacgccggcgtgggatcccttcctgcttccggaacgtgctgcttga

SEQ ID NO: 164 - DNA, 501 bp, unidentified
  source 1..501: mol_type = genomic DNA; note = Pseudarthrobacter chlorophenolicus
atgcgcactctcgttctgaatgctggatatgaaccgctggcggtcatcactttccgccgggcgctggtgcttgtgctcacgggcaaggcgagcgtggtggccgaaggggaggaccccgtagtgggccccaccgatgtcctgggccgcccgtccgtgatcctcctcaaccgctacatccggccccggtacaaccactccacagccgtcagccgcaggggtgtgctccggcgcgacgggcacaagtgcgcatactgcggcaaagcagcgcacacgatcgaccacgtacatcccaagtcgcgcggcggggccgacacatgggagaacctggtggctgcctgcctgcggtgcaacaacgtcaagggtgaccacactccggccgagatggggtggagcctccggttcgtacccgagcccccgcacggaaccatctggcagatcaaggaactggagaagcccacgcctgcctgggaccccttcctgcttccggagcgcgccgcctag

SEQ ID NO: 165 - DNA, 501 bp, unidentified; identical to SEQ ID NO: 108
  source 1..501: mol_type = genomic DNA; note = Pseudarthrobacter phenanthrenivorans

SEQ ID NO: 166 - DNA, 4290 bp, unidentified
  source 1..4290: mol_type = genomic DNA; note = Chryseobacterium glaciei
atggcaaaaaatatacttggattagacttgggaacgaactcgataggttgggcattaataaaacatgatttcgcaaacaaacaaggcaatattcttggcatgggaagcagaataatccctatgtctcaagatgttttgggcgatttcggaaaagggaattccgtttctcaaactgctgcaagaacaggttatcgaggtgtgagaagattacgtgaaagatttcttttaaggagagaaagacttcacagagttttgaatatcattaattatttaccagaacattatgcttatcaaattgattttgaaaaaagatttgggaaatttaaagaagaaacagagccaaaattagcttataataaagatgaatttattttcaaaaagtctttcgaagaaatgcttgctgattttaaaaataatcaaccaaaacttttagaaaacgataaaaaaattccttacgactggacaatttattatctccgtaaaaaagcgttttctcaaaaaatagaaaaagaagaactggcttggattcttttgaattttaatcagaaacgcggttattatcagttgcgtggtgaggaagaagaagagaatcccaacaagttggtagaattttattctttgaaaattgtagatgttgtagcggatgaaccgcaaaagggaaaatctgatatctggtattctttagttttagaaaacggctgggtttatagacgtgcgagtaaaacgcctttatttgattggaaagataaaactagagattttattgttactactgacctgaatgatgatggaagtattaaaacagataaagatggaaatgaaaaaagaagtttcagagctcctgcggaaacagattggactttagttaaaaagaaaactgaacaggaaattgataaatctcataaaaccgtcggtgtctatatttatgaagcgcttcttcaaaatccaaagcaaaaaattaaaggaaaattggttcgtacgattgagcgaaaattttataaagatgaacttaaacagattttagaaaagcagaaagaatttcatcaagagttaaaaaatgatgatttatacaacgactgcgttcgtgaattgtaccgaaataatgaagcgcaccaattgactttgagtaaaaaagaattcgtacatcttttgatggaggatataattttttatcaaagacctttaagaagtcagaaatcgtccatttcaaactgtagtttagaatctcgaaaatacaaagatgaaaatggagtagagcatattcaatttttaaaagccattccgaaatccaatccgtattatcaggaatttcgcatctggcaatggatttttaatcttaatatttacaaaaaagatgatgatgcgaacgttaccaaagaatttttaaatacaaccgaaaacttcgaaagtctgtttgaatttctaaataacagaaaagaagttgaccaaaaagcattgttgaaacattttaaattaaatgaaaaaacgtatcgctggaattttgtagaagataaaaaatatccttgtaacgaaaccaaaacaatgatttcgtcacgtttggataaagttgaaaatatttctgatgatttcttcacaagagagactgagcagaagatttggcatattatctattctgtaaacgataaaatggaatatgaaaaagctttaaaatcttttgccagaaaaaataatttagatgaaaattctttctttgaagcgtttaaaaaattcccgccattcaaaagtgaatacggctctttttccgaaagagcgattaagaaattgttgcctttgatgcgattgggaaaatattggaattacgaaaatatcagcgatgattcaaaagaaagaatccagaaaattattactggtgaatattatgaaaagatcaaagatagagtgagagaaaaagcaattcatctcacatctgaaaataatttccaaggtttgcagttgtggctggctcaatatgtagtttacgatagacattcagaagcgtcgatgattggaaagtggaattctgcagatgatttggaagagtttttaaaagaatttaagcaacattcccttcgaaatccaattgtagaacaggtgattacggaaacgcttcgtgtggtaaaagatatttggataaaatatggaaatggagcaaaagatttcttcaatgaaattcatattgaacttgggcgagaaatgaaacttcctgctgacgaaagagagcaactgacaaggcaaatttcagaaaatgaaaataccaatcttcgtatcaaagcgttattggctgaaatgatgaatgataattcggttgaaaatgttcgtccatattctccaatgcaacaggaaattttgaaaatttatgaagatggaattttaaaatccgacatcgaaattgaagatgatattctgaagattagcaaaacagcacaaccttcttcatcagatttaaaaagatacaaactttggctcgaacagagatataaatcgccttatactggacagattattccattaaataaactatttaccccagaatatgaaattgaacacattattccgcaaagccgatattttgatgacagttttagcaataaaattatttgcgaatcggcggttaataaattgaaagataattatatcggattaggatttattaagcaatttggtggaactattgttgaatgtggtttagggaaaaatgtaaaagtttttgaagttgatgaatatgaagattttgttaaaaaacactacgccaataaccgaggtaaaagaaataaacttcttttggaagagattcctgagaaaatgattgaacgacaactgaacgatacacgtcatatcagcaaatatatttcaggtgttctttccaacattgttcgtgtagaagatggaagcgatgaaggtgtaaattctaaaaatattgttccgggaaatggaaaaattacgacacaacttaagcaagattggggattaaatgatgtttggaatgatttgattcttccacgttttgaaaggatgaatcaacttaccaattcaacagattttaccgcctggaacgaaaatcatcaaaagtatttaccaactgttccgataaaagaatctaaaggattttcaaagaaaagaatagaccatcgccatcacgctttagatgctttggtcattgcttgtgcaacaaaagaccacgtgaatttattgaataatcaatcggcaaaatcggatactaaacgttacgacttgaaaaagaaactgatgaagtttgagaaagcggtttacaagcatccacaaactggcgaaagaatagaaagagatgttccgaagcaatttttaaaaccttgggaaagctttacaattgatgctaaaaataatttagataaaatcatagttagttttaagcaaaacttaagagtcattaataaagctaccaattattatgaaaaatatgtagagaaagatggcatcaaaactaaagaaaaaatacaacagaccggaacaaattgggcgattagaaaaccaatgcataaagaaacagtttccggaaaaattgatttgccttgggtgaaagttccgaaagggaaaatattaaccgcaacaagaaaaaatcttgatacatcttttgatttgaaatcgattaattctatcacagacactggaattcaaaaaattcttagaagttatttggaatttaaaggcagtgctgaagctgcattttcgcctgaaggaattgaagatatgaataaaaatatcgagaaatataatgatggaaaacttcatcaacctatcaataaagtgagagtttttgaattaggaagtaaatttcaagttgggcaaactggcaataaaaaagataaatacgtggaagctgccaaaggaacaaatttgttttttgcagtttacgaagataaaaatggaaaaagaagttatgaaacaattcctttgaatattgttattgaaagacaaaaacaaggattgtctgttgtcgacttaaaaggagtaaatgatttctatctgtgtccaaatgatttagtttatattccttcggaagacgagcttgaaaacgtaaataacattgattttaaaaatttcacaaaagaaaaaaatgaaagaatttataaagttgtaagtttttcgggtagtcaaatattttttgtgagacaagatgtttctacatcagttgttaataaagcagagttttcaactttaaacaaaatggaacgtgcaattgatggttctatgattaaagagcattgtttaaaactaaatgttgaccgtttaggaaatatttcaaaaacataa

SEQ ID NO: 167 - DNA, 501 bp, unidentified
  source 1..501: mol_type = genomic DNA; note = Pseudarthrobacter phenanthrenivorans
atgcgcactctcgttctgaatgctggatatgaaccgctggcggttatttctttccgccgggcgctggtgcttgtgctgacgaacaaggcgagcgttgtggccgaaggggacgatcccgtagtgggtccccaggagatactgggccgcccgtccgtgattctcctgaaccgctacatccgccccagatacaaccagtcaacggctgtgagcagacgcggcgtgctccgacgtgacgggcacaaatgcgcctactgcggaaaagccgcgcacaccattgaccatgtccaccccaaatcccgcggtggcgcggattcatgggagaacctggtggctgcgtgccttcgctgcaacaacgcgaagggtgaccacacccccgccgagatgggctggaagctgcggttcgtgcccgaacctccgcatggcgccatctggcagatcaaggaactcgagaagcccacacctgcctgggatccgttcctgctgccggagcgcgctgcctga

SEQ ID NO: 168 - DNA, 357 bp, Azospirillum thiophilum
  source 1..357: mol_type = genomic DNA
atggatgagcgcaaactcaaacccggcgatccgaaactgctggcgatcagccagcagggacggtgcttctactgcggcgaaccggtcggcgcaaaagcgacctacgaccacttcatcccgcaatgctacggcggcatcgacgacatagccaacatcgtcctgtcccaccgccgctgcaacatcctgaaaggcgaccgcctgccgactcccgacgagatcgacagcttcctgatgctccgccggaacgctcgcctgccggtctggccgccgatcctggcgctggccgatgccgaggaaggcgaggagtggatcgtggtggcaaaggcggtggccgccgagggcggatggaaggggtag

SEQ ID NO: 169 - DNA, 564 bp, Azospirillum thiophilum
  source 1..564: mol_type = genomic DNA
ttggcaccaccacccgatcactgtccggcgctggtactgaatgcggactttcgtccgctgagctacttccctttgtctctgtggtcctggcaggaagctgtcaaggccgtgttcctcgaccgcgtcaacatcatttccgaatatgatcgagtcgtcaggtcgcccaacttcgagataaagctgcccagcgtcatctcattgaaggaatacatcccggcgacccgtcgcccggccttcacccgcttcaacgtcttcctgcgcgaccggttttcgtgccagtactgcaacgagccgttcccgactcaggaactgacgttcgaccacgtgatacctcgctccaaaggcgggcgaactacatgggaaaacgtcgtgacgtcctgttccgcctgtaacctcaggaaagggaacaggctccccaagcagtgtggcatgataccgttttacccgccgttccagcccacggcgtttcagcttcaggaaaacggacgatccttcccgccaaacttcttgcatgaaagttggcgggattttctttactgggataccgaactcgatcctttctga

SEQ ID NO: 170 - DNA, 501 bp, Rathayibacter tritici
  source 1..501: mol_type = genomic DNA
atgcgcacactcgtcctcaacgccggctacgagccgctcgcggtggtgtcgttcaagcgggcgctggtgctgctgatgaccgagaaggcgatgatcatcgtcgccgacgaggagcacccggtgcacggagcgaccggcgactacgtccggccctcggtgatcgtgctgacgcgctacgtccgcatcccgagctcgcgcagcgtgccggtctcgcgccgcggggtgctgcggcgcgacaaccaccgctgcggctactgcggccagcacgcgaacacgatcgaccacatccagccgaagtcgcgcggcgggcgggacacctgggagaacctcgtcgcctgctgccaccgctgcaacaacgtcaagagcgaccgcacgccgctcgagatgggctggcagctgcgctccgtgccgcggatgccgcgcgccggctcctggctcgtgcgcggtgccgagcgcacccagcccgagtgggacgactacctcgcggcggcggccgcctga

SEQ ID NO: 171 - DNA, 501 bp, unidentified
  source 1..501: mol_type = genomic DNA; note = Pseudarthrobacter phenanthrenivorans
atgcgcactctcgttctgaatgctggatatgaaccgctggcggttatcacgttccgccgggcgctggtgcttgtgctgacaggcaaggcgagcgtcgtggccgaaggggatgacccggtggtgggacccaccgacgtcctgggccggccgtccgtgatcctcctcaaccgctatatcaggccccggtacaaccactccacggcagtcagccgcaggggcgtcctccggcgcgacgggcataaatgcgcctactgcggcaaggcggcgcacaccatcgaccacgtccacccaaagtcacggggcggcgccgattcgtgggagaacctggtggccgcctgcctccggtgcaacaacgccaagggggaccatacgcctgccgagatgggctggacccttcggttcgtccccgaaccgccgcacggcacgatctggcagatcaaggaactggaaaaaccgacccccgcatgggatccgttcctgctgccggagcgcgctgcctga

SEQ ID NO: 172 - DNA, 501 bp, unidentified; identical to SEQ ID NO: 108
  source 1..501: mol_type = genomic DNA; note = Pseudarthrobacter chlorophenolicus

SEQ ID NO: 173 - DNA, 501 bp, Arthrobacter sp.; identical to SEQ ID NO: 131
  source 1..501: mol_type = genomic DNA

SEQ ID NO: 174 - DNA, 501 bp, unidentified; identical to SEQ ID NO: 167
  source 1..501: mol_type = genomic DNA; note = Pseudarthrobacter phenanthrenivorans

SEQ ID NO: 175 - DNA, 501 bp, Arthrobacter sp.; identical to SEQ ID NO: 131
  source 1..501: mol_type = genomic DNA

SEQ ID NO: 176 - DNA, 501 bp, Arthrobacter sp.
  source 1..501: mol_type = genomic DNA
atgcgcactctcgttctgaatgctggatatgaaccgctggcggtcatcacgttccgccgggcactggtgcttgtgctcacgggcaaagccagtgtggttgccgagggtgacgatcccgtggtggggccccaggagattttgggccgcccgtccgttatcctcctgaaccgctatatccgccccaggtacaaccaggccaccgctgtcagccgccgcggcgtgttgaggcgcgatggccaccgttgcgcctactgcgggaaagcggcccacaccattgaccatgtccagcccaagtcccgcggtggtgccgattcctgggagaacctggtggccgcctgcctgcgttgcaacaacgtcaagggcgaccacactcccgctgaaatggggtggaagctgaggttcgtcccggagcccccgcacggaaccatctggcagatcaaggaactcgaaaaacccacccctgcctgggacccgttcctgctgcccgagcgcgcagcctga

SEQ ID NO: 177 - DNA, 498 bp, Microbacterium sp.
  source 1..498: mol_type = genomic DNA
atgcgcacgctggtgctgaacgcgggctacgagcccctcgccgtggtgtcgttcaagcgggccctggtgctcgtgatgaacgagaaggcgaccgtcatcgaacacgacgcggaggagcccgtctggggcacccgagcctcctacgaccgccccgcggtgatcgtgctgacccgctacgtccgggtgccggtctcgcggcgcatcccggtgacccgtcgcggcgtgctgcgccgcgactcctaccggtgcgcgtactgcgccaagccggcgtcgaccatcgaccacatcctgccccgctcgcgcggcggcgccgattcgtgggagaacctggccgcctgctgcctgcgctgcaacaacatcaagagcgaccgcacgccgcaggagatggggtgggagctgcgcatcacgcctcggccgccgcacggcacggcctggagcgtgcggggcgcggagcgcgccgatccgcgctgggagccgtacctcgccctcgcggcctga

SEQ ID NO: 178 - DNA, 501 bp, Arthrobacter crystallopoietes
  source 1..501: mol_type = genomic DNA
atgcgcactctcgttctgaatgctggatacgaaccactggcggtggtgaccttccgccgggcgctcgtccttgttctcacagggaaggccagcgtggtcgcagaaggggaggatccggtggtcggcccgaacgaagtgctgtcccggccctccgtcatcctccttaataggtatgtgaagattccctaccgcaatagtgctgtggccacccggcgcggcgtgctccgacgtgacaaccacctgtgcgcgtactgcggcaaggccgcctccaccatcgaccacgttattccgcgctcccgcggggggaaagacagctgggagaatctagtggcatgctgcctccgctgcaacaacgcgaagggtgataagacccttgcgtccctggggtggtccctgcgcctcgttcccaaaccgccgcgaggcacgtcctggcagatccgtgagctggaaaagccgctgccgcagtggagcgagttcctgcccgcagacaacgccgcgtga

SEQ ID NO: 179 - DNA, 501 bp, unidentified; identical to SEQ ID NO: 99
  source 1..501: mol_type = genomic DNA; note = Pseudarthrobacter chlorophenolicus

SEQ ID NO: 180 - DNA, 501 bp, unidentified; identical to SEQ ID NO: 108
  source 1..501: mol_type = genomic DNA; note = Pseudarthrobacter phenanthrenivorans

SEQ ID NO: 181 - DNA, 975 bp, Bacillus megaterium; identical to SEQ ID NO: 116
  source 1..975: mol_type = genomic DNA

SEQ ID NO: 182 - DNA, 975 bp, Bacillus megaterium; identical to SEQ ID NO: 116
  source 1..975: mol_type = genomic DNA

SEQ ID NO: 183 - DNA, 537 bp, unidentified
  source 1..537: mol_type = genomic DNA; note = Glutamicibacter arilaitensis
atgccagatggtagctatcggttcaagaaggaagtacgacaaatgcgaacactggtattaaatgcaggatacgagcccttagccgtcgtgacttttcgacgagcactggtaatggtcatggccgataaagcgaccatcgttctccaggatgacgactcgcctgttcgcagctcacacggacagttacccagacccactgtcattgtcttaaaacgctatgtacgacgcccctaccgcgacatgcttcatgtcacgcgtaggggcgttttgcgtcgagacgcgtatacctgcgcgtactgcgggaaatcggcgaatacgatcgaccacgtcattccgcgaagcaggggagggaagaacacgtgggagaacttggtggctgcttgcctgcactgcaacagcaggaaagccgacaagatgctctctcagttgggctggcagctgcggatcacgccgcatcgccctcggatgggcggacatctggtctccggtcttcagcatattgatgccgcatgggacgacttccttcaagcaagctga

SEQ ID NO: 184 - DNA, 501 bp, Arthrobacter sp.; identical to SEQ ID NO: 176
  source 1..501: mol_type = genomic DNA

SEQ ID NO: 185 - DNA, 501 bp, unidentified
  source 1..501: mol_type = genomic DNA; note = Pseudarthrobacter phenanthrenivorans
atgcgcactctcgttctgaatgctggatatgaaccgctggcggttgtgactttccgccgggcgctggtgcttgtgctcacgggcaaggcgagcgtggtggccgaaggggacgaccccgtggtgggtccacaggaaatactgggccgcccctcggtaatcctcctcaaccgctacatccgcccgcgctacaaccaggccaccgccgtcagccggcggggtgtcctgagacgcgacgggcacaagtgcgcatactgcggcaaggctgcgcacacgatcgaccacgtccacccaaaatcccgcggcggagcagactcctgggagaacctcgtggcagcctgcctccgctgcaacaatgccaagggtgaccacactcccgcggaaatgggctggaagctgcggttcgtaccggagcctccgcgcggcaccatttggcagatcaaggaactggagaagcccacccccgcctgggacccgttcctgctgccagagcgcgctgcctga

SEQ ID NO: 186 - DNA, 501 bp, unidentified
  source 1..501: mol_type = genomic DNA; note = Pseudarthrobacter phenanthrenivorans
atgcgcactctcgttctgaatgctggatatgaaccgctggcggtcatcactttccgccgggcgctggtgcttgtgctcacgggcaaagcaagcgtggtggccgagggtgacgatcccgttgtgggtccgcaggagatcctgggccggccgtccgtgatactccttaaccgctacatccgtcccagatacaaccaggccacggctgtgagccggcggggagtgctgcggcgtgatgggcacaaatgcgcctactgcggaaaagcggcacacaccatcgaccatgtccaccccaagtcccgcggtggtgcggattcgtgggagaacctcgtggcggcctgcctgcgctgcaacaacgcgaagggcgaccacacacccgccgaaatggggtggaaactccggttcgttcccgaaccgccgcacggaagcatgtggcagatcaaggaactcgagaagccgacgccggcgtgggacccgttcctgctgccggagcgcgccgcctga

SEQ ID NO: 187 - DNA, 459 bp, unidentified
  source 1..459: mol_type = genomic DNA; note = Pseudarthrobacter phenanthrenivorans
atggagagcgttctggttctcaacgcagggtacgaacccctgcaccgggtgtcgctgaggcacgccatcaggatggtccaccggggtgtggccgaggtcctcaaggacgacggcgagctgttcggccctttcgcccgcccggtggtgctgcgcctcatccggtacgtgaagatgacctggcggacacgggccacgtgcaccaaggaggccgtgaagcgccgtgacggccgctgcgcctactgcacgaagggcaaggcggagactgttgaccacgtcatcccgcagtcccgcggcggccagaacacctggctcaacctggtgggctgctgcttcaagtgcaaccagtacaaggccgaccggaccccggctgaggccggcatgaagctgctgctgaccccgtacgcgcccaaggaccggccgccggtgatgcccgcgttcgacttcgccgagctgacctga

SEQ ID NO: 188 - DNA, 984 bp, Paenibacillus xylanexedens
  source 1..984: mol_type = genomic DNA
gtgaacttcatagaaaaatggctacatatcattcggaattgttcatttgataacacctacaaaatggcttgggctaaagcgattactgaaattgccgtagagcaagaagaagcattaccaacggaagaagtaattcaagttgaaatccagttaagagatattgcagaaaaggtacttcgctattattttgagcaaaccatattctttgctcttcagcaaagttcaaatcctgtaaagccacctgttatggtgaccattgcaaagcagctgataagcgcttatcaggctcgtacaggatcaatgaaaccagttaaatggtttaggtctaatattgagtccgtatgcagcgtagagtttgagaaggctgtttcttctacagtgaaagcgctaaaatcggatgtgagttaccgcttcttacgtgtggaaggaaaagaagtagaaggtgtttatcaatatcaacagaaagcggatagtctattcatgcaaagaagggatttacttacgcttaagaggaaatgcctcattgtgtttgatgctattaattatcgatggacgcagatgttagaaaacttcaatcactcgccccgattgtcaaagaaagtaagaatcattgacgaagatcagatacgccggaatccgcttggtaaatttgcagagttcattgaagctgagaacccggaatgtcgttgctttttatgtagtgaaaagatagaagctgaaacaccagcaattgatcatgtcatcccatggtcgtacttgtactcggatgatctctggaatttggtgtttgcacatcaatcttgtaacagtagtaaatccaatgtgataccgagtgaggacatgattcttcgactagaagcaagaaaccaagcacttctagatagtttgaaagaaaataagaaactaaataaacatgtttctgatctggaactagctatagaaagtaatcttgtacgaaagttctggattgcatgccagggctga

SEQ ID NO: 189 - DNA, 495 bp, Arthrobacter sp.
  source 1..495: mol_type = genomic DNA
atgcgaacactggttttgaatgccggctacgagcccctggccgtggtgaccttccgtcgagcccttttgctggtgatggcggagaaagccactatcgtcctgcaggatgactcgtcgcccgtgcgcagcgctcacggcgagttaccgcggccaacggtaatcgtcttaaagcgctatgtacgacgcccctacagggacatgaatcatgtcacccgtaggggcgttctccgtagggacaggtttacctgcgcctactgcggcaagtccgccaataccatcgaccatgtcattccccgcagcaggggcggtcagaatacctgggagaacctggtagcggcctgtctggcctgcaacagccgcaaggccgacaagatgctcgcacagctcggctggcagctccgcgtcaccccgcaccgcccgcgctatggcgggcatctggtctcggggctcaaccagatccacgattcgtgggatgtctacctccaggcgggctga

SEQ ID NO: 190 - DNA, 495 bp, Arthrobacter sp.; identical to SEQ ID NO: 189
  source 1..495: mol_type = genomic DNA

SEQ ID NO: 191 - DNA, 537 bp, unidentified
  source 1..537: mol_type = genomic DNA; note = Glutamicibacter arilaitensis
atgccagatggtagctatcggttcaagaaggaagtacgacaaatgcgaacactggtattaaatgcaggatacgagcccttagcagtcgtgacttttcgacgagcactggtaatggtcatggccgataaagcgaccatcgttctccaggatgacgactcgcctgttcgcagctcccacggacagttacccagacccactgtcattgtcttaaaacgctatgtacgacgcccctaccgcgacatgcttcatgtcacgcgtaggggcgttttgcgtcgtgacgcgtatacctgcgcgtactgcgggaaatcagcgaatacgatagaccacgtcgttccgcgaagcaggggagggaagaacacttgggagaacttggtggctgcttgcctgcactgcaacagcaggaaggccgacaagatgctctctcagttgggctggcaactgcggatcacgccgcatcgccctcggatgggcggacatctggtctccggtcttcagcatattgatgccgcatgggacgacttccttcaagcaagctga

SEQ ID NO: 192 - DNA, 495 bp, Arthrobacter sp.; identical to SEQ ID NO: 189
  source 1..495: mol_type = genomic DNA

SEQ ID NO: 193 - DNA, 495 bp, Arthrobacter sp.; identical to SEQ ID NO: 189
  source 1..495: mol_type = genomic DNA

SEQ ID NO: 194 - DNA, 501 bp, unidentified
  source 1..501: mol_type = genomic DNA; note = Pseudarthrobacter chlorophenolicus
atgcgcactctcgttctgaatgctggatatgaaccgctggcggtcattactttccgccgggcgctggtgcttgtgctcacgggcaaggcgagcgtcgtggccgaaggggatgaccctgtcgtgggacccacggacgtcctgggccgcccgtccgtgatcctcctcaaccggtacatccggccccgatacaaccactccacggctgtcagccgcaggggcgtactcagacgcgacggccataaatgcgcatactgcggcaaggcagcgcataccatcgaccatgtgcaccccaagtcccggggcggggctgactcgtgggagaacctggtggcagcctgccttcgctgcaacaacgtcaagggcgaccacactccgaccgagatgggctggagcctgcgctttgtgccggagcctccgcgcggcaccatctggcagatcaaggaactggaaaagcccacgcccgcgtgggatccgttcctcctgcccgaacgcgccgcctga

SEQ ID NO: 195 - DNA, 495 bp, Arthrobacter sp.; identical to SEQ ID NO: 189
  source 1..495: mol_type = genomic DNA

SEQ ID NO: 196 - DNA, 501 bp, unidentified
  source 1..501: mol_type = genomic DNA; note = Pseudarthrobacter chlorophenolicus
atgcgcactctcgttctgaatgctggatatgaaccgctggcggttattactttccgccgggcgctggtgcttgtgctgacaggcaaggccagtgtggtggccgaaggggacgaccccgtggtgggccccaccgacgtcctggggcgcccgtccgtgatcctcctcaaccgctacatccgaccccggtacaaccattccactgccgtcagccgcaggggagtgctccggcgcgatgggcataaatgcgcctactgcgggaaggctgcgcacaccattgaccacgtgcatccgaaatccaggggcggggcagattcgtgggagaacctggtggccgcctgcctaaggtgcaacaacgtcaaaggcgaccacactcccgccgagatgggatggaccctgcggttcgtgccggcacctccgcatggaaccatctggcagatcaaggagctggagaaacccacaccggcgtgggatcccttcctgcttccggaacgcgctgcctga

SEQ ID NO: 197 - DNA, 501 bp, unidentified
  source 1..501: mol_type = genomic DNA; note = Pseudarthrobacter phenanthrenivorans
atgcgcactctcgttctgaatgctggatatgaaccgctggcggttatttctttccgccgggcgctggtgcttgtgctgacgaacaaggcgagcgttgtggccgaaggggacgatcctgtagtgggtccccaggagatactgggccgcccgtccgtgattctcctgaaccgctacatccgccccagatacaaccagtcaacggctgtgagcagacgcggcgtgctccgacgtgacgggcacaaatgcgcctactgcggaaaagccgcgcacaccattgaccatgtccaccccaaatcccgcggtggcgcggattcatgggagaacctggtggctgcgtgccttcgctgcaacaacgcgaagggtgaccacacccccgccgagatgggctggaagctgcggttcgtgcccgaacctccgcatggcgccatctggcagatcaaggaactcgagaagcccgcgccggcctgggatccgttcctgctgccggagcgcgctgcctga

SEQ ID NO: 198 - DNA, 501 bp, unidentified; identical to SEQ ID NO: 115
  source 1..501: mol_type = genomic DNA; note = Pseudarthrobacter phenanthrenivorans

SEQ ID NO: 199 - DNA, 501 bp, unidentified; identical to SEQ ID NO: 115
  source 1..501: mol_type = genomic DNA; note = Pseudarthrobacter phenanthrenivorans

SEQ ID NO: 200 - DNA, 537 bp, unidentified
  source 1..537: mol_type = genomic DNA; note = Glutamicibacter arilaitensis
atgccagatggtagctatcggttcaagaaggaagtacgacaaatgcgaacactggtattaaatgcaggatacgagcccttagccgtcgtgacttttcgacgagcactggtaatggtcatggccgataaagcgaccatcgttctccaggatgacgactcgcctgttcgcagctcccacggacagttacccagacccactgtcattgtattaaaacgctatgtacgacgcccctaccgcgacatgcttcatgtcacgcgtaggggcgttttgcgtcgagacgcgtatacctgcgcgtactgcgggaaatcggcgaatacgatcgaccacgtcattccgcgaagcaggggagggaagaacacgtgggagaacttggtggctgcttgcctgcactgcaacagcaggaaagccgacaagatgctctctcagttgggctggcagctgcggatcacgccgcatcgccctcggatgggcggacatctggtctccggtcttcagcatattgatgccgcatgggacgacttccttcaagcaagctga

SEQ ID NO: 201 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattctttgctaatggtcactttaccacaacgtataaatatggattaatgaaagctttaattgaaaacctatataacgttgacaatcggttagtactcacgtttgatcaaatatatttttcatttgctaaaatatattggaatttggttattcatcatgatttaaatcaattaaatacacccaatagacaatctggaattcaaaaagagttaaaggaatttcagttaatgcatggtgtccccaataaagtagtatttgatcgtttaccaagtaattttcaattgcagttagtagaaagaactaaaaaagtgggggcaaggtatgtagtaggggcgttatacggagatatggaagcttctatttatgagtttgacaaaagaacagaatacataaagtttaatagcagtatctatatttttttacaaaaatatagacaaattgttactcatttaaccaattatcatttggcgaaatttttagaaaagcataatgacaaaaataagttagaagatgtcttgttgaaagtagaaaatatttcaaaacgcgaatcgctgcatgcattctataacatactttggaaatatgaaacaaaagaatgtttttattgtgggaaagttctttctgataaacgcatcgctactcatgtagatcattttattccatggagttatattcaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgagtatgctggaatgaatggatacgtagaaaactggtctccatag

SEQ ID NO: 202 - DNA, 975 bp, Bacillus megaterium; identical to SEQ ID NO: 11
  source 1..975: mol_type = genomic DNA

SEQ ID NO: 203 - DNA, 975 bp, unidentified; identical to SEQ ID NO: 9
  source 1..975: mol_type = genomic DNA

SEQ ID NO: 204 - DNA, 537 bp, unidentified
  source 1..537: mol_type = genomic DNA; note = Glutamicibacter arilaitensis
atgccagatggtagctatcggttcaagaaggaagtacgacaaatgcgaacactggtattaaatgcaggatacgagcccttagccgtcgtgacttttcgacgagcactggtaatggtcatggccgataaagcgaccatcgttctccaggatgacgactcgcctgtttgcagctcccacggacagctacccagacccactgtcattgtcttaaaacgctatgtacgacgcccctaccgcgacatgcttcatgtcacgcgtaggggcgttttgcgtcgagacgcgtatacctgcgcgtactgcgggaaatcggcgaatacgatcgaccacgtcattccgcgaagcaggggagggaagaacacgtgggagaacttggtggctgcttgcctgcactgcaacagcaggaaagccgacaagatgctctctcagttgggctggcagctgcggatcacgccgcttcgccctcggatgggcggacatctggtctccggtcttcagcatattgatgccgcatgggacgacttccttcaagcaagctga

SEQ ID NO: 205 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcctttgctaaaatatattggaatttggttattcatcatgatttaaatcaattaaatacacacaatagacaagctggaattcaaaaagagttaaaggaatttcagttaatgcatggtgtcccaaataaagtagtatttgatcgtttaccaagtaatcttcaattgcagttagtagaaagaactaaaaaagtgggggcaaggtatgtagtaggggctttatacggagatatggaaggttccatttatgagtttgataaaagaacagaatacataaagtttaatagcagtatgtatatttttttgcaaaaatatagacaaattgttactcatttaaccaattatcatttggcgaaatttttagaaaagcataatgacaaaaaaaagttagaagatgtcttgttaaaagtggagaatatttcaaaacgcgaatcgctgcatgcattctataacatactttggaaatacgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtttgctggaatgaatggatacgtagaaaactgggctccttaa

SEQ ID NO: 206 - DNA, 975 bp, unidentified
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcctttgctaaaatatattggaatttggttattcatcatgatttaaatcaattaaatacacacaatagacaagctggaattcaaaaagagttaaaggaatttcagttaatgcatggtgtcccaaataaagtagtatttgatcgtttaccaagtaatcttcaattgcagttagtagaaagaactaaaaaagtgggggcaaggtatgtagtaggggctttatacggagatatggaaggttccatttatgagtttgataaaagaacagaatacataaagtttaatagcagtatgtatatttttttgcaaaaatatagacaaattgttactcatttaaccaattatcatttggcgaaatttttagaaaagcataatgacaaaaataagttagaagatgtcttgttaaaagtggagaatatttcaaaacgcgaatcgctgcatgcattctataacatactttggaaatacgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtttgctggaatgaatggatacgtagaaaactgggctccttaa

SEQ ID NO: 207 - DNA, 537 bp, unidentified
  source 1..537: mol_type = genomic DNA; note = Glutamicibacter arilaitensis
atgccagatggtagctatcggttcaagaaggaagtacgacaaatgcgaacactggtattaaatgcaggatacgagcccttagcagtcgtgacttttcgacgagcactggtaatggtcatggccgataaagcgaccatcgttctccaggatgacgactcgcctgttcgcagctcccacggacagctacccagacccactgtcattgtcttaaaacgctatgtacgacgcccctaccgcgacatgcttcatgtcacgcgtaggggcgttttgcgtcgagacgcgtatacctgcgcgtactgcgggaaatcggcgaatacgatcgaccacgtcattccgcgaagcaggggagggaagaacacgtgggagaacttggtggctgcttgcctgcactgcaacagcaggaaagccgacaagatgctctctcagttgggctggcagctgcggatcacgccgcatcgccctcggatgggcggacatctggtctccggtcttcagcatattgatgccgcatgggacgacttccttcaagcaagctga

SEQ ID NO: 208 - DNA, 501 bp, Arthrobacter sp.
  source 1..501: mol_type = genomic DNA
atgcgcactctcgttctgaatgctggatatgaaccgctggcggtagtcaccttccgtcgggcgctggtccttgtattgaccggaaaggccagcgtggtggccgaaggggacgacccggtggtgggcccgcaggacatcctcggccgcccgtcggtgatcctgctgaaccgctacatccggccgcgctacaacaccctgacggctgtcagccgcaggggagtgctccgccgcgacagccaccgctgcgcctactgcggcaaggctgcccacaccattgaccatgtacagcccaaatcgcgcggcggcgcggactcgtgggagaaccttgtggccgcgtgcctgcgctgcaacaacgccaagggagaccacaccccggcggagatgggctggaagctcggtttcgttcccgggccgcccaagggcaccatctggcagatcaaggaactggagaagccgaccccggcctgggacccgttcctgcttcccgaaaaggcgcactga

SEQ ID NO: 209 - DNA, 495 bp, unidentified
  source 1..495: mol_type = genomic DNA; note = Glutamicibacter arilaitensis
atgcgaacactggtattaaatgcaggatacgagcccttagccgtcgtgacttttcgacgagcactggtaatggtcatggccgataaagcgaccatcgttctccaggatgacgactcgcctgtttgcagctcccacggacagctacccagacccactgtcattgtcttaaaacgctatgtacgacgcccctaccgcgacatgcttcatgtcacgcgtaggggcgttttgcgtcgagacgcgtatacctgcgcgtactgcgggaaatcggcgaatacgatcgaccacgtcattccgcgaagcaggggagggaagaacacgtgggagaacttggtggctgcttgcctgcactgcaacagcaggaaagccgacaagatgctctctcagttgggctggcagctgcggatcacgccgcttcgccctcggatgggcggacatctggtctccggtcttcagcatattgatgccgcatgggacgacttccttcaagcaagctga

SEQ ID NO: 210 - DNA, 537 bp, unidentified; identical to SEQ ID NO: 204
  source 1..537: mol_type = genomic DNA; note = Glutamicibacter arilaitensis

SEQ ID NO: 211 - DNA, 501 bp, unidentified; identical to SEQ ID NO: 108
  source 1..501: mol_type = genomic DNA; note = Pseudarthrobacter phenanthrenivorans

SEQ ID NO: 212 - DNA, 3249 bp, unidentified
  source 1..3249: mol_type = genomic DNA; note = Niveispirillum cyanobacteriorum
atgcgctggcggttgggcctggacatgggcgcggggtcgttgggttggtgcgccatcctcctggatgaacagggttttccaagcggcatcctggccgctggtagccacatcttcggtgatggtcgggccccatcgcggggggaccagcagggaacgccattgggggttgaacggcgggcagcgcgggccatgcggcggcgccgcgaccggtttctagggcggcaggtcaagctgctggcgcggctgaccctggccggcctgttccccgccgatcccgccagggccaaggcgctggaactgctggacccctatgccctgcgcgctcgtgccctggatgaggtcctgctgccccaccatctgggccgtgccttcttccatctgaaccagcgtcggggtttcaagtccaaccgcaagaccgacaggggcgtggaggatgacgcgggcaaggttgcgcttggcatcaaccggctgcgcgaagccatcgagcaagatggcgctcgcaccatgggcgaattcctgcatcgccgccgacagtcggcaccggatgagaaccgcattcccagcgtgcgtacccgcctgcgccctgaaaccgggggggaggcgaagggtgatggctatgatttctatcccggccgagaactggtggaggaggagttcgaggccatctgggacgcgcagacctcgcatcatccgactttattgacgccggagaacagggtgctgctgcatgaggcgatcttctaccagcgcccgttgaaagagcccaggatcggcaaatgcaccctgctcaacagcgagccgcgcctgcccaaggcccatccgcttttccagcgccgccggctgctggaagaggtgaacgcgttgcgggtcgtgcggccgggggaggtggcgacgaagctgacgcgcgagcagcgcgacctgctattgttgaagctgaaggacaaggccaaggtcagcttcgagagcctgcgcagcaaggtgttgaggctggaacccgaggcccggttcaacaaagaaggggagaaccgcagggacctggccggcgacgaggtcttcgcccagatggcggccaagacccgcttcggatcgcgctgggcgcatttctcgctggaaaagcaatggtccatcctgtcccgcctgatcgacgagcaggatggcgcactcttggatgtctggctgcgcaaagagcatgggctggacgcggagcaggccagagcggtggctgcggcccatttgccctcgggcttcggccggttcggggaaacggcgacgcgacggctgatcgaggcgctgcaggccgatgtgatcgtttatagcgaggcggtggaggtggctggattgggccatcacagcgacatgcgcacgggcgaggtgctggccgatctgccttattacggcgtcaccctggaacgccacttgttgccaggcacgggcgagaagaccgatccggaggaaatgcgggtcggccggctgaccaaccccacggtgcatatcgcgctgaaccggttgcgccgcatcgtcaaccgactgatcaagacctatggcaggccggagcagatcgctatcgaactgggccgcgagttgaagctgagcgaggaagagaaacagcgcctgtcgcgtgaaaacaatcgcaaccgcgtcaatgcggaggcacggtcaaaaaagctgaccgaactgggccagcccgacaccggcgccaaccgcgctcggctgaaactgtgggaggagttgaacgacgagaattgcctggatcgccgctgcgtctatagcggggtgcagatttcccccaccatgctgttttccgacgccgtggaggtggaccatatcctgcccttcgacgccacctgggacgatggcaacggcaacaagatcctgtgcctgcgcgaatggaaccgtcgcaaacggaaaatgtcgccctatgaggcctggggccataccgacctttggcccgacatcgccgcgcgcgccgcgcgcctgcccaaggagaagcgctggcggttcgaacccgatgcgatgaagcacttcgacgagaagggggggttcatcgcccggcacctggtggatacccagtacatgtcacggctggcacgtgagtatttggaggcactctatccggagaagggagagggcagcggccatgtctgggtctcccctggccggctaacggaattgatccggcgcaagctggggctgaacgagctgttgcccgatcataatttcgccggcaccaagaaccgcctggaccatcgccaccacacgatcgacgccttcgtcgtagccgtcaccgaccgctcgcttttgcagggcatatcccgcgcctcggcactggaaggggaggaggggcgggaacgggtgatcgtcccggacccgtgggagggtttccgggaggaactgcgccgcaccctggcctccatcaccgtctggcaccgtccggaccatggcacggcctccaaggcgggcttggccaagggccgcgatcagacggcgggcaagctgcacaatgatacggcctatgggctggccggaccggcggacaaggaggggcgccatctggtggtcgtccgcaagcccttgatggggctgaagccgggcgatatcgacgatgtgcgcgacccggacctgcgtgccgccctgcggcaattcacgcaaggcaaggaaggtaaggagttcgagaaggcgctttcgcgctttcccgaatttggccccctgcatttccggggcatccggcgtcttcggctgacggggcggttgaaggttattcctatcaccggacccgatggcgtgccttacaaggcttacaagggtgacagcaattatcgctacgatgtttgggaattgccggatggaagctggaaggccgatgtcgtcaccatgttcaacgcccaccagcccggatggatatccaccatccgccgcgactgcccgacagcgcgcaaggtgctgagcctgcagcaggacgacctgattgcgatcgagcccgatggcggtggccggcgcctgctgcgtgtggtgaaattcgggcagggcggggagatcgtgctggccgaaccgaacgaggccggcaagctgaaggaacgccacgggttgcccaacgatagtgatccgttccgttatgtctttgcagccgcaggcagcttgtcgcggcagatggcgcggcaggtgcgggtggacgagttgggccgcgtccaggatcccggcttccccgcccgcaagacccgccgccggacgcggctgaagccggaaagctga

SEQ ID NO: 213 - DNA, 537 bp, unidentified; identical to SEQ ID NO: 204
  source 1..537: mol_type = genomic DNA; note = Glutamicibacter arilaitensis

SEQ ID NO: 214 - DNA, 348 bp, Azospirillum lipoferum
  source 1..348: mol_type = genomic DNA
atgcccgtcacgccggacgatgccgaactgatggacgtccagcagggcgggcgctgcttcttctgcgatgggccggtgggggcgaaggcgacgttcgaccacctgatcccccaggcctatggcgggatcgacgatccggccaacgtcgtgctggcgcatcgccgctgcaaccagcgcaagggcgaccgcctgccgaccgacgatgagctcgaccgcttcttcgcgctgcgccgcttcagccggctcggcgtctggccccccttgcgcgcgctgcgcgacggggatgagggagccgacgaggaggagcggtggatggcggtggctcaggcgatcgcgcgggaacggtaa

SEQ ID NO: 215 - DNA, 564 bp, Azospirillum lipoferum
  source 1..564: mol_type = genomic DNA
ttggctccaccacccgatcactgtccggctctggtgctgaacgcggatttccgcccgctcagttacttccccttgtcgctatggtcctggcaggaagcggtcaaggcggtctttctggaacgggtcaacatcgtatcgcactatgaccgggtcgtcagatccccaagtttcgaagtccggttgcccagcgtcatctctctgaaagagttcattccggcaacgcgccgaccggccttcacccgcttcaacgtcttcctgcgcgaccgcttcacctgccagtattgcggccgccccttccccacccacgatctgaccttcgaccatgtgatcccgcgctcccgcggcggtcggacgacgtgggaaaacgtcatcacctcctgttccgcctgcaacttggcgaagggcgaccgccttccccatgtctgcggcatgatcccgctcagtccgcccttccagcccaccgcctacgagctgcaggagaacggacgcgccttcccgccaaacttccttcacgagagttggcgggattttctttattgggattcggaactggacccgatgtaa

SEQ ID NO: 216 - DNA, 3228 bp, Azospirillum lipoferum
  source 1..3228: mol_type = genomic DNA
atgcagccttaccgcctttcccttgatcttggcaccgcttcccttggctggtgcgtgctggaccttgatcatgatggaaagccgaagggcatccggcgcatgggcgtgcgcatcttcaccgatgggcgcaacccgcaggacaagaactcgctggcggtggcgcggcggatcgcgcggcagatgcggcggcggcgcgaccggacgctggtgcgccgcgcgcggctgatggcggggctgatccgcttcggcctgatgcccggcgaccgggcggcacgcaaggcgctggagcggctcgacccgtacgaccttcgcgcccgcgggctgaccgaacggctggagccgcaggagatcggccgcgcgctgttccacatcaaccagcgccgcggcttcaaggccatgcgcggcaccggcgacgaggaggaggcgggcaagatccgctccggcatcgacaggctggacatgcagatggcggaggccggcacgccgacgctgggcgccttcttcgcgtggctgcgcgcgaacgggcgcagcgtgcgcgcccggctggtcgggcgcggggccaaggccgaataccccttctacccggcgcggccgatgctggaggcggagttcgatgcgctgtgggcggcgcaggcgcgccatcacccggacctgttgacggaggaggcgcggagcacgctacgccagcgcatcttccaccagcgcccgctgaagccgtcgcaggtcggcaaatgttccctgatcccggaggacgagcgcgccccgcgcgcgctgccctcggcacagcgcttccgcctctacaccgaactcgccaacctccggatcatcatgccggaccgcagcgagcggcccctgaccatggaggaacgcgacctgatcctgcgggcggcgcgcgcgaagccggcccgcaaggccatcggcttcaaggacatacgcaaggtgctgaaactcggctccgacgtcgggttctcccacgaatccacccgcaggccggaactggcgaccgacgagacctcggcggtgctggcggacaagaagcgcttcggcccgcgctgggcgcggctgaccctggccgaacaggacgaggtggtcgaacggctgctgtcggaaaccgacgatggggcgctgatcgactggctggtcgcgcgctgggatctggagccggacgccgcggcccgtgtcgccgatgcgcggttgcccgatttccatctgcgcgtcggccggaaggccctggccgccctgctgccggtgctggagtccgaatcgcgcgagggacggcccatccgctatgacgaggccgtgccggttgccttccccggcgcccaccattcggaccgcaggccgggcgagcggctcgaccggcttccctattatgcgaaggcgctggaacgccatgtcgccttcggcaccggcgagcccgatgatccggaggagaagcggctcggccggctcgccaacccaacggttcacatcgggctcaatcaggtccggacggtggtcaatgcgctgatcgagcgctatggtccaccgcacgagatcgtcgtggaactggcgcgcgaggtgaagcagacccaggagaagcgtcgccggacggagaaggagcaggcggaaaaccagcgccggaacgaggagcgcaaggccctgctgcgcagcgtcaacgcgccagtcaacggccgcaacctgatgaagctccggctgtgggaggagcaggggagcggggtcaaccgtcgctgcccctacaccggcgagaccatcgggctggagatgctgctgtcggaaaaggtcgacatcgaccacatcctgcccttctcccgcacgctggacgacagcgccgccaacaaggtggtctgtctggccgcggccaaccgcgacaagcgcaacctgacgccatacgagcgctggcacggcgatgaggcgcgctggtcgcggattctcggctgcgtcgaggacctgccgcccaacaagcgctggcgctttgccgaaaacgccatgaaccgctgggatgaggagggcggcttcctcgcccggcagatcaccgacacgcaatatctggcccggctggcgcgggaatatctgcgccatgtctgccatcccgaccgcgtgcgcgtctcgccggggcggctgaccgccctgctgcgccggcgctggggcgtggattccatcctgcgcgaccacaaccgcgggggcggcgaagcgccggtcgacgcgaccttcgccaagaaccgcgacaaccacctgcaccacgcgctggacgcggcggtgatcggctgcatcgaccagggcatggtgcagaagcttgcgcaagcggcatcccgtgccgaggatctggggctcgaccgtctgatggacgacatcgacgatccctgggacggcttccgcgaagagttggcggcgcgggcacgcggcgtgatcgtctcgcacaagccggagcacggcaccggtggccgcctgcacgaggacacggcctatggcccgaccctggaggcggagcgggcgctcggcttcaacctcgtctaccgcaagccgctggacggcctgacggagaaggaggtcgcgcgcatccgggcccccgacctgcgcaaagccatgcaggaacgcgtcgccttccaccggaacggccccgaccggcttgacgccaagcaggcggtggcgaaggcggcggaggatctggcggcgaccgaaccctgggtgggcatccgccgtgtccgcctgctggctaaggaggccgatccgatctggctcgctgggccggacggcgaaccctacaaggggctgatcgccggcgacgtgcaccacatcgacatcgtggcgcttccggacggccgctgggccgggcgcgccgccaccttgttcgaggcccgccggtccgccctgccggacggccgtgccgcaccctatgtgccggccgacggcgaacgcttcgtcatgcgcctgcacaagggcgatctcgtcaggctggagcatgacgggcgcctgcgcatcatgcgcgtggcccggttggagcccgccaacaaccgcctgcgcatggtcgagcatgtggacgccggggaactggacaagcgtcacgccgatccggacgacccgttccgctggctgatcgtcgccttcgacaagctgcgcatgcgcagggcgcggcgggtgaccgtcgatccgctgggcacggtgcgcgatcccggattcccgaaatgggcggcgttgcccgtttccggcgaggcggagcccgcctga

SEQ ID NO: 217 - DNA, 501 bp, Arthrobacter sp.; identical to SEQ ID NO: 160
  source 1..501: mol_type = genomic DNA

SEQ ID NO: 218 - DNA, 501 bp, unidentified
  source 1..501: mol_type = genomic DNA; note = Pseudarthrobacter chlorophenolicus
atgcgcactctcgttctgaatgctggatatgaaccgctggcggttattactttccgccgggcgctggtgcttgtgcttacaggcaaggccagtgtggtggccgaaggggacgaccccgtggtgggccccaccgacgtcctggggcgcccgtccgtgatcctcctcaaccgctacatccgcccccggtacaaccactccacggccgtcagccgccggggagtgctccgacgcgacgggcataaatgtgcctactgcgggaaagcggcgcacaccattgaccatgtgcacccgaaatccaggggcggggctgattcgtgggagaacctggtggccgcctgcctgcggtgcaacaacgtcaagggcgatcacactcccgccgagatgggatggaccctgcgcttcgtcccggaacctccgcacgggaccatttggcagatcaaggagctggagaagcccaccccggcctgggatccgttcctgcttccggaacgggctgcctga

SEQ ID NO: 219 - DNA, 501 bp, unidentified
  source 1..501: mol_type = genomic DNA; note = Pseudarthrobacter phenanthrenivorans
atgcgcactctcgttctgaatgctggatatgaaccgctggcggttatttctttccgccgggcgctggtgcttgtgctgacgaacaaggcgagcgttgtggccgaaggggacgatcccgtagtgggtccccaggagatactgggccgcccgtccgtgattctcctgaaccgctacatccgccccagatacaaccagtcaacggctgtgagcagacgcggcgtgctccgacgtgacgggcacaaatgcgcctactgcggaaaagccgcgcacaccattgaccatgtccaccccaaatcccgcggtggcgcggactcatgggagaacctggtggctgcgtgccttcgctgcaacaacgcgaagggtgaccacacccccgccgagatgggctggaagctgcggttcgtgcccgaacctccgcatggcgccatctggcagatcaaggaactcgagaagcccacacctgcctgggatccgttcctgctgccggagcgcgctgcctga

SEQ ID NO: 220 - DNA, 501 bp, unidentified
  source 1..501: mol_type = genomic DNA; note = Pseudarthrobacter phenanthrenivorans
atgcgcactctcgttctgaatgctggatatgaaccgctggcggtcattactttccgccgggcgctggtgcttgtgctcacgggcaaggcgagcgtcgtggccgaaggggatgaccctgtagtgggacccaccgacgtcctgggccgtccgtccgtgatcctcctcaaccggtacatccggccccgatacaaccactccacggctgtcagccgcaggggtgtactcagacgcgacggccataaatgcgcatactgcggcaaggcagcccacaccatcgaccatgtgcaccccaagtcccggggcggggctgactcgtgggagaacctggtggcagcctgccttcgctgcaacaacgtcaagggcgaccacaccccggccgagatgggctggagcctgcgctttgtgcccgagcctccgcgcggcaccatctggcagatcaaggaactggaaaagcccacgcccgcgtgggatccgttcctcctgcccgaacgcgccgcctga

SEQ ID NO: 221 - DNA, 501 bp, Arthrobacter sp.
  source 1..501: mol_type = genomic DNA
atgcgcacactcgttctgaatgctggatatgaaccgctggcggtagttaccttccgccgggcgctggtccttgtgctcaccggaaaagcaagcgtagtggccgaaggcggcgaacctgtcgtcgggccgcaggagattctgggacgcccgtccgtgatcctcctcaatcgctacatccggccgcggtacaacaggatcacggccgtcagccggcgcggagttctccgccgcgacggccaccgctgcgcctactgcggtaagacagcccacaccatagaccacgtccaccccaaatccaggggcggcgcggattcctgggaaaacctggttgcggcgtgcttgaaatgcaataacgccaagagcgaccacacgccggcagagatgggttggaaactccgtttcacacccggcgtcccctccggaaccatgtggcagatcaaagaactcgagaagcctgcgccggactgggatccgttcctcttgccggaatccgctgcctga

SEQ ID NO: 222 - DNA, 501 bp, Arthrobacter sp.
  source 1..501: mol_type = genomic DNA
atgcgcactctcgttctgaatgctgggtatgagcccctggcggtgatcactttccgccgggcgctggtgcttgtgctcacgggcaaggccagcgtggttgccgaaggcgatgatcccgtggtgggaccgcaggaaatcctgggccgtccctcggtgattctcctgaaccgttacatccggccgaggtacaaccaggccactgctgtcagccgccgcggagtgctcaggcgcgacggccacagatgcgcgtactgcggcaaagcggcccacaccatcgaccacgtccaccccaagtcccggggcggcgaggactcctgggagaacctggtggcggcctgcctgcgctgcaacaacgtcaagggcgaccatacgccggcggaaatgggctggaaactgcgtttcgtcccggagccaccgcatggaaccatctggcagatcaaggaacttgagaagcccactccagcctgggatccgttccttctgccggagcgggccgcctga

SEQ ID NO: 223 - DNA, 501 bp, Arthrobacter sp.
  source 1..501: mol_type = genomic DNA
atgcgcactctcgttctgaatgctggatatgaaccgctggcggttatcacgttccgccgggcactggtgcttgtgctcacgggcaaagccagtgtggttgccgagggggacgatcccgtggtggggccccaggagatcctgggccgcccgtccgttatcctcctgaaccggtatatccgacccaggtacaaccaggccaccgctgtcagccgccgcggcgtgctgaggcgcgatggccaccggtgcgcctactgcggaaaagcggcgcacaccatcgaccatgtccagcccaagtcccgcggcggtgccgattcctgggaaaacctggtggccgcctgcctgcgttgcaacaacgtcaagggcgaccatactcctgctgaaatgggttggaagctgaggttcgtcccggagcccccgcacgggaccatctggcagatcaaggaacttgaaaaaccgacccctgcctgggacccgttcctgctgcccgagcgcgcagcctga

SEQ ID NO: 224 - DNA, 501 bp, unidentified; identical to SEQ ID NO: 223
  source 1..501: mol_type = genomic DNA; note = Pseudarthrobacter phenanthrenivorans

SEQ ID NO: 225 - DNA, 501 bp, Arthrobacter crystallopoietes
  source 1..501: mol_type = genomic DNA
atgcgcacactcgttctgaacgctggatacgaaccgctggcggtagttaccttccgccgggcgctgcttcttgtgctcacagggaaggcaagcatcatcgctgaggacggcgaacccgtcgtcgggcccaacgacatattcggcaggccgtcggtcatcctgctcaaccggtacatcaagattccctgtcctcaggacacgacggtgacacgccggggcgtgctgcgccgggacaattacgagtgcgcctactgcgggaagacggccaacaccgtggaccatgtgaagccgcgttcgcgcggcggaatcgacagctgggagaacctcgtcgcctgttgcctgcgctgcaataatgccaagggggaccggacactgaagcagatgggctggaatttgcgcattaccccgcgggctccccgcggcagccactggcggatccgtgaactggagcgccccgcaccggcgtggagcgagttcctcgactccaatccggcggcttga

SEQ ID NO: 226 - DNA, 501 bp, Arthrobacter sp.; identical to SEQ ID NO: 223
  source 1..501: mol_type = genomic DNA

SEQ ID NO: 227 - DNA, 501 bp, unidentified; identical to SEQ ID NO: 223
  source 1..501: mol_type = genomic DNA; note = Pseudarthrobacter phenanthrenivorans

SEQ ID NO: 228 - DNA, 501 bp, unidentified; identical to SEQ ID NO: 185
  source 1..501: mol_type = genomic DNA; note = Pseudarthrobacter phenanthrenivorans

SEQ ID NO: 229 - DNA, 501 bp, unidentified; identical to SEQ ID NO: 99
  source 1..501: mol_type = genomic DNA; note = Pseudarthrobacter chlorophenolicus

SEQ ID NO: 230 - DNA, 423 bp, unidentified
  source 1..423: mol_type = genomic DNA; note = Bacillus weihaiensis
atgttagagggatttaattatagtccacggatttcaagaaaggtaagggcaatagacgaaaataacattaaacggacttctttaaaaaagtttcatcagcaccttgatttaatgtttgaggacggtaagagaaaatgtttttattgcggtgaaaacatagctgatcaggaaatttctgttgaccatgttataccatggtcttatatgttttcggatgatttatggaacttagtatactgccataaaggggaaaactccgaaaagtcaaaccgcttaccttcagaagaagacgtaactcgtttagcaaagcgaaataaacagttgttagaccgtgtgcaaaataacaccaaggattatgatcagctgcaaattgccgttgaaaaggattacgttaaaaagttttggatttcattcaagggatag

SEQ ID NO: 231 - DNA, 990 bp, unidentified
  source 1..990: mol_type = genomic DNA; note = Bacillus weihaiensis
ttgagtcataaattaaaaataggtgagttaaatgagatatatttaacggatgaagatatttggagaatatacacaatcgttttatccagcaagtctgttaaatcctctacttataagtatgctctcataaaagctttgatagaaaatttgtatcaaattaacgaagattttgagttaacttacgatcaactagcttactcatttactaagatctattggaatttaattgtccatcataacttattaaatcaaaacagagggaaaatggctagagtagtttcaatcataaaagaggaacaaatcaaaaatggtataccttctgaaatggtatttgataaggtcgatgacacaatacaggtaaagctagttaataaagttaagacaacaatgaaagaaaatgtatatggagcactttatggagatacaagaggatgcttttattcatttgatcataaaaaagaggatttaaaactaaaccctgttgttcacagttttatgttgaactaccaaaggcttattgttaaccttacaaattaccatatggcagcaatgattgaacaactaaatgaagtaccaagtattaattatcttttaggaaaagtagagagtattgcaaagagatcttcattaaaaccttttgaaaaaattctattaacttattttaaagcggaatgcttctattgtagtaagaaattatccgctagcaaaagagaaactcacgttgatcattttattccttggtcatttgttcagtcagataatatatggaatctcgttttatcttgtaataaatgtaacagttctaaaagtgatagattaccaactagaagctatcttgaagacattattgaaagaaatcataagttaagcgatgaaagagagaatgctgcgattactacacttatgcaaaattataggtcaaagaagattatcatgctgtatgattattcaattaagaatgggtttgattcaatatggataccgaggtaa

SEQ ID NO: 232 - DNA, 990 bp, Bacillus oceanisediminis
  source 1..990: mol_type = genomic DNA
atgagtcataaattaaaggtaggtgaactgagagagtcatatttaacggatgaagatatatggagaatatatacgattttattatcaagtaagtcagttaaatcatctacgtataagtatgccctcataaaagctatcattgaaaacctttatcaagtaaacgatcaatttgagttaacatacgatcaattggcgttttcatttacaaaaatctactggaatctaattgttcagcacagcttattgcatcaaaatagaggtaaaacagcaagggttgtcacaataattaaggaagaacaaacaaaaaatgaaatcccatctgagatgatatttgataagctcaatgaaacactacagataaaattaattagtaaaataaaaactacaatgaaagaaaatgtatatggtgcactttatggtgatacaagaggtagcttttatgcgtttgatcacaaaaaagagattttaatgttaaacccagccgttcacagttttatgttgaattatcaaagactcatagttaaccttgctaattaccatatggcagccatgattgaagaactgaatgaagttcctaatacaaattatcttcttggaaaggtagagagtatagggaaacgatcatcattgaaaccatttgaaaagattttattaaaatattttatagccgaatgtttttattgtaatagaacattaactggaattaatatagaaactcaagttgatcattttattccttggtcgttcgtacaatcagatcagatctggaaccttgtattgtcatgtaataaatgtaacagttcaaaaagtgacaagttaccggcaaggtcttatcttgaatacataatttatagaaacaatacattatgtgatagaaaagacgatgttactattgcaaatcttatggaaaattacaaatctaaaaagatagttatgctttatgattattctattaagaatggttttgattcaatatggttaccaagatga

SEQ ID NO: 233 - DNA, 501 bp, unidentified; identical to SEQ ID NO: 101
  source 1..501: mol_type = genomic DNA; note = Pseudarthrobacter chlorophenolicus

SEQ ID NO: 234 - DNA, 501 bp, unidentified
  source 1..501: mol_type = genomic DNA; note = Pseudarthrobacter chlorophenolicus
atgcgcactctcgttctgaatgctggatatgaaccgctggcggttatcactttccgccgggcgctggtgcttgtgctcacgggcaaggcaagcgtggtggccgaaggggatgacccggtggtagggcccaccgatgtcctgggccgcccgtccgtgatcctcctcaaccgctacatccggccccggtacaaccactcgacggcagtcagccgcaggggtgtgctccggcgcgacggacataaatgtgcctactgcggcaaggcggcgcacacaatcgaccacgttcacccgaagtcccggggcggtgccgattcatgggagaaccttgtggccgcctgcctgcggtgcaacaacgtcaagggcgaccacactcccgcggaaatgggttggacccttcggttcgtgcccgagccgccccacggaacgatctggcagatcaaggaactggagaagccgacgccggcgtgggatccgttcctgctcccggagcgcgccgcctga

SEQ ID NO: 235 - DNA, 975 bp, Bacillus megaterium; identical to SEQ ID NO: 9
  source 1..975: mol_type = genomic DNA

SEQ ID NO: 236 - DNA, 501 bp, unidentified; identical to SEQ ID NO: 223
  source 1..501: mol_type = genomic DNA; note = Pseudarthrobacter phenanthrenivorans

SEQ ID NO: 237 - DNA, 501 bp, unidentified; identical to SEQ ID NO: 223
  source 1..501: mol_type = genomic DNA; note = Pseudarthrobacter phenanthrenivorans

SEQ ID NO: 238 - DNA, 348 bp, Arthrobacter sp.
  source 1..348: mol_type = genomic DNA
gtgatcctcctgaaccgctacatccgcccgcggtacaacaccatcaccgccgtgagccgccggggcgtgctgcgccgcgacggccaccgctgcgcctattgcggtaaagcagcgcacaccattgaccacgtccatcccaaatcgaggggcggtgccgactcgtgggaaaacctggtggccgcctgcctcagatgcaacaacgtgaagggcgaccacaccccggccgagatgggctggaagctcaggtttgtgccggcgcctccgcacggcaccatctggcagatcaaggagctggaaaagcccactcccgcgtgggatcctttcctgctgcccgaatccgccgcctga

SEQ ID NO: 239 - DNA, 501 bp, unidentified
  source 1..501: mol_type = genomic DNA; note = Pseudarthrobacter sulfonivorans
atgcgcactctcgttctgaatgctggatatgaaccgctggcggtagtcaccttccgccgggcgctggtccttgtgctcacggggaaggcaagcgttgtcgcggagggcgaggaccctgtagtaggaccgcaggagatcctgggccgcccgtccgtgatcctcctgaaccgttacatccgcccccgatacaaccagaccaccgctgtgagccgccggggagtgctgcgccgggacggacacaggtgcgcatactgtggaaaagcggcccacaccattgaccacgtccagcccaagtcccgaggcggtgcagattcctgggagaatctcgtcgccgcgtgcctgcgctgcaataacgtcaagggcgaccacacacccgcggagatgggctggaaactccggttcacaccggaaccgccgcatggcaccatctggcagatcaaggagctggagaagcccacgcctgcctgggatcccttcctgctgccagaacgggctgcctga

SEQ ID NO: 240 - DNA, 501 bp, unidentified
  source 1..501: mol_type = genomic DNA; note = Pseudarthrobacter chlorophenolicus
atgcgcactctcgttctgaatgctggatatgaaccgctggcggtcattactttccgccgggcgctggtgcttgtgctcacgggcaaggcgagcgtcgtggccgaaggggatgaccctgtcgtgggacccacggacgtcctgggccgcccgtccgtgatcctcctcaaccggtacatccggccccgatacaaccactccacggctgtcagccgcaggggcgtactcagacgcgacggccataaatgcgcatactgcggcaaggcagcgcataccatcgaccatgtgcaccccaagtcccggggcggggctgactcgtgggagaacctggtggcagcctgtcttcgctgcaacaacgtcaagggcgaccacaccccgaccgagatgggctggagcctgcgctttgtgccggagcctccgcgcggcaccatctggcagatcaaggaactggaaaagcccacgcccgcgtgggatccgttcctcctgcccgaacgcgccgcctga

SEQ ID NO: 241 - DNA, 975 bp, Bacillus megaterium; identical to SEQ ID NO: 9
  source 1..975: mol_type = genomic DNA

SEQ ID NO: 242 - DNA, 975 bp, Bacillus megaterium; identical to SEQ ID NO: 9
  source 1..975: mol_type = genomic DNA

SEQ ID NO: 243 - DNA, 975 bp, Bacillus megaterium
  source 1..975: mol_type = genomic DNA
gtgagttttaatttaaaagtaggagaaataaaaaaagcttatttaaaagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcctttgctaaaatatattggaatttggttattcatcatgatttaaatcaattaaatacacacaatagacaagctggaattcaaaaagagttaaaggaatttcagttaatgcatgatgtcccaaataaagtagtatttgatcgtttaccaagtaatcttcaattgcagttagtagaaagaactaaaaaagtgggggcaaggtatgtagtaggggcgttgtacggagatatggaaggttctatttatgagtttgataaaagaacagaatacataaagtttaacagcagtatgtatatttttttgcaaaaatatagacaaattgctactcatttaactaattatcatttggcgaaatttttagaaaagcataatgacaaaaataagttagaagatgtcttgttaaaagtggaaaatgtttcaaaacgtgaatcgctgcgtgcattttataacatcctttggaaatatgaaacaaaagaatgtttttattgtgggaaagttctttctgataaacgcatcgctactcatgtagatcattttattccatggagttatatccaatcagataatatgtggaatttggtgttaagctgtcaaagctgcaacacaaaaaagagcaataaacttgctaagagtgtatacctcgagaagatgtttgttcgtaacgataatcttaaattaacagcagttcacaatgattttaactattattcggagcgtaagttagaagctctttatgagtttgctggaatgaatggatacgtagaaaactgctctccttaa

SEQ ID NO: 244 - DNA, 975 bp, Bacillus megaterium
  source 1..975: mol_type = genomic DNA
atgaatgactatattaaacaatggcatagtttaattttaaattgcaactttgataacacatataaaatggtttggagcaaagcattagtagaattgtcggttaatgttagtgacgaagttgataaagatataataatttttgattttgacgatattgcaaaactttgcttaaaatattattggaatcaaactatttactttgatttaatacaaagtcctaacttaaagaaacctccagaaatacttaatcatattaaatccttaattacccatttctttgatgcgaaaacttctgttcatccagttagatttgaaaagatagattttaacagacttcaattaatagataagtacgaagaaaccattaaaagaatagtaacaacgttaaagaaagatgtttgctggcgatttaaaattcttaattctaacacatacgatatatatgaattagaccgtaaaaaaggtaaggtgtattttaaaagacaagacatattaacgcttaaacaaaatagtgacttactatttacctttataaattacagatggacacaaatgttagaggggttcaattatagtccacggatttcaagaaaagtacgtgcgatagatgaaaacaatattaagcgtatgccgttaaaaaagtttcataagtatcttgatttaatgtttgaagacagtaaacgtaaatgtttttattgtggtgaagaaatagatgatagcaatctgtcggttgaccatgttataccttggtcttacatgttttcagatgatttatggaatttagtttactgtcataaagatgaaaactccgaaaaatctaatcgtctcccattggaagaagatatattaagattagaaaataggaatatagctttactagaacgtatggagaacgcaaccaaagactacgaccaattacaaatagcccttgagaaaaattatgttaaacagttttggatttcatttaaagggtaa

SEQ ID NO: 245 - DNA, 975 bp, Bacillus megaterium; identical to SEQ ID NO: 9
  source 1..975: mol_type = genomic DNA

SEQ ID NO: 246 - DNA, 975 bp, Bacillus megaterium; identical to SEQ ID NO: 9
  source 1..975: mol_type = genomic DNA

SEQ ID NO: 247 - DNA, 975 bp, Bacillus megaterium; identical to SEQ ID NO: 9
  source 1..975: mol_type = genomic DNA

SEQ ID NO: 248 - DNA, 996 bp, Bacillus sp.
  source 1..996: mol_type = genomic DNA
atgagccacaaattacaagtgggagaaatgaaaacttcctatttaaccgataaggaaatatgggcacattttaattacattttctcgtcaaaatcaaaaaactcaactacctacaaatttgttttgattaaatccattctagagaacttatacaatgttaatgaaaatctggagttaaattattcagttttgttttcgagtttttccaaaatctattggaatctggttatccaccataatcttaatcaaattaatatgactgataaaaaagcagaggtacaaagaatccttatggatgtccagaagaagcatcaaattccaaatacactagtctttgataggctttcgaatgaattgcagattgaaataataagtaaggtaacgaaacgatgtaaaattaatgtaatgggtgctttatatggagacactagctgtacaatatacgattttgataattcgaaggaacagctgagactaaatagtactttttattcctttatgcagcggttccaactcgtgttaaattatttaaccaattatcatcttgctttatttttagaaaaattcaatgaaaaaggtgacaccacgaaccttttattaaaggttgaaaacgtctctaagcggtcttctttggatcagttttaccaagtcctttcctccttctataacgggaaatgtttttattgtggaaaagcaatcaagaaagaaaatgcacatgtggatcactttattccttggagctttgtgcaagcagatcaactttggaatcttgttatcgcgtgtagtgcttgtaacttatcaaaaagtgataaattggcaaataggctgtttttagaatccgtaatagaacgaaatgataaccttataaccataccagagctaagcaaaagggaagacatgaaagtatacaccagcaaaaaaatgcaagacctatattattactcagttgaaaacgggtttactgatatttggttgccgaaaaaaataataatataa

SEQ ID NO: 249 - DNA, 306 bp, Arthrobacter sp.
  source 1..306: mol_type = genomic DNA
atggcaacctcacgcactggcacatcccagtacaagaaggcccgcaagcgcacactccaccaggccaaagcccaaggcatagagcgctgcccatactgcaaggtcaagctcgactacgacgtaagccgcaagcccaacagtgcagagacagaccacgtcatcgcacacgcaagaggcggacaagaccacgtcagtaacctcgcagtgtgctgcagacgatgcaaccagtccaaaggcgacagaccagcacccaagacgtccaccgtcctcgctgcgaaaccgctcaagacgagccgtcgctggtag

SEQ ID NO: 250 - DNA, 501 bp, Arthrobacter sp.
  source 1..501: mol_type = genomic DNA
atgcgcactctcgttctgaatgctggatatgaaccgctggcggtcatcacgttccgccgggcactggtgcttgtgctcacgggcaaagccagtgtggttgccgagggtgacgatcccgtggtggggccccaggagattttgggccgcccgtccgtaatcctcctgaaccgctatatccgccccaggtacaaccaggccaccgctgtcagccgccgcggcgtgttgaggcgtgatggccaccgttgcgcctactgcgggaaagcggcccacaccattgaccatgtccagcccaagtcccgcggtggtgccgattcctgggagaacctggtggccgcctgcctgcgttgcaacaacgtcaagggcgaccacactcccgctgaaatggggtggaagctgaggttcgtcccggagcccccgcacggaaccatctggcagatcaaggaactcgaaaaacccacccctgcctgggatccgttcctgctgcccgagcgcgcagcctga

SEQ ID NO: 251 - DNA, 501 bp, unidentified
  source 1..501: mol_type = genomic DNA; note = Pseudarthrobacter chlorophenolicus
atgcgcactctcgttctgaatgctggatatgaaccgctggcggttgtgactttccgccgggcgctggtgcttgtgctcgcgggcaaggcgagcgtggtggccgaaggtgacgaccccgtggtgggccccaccgacgtgcttggccggccgtccgtgattctcctcaaccgctatatcaggccccgctacaaccattccacggccgtcagccgcaggggagtgctccggcgcgacgggcataaatgcgcctactgcggcaaggcagcgcacactatcgaccacgtccaccccaagtcacggggcggggccgattcctgggaaaacctggtggcagcatgcctgcggtgcaacaacgtcaagggcgaccacacccctgccgagatgggatggaccctgcgcttcgtccccgagccgccgcatggaaccatctggcagatcaaggaactggagaagccgacgcctgcctgggatccgttcctgcttccggagcgcgctgcctga

SEQ ID NO: 252 - DNA, 501 bp, unidentified; identical to SEQ ID NO: 234
  source 1..501: mol_type = genomic DNA; note = Pseudarthrobacter chlorophenolicus

SEQ ID NO: 253 - DNA, 501 bp, unidentified; identical to SEQ ID NO: 223
  source 1..501: mol_type = genomic DNA; note = Pseudarthrobacter phenanthrenivorans

SEQ ID NO: 254 - DNA, 501 bp, unidentified; identical to SEQ ID NO: 234
  source 1..501: mol_type = genomic DNA; note = Pseudarthrobacter chlorophenolicus

SEQ ID NO: 255 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattttttgctaatggtcactttaccacaacgtataaatatggattaatgaaagctttaattgaaaacctatataacgttgacaatcggttagtactcacgtttgatcaagtatatttttcatttgctaaaatgtattggaatttggttattcatcatgatttaaatcaattaaatacacccaatagacaagctggaattcaaaaagagttaaaggaatttcagttaatgcatggtgtccccaataaagtagtatttgatcgtttgccaagtaattttcaattgcagttagtagaaagaactaaaaaagtgggggcaaggtatgtagtaggggcgttatacggagatatggaagcttctatttatgagtttgataaaagaacagaatacataaagtttaatagcagtatctatatttttttacaaaaatatagacaaattgttactcatttaaccaattatcatttggcgaaatttttagaaaagcataatgacaaaaataagttagaagatgtcttgttgaaagtagaaaatatttcaaaacgcgaatcgctgcatgcattctataacatactttggaaatatgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgagtatgctggaatgaatggatacgtagaaaactggtctccatag

SEQ ID NO: 256 - DNA, 966 bp, Bacillus megaterium
  source 1..966: mol_type = genomic DNA
gtgagttttaatttaaaagtaggagaaataaaaaaagcttatttaacagaacaagaaatttggaagatcattaatcaattttttgctaatggtcactttaccacaacgtataaatatggattaatgaaagctttaattgaaaacctatataacgttgacaatcggttagtactcacgtttgatcaagtgtatttttcatttgctaaaatatattggaacttggttattcatcatgatttaaatcaattaaatacacccaatagacaagctggaattcaaaaagagttaaaggaatttcagttaatgcatggtgtccccaataaagtagtattcgatcgtttaccaagtaattttcaattgcagttagtagaaagaactaaaaaagtgggggcaaggtatgtagtaggggcgttatacggagatatggaaggttctatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagaagatgtcttgttgaaagtggaaaatataacaaaacgcgaatcgttgcatgcattctataacatactttggaaatacgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgagtatgctggaatgaatggatacgtagaaaactga

SEQ ID NO: 257 - DNA, 501 bp, unidentified; identical to SEQ ID NO: 223
  source 1..501: mol_type = genomic DNA; note = Pseudarthrobacter phenanthrenivorans

SEQ ID NO: 258 - DNA, 585 bp, Paenibacillus xylanexedens; identical to SEQ ID NO: 126
  source 1..585: mol_type = genomic DNA

SEQ ID NO: 259 - DNA, 975 bp, Bacillus megaterium; identical to SEQ ID NO: 9
  source 1..975: mol_type = genomic DNA

SEQ ID NO: 260 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattttttgctaatggtcactttaccacaacgtataaatatggattaatgaaagctttaattgaaaacctatataacgttgacaatcggttagtactcacgtttgatcaaatatatttttcatttgctaaaatatattggaatttggttattcatcatgatttaaatcaattaaatacacccaatagacaatctggaattcaaaaagagttaaaggaatttcagttaatgcatggtgtccccaataaagtagtatttgatcgtttgccaagtaattttcaattgcagttagtagaaagaactaaaaaagtgggggcaaggtatgtagtaggggcgttatacggagatatggaagcttctatttatgagtttgacaaaagaacagaatacataaagtttaatagtagtatctatatttttttacaaaaatatagacaaattgttactcatttaaccaattatcatttggcgaaatttttagaaaagcataatgacaaaaataagttagaagatgtcttgttgaaagtagaaaatatttcaaaacgcgaatcgctgcatgcattctataacatactttggaaatatgaaacaaaagaatgtttttattgtgggaaagttctttctgataaacgcatcgctactcatgtagatcattttattccatggagttatattcaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgagtatgctggaatgaatggatacgtagaaaactggtctccatag

SEQ ID NO: 261 - DNA, 501 bp, unidentified; identical to SEQ ID NO: 234
  source 1..501: mol_type = genomic DNA; note = Pseudarthrobacter chlorophenolicus

SEQ ID NO: 262 - DNA, 537 bp, unidentified
  source 1..537: mol_type = genomic DNA; note = Glutamicibacter arilaitensis
atgccagatggtagctatcggttcaagaaggaagtacgacaaatgcgaacactggtattaaatgcaggatacgagcccttagcagtcgtgacttttcgacgagcactggtaatggtcatggccgataaagcgaccatcgttctccaggatgacgactcgcctgttcgcagctcccacggacagctacccagacccactgtcattgtcttaaaacgctatgtacgacgcccctaccgcgacatgcttcatgtcacgcgtaggggcgttttgcgtcgagacgcgtatacctgcgcgtactgcgggaaatcggcgaatacgatcgaccacgtcattccgcgaagcaggggagggaagaacacgtgggagaacttggtggctgcttgcctgcactgcaacagcaggaaagccgacaagatgctctctcagttgggctggcagctgcggatcacgccgcatcgccctcggatgggcggacatctggtctccggtcttcagcatattgataccgcatgggacgacttccttcaagcaagctga

SEQ ID NO: 263 - DNA, 501 bp, unidentified
  source 1..501: mol_type = genomic DNA; note = Pseudarthrobacter chlorophenolicus
atgcgcactctcgttctgaatgctggatatgaaccgctggcggtcatcactttccgccgggcgctggtgcttgtgctcacaggcaaggcgagcgtggtggccgagggggatgaccccgtggtaggccccaccgacgtcctgggccgcccgtccgtgattctcctcaaccgctacatccggccccgatacaaccactccacggccgtgagccgcaggggtgtgctccggcgcgacgggcataaatgcgcctactgcggcaaggctgcgcacaccattgaccatgtgcacccgaaatcccggggtggggcggattcgtgggagaacctggtggccgcctgcctgcggtgcaacaacgtcaagggcgaccacactcccgccgaaatgggctggaccctgcggtttgttccggagccgccgcacggaaccatctggcagatcaaggaattggagaagcccacgccggcgtgggatccgttcctgcttccggagcgcgctgcctga

SEQ ID NO: 264 - DNA, 975 bp, Bacillus megaterium; identical to SEQ ID NO: 9
  source 1..975: mol_type = genomic DNA

SEQ ID NO: 265 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcctttgctaaaatatattggaatttggttattcatcatgatttaaatcaattaaatacacacaatagacaagctggaattcaaaaagagttaaaggaatttcagttaatgcttggtgtcccaaataaagtagtatttgatcgtttaccaagtaatcttcaattgcagttagtagaaagaactaaaaaagtgggggcaaggtatgtagtaggggctttatacggagatatggaaggttccatttatgagtttgataaaagaacagaatacataaagtttaatagcagtatgtatatttttttgcaaaaatatagacaaattgttactcatttaaccaattatcatttggcgaaatttttagaaaagcataatgacaaaaataagttagaagatgtcttgttaaaagtggagaatatttcaaaacgcgaatcgctgcatgcattctataacatactttggaaatacgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtttgctggaatgaatggatacgtagaaaactgggctccttaa

SEQ ID NO: 266 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattttttgctaatggtcactttaccacaacgtataaatatggattaatgaaagctttaattgaaaacctatataacgttgacaatcggttagtactcacgtttgatcaagtatatttttcatttgctaaaatatattggaatttggttattcatcatgatttaaatcaattaaatacacccaatagacaatctggaattcaaaaagagttaaaggaatttcagttaatgcatggtgtccccaataaagtagtatttgatcgtttaccaagtaattttcaattgcagttagtagaaagaactaaaaaagtgggggcaaggtatgtagtaggggcgttatacggagatatggaagcttctatttatgagtttgacaaaagaacagaatacataaagtttaatagcagtatctatatttttttacaaaaatatagacaaattgttactcatttaaccaattatcatttggcgaaatttttagaaaagcataatgacaaaaataagttagaagatgtcttgttgaaagtagaaaatatttcaaaacgcgaatcgctgcatgcattctataacatactttggaaatatgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgattatgctggaatgaatggatacgtagaaaactggtctccatag

SEQ ID NO: 267 - DNA, 564 bp, Azospirillum lipoferum
  source 1..564: mol_type = genomic DNA
ttggctccaccacccgatcactgtccggctctggtgctgaacgcggatttccgcccgctcagttacttccccttgtcgctatggtcctggcaagaagcggtcaaggcggtctttctcgaacgggtcaatatcgtatcgcactatgaccgggtcgtccgatccccaagtttcgaagtccggttgcccagcgtcatctcgctgaaagagttcatcccggccacgcgccgtccggccttcacccgcttcaacgtcttcctgcgcgaccgcttcacctgccagtattgcggccgccccttccccacccacgacctgaccttcgaccatgtcatcccgcgatcccgcggcggtcggacgacgtgggagaacgtcatcacatcctgttcggcctgcaatctggcgaagggcgaccgccttccccacgtctgcggcatgatccccctcagtccgcccttccagcccagtgcctacgagctgcaagagaacggacgcgccttcccgccaaacttcctccatgaaagttggcgggattttctttattgggactcggaattggacccgatgtag

SEQ ID NO: 268 - DNA, 345 bp, Azospirillum lipoferum
  source 1..345: mol_type = genomic DNA
atgcccgtcacgccggacgatgccaggctgatggacgtccagcagggcgggcgctgcttcttctgcgacgggccggtgggggtgaaggccaccttcgaccatctgatcccccaggcatacggcgggatcgacgatccgtccaacgtcgtgctggcgcaccgccgctgcaaccagcgcaaggaagaccggctaccgaccggagaggaactcgaccgcttcttcgcgctgcgccgctccagccggctcggcgtctggccccccttgcgcgcgctgcgcgatgcggacgagtgggcggaggaggagcggtggatggcggtggcccaggcgattgcccggcaaagctga

SEQ ID NO: 269 - DNA, 564 bp, Azospirillum lipoferum
  source 1..564: mol_type = genomic DNA
ttggctccaccacccgatcactgtccggctctggtgctgaacgcggatttccgcccgctcagttacttccccttgtcgctatggtcctggcaggaagcggtcaaggcggtctttctggaacgggtcaacatcgtatcgcactatgaccgggtcgtccgatccccaagtttcgaagtccggttgcccagcgtcatctcgctgaaagagttcatcccggccacgcgccgcccggccttcacccgcttcaacgtcttcctgcgcgaccgcttcacctgccagtattgcggccgccccttccccacccacgatctgaccttcgaccatgtgatcccgcggtcccgcggcggtcggacgacgtgggaaaacgtcatcacatcctgttcggcctgcaacctggcgaagggcgaccgccttccccacgtctgcggcatgatccccctcagtccgcccttccagcccagtgcctacgagctgcaagagaacggacgcgccttcccgccaaacttcctccatgaaagttggcgggattttctttattgggactcggaactggacccgatgtag

SEQ ID NO: 270 - DNA, 564 bp, Azospirillum lipoferum; identical to SEQ ID NO: 269
  source 1..564: mol_type = genomic DNA

SEQ ID NO: 271 - DNA, 345 bp, Azospirillum lipoferum; identical to SEQ ID NO: 268
  source 1..345: mol_type = genomic DNA

SEQ ID NO: 272 - DNA, 975 bp, Bacillus megaterium
  source 1..975: mol_type = genomic DNA
gtgagttttaatttaaaagtaggagaaataaaaaaagcttatttaacagaacaagaaatttggaaggtcattaatcaatttttcgctaatggtcactttaccacaacgtataaatatggattaatgaaagctttaattgaaaacctatataacgttgacaatcggttagtactcacgtttgatcaagtgtatttttcatttgctaaaatatattggaatttggttattcatcatgatttaaatcaattaaatacacacaatagacaagctggaattcaaaaagagttaaaggaatttcaattaatgcatggtgtcccaaataaagtagtgtttgatcgtttaccaagtaatcttcaattgcagttagtagaaagaactaaaaaagtgggggcaaggtatgtagtaggggcattatacggagatatggaaggttctatttatgagtttgataaaagaaaaggatacataaagtttaacagcagtatgtatgtgtttttacaaaaatatagacaaattgttactcatttaacgaattatcatttagctaaatttttagaaaagcataatgacaaggataagttagaagatgtcttgttgaaagtagaaaatatttcaaaacgcgaatcgctgcatgcattctataacatactttggaaatatgaaacaaaagaatgcttttattgtggaaaacgtctttctgataaacgcatcgctactcatatagatcattttatcccatggagctatatccaatcagataatatgtggaatttggtgctaagctgtcaaaactgcaacacacaaaaaagcaataaactagccaaagatgtatatcttgagaaaatgtttattcgtaataacaatcttaaattaacagcagttcacaatgattttaactattactcggagcgtaagttaaaagctctttatgagtatgctggaatgaatgggtacgtagaaaactggtctccttag

SEQ ID NO: 273 - DNA, 501 bp, unidentified; identical to SEQ ID NO: 263
  source 1..501: mol_type = genomic DNA; note = Pseudarthrobacter chlorophenolicus

SEQ ID NO: 274 - DNA, 501 bp, unidentified; identical to SEQ ID NO: 263
  source 1..501: mol_type = genomic DNA; note = Pseudarthrobacter chlorophenolicus

SEQ ID NO: 275 - DNA, 501 bp, unidentified; identical to SEQ ID NO: 263
  source 1..501: mol_type = genomic DNA; note = Pseudarthrobacter chlorophenolicus

SEQ ID NO: 276 - DNA, 501 bp, unidentified; identical to SEQ ID NO: 263
  source 1..501: mol_type = genomic DNA; note = Pseudarthrobacter chlorophenolicus

SEQ ID NO: 277 - DNA, 501 bp, unidentified; identical to SEQ ID NO: 99
  source 1..501: mol_type = genomic DNA; note = Pseudarthrobacter phenanthrenivorans

SEQ ID NO: 278 - DNA, 501 bp, Arthrobacter sp.
  source 1..501: mol_type = genomic DNA
atgcgcactctcgttctgaatgctggatatgaaccgctggcggtcatcacgttccgccgggcgctggtgcttgtgctcacaggcaaagccagtgtggttgccgagggtgacgatcccgtggtgggaccccaggagatattggggcggccgtccgttatcctgcttaaccgctatatccgtcccagatacaaccaggccacggctgtgagccgccgcggagtgctgaggcgtgatggccacaggtgcgcgtactgcggaaaagcggcccacaccatcgaccatgtccagcccaagtcccgcggcggtgccgactcttgggagaacctggtggcggcctgcctgcgctgcaacaacgtcaagggcgaccacactccggccgagatgggatggaagttgcgcttcgtcccggagcctccgcacggaaccatctggcaaatcaaggaactcgaaaagcccacccccgcctgggacccgttcctgctgcccgagcgcgccgcctga

SEQ ID NO: 279 - DNA, 501 bp, Arthrobacter sp.
  source 1..501: mol_type = genomic DNA
atgcgcactctcgttctgaatgctggatatgaaccgctggcggtcatcacgttccgccgggcactggtgcttgtgctcacgggcaaagccagtgtggtcgccgagggtgacgatcccgtcgtggggccccaggagatattgggccgcccgtccgttatcctcctgaaccgctatattcgccccaggtacaaccaggccaccgctgtcagccgccgcggcgtgctgaggcgcgatggccaccggtgcgcctactgcggaaaagcggcccacaccatcgaccatgtccagcccaagtcccgcggcggtgccgactcctgggagaacctggtggccgcctgcctgcgttgcaacaacgtcaagggcgaccacactcctgctgaaatggggtggaagctgaggttcgtcccggagcccccgcacggaaccatctggcaaatcaaggaactcgaaaaacccacccctgcctgggacccgttcctgctgcccgagcgcgcagcctga

SEQ ID NO: 280 - DNA, 975 bp, Bacillus megaterium; identical to SEQ ID NO: 206
  source 1..975: mol_type = genomic DNA

SEQ ID NO: 281 - DNA, 501 bp, Arthrobacter sp.; identical to SEQ ID NO: 112
  source 1..501: mol_type = genomic DNA

SEQ ID NO: 282 - DNA, 501 bp, unidentified
  source 1..501: mol_type = genomic DNA; note = Pseudarthrobacter chlorophenolicus
atgcgcactctcgttctgaatgctggatatgaaccgctggcggtcattactttccgccgggcgctggtgcttgtgctcacgggcaaggcgagcgtcgtggccgaaggggatgaccctgtcgtgggacccacggacgtcctgggccgcccgtccgtgatcctcctcaaccggtacatccggccccgatacaaccactccacggctgtcagccgcaggggcgtactcagacgcgacggccataaatgcgcatactgcggcaaggcagcgcataccatcgaccatgtgcaccccaagtcccggggcggggctgactcgtgggagaacctggtggcagcctgtcttcgctgcaacaacgtcaagggcgaccacaccccgaccgagatgggctggagcctgcgctttgtgccggagcctccgcgcggcaccaactggcagatcaaggaactggaaaagcccacgcccgcgtgggatccgttcctcctgcccgaacgcgccgcctga

SEQ ID NO: 283 - DNA, 501 bp, Arthrobacter sp.
  source 1..501: mol_type = genomic DNA
atgcgcactctcgttctgaatgctggatatgaaccgctggcggttatttcattccgccgggcgctggtgcttgtgctgaccaacaaggcgagcgtggtggccgagggggatgatcccgtggtgggtccccaggagatcctgggccgcccgtccgtgattctcctgaaccgctacatccgtcccagatacaaccacgccatggccgtcagccggcggggtgtgctcaggcgggacgggcacaagtgcgcgtactgcggaaaagcggcgcacaccatcgaccacgtccatccaaaatcccgtggtggcgccgattcgtgggagaacctggtggccgcctgcctccgctgcaataacgccaagggtgaccatacccccgctgaaatgggctggaagctgcggttcgtaccggagcctccgcacggcactatctggcaaatcaaggaactggaaaagcccacgccggcgtgggatcccttcctgttgcctgaacgcgctgcctga

SEQ ID NO: 284 - DNA, 975 bp, Bacillus megaterium; identical to SEQ ID NO: 255
  source 1..975: mol_type = genomic DNA

SEQ ID NO: 285 - DNA, 501 bp, unidentified; identical to SEQ ID NO: 147
  source 1..501: mol_type = genomic DNA; note = Pseudarthrobacter chlorophenolicus

SEQ ID NO: 286 - DNA, 975 bp, Bacillus megaterium; identical to SEQ ID NO: 11
  source 1..975: mol_type = genomic DNA

SEQ ID NO: 287 - DNA, 501 bp, Arthrobacter sp.; identical to SEQ ID NO: 107
  source 1..501: mol_type = genomic DNA

SEQ ID NO: 288 - DNA, 1011 bp, unidentified
  source 1..1011: mol_type = genomic DNA; note = Fictibacillus arsenicus
atggtggcattgagccataaattacaagtaggtgaaatgaaggttaaatatttgactgaccaggaaatatggggacacttcaattatatcttttctccaaaatctaaaaattccacaacatataaattcgtcttgattaaatcaattttggagaatctttataatgttaatgaaaaattagaattaacctatgatcttttatttaacagcttttctaaaatatattggaatctagtaattcatcataaccttaaccaaattaatatgaccggaaaaaaagctgaggtacaaaaattacttatagagattaaagaaaagtataatgtaccaaatacatttgttttttataagctagctgaagagctacaaattgaaatcattacaaaagtaaaaaagaaagctaagctgaatgttatgggtgctatttatggtgatacaaacagtacaatttatgatttcgataataagaaggaatttatcaaaataaatcatagcttttactcgtttatgcaacgtttccaaaaagtattaagttacttaacaaactatcaccttgcattatttttagagaaattcaatcaaaaaggtgatatcaccaatcttcttatgaaagttgaaaatgtctccaaaagatcttcgttaaatgaattttactttcttctatcatcaatatataatgacaaatgtttttattgcggaaaagaaattaagacaaaagggtccatacatatcgatcatttcattccatggagctttgtgcaaactgatcaattatggaacttggttatttcctgtagttcttgtaatttagctaaaaatgatatgttagcagtagatacatttctacactttctcatagatagaaatgtggaattaaaaaataatccaaccttaaatcaaagaccagacatgaaggtttataatgaaaaaaaattaacagagctttatcagtactctaaagaaaatgggtttactgatatttggactcctcaaaagaacattcaattataa

SEQ ID NO: 289 - DNA, 1755 bp, Paenibacillus xylanexedens
  source 1..1755: mol_type = genomic DNA
ttgaataatagatacagtaaaaaaaatgacatactaagagctgcgctttatttggaatatgaggggaattgtttttatgaaggtttaaaaattagattcaatgatatgcatatagatcatattattcctgagtcgataagtgaagaagctcttcaagatataatttcctctctcggcctgccaaaagattttaatgtaaacagtttatataaccttgttccttgttctcctaatgttaatcaagttaaaaataaaaaccagtatccaataaaattcttaggtcattgtattttcaatgaaacatctaaaaaagtagaaggtgttaagaaacgaattgatcaactatctaaagagttcaaaagtgataaagatgttgcaaaattaatagcacgcttaaatatctctgataataaaagagaattagagaacgtatataatgcgctatccaaggaaaagccttttgatataaaaagagcggttgagaaaagagggagattttatagttttgaaagaagtttctctaatgttaggttagctggtcaaatacctacatatcctgatatgaaaggtaattgtatgattacttttagtaatttaagactaagagattgtatgattagtttgaatcactcacaaataatggaacagttatttgaaggtgcccatactgacatcaataataatcttaggaattttataatatattccgaagaaatagatcccgatatctattatgttactctggccaacgttcgtcttcctcttgaaaaagatgaagttatacaactcttggaaataattgatgacttttacgatctgtacatagaagaaagtaaagaattctatgaagttttccgaagagattttttcataaacggttataagaagaaaaatgatataagactttttaaaataaaaaaagatttatggtaccttattcgtgaatttagcagaaagtttgattatgaaaacggaaactcggattggcatatatttgatgcacatgggtctatactcaaagtttatgatcaaacagtaggtgattttatggtttttatttttccgcaatcagaacaagttgagtactttgtttataatgaagatgaagtatggttagtatggactgatgattattttattaatagtagcttagaaagttttaaatttaatctgcattgggggccgtttttcacttatcattggttaataaacgaactcatccctcaagtgatgtattggcatgagagtcaaaataaaaaaatgttcaaaaagcaaaaatctttaaatgaatttaaaaactcatttgtaatatctgactatgtaagttatttgcatgaatcagagagtaacgacaagttgttggctcgtgcttctgacctgcaagagtttttccatgttagaccacataatatgtatcaaattaaggatttgtctaaagtatatgaatgcctaattgaaattataaataaatcgaatctcagtgaatccgaattttattatatatctggaaagcttaatgtgagagagtttcagactaaagaattattaatgcaaggtatttcggaattaatgttgcagagtgaacaagagagagttatttcttcttcttcactagacttaatttatagatgtatggtagttgtattgatagactgcaataactctatgaatgatcatgatcggaaccaattgatagaaaaacttcagttccagtatgatttgttgactcaagaaagaataagagaagagatataa

SEQ ID NO: 290 - DNA, 936 bp, Bacillus sp.
  source 1..936: mol_type = genomic DNA
ttgaattgtagctttgataatacatataaaatggcatggtctaaggcattagttgaattgatcgttaataccaacgaacctaaagaggatattgttatatttgattttaacgatattgcaaagctttgtttaaagtattattggaatcaaacaatttactttgatttaatacaaagtccaaacataaagaagcctcctgaaattatttcctctactaaggatttaataaggtcatttttttatcaaaagcaatctgtacagccaattagatatgaaaaaatagactttaacacgattggattagcacgggaatatgaattaacaataaaaaatattgttagaacactgaagcaagatgtatgttggcgttttaagaaccttaaccgtgatacatacgaaatatatgaattggaccgtaatgatggtaaggtattttttaataaaaaccatgtattaactcttaacgagtatagtgatcttttattcaccgtaataaattaccgatggacacaaatgttagagggctttaattacagtccacggatttcaagaaaagtaagggcaatagacgaaaataacattagacgaacttctttaaaaaggtttcatcagcaccttgatttaatgtttgaggacggcaaaacaaagtgtttttattgcggtgaaaacatagctgataacgagatttctgttgaccatgttattccgtggtcttatatgttttcagatgatttatggaacttagtttactgtcataaaggggaaaactccgaaaagtcaaaccgcttgccttcagaagaagacataactcgtttagaagtgcgaaataaacagttgttagaccgaatgcaaaataatactaaggactatgatcagttacaaattgccgttgaaaaggattacgttaaaaagttttggatttcatttaagggatag

SEQ ID NO: 291 - DNA, 996 bp, Paenibacillus sp.
  source 1..996: mol_type = genomic DNA
atggagcgaaatatggagtttatagataagtggcatcatattttgagatgctgtagttttgataacacctacaaaatggcatgggctagagccattgtagaaatagcagtagagcaagaagacttatccagtaaagataaatcaactcttgttgaagttcacttacatgacatcgcagtaaaggttcttcgatattatttcgaacagacaattttttttgggctgaaacaaagttcaaatccatttaagcccccagttgttgtgacagttgtgaaacagttgattgcagcatatcaggctcgtacaggttcgatgaagcctgtgaaatggttcaggtcaaatattgaagcagtgtgcagtacagaatatgcaaaggctgtagttgcggtgataagggcgctgaaggcagatgtaagttatcgcttcttgcgagttgaaggtaaagaaattacaggtttatatcaatatgaacagaaagcggaaagtttgtttatgcgagcaaacgacttaaacacacttaaagataatcgcttgatcatgttagatgccatcaactatcggtggacacaaatgctagagaacttcaatcactctcctaggctgtcgaagaaagtgcggataattgacgaagatcaggtaaggagaaaaccactgggtaagtttgcttcatatatggaagttgaaaattcagaatgtctctgctttctttgtagtgggaggattgaagatcagacaccagcgattgaccatgtaattccgtggtcatatctatattcagatgatttatggaatctggtatttgcacaccagtcgtgcaatagtagtaagtctaatgtaataccaagtgaggaaatgatacaacggcttgaagtaaggaataaggcactgcttcaactattgaaggaacagggaaaagtggataagcatgtttccgaacttcaaatggcagtggacagtaatcttgtccgcaagttctggatagcatgtcagggatga

SEQ ID NO: 292 - DNA, 495 bp, unidentified
  source 1..495: mol_type = genomic DNA; note = Glutamicibacter arilaitensis
atgcgaacactggttttaaacgcaggatacgagcccttagccgtcgtgactttccgacgagcactgttgatggtcatggccgacaaggcgaccatcgttctccaggatgaaggctcgcctgttcgcagctcacacggacagttacccagaccaaccgtcattgtcttaaaacgctatgtacgacgcccctaccgcgacatgcttcatgttacgcgtaggggcgttttgcgtcgtgatgcctacacctgtgcctactgcggaaaatccgctaatacgatcgaccatgtcattccacggagcaggggaggcaagaacacttgggagaacctcgtcgctgcatgcctgcactgcaacagccgcaaggctgacaagatgctctcccagctgggctggcagttgcgcattacaccgcatcggccgcggatgggcggccatctcgtatctgggctccagcatatcgatgccgcgtgggacgaattcctgcaagcaagctag

SEQ ID NO: 293 - DNA, 501 bp, unidentified; identical to SEQ ID NO: 282
  source 1..501: mol_type = genomic DNA; note = Pseudarthrobacter chlorophenolicus

SEQ ID NO: 294 - DNA, 501 bp, unidentified; identical to SEQ ID NO: 263
  source 1..501: mol_type = genomic DNA; note = Pseudarthrobacter chlorophenolicus

SEQ ID NO: 295 - DNA, 501 bp, unidentified; identical to SEQ ID NO: 32
  source 1..501: mol_type = genomic DNA; note = Pseudarthrobacter chlorophenolicus

SEQ ID NO: 296 - DNA, 501 bp, unidentified
  source 1..501: mol_type = genomic DNA; note = Pseudarthrobacter phenanthrenivorans
atgcgcactctcgttctgaatgctggatatgaaccgctggcggttatttctttccgccgggcgctggtgcttgtgctgacgaacaaggcgagcgttgtggccgaaggggacgatcccgtagtgggtccccaggagatactgggccgcccgtccgtgattctcctgaaccgctacatccgccccagatacaaccagtcaacggctgtgagcagacgcggcgtgctccgacgtgacgggcacaaatgcgcctactgcggaaaagccgcgcacaccattgaccatgtccaccccaaatcccgtggtggcgcggattcatgggagaacctggtggctgcgtgccttcgctgcaacaacgcgaagggtgaccacacccccgccgagatgggctggaagttgcggttcgtgcccgaacctccgcatggcgccatctggcagatcaaggaactcgagaagcccacacctgcctgggatccgttcctgctgccggagcgcgctgcctga

SEQ ID NO: 297 - DNA, 975 bp, Bacillus sp.
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcatttgctaaaatatattggaatttggttattcatcatgatttaaatcaattaaatacacacaatagacaagctggaattcaaaaagagttaaaggaatttcagttaatgcatggtgtccccaataaagtagtatttgatcgtttaccaagtaatcttcaattgcagttagtagaaagaactaaaaaagtgggggcaaggtatgtagtaggggcgttatacggagatatggaaggttctatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagatgatgtcttgttgaaagtggaaaatatttcaaaacacgaatcgctgcatgcattctataacatactttggaaatacgaaacaaaagaatgtttttattgtggaaaacgtctttctgataaacgcatcgctactcatgtagatcattttattccatggagttatatccaatcagataatatgtggaatttagtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 298 - DNA, 501 bp, unidentified
  source 1..501: mol_type = genomic DNA; note = Pseudarthrobacter phenanthrenivorans
atgcgcactctcgttctgaatgctggatatgaaccgctggcggttatttctttccgccgggcgctggtgcttgtgctgacgaacaaggcgagcgtcgtggccgaaggggacgatcccgtagtgggtccccaggagatactgggccgcccgtccgtgattctcctgaaccgctacatccgccccagatacaaccagtcaacggctgtgagcagacgcggcgtgctccgacgcgacgggcacaaatgcgcctactgcggaaaagccgcccacaccatcgaccatgtccaccccaagtcccgcggcggcgcggattcatgggagaatctcgtggctgcgtgccttcgctgcaacaacgcgaagggtgatcacaccccggcggagatgggctggaagctgcgcttcgtgcccgaacctccgcgtggcgccatctggcagatcaaggaactcgagaagcccacacctgcgtgggatccgttcctgctgccggagcgcgctgcctga

SEQ ID NO: 299 - DNA, 501 bp, unidentified; identical to SEQ ID NO: 147
  source 1..501: mol_type = genomic DNA; note = Pseudarthrobacter chlorophenolicus

SEQ ID NO: 300 - DNA, 501 bp, unidentified; identical to SEQ ID NO: 282
  source 1..501: mol_type = genomic DNA; note = Pseudarthrobacter chlorophenolicus

SEQ ID NO: 301 - DNA, 501 bp, unidentified; identical to SEQ ID NO: 282
  source 1..501: mol_type = genomic DNA; note = Pseudarthrobacter chlorophenolicus

SEQ ID NO: 302 - DNA, 537 bp, unidentified; identical to SEQ ID NO: 262
  source 1..537: mol_type = genomic DNA; note = Glutamicibacter arilaitensis

SEQ ID NO: 303 - DNA, 501 bp, unidentified; identical to SEQ ID NO: 147
  source 1..501: mol_type = genomic DNA; note = Pseudarthrobacter chlorophenolicus

SEQ ID NO: 304 - DNA, 501 bp, unidentified; identical to SEQ ID NO: 108
  source 1..501: mol_type = genomic DNA; note = Pseudarthrobacter chlorophenolicus

SEQ ID NO: 305 - DNA, 501 bp, unidentified; identical to SEQ ID NO: 282
  source 1..501: mol_type = genomic DNA; note = Pseudarthrobacter phenanthrenivorans

SEQ ID NO: 306 - DNA, 501 bp, unidentified; identical to SEQ ID NO: 282
  source 1..501: mol_type = genomic DNA; note = Pseudarthrobacter chlorophenolicus

SEQ ID NO: 307 - DNA, 501 bp, unidentified
  source 1..501: mol_type = genomic DNA; note = Pseudarthrobacter chlorophenolicus
atgcgcactctcgttctgaatgctggatatgaaccgctggcggtaatcaccttccgccgggcgctggtgcttgtgctcacgggcaaggcgagcgttgtggccgaaggggacgaccccgtcgtgggcccaaccgacgtccttgggcgcccgtccgtgatcctcctcaacagatacatccggccccggtataaccactccacggcggtcagccgcaggggcgtgctccggcgcgatggacacaaatgtgcgtactgcggtaaggcggcgcacacgatcgaccacgtgcacccaaaatcacgtggcggggccgattcgtgggagaatctggtggccgcctgcctgcgctgcaacaacgtcaagggtgaccacacccctgctgagatgggttggaccctgcggtttgtgcccgagccgccgcggggaacgatttggcagatcaaggaactcgagaagccaaccccggcatgggatcccttcctgcttcccgagcgcgctgcctga

SEQ ID NO: 308 - DNA, 501 bp, unidentified; identical to SEQ ID NO: 282
  source 1..501: mol_type = genomic DNA; note = Pseudarthrobacter phenanthrenivorans

SEQ ID NO: 309 - DNA, 501 bp, Arthrobacter sp.
  source 1..501: mol_type = genomic DNA
atgcgcactctcgttctgaatgctggatatgaaccgctggcggtcatcacgttccgccgggcactggtgcttgtgctcacgggcaaagccagtgtggttgccgagggtgacgatcccgtggtggggccccaggagattttgggccgcccgtccgttatcctcctgaaccgctatatccgccccaggtacaaccaggccaccgctgtcagccgccgcggcgtgttgaggcgcgatggccaccgttgcgcctactgcgggaaagcggcccacaccattgaccatgtccagcccaagtcccgcggtggtgccgattcctgggagaacctggtggccgcctgcctgcgttgcaacaacgtcaagggcgaccacactcccgctgaaatggggtggaagctgaggttcgtcccggagcccccgcacggaaccatctggcagatcaaggaactcgaaaaacccacccctgcttgggacccgttcctgctgcccgagcgcgcagcctga

SEQ ID NO: 310 - DNA, 501 bp, unidentified
  source 1..501: mol_type = genomic DNA; note = Pseudarthrobacter phenanthrenivorans
atgcgcactctcgttctgaatgctggatatgaaccgctggcggttatttctttccgccgggcgctggtgcttgtgctgaccaacaaggcgagcgtcgttgctgaaggggacgatcccgtagtggggccccaggagattctgggccgcccgtccgtgattctcctgaaccgctacatccgtcccagatacaaccagtccacggctgtgagcagacgcggcgtgctccgacgcgacgggcacaaatgcgcctactgcggaaaagccgcgcacaccatcgaccatgtccaacccaagtcccgcggcggcgcggattcctgggagaacctcgtggcggcgtgccttcggtgcaacaacgccaaaggcgatcacaccccggcggagatgggctggaagctgcggttcgtgcccgagcccccgcacggcgccctctggcagatcaaggaactcgagaagcccacacctgcctgggacccgttcctgcttcccgagcgcgctgcctga

SEQ ID NO: 311 - DNA, 996 bp, Bacillus simplex
  source 1..996: mol_type = genomic DNA
atgagccacaaattacaagtgggcgaaatgaaagccgtttatttaaccgataaagaaatatgggcacattttaattacattttctcatcaaaatcaaaaaattcgactacatacaaatttgttttgattaaatccatttcagaaaacctttacaatgttaatgaaaagctggagttaaattattcagtcttgttttcaagtttttccagaatatattggaatctggttatccatcataatctaaatcaaattaatcagacggataaaaaggcagaggtacaaaatattcttcataatgttcaatcgaaatataaaatcccgaatacactagtttttgataagctctctaatgatttgcagattgaaatcattaacaaggtgacaaaaagatgtaagattaatgtaatgggagccatttatggtgacaccggcagtactatttatgattttgataatagcaaggaacagctaagactgaatagtactttttattcttttatgcaacggttccaacgggtgttaaattatgtaacgaactatcatcttgctttatttttggaaaaatttaatgaaaaaggtgacacaactaaccttttattaaaggtcgaaaatgtctcaaagaggtcttcgttggatcagttttaccaagtgctctcctccttttatagtggaaaatgtttttattgtggaaaagcgattaagaggcataatgcccatgttgaccactttattccctggagctttgtacaagcagatcagctctggaatcttgttattgcgtgcagcgcttgtaatttatcaaagagtgataaattggccaataagttgtatttagaaacagtaattgaacgaaacaataacctgattacgattccggaacttagcaaaagagatgacatgaaagtatataccagcaaaaaactgatagatttatatcattattcagttgaaaacgggtttactgatatttggatgccaaaaaaaataatcatataa

SEQ ID NO: 312 - DNA, 501 bp, unidentified; identical to SEQ ID NO: 147
  source 1..501: mol_type = genomic DNA; note = Pseudarthrobacter chlorophenolicus

SEQ ID NO: 313 - DNA, 501 bp, unidentified; identical to SEQ ID NO: 282
  source 1..501: mol_type = genomic DNA; note = Pseudarthrobacter phenanthrenivorans

SEQ ID NO: 314 - DNA, 435 bp, Bacillus megaterium
  source 1..435: mol_type = genomic DNA
ttggcgaaatttttagaaaagcataatgacaaaaataagttagatgatgtcttgttgaaagtggaaaatatttcaaaatgcgaatcactgcatgcattctataacatactttggaaatacgaaacaaaagaatgtttttattgtggaaaacgtctttctgataaacgcatcgctactcatgtagatcattttattccatggagttatatccaatcagataatatgtggaatttagtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 315 - DNA, 501 bp, unidentified; identical to SEQ ID NO: 147
  source 1..501: mol_type = genomic DNA; note = Pseudarthrobacter chlorophenolicus

SEQ ID NO: 316 - DNA, 537 bp, unidentified; identical to SEQ ID NO: 200
  source 1..537: mol_type = genomic DNA; note = Glutamicibacter arilaitensis

SEQ ID NO: 317 - DNA, 975 bp, Bacillus megaterium; identical to SEQ ID NO: 9
  source 1..975: mol_type = genomic DNA

SEQ ID NO: 318 - DNA, 501 bp, unidentified
  source 1..501: mol_type = genomic DNA; note = Pseudarthrobacter chlorophenolicus
atgcgcactctcgttctgaatgctggatatgaaccgctggcggttgtgactttccgccgggcgctggtgcttgtgctcgcgggcaaggcgagcgtggtggccgagggtgacgaccccgtggtgggccccactgatgtgctgggccgtccgtccgtgatcctcctcaaccgctacatcaggccccggtacaaccactccacggccgtcagccggcggggagtgcttcggcgggatgggcataagtgcgcctattgcggcaaggcggcccacaccatcgaccacgtgcaccccaagtcgcggggcggcgccgattcgtgggaaaacctggtggccgcctgcctgcggtgcaacaacgtcaaaggggaccatacgcccgcggagatgggttggaccctgcgtttcgtcccggagccgccacatggaacgatctggcagatcaaggaactcgagaagccaacgccagcgtgggatcccttccttcttccggagcgggctgcctga

SEQ ID NO: 319 - DNA, 498 bp, Microbacterium testaceum
  source 1..498: mol_type = genomic DNA
atgcgtactctggtgctgaacgcgggctacgaaccgctcgccgtcgtgtcgttcaaacgagcgctcgtgctcgtgatgaacgagaaggccaccgtcgtcgaatgcgtcgacgaagatccggtctgggcggcgggaggaacctacgatcgcccggcggtgatcatcctcacgcggtacatccgggtgccgggtgcgcgccaggtgccggtcacgcggcgaggggtgctccgccgcgacgcgcatcgctgcggatactgcggcaagacggcctcgaccatcgaccatgtgctgccgcgttcgcgcggggggaaggacacgtgggagaacctcgtggcctgctgcctgcggtgcaacaacgtcaagggcgatcgcacgccgcaggagatgtcgtgggagctccagatcatcccggcgcctccgcggggctcgtcctggacggtgcgtggagtcgacaagagcgacccgcggtgggagccgtatctcgcgctcgcggcgtga

SEQ ID NO: 320 - DNA, 498 bp, Microbacterium testaceum; identical to SEQ ID NO: 319
  source 1..498: mol_type = genomic DNA

SEQ ID NO: 321 - DNA, 498 bp, Microbacterium testaceum
  source 1..498: mol_type = genomic DNA
atgcgtactctggtgctgaacgcgggctacgaaccgctcgccgtcgtgtcgttcaaacgagcgctcgtgctcgtgatgaacgagaaggccaccgtcgtcgagcgcgtcgacgaagatccggtctgggcggcgggagggacctacgatcgtccggccgtgatcatcctcacgcggtatatccgggtgccgggtgcgcgtcaggtgccggtcacccggcgaggggtgctccgccgcgacgcccaccggtgcgggtactgcggcaagacggcctctaccatcgaccacatcctgccgcgttcgcgcggggggaaggacacgtgggagaacctcgtggcctgctgcctgcgatgcaacaacgtcaagggcgaccgcacgccgcaggagatgtcgtgggagctgcgggtgatccccgccccgccgcgggggtcgtcgtggacggtgcgcggtgtcgacaagagcgacccgcggtgggagccgtatctcgcactcgcagcgtga

SEQ ID NO: 322 - DNA, 699 bp, Bacillus sp.
  source 1..699: mol_type = genomic DNA
atgtatttgcagaaacaaggcgatatttgcgaattatataaaaaatgtgacacatgcaataaaagtttacatggttcaaaattcaaatatggaacttggatgacatgtcaaagttgtaagaaaaagtattataaaaagcaacatgacattagaaggaatgaacttacttacgattatgttttaaacacgatgaaaaaataccgagaagctaatgatcaagaaatacatttacaagagaatgaaaaaggaaaaatacatttattatcttggaatcttttagataatagggaagttccaattgaaacagctataacttatctaaatagtggtgacgcaaaaataataaataaaaatacaatatattcattaacaaatactaatagcaaatttttaagggatctagtttatgataaatttaaaaacgtttgtttttattgtggtaaatatggaaacacaattgatcatgtaattcctatttcaaaaggaggaatgaatatattagaaaatatggtttgctgttgtgaaaaatgcaataagattaaaggtaaccaaatgaatattgatacaatgaagaagatcggagatttaacatctagagagcatatagattataaattaaaagaaatgaaagataataagttaaatacatttgaagacatgttgaaagatgccttaaaaagtaaaaacaagtggaattaa

SEQ ID NO: 323 - DNA, 975 bp, Bacillus megaterium; identical to SEQ ID NO: 9
  source 1..975: mol_type = genomic DNA

SEQ ID NO: 324 - DNA, 3192 bp, Lysinibacillus sphaericus
  source 1..3192: mol_type = genomic DNA
atgggtaaagttttagggctagatatcggaatttcatctattggatggggatttattgataatgattctatggagataatcgctgcaggtgtgagactttttgaagaatcaacgagaaatgctaatgaagaacgtagaggtttcagaggttcaagaagattgaaacgtagaagaattcatagattagaaagaactaagaaattatttgaagagtatggattacctataagtgcaattggcactggaaatccttatgaaattcgttgtaaagcattaaatgaagaagtttctcttgaggaattagtaatagctttataccatttagtaaaaagaagagggacagttattgatacgccgattgaagaagaatctaaaggaaatgaattgtcaactaaagaacaattgaaacgtaatagtaaagaacttgaatcaaaatttatatgtcaagttcaaatggaaaggttaaaaaacggtggtgttcgttcgcatctgaatcgttttagaacagaagactatataaatgaagctagagctattcttaataaacaaagtgaatattatgctgaattaaatgaagaatttattgatagatatattatattgcttaattcgagaagagcatattatgacgggccaggatcggaaaaatctccaacaaattatggtcgattttatataaacaatggaaaattacaatgtatgtcaatgatagataaaatgagagggaaatgtacatatttcccagacgaattacggattcctaaaatggcatatacagcagatttgtttaatttattaagtggtgatttaaataaaattcaattaaatggtgattatcttacttttgaagacaaacaatatattgtagaacagataattaaaaaaggcgaaaatcttacaattaagaaaatactgaagtacaaagggctaccagatgacactaacgtaataggttgtagagtagatttgaaaagtggaaatccaacatttacagagtttaaggggtataagaaattacttaaaattgtacaagataataacctgcctagtgatatgttagataatgtagatttgatagatgaaattgttaatattcttacggcagaaaaatcatatgaacgtcgagagactcaattactcgaaaaaataaatgtgttgactgactatactgaagaaactattaaaagaataattatcgcattaaaagaaaacacagattttgttggttaccatgcactatctaagagggcaattaacttacttttgcctgatttgtgggcaacaaataaaaatcagatggaattgtttagtgaattaggactagaaagtagacgtttaaagacattatcacagagtaaaaatatacagttcgatgataatgcaattttaagtacggtagccaaacgggcacatagagaggctattaaaattgtaaataaaattagagaaaaatatggtgagttagaatcaattgttgttgagacagctaaagaaaaaaatagtgatgaaaagcgaaaaatgtatcaagacttccaaaagaaacaagggaaatttgaaaaagagatggctaatttgttaggggtgaaatctttaaaagagttaaaacttaatagtaaacaacacctagctttaaagctttggcaagcacaggatggtaaatgtttgtactctatgaagtcaattgggattcatgatattataaaagacttttcaaaatttgaaattgatcatattgttccgatttccatttctttcgatgatagtcaagctaataaggtattatgctatcgtttagaaaatcaacaaaagggtcaaagaactccgtttcaatatctttcaagttctactgaggctagtatttcattcgaagaatataaagcggaagttataaaactatttaaaagtagaaaaatttctaataagaaaaaggaatatttattagaacaaagggacataatgaataatgaggaattgaaaaagcaattcattaatcgtaatttagtagacactagatatgcaatgaggagtttttcgatgactctacgttcatattttaagcaccatgacattccaacttcagttctatcgataaggggggaattcactgcagcaataagaagacgtgcaaaaataaacaaagaccgtgatgcttcatatgcacatcatgccattgatgcactaatagtggcaactattgggaaaatgcctatttttaatttttttaagaattttggtgtaaatgaacttggagtatattatgatcgtgcaacaggtgaaattctaacagaggatcagttctttaatgataaaatgtacaaatttgttcgaagtttaatgaactatgaatctaaaattaagtattctcacaaggttgatagaaaagggaatcgtacaatgactaatgagacgatttatagtactcgaaatatagaaggtaaagaatatgtcattggaaaatataaaaatatatatgagttagataaggacaaattaaaaactttaaaagataaaatattaaaaaaaccagaaagcttattattatctaaagctaaccctcagctacttagtttattaatagacatagtaaaacactatgataaatcggataatccttttaaagcctatttcaatgaaaatggatacattcttaaagatggaaagattccagtgaaaagcattaaatataaagaagaagagataggagtatatttggatatcacacacaaatatccaaaatcggataaaagggtaattttgaaaaacattaaaagtgttcgaattgatgtatataaaaataaggataacatatttaagtatctcggagtaccataccattggttttcattgaaagggaaaaaattattactaaataacgaaaaatatgctttaggactaaaatcaaaacaaatagattgtggagattataaatttcaattcagtttatacaaaaacgatttgtttacaatagaaaaacaaggtcaaattttcgaatatgttttccgtggagacaataatccaaggcaaaacaaaatagaggcaaactatgttcatcggaatagaaattcaaaagaaaaaagggttattttgacaattgggccatctatcaacaatattacaaagttttatgtcgatgttctaggaaatcgctttaaagttgaaaaagaaaagtttgtaaactatttacaaaaatga

SEQ ID NO: 325 - DNA, 999 bp, Bacillus simplex
  source 1..999: mol_type = genomic DNA
atgagccataaactgcaagtaggggaaatgaaaactgaatatttaactgatcaagatatttggagtcagtttaattttattttctctagtaaatcaaaaaattcgactacatataagtttgtgttaatcaaatccctgcttgaaaatttgtataacgtaaacgaaaagctagagctgtcttatgatatcttgtttagcagtttttcaaaaatttattggaacttagttattcatcataatcttaatcaaattaatatgataggaaaaaaagcggaagttcaaaatgtgcttgaggctattcaatccagttatcagattcctgatatattcgtttttgataagctttcttcagaaattcaagtgcaggtcattactaaagtgaaacgaaagtgcaaaatcaatgtgatgggtgcactatacggtgataccgattctactctttatgattttgataataaaaaggaatatatacgaatcaatccttcttattattcttttatgcaacgatttcagagagtacttcactatttaacgaattatcagcttgctcttttcctggagaaattcaataacgaaggggatacaacgaatttattattgaaggtagagaatgtttccaagaggtcttccttagatcaattttatcaagtgttggaatcattctatgatgggaaatgtttttattgtggaaaagcaattaagaaaaaagcggcagcgcacgtggatcacttcattccgtggagctttgtgcaaaacgatcagttatggaatttagtgattgcctgtagcacgtgtaatctctcgaaaaatgataagttagcctctggctatttcttggaagctttaatcgatcgaaatgaagaatttttgatgcagtccgatctttttcaaagggaagatatgcaggtatatagtgagaagaagttaacggagctttataaatattcggttgataatggctttaccgatatttggatgccgaagaaaactatattatag

SEQ ID NO: 326 - DNA, 420 bp, unidentified
  source 1..420: mol_type = genomic DNA; note = Paenibacillus yonginensis
atgattacggtactgaatacagatggtcgggaattaaccccttgtaccctggaccgagcttggcgggaggtgaatcgatcgcgtgccgtttggattaatgagcagaccattcagttgttatacaacccttttctgttccggaaatatcggaagatcgccttaaaaagagaccgttacacatgcctatggtgcggacagcctgcaacaacggtggatcatctgatcccctccagcaagggcggttcggatctgccgcgcaaccttattgcctcctgcggcgagtgcaactcaagacgggggaatcgcccggcgtgggagtattttagagagaagatgggtttcattccccgtcccctcaagcttggttttcatattttgatagctttaggctgctcaaagatcaaaccaagatctcattaa

SEQ ID NO: 327 - DNA, 420 bp, unidentified
  source 1..420: mol_type = genomic DNA; note = Paenibacillus yonginensis
atgattacggtactgaatacagatggtcgggaattaactccttgtaccctggaccgagcttggcgggaggtgaatcgatcgcgtgccgtttggattaatgagcagaccattcagttgttatacaacccttttctgttccggaaatatcggaaggtcgccctaaaaagagaccgttacacctgcctgtggtgcggacagcctgcaacaacggtggatcatctgatcccctccagcaagggcggttcggatctgccgcgcaaccttattgcctcctgcggcgagtgcaactcaagacgggggaatcgcccggcgtgggagtattttagagagaagatgggtttcattccccgtcccctcaagcttggttttcatattttgatagctttaggctgctcaaagatcaaaccaagatctcattaa

SEQ ID NO: 328 - DNA, 999 bp, Bacillus thermoamylovorans
  source 1..999: mol_type = genomic DNA
atgagccataaattacaaataggtgaaatgaaagctaactatttaactgataaagatatttggggctgtttcaattatattttttcacctcgttcaaagaattcaactacctataaatttgtgttgataaaaagtattattgaaaatttatataatgtgaatgctcagctggaattgggatatgatcaattattcagcagctttgcaaaaatatattggaatctagtcattcaccatgatcttaatcaaattaatgtgattggaaaaaaatcagaagttcaaaacatccttttagatgtacaaactaagtattcaatcccaaatacagttgtattcgataaattatcaagtgaactacaattaaatattattttgaaagttaaaaaacgatgtaaattaaatgtaatgggtgctatatacggggatactaaggggacaatctataattttgataataaaaaaggatatctgaaattaaacggttcattttattcattcatgcaacaatttcagagggtattaacctatttaacaaactaccatttagcgatgtttctagagaaatataatgatcaaggcgatacaactaatcttcttttaaaagttgagaacgtatcaaggcgatcttctttagctcagttttatcaattattggagtcttacttaaacagaaactgtttctattgtgggaaagcaatcaataagaaggatggagttcatgtagatcattttattccatggagttttgttcaagctgatcaattatggaatttagttctatcatgtagggtgtgtaatctttctaaaagtgataggttaacagataatatatatttaaatgcgctaattgaccgaaatgataaactgctaactatttcctcgcttcaaatgagaaaagatatgcaggtttatacatcagataaattgaagcagctttatcaattcacaattgataacggatacacagatatctggcggccacagaagcgggtaaattaa

SEQ ID NO: 329 - DNA, 975 bp, Bacillus sp.; identical to SEQ ID NO: 116
  source 1..975: mol_type = genomic DNA

SEQ ID NO: 330 - DNA, 498 bp, Microbacterium sp.
  source 1..498: mol_type = genomic DNA
atgcgcacactggtcctgaacgccggatacgagcccctcaccgtggtctcgttcaagagagccatggtgctcgtgatgaacgacaaagccagcgtgatcgagcacgtggagggcgatcccgtctgggcgggacgcaacgcatacgaccggccggtcgtgatcctgctcacccgctatgtgcgcgtgccgtcatcgaggcgcgttcctgtcacccggcggggcgtgctccgccgggacgaccaccgctgcggctactgcggacggagtgcgtcgacgatcgaccacatccttccgcgctcgcggggaggggcggacagctgggagaacctcgtcgcctgctgcctgaagtgcaacaacatcaagagcgaccgcaccccgcaggagatgggctgggaactgcggatgacgccgcgaccgccgcacggcacggcgtggacggtgcgcggcggtgagcgcggtgacccgcgctgggagccgtacctcgcgctcgcggcgtag

SEQ ID NO: 331 - DNA, 501 bp, unidentified
  source 1..501: mol_type = genomic DNA; note = Pseudarthrobacter chlorophenolicus
atgcgcactctcgttctgaatgctggatatgaaccgctggcggtcatcactttccgccgggcgctggtgcttgtgctcacgggcaaggcaagcgtggtggccgaaggggatgacccggtggtagggcccaccgacgtcctgggccgcccgtccgtgatcctcctcaaccgctacatccggccccggtacaaccactcgacggcagtcagccgcaggggtgtgctccggcgcgacggacataaatgtgcctactgcggcaaggcggcgcacacaatcgaccacgttcacccgaagtcccggggcggtgccgattcatgggagaaccttgtggccgcctgcctgcggtgcaacaacgtcaagggcgaccacactcccgcggaaatgggctggacccttcggttcgtgcccgagccgccgcacggaacgatctggcagatcaaggaactggagaagccgacgccggcgtgggatccgttcctgctcccggagcgcgccgcctga

SEQ ID NO: 332 - DNA, 501 bp, unidentified; identical to SEQ ID NO: 331
  source 1..501: mol_type = genomic DNA; note = Pseudarthrobacter chlorophenolicus

SEQ ID NO: 333 - DNA, 498 bp, Microbacterium sp.; identical to SEQ ID NO: 40
  source 1..498: mol_type = genomic DNA

SEQ ID NO: 334 - DNA, 501 bp, unidentified
  source 1..501: mol_type = genomic DNA; note = Pseudarthrobacter chlorophenolicus
atgcgcactctcgttctgaatgctggatatgaaccgctggcggttatcactttccgccgggcgctggtgcttgtgctcacgggcaaggcaagcgtggtggccgaaggggatgacccggtggtagggcccaccgacgtcctgggccgcccgtccgtgatcctcctcaaccgctacatccggccccggtacaaccactcgacggcagtcagtcgcaggggtgtgctccggcgcgacggacataaatgtgcctactgcggcaaggcggcgcacacaatcgaccacgttcacccgaagtcccggggcggcgccgattcatgggagaaccttgtggccgcctgcctgcggtgcaacaacgtcaagggcgaccacactcccgcggaaatgggctggacccttcggttcgtgcccgagccgccgcacggaacgatctggcagattaaggaactggagaagccgacgccggcgtgggatccgttcctgctcccggagcgcgccgcctga

SEQ ID NO: 335 - DNA, 420 bp, unidentified
  source 1..420: mol_type = genomic DNA; note = Paenibacillus yonginensis
atgattacggtactgaatacagatggtcgggaattaaccccttgtaccctggaccgagcttggcgggaggtgaatcgatcgcgtgccgtttggattaatgagcagaccattcagttgttatacaacccttttctgttccggaaatatcggaagatcgccttaaaaagagaccgttacacgtgcctatggtgcggacagcctgcaacaacggtggatcatctgatcccctccagcaagggcggttcggatctgccgcgcaaccttattgcctcctgcggcgagtgcaactcaagacgggggaatcgcccggcgtgggagtattttagagagaagatgggttccattccccgtcccctcaagcttggttttcatattttgatagctttaggctgctcaaagatcaaaccaagatctcattaa

SEQ ID NO: 336 - DNA, 501 bp, unidentified; identical to SEQ ID NO: 53
  source 1..501: mol_type = genomic DNA; note = Pseudarthrobacter chlorophenolicus

SEQ ID NO: 337 - DNA, 498 bp, Microbacterium sp.; identical to SEQ ID NO: 40
  source 1..498: mol_type = genomic DNA

SEQ ID NO: 338 - DNA, 501 bp, unidentified; identical to SEQ ID NO: 147
  source 1..501: mol_type = genomic DNA; note = Pseudarthrobacter chlorophenolicus

SEQ ID NO: 339 - DNA, 420 bp, unidentified; identical to SEQ ID NO: 326
  source 1..420: mol_type = genomic DNA; note = Paenibacillus yonginensis

SEQ ID NO: 340 - DNA, 420 bp, Paenibacillus sp.
  source 1..420: mol_type = genomic DNA
atgattacggtactgaatacagatggtcgggaattaaccccttgtaccctggaccgagcttggcgggaggtgaatcgatcgcgtgccgtttggattaatgagcagaccattcagttgttatacaacccttttctgttccggaaatatcggaagatcgccttaaaaagagaccgttacacttgcctatggtgcggacagcctgcaacaacggtggatcatctgatcccctccagcaagggcggttcggatctgccgcgcaaccttattgcctcctgcggcgagtgcaactcaagacgggggaatcgcccggcgtgggagtattttagagagaagatgggtttcattccccgtcccctcaagcttggttttcatattttgatagctttaggctgctcaaagatcaaaccaagatctcattaa

SEQ ID NO: 341 - DNA, 420 bp, unidentified; identical to SEQ ID NO: 335
  source 1..420: mol_type = genomic DNA; note = Paenibacillus yonginensis

SEQ ID NO: 342 - DNA, 420 bp, unidentified; identical to SEQ ID NO: 335
  source 1..420: mol_type = genomic DNA; note = Paenibacillus yonginensis

SEQ ID NO: 343 - DNA, 420 bp, unidentified; identical to SEQ ID NO: 326
  source 1..420: mol_type = genomic DNA; note = Paenibacillus yonginensis

SEQ ID NO: 344 - DNA, 537 bp, unidentified
  source 1..537: mol_type = genomic DNA; note = Glutamicibacter arilaitensis
atgccagatggcggccatcggttcatgaaggtagtacgagcaatgcgaacactagtattaaacgcaggatacgagccattagccgttgtgacattccgtcgagctctagtgcttgttatgagcgataaagcgagcattgtgctccaagatgatttctcacccatccgcagttcccatggcgagttaccaaggcccacggtcattgttctcaaacgttatgtacgacgcccctacagggacatgcttcatgtcacccgtaggggcgttttgcgtagggacgcctttacctgcgcttattgcggcaaatccgccaatactattgatcacgtcattccgcgaagcaggggagggcagaacacctgggagaacttggttgctgcatgcctgcactgcaatagcaggaaggcggacaagatgctctcgcaattgggctggcaattacgtatcaccccgcaccgtccgagagtcgccggccacctcgtttcggggttgcaacatgtcgatgcagcatggaacgagtacctgcacgcgagctag

SEQ ID NO: 345 - DNA, 420 bp, unidentified; identical to SEQ ID NO: 326
  source 1..420: mol_type = genomic DNA; note = Paenibacillus yonginensis

SEQ ID NO: 346 - DNA, 420 bp, unidentified
  source 1..420: mol_type = genomic DNA; note = Paenibacillus yonginensis
atgattacggtactgaatacagatggtcgggaattaaccccttgtaccctggaccgagcttggcgggaggtgaatcgatcgcgtgccgtttggattaatgagcagaccattcagttgttgtacaacccttttctgttccggaaatatcggaaggtcgccctaaaaagagaccgttacacctgcctgtggtgcggacagcctgcaacaacggtggatcatctgatcccctccagcaagggcggttcggatctgccgcgcaaccttattgcctcctgcggcgagtgtaattcaagacgggggaatcgcccggcgtgggagtattttagagagaagatgggtaccattccccgtcccctcaagcttggttttcatattttgatagctttaggctgctcaaagatcaaaccaagatctcattaa

SEQ ID NO: 347 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattttttgctaatggtcactttaccacaacgtataaatatggattaatgaaagctttaattgaaaacctatataacgttgacaatcggttagtactcacgtttgatcaagtatatttttcatttgctaaaatatattggaatttggttattcatcatgatttaaatcaattaaatacacccaatagacaatctggaattcaaaaagagttaaaggaatttcagttaatgcatggtgtccccaataaagtagtatttgatcgtttaccaagtaattttcaattgcagttagtagaaagaactaaaaaagtgggggcaaggtatgtagtaggggcgttatacggagatatggaagcttctatttatgagtttgacaaaagaacagaatacataaagtttaatagtagtatctatatttttttacaaaaatatagacaaattgttactcatttaaccaattatcatttggcgaaatttttagaaaagcataatgacaaaaataagttagaagatgtcttgttgaaagtagaaaatatttcaaaacgcgaatcgctgcatgcattctataacatactttggaaatatgaaacaaaagaatgtttttattgtgggaaagttctttctgataaacgcatcgctactcatgtagatcattttattccatggagttatatccaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgagtatgctggaatgaatggatacgtagaaaactggtctccatag

SEQ ID NO: 348 - DNA, 975 bp, Bacillus megaterium
  source 1..975: mol_type = genomic DNA
gtggatagttatattaaacaatggcatgatttaataataaactgtaattttgacaacacatataaaatggcatggagtaaggctctggtggagttatcgctaagcattaaggaagagtccggagaggaaactattgcatttgattttagtgagatagcgaaactttgcttaaagtattattggaatcaaactatttactttgaccttatacaaagtcctagcttcaagaaaccacctgagatgataacatctactaaagaactgattaaagttttcttcaaaaagaaaggtaaggtacagccagtaaggtttgaaaggatagacttccactcacttaaattagaaaaccaatataaagaaacagtcaaaaaaatagcaaacacattaaaacaagatgtctgttggcgttttaaaaatcttaatggtactacctatgaaatatacgacttagatagtaacagaagaatagttcattttaataagcagcatgcattaaaacttaaagaacatagtgattttctatttacatttataaattatagatggacacaaatgttagagggatttaactatagtccaaggatttcaaggaaggtaagagcaatagacgaggataacattaaacgatcctctttaaaaaaatttcataagaaccttgatttaatatttgaagatggacaacgcaaatgcttttattgtaatgaagtcatagatgatacagatgtatctgtggatcatgttataccatggtcttatatgttttccgatgatctttggaatttagtttactgtcatagaggggaaaattccataaagtctaatcgtcttcccttagaagaggatgtcataaggttagaaaatagaaatgtactactacttgaccgtatggaaccaaaatcccgcgattacgatcagctgaagattgctgttgaaaaagattatgttaaaaagttttggacttcatttaaaggttga

SEQ ID NO: 349 - DNA, 495 bp, unidentified; identical to SEQ ID NO: 292
  source 1..495: mol_type = genomic DNA; note = Glutamicibacter arilaitensis

SEQ ID NO: 350 - DNA, 4290 bp, Chryseobacterium sp.
  source 1..4290: mol_type = genomic DNA
atgactaaaaatgtactcggattagacttaggtacaaactcaatcggctgggcattggttcagcaggattttgaaaataaaaagggacaaatcctaggaatgggaacgaggattattccaatgtctcaggatgttttaggcgaatttggaaagggtaattctgtttctcaaaccgctgaacgcacagcttacagaggtgtaagaagattacgggaaagatttttattacgtagggaaaggcttcatcgggtattgaatatcttacactttttaccagaacgttatgcttctcagatagatttcgagaaaagatttgggaaattcaaagcggaaacggagcctaaattagtgtacgatggcagtagatttatttttaaaagttcttttgaagaaatgctggcagatttcaaaaaacatcagctacagcttttggaagataataagaaaattccttatgactggactatttattatcttcgtaaaaaagcgcttactcagaaaattgaaaaagaggaactagcctggattcttctaaattttaatcaaaagcgtggttattaccagttgaggggagaagagaaagaggaaaactctaataaattagttgagttctattctatgaaaattgtggatgtcttggctgatgaaccccaaaaaggaaaatctgatatttggtattcattggttttagaaaatggttgggtttacagacggccaagtaaaactcctttatttgactggaaaaataaaacgagagattttatcgtaactactgatttaaatgatgatggaagtattaaaacggataaagacggaaatgagaaaagaagctttcgtgcaccaaacgaggatgactggacgcttgtaaagaagaaaacagaacaggacatcgaccaatctcataaaacggtcggaacatatatttatgaagctttgcttcaaaatcccaaacagaaaattaaagggaaactgattcgtacaattgaaaggaaattctataaaactgaactcaaacaaattctggaaaaacagaaagagtttcatccggaactgcaaaacgatgatctatacgctgattgtgtaagagaattataccgtaataataaggatcatcaattaacattgagtaaaaagaattttgttcatttgtttttgaatgatattattttttatcaacgacccttaaaaagccagaaatcttctatttctaattgtactttagagttcaggaaatataaagatgaaaatggagtggagcacacacaatatttaaagactattccaaagtcaaatccgtattatcaggaattccgtatttggcagtggatgtttaatctaaatatttacaaaaaagatgatgatataaatgtcactaaagaatttttaaataccgttgctgattatgaaatcctgtttgactttttgaataacaaaaaggaagttgatcaaaaaacattgttaaagcattttaaattaaacgaaaaaacacatcgctggaattatgtagaagataaaaaatatccctgcaacgaaatcaggacaatgatttcgacaaggttggaaaaggtggaaaattgtctgcctgattttcttacaaaagaaatggaacaaaatatatggcacattatttattccgtgaacgataaaacagaatacgaaaaagcattgaaatcatttgccagaaaaaataatctggatgaaggctctttttttgaagcttttaaaaaatttccgccattcaaaagcgaatacggttctttttcagaaaaagcgattaagaaattgttgcctttaatgcgttgggggaaatattgggattgggaaagtattgatgaaaaatctcagaagagaattgaaaaaatcactacaggtgaatttgatggaaatattaaggataagatcagagaaaaggcattacatcttacgcaggaaaatcattttcagggtttagcattatggttggcacaatatgtggtttacggacgtcattcagaagcttcggttgcaggtaaatggaattccgcagatgatttggaagagtttttaaaagattttaaacaacattcgcttcgaaacccgattgttgaacaggtgattacagaaactttacacgtggtaaaagatatttgggtaaaatatggaaatggtgcaaaagatttctttgatgaaattcatattgaattaggacgagatatgaaaaacacagcagaagaccgcaaaaggctgactaatatagttacggaaaacgaaaacaccaatcttcgtattaaagctttactagctgaaatgatgaatgatggccatgtggaaaatgttcgccctttttcacctatgcagcaggaaattcttaaaatttacgaagagggtgtgttaaactctgaaattgaaattgaagatgagtatttaaaaatcagtaaaacagcacagccatcctcctcagatttgaaaagatacaagctttggcttgaacaaaagtatcgttcgccttatacaggagaaattattccattgaacaaattgtttacaccggaatatgaaattgagcatattatccctcaaagccgttatttcgatgatagctttagcaataaaattatctgtgaatctgcggtcaacaaattaaaagataactatatcggactcggtttcatcaaacagtttcatggagccacggttgaatgcggctttgggaaaactgtaagggtctttgaggtagatgcttatgaagattttgtaaaaaaacattacgctaaaaaccgtagcaaaagaatgaaacttcttctggaggaaattcctgaaaaaatgatagaacgtcagctgaatgatactcaatacatcagcaaatatatttcaggagtactttcgaatattgttcgggtggaagacggaaccgatgaaggggtgaattctaagaatattgttcaagtaaacgggaaaattacaactcagttgaaacaggattggggattgaatgatgtatggaatgaattaatactttcccgttttgaaagaatgaatcaactgaccaattctaatggttttacgatgtggaacgaaaaccatcagaagttcttacccaccgttccgattgaactttcaaaagggttctctaagaaaaggatagaccatcgccatcatgctatggacgctctggtaattgcttgtgcaaccaaagaccacattaatcttcttaataatcaatctgcaaaatccgacataaaaagatatgatttaaaaagaaagctgatgagatttgaaaaagtaatttaccatcatcctcaaacaggtaagagaattgaaaaagaaattcctaaacaatttttaaagccttgggataattttacagttgatgctaaaaacaaattagagataataattgtaagttttaagcaaaacttacgagtgatcaataaagctactaattattatgaaaagtgggttgaaatagagggagataaaagaaaagcattagtaaaacaagatggaataaactgggctatacgaaagtcgatgcataaagaaactgttgcaggaaaaatagatttaccctgggttaaaattccgaagaggaaaatattaactgctactcgaaaaagtttagatatttcatttgatgtgaaaacaatcaattcaattacagatacgggaattcagaagattttaaagaattatttagaatttaaagggaactctgaattggcattttcaccggaagggattgaagatttaagtaagaatatagaaaagtttaatgacggtaaagcgcatcaaccaatcaataaggttaggatttttgaaataggaagtaagtttcctgtaggtcaagcaggaaataaaaaagataaatatgtggaggctgcgaaagggacaaatctattttttgcagtatatgtagatgatcagggaaaaagaaaatatgaaagcattccactaaatgaagtgattgaaagacaaaaacaagggttagaccctgtttcatttaaaaataaagaagatttcttactttgcccgaatgatttggtgtacatcccttctgaagatgagcttgagaatatgcataacattgatttcaagagcttcacaaaagaaaaaacaaccaatatttataaagtagtcagtttctcaggaagccagatattttttgttagacatgatattgctacatctattgttaataaaacggaattttcaactttaaataaaatggaacgtgcaattgacggatcaatgattaaagagaattgtattaaactgaaaacagatcgtttaggtaatatatcaaaagcataa

SEQ ID NO: 351 - DNA, 678 bp, Bacillus sp.
  source 1..678: mol_type = genomic DNA
atgatcctcagattctttgctctttcagaacgtacatatttaaactataaaccaagtatgaagcagttctgtaataaagaattacgtgataacagatatatcggtaaagaaaaatacgaagaatataagaagagattccttaaaaaagttgacctagtaaaattagtttttggtgataaagcctttagaagatttattcctggagattctgtaaatgtaaacggaaaatggtcaacaaatgttcttaatatggcattatttgatgttcaaatgtgtggttttgtaaattatgagaaacatcaaattatttctaaagcagatgaaatcagagaaaaattaattaaattgatgagtcaaaatgaagaatttatcaattccattgaaattaaaactagtgataaaaatgttttaactaaaagatttagaatttggtatgaagctctagaggagattgttggcagaccttcaagtgaaaatcgaatatttgatttcaatattaaaaggcaacttttcgaaacaaatccgacatgtcaaatttgtcatcaacaaattttagcacttgatgatgcagaagttgatcatgttgttccttattcagaaggtggtagaacagtcctgagtaatgcccaattagtacatagatattgcaaccgaagaaagagtaataatattaattaa

SEQ ID NO: 352 - DNA, 975 bp, Bacillus megaterium; identical to SEQ ID NO: 9
  source 1..975: mol_type = genomic DNA

SEQ ID NO: 353 - DNA, 975 bp, Bacillus megaterium; identical to SEQ ID NO: 201
  source 1..975: mol_type = genomic DNA

SEQ ID NO: 354 - DNA, 975 bp, Bacillus megaterium; identical to SEQ ID NO: 201
  source 1..975: mol_type = genomic DNA

SEQ ID NO: 355 - DNA, 501 bp, unidentified
  source 1..501: mol_type = genomic DNA; note = Pseudarthrobacter sulfonivorans
atgcgcactctcgttctgaatgctggatatgaaccgctggcggtagtcaccttccgccgggcgctggtccttgtgctcacggggaaggcaagcgttgtcgcggagggtgaggaccctgtagtgggaccgcaggagatcctgggccgcccgtccgtgatcctcctgaaccgctacatccgcccccgatacaaccagaccaccgctgtgagccgccggggagtactgcgccgggacggacacaggtgcgcatactgtggaaaagcggcccacaccattgaccatgtccagcccaagtcccgaggcggtgcagattcctgggagaatctagtagccgcgtgcctgcgctgcaataacgtcaaaggcgaccacacacccgcggagatgggctggaaactccggttcacgccggaaccaccgcatggcacgatctggcagatcaaggagctggagaagcccacgcctgcctgggaccccttccttctgccagaacgggctgcctga

SEQ ID NO: 356 - DNA, 498 bp, Leifsonia xyli
  source 1..498: mol_type = genomic DNA
atgcgcacactggttctgaatgccggctacgagccgctggccgtggtgtcgtttcgacgggcgcttgccctggtgatgaatcagaaggccacgatcatcgagatcgacgacgagcatccggtgtggggcatccgctcggtgtacgaccggccgtcggtgatcgtgctgacccggtatgtgcacattccgcgcggacggtccgtgccggtcagtcgtcgcggcgtgctgcgtcgcgacgcgcaccgttgcgcatactgcggggtgaccgcgacgacgatcgaccacgtgatgccgcgttcccgcggcggcgcggacagctgggagaacctggtcgcctgctgcttgcgctgcaacaacgtcaagggtgacaggaccctggccgacctcggctggcggctgcggatcacgccgcgcatgccgcacgatccgggctgggccgtgcgcggtgtggagagaatggcgccgcagtgggagggatacctcgcacgtgccgcgtga

SEQ ID NO: 357 - DNA, 537 bp, unidentified; identical to SEQ ID NO: 262
  source 1..537: mol_type = genomic DNA; note = Glutamicibacter arilaitensis

SEQ ID NO: 358 - DNA, 564 bp, Azospirillum lipoferum
  source 1..564: mol_type = genomic DNA
ttggctccaccacccgatcactgtccggctctggtgctgaacgcggatttccgcccgctcagttacttccccttgtcgctatggtcctggcaggaagcggtcaaggcggtctttctcgaacgggtcaacatcgtatcgcactatgaccgggtcgtccgatccccaagtttcgaagtccggttgcccagcgtcatctcgctgaaagagttcatcccggccacgcgccgcccggccttcacccgcttcaacgtcttcctgcgcgaccgcttcacctgccagtattgcggccgccccttccccacccacgatttgaccttcgaccatgtgatcccgcggtcgcgcggcggtcggacgacgtgggaaaatgtcatcacatcctgttcggcctgcaatctggcgaagggcgaccgccttccccacgtctgcggcatgatccccctcagtccgcccttccagcccagtgcgtatgagctgcaagagaacggacgcgccttcccgccaaacttcctccatgaaagttggcgggattttctttattgggactcggaattggacccgatgtag

SEQ ID NO: 359 - DNA, 564 bp, Azospirillum lipoferum; identical to SEQ ID NO: 358
  source 1..564: mol_type = genomic DNA

SEQ ID NO: 360 - DNA, 3207 bp, unidentified
  source 1..3207: mol_type = genomic DNA; note = Bacillus toyonensis
ttgcgatatgtattaggattggatattggaatcgcatcttgtggttgggctgtaatcaatcaagaaaaagagagaattgaagatttaggggttcgtatttttgataaagctgaaaatccgaaagacggaaaatctttggctactcctaggcgtgacgcacgctctactaggagaaccctacgccgtaaaaaacatagaatgctacggataaagatacttttagtaaaacaaggcttactatctaagaacgaacttaaccatctatatgaaagtactactgaagtcgatgtttggatcttacgtttagatgctctagaacgcaaattgaatccaaaagagtttaccagagttcttattcatcttgctaaaagaagaggatttaaaactaatagaaaagaaaccactctctcagaaaacggacaaatcttagaaagcatttcagagaatcaacagattatggagcaacaaaattatagaactgttggagaaatgattctaaaagataaaaaatttgaaaaccataaaagaaacaaagatggtacttatatcgggaccgttacccgtcaacaacttcaagaagaaatacaaatgatattcactgcacaacgtttgtacaaaaacgactatgcaacagaggaatttgaaagtcgctacttagaaatctgggcatctcaacgtccctatgcttctaaagatcaaattgaaaaaatgattggctactgtaccttagaattaaaagaaaaaagagtccctaaggcttcttggtcatttcaatattttgtaacgttacaaaccataaacaatctcagactgattagtaaagacagaatagaagagctctcttttgaagaaaagaaacaagttataaatctagcacttgaaaaaactattgttaaatacattgatatccgaaaattattatccattcctaatgaacttcattttaataacctattatattcagctgatacagtagatacaacggtggaaaacaaaaaatgtatagaactcaaagaatatcactctattaacaagctatataaacaagtttatgggaagagcgttccaaatttattacctatagattacgatacaattacatacggattaactatctttaaagatgataaagatatcctacagtatttaaaaaataaatatgtaaatgcaaaaggaaaacctataagtaacttagcaaaaaagacctacgatgaaacatttattcaggcattattgactctgaatttcacgaaaatgggacatttatcatttaaagctttaaaaaatattattccctttttagaagaagggctatcgtacgataaagcatgtgagaaggcaggatacaactttaagggaacatcctacacagaacaaacaaagtacctacctgttatccctcagaataccaaccctgttgtacatcgtgctctttcacaaacaagaaaagttataaatgccattataaaaaaatatggttctccaaacgcaatccatattgaaaccgcaagagaattatcgaaaacattccaagaaagaaaagaaatagatagtatgtaccaggataactctaaaaagaatgaacacgccattcataagttaaaagaattggggaacattaacccctcaggaataaatattgtgaaatttaagctttggattgagcaagatggtagatgtatgtattctggaagatatatcgaaccacatcggttatttgaagaaggttatacggaagtagatcacatactcccctatagccgtagcttagatgacagctataacaacaaaacattaacactaggaacagaaaatcagcgtaaaggaaacaaaaccccttatgaatacatggggaatacttcaattttgaatgactttgaaacaagggtacagtcaaataaaaaaatcaacaaaaagaaacaacaaaaattacttttacaacacttttcatatgcgagagaacaagaatttattgaacgcaatctcaatgatacccgttatgccacaatatatctatcatcccttattcaacaacacctcatattctctgaaagctctcgtaagaaaaaagttcatacagttagtggaattataacatctcatttacgtagtagatggggatttaacaaagatcggaaagagggacatattcatcatgctttagatgcagtaatagttgctgtcacatccgatcacatgattcaaagagtaacaaagtattataagttaaaagagttaaatagaaaccttcaagccaaacagatgctgtttcctgaaccttgggaaggatttcgtttagagttagaagcaaggatttctccaaatacccagcaatatttaaagggccttcattttaaaaattacgccgatgtcaatctatccgaaatcaaacctatctttgtttcaagaatgcctaagcgctctatcactggtgaattacatcaagaaacgattcgaaaatttattggctataacgaaaaagggaaagtattaaccgcaataaaaacaaagttagaagatattccttttgatgccaatggtaactttcccatgtatgggaaagaaactgatttatacacgtataatgccataaaagaacgttatttaagtcataaaaaagataaacgcaaatcattccaagaacctttatacaaacccacaaaatcaggaggaattggaccacttataaaatctattaaaataatggatacgagaaccattgttaatcctgttaatcaaggtaaaggtgtcgtgtataatagtaaaattgctcgtactgatgtattcaaaaaggatgagaagtactatttaattcctatttacacaattgatcttatgaaaaatatcttacctcaaaaagcaattacagcaggaaaaggatatgaggattggacaaccattgatcctagctttacttttttatttagtctttttcccaatgatctaattcaaattgtcccctcaaaaaataaaacaattaaagctagaacaactgtatcgaaaaaagaggtattactaccctcactcacaggatattttaaaggtgttcatagtggaactgcggggattactgttgaaacacacgatggcagtgttatagcaaatgtaggctcaaagcaattactattaattgagaaatatcaagtagatgtacttggacattatacaaaaataaaggaggaaaaacgtattgggatggcgatatag

SEQ ID NO: 361 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcatttgctaaaatatattggaatttggttattcatcatgatttaaatcaattaaatacacacaatagacaagctggaattcaaaaagagctaaaggaatttcagttaatgcatggtgtccccaataaagtagtatttgatcgtttaccaagtaatcttcaattgcagttagtagaaagaactaaaaaagtgggggcgaggtatgtagtaggggtgttatacggagatatggaaggttctatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagatgatgtcttattgaaagtggaaaatatttcaaaacacgaatcgctgcatgcattctataacatactttggaaatacgaaacaaaagaatgtttttattgtggaaaacgtctttctgataaacgcatcgctactcatgtagatcattttattccatggagttatatccaatcagataatatgtggaatttagtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 362 - DNA, 501 bp, unidentified; identical to SEQ ID NO: 108
  source 1..501: mol_type = genomic DNA; note = Pseudarthrobacter chlorophenolicus

SEQ ID NO: 363 - DNA, 501 bp, Arthrobacter sp.; identical to SEQ ID NO: 106
  source 1..501: mol_type = genomic DNA

SEQ ID NO: 364 - DNA, 564 bp, Azospirillum lipoferum; identical to SEQ ID NO: 103
  source 1..564: mol_type = genomic DNA

SEQ ID NO: 365 - DNA, 558 bp, unidentified
  source 1..558: mol_type = genomic DNA; note = Mesorhizobium oceanicum
gtgacagttcatgtctcgccagatggtttgccggcactggtgctgaacgcggactaccgtccgctcagctattacccgctgtcgctctggtcctggcaggacgcgatcaaggccgtgttcctcgatcgggtgaacatcgtcgccgaatatgagcattccgtctcgtcgccgaccttctcgatgaggctgccttcggtggtctgcctgaagagttacgtgaagccgtcgcgcaatccggccttcacccgcttcaacgtgttcctgcgcgaccgcttccagtgccagtactgcggctcgcccgaggatctcaccttcgaccatgtcatcccgcgccgctgcggcggggcgacgacctgggagaacgtcgtggcggcctgctcgccctgcaatctccggaagggcggcatgctgccggctcaggcgaagatgtggccgatgcagaagcccttccacccgaccgtgcacgacctgcacaacaatggccggctcttcccgcccaaccatctgcacgaaagctggatggactatctctactgggacgtggaactggaaccctag

SEQ ID NO: 366 - DNA, 501 bp, Arthrobacter sp.; identical to SEQ ID NO: 131
  source 1..501: mol_type = genomic DNA

SEQ ID NO: 367 - DNA, 495 bp, unidentified; identical to SEQ ID NO: 292
  source 1..495: mol_type = genomic DNA; note = Glutamicibacter arilaitensis

SEQ ID NO: 368 - DNA, 495 bp, unidentified; identical to SEQ ID NO: 292
  source 1..495: mol_type = genomic DNA; note = Glutamicibacter arilaitensis

SEQ ID NO: 369 - DNA, 501 bp, unidentified
  source 1..501: mol_type = genomic DNA; note = Pseudarthrobacter phenanthrenivorans
atgcgcactctcgttctgaatgctggatatgaaccgctggcggttattactttccgccgggcgctggtgcttgtgctcacgggcaaggcgagcgtcgtggccgaaggggacgatcctgtcgtgggaccccaggagattctggggcgcccgtccgtgatcctcctgaaccgctacatccggcccagatacaaccagtccacggcagtcagtagacgcggggtgctcaggcgcgacgggcacaagtgcgcctattgtggaaaagctgcgcacactatcgaccacgtccaccccaagtcccgcggcggagcggattcctgggagaacctcgtggctgcatgccttcggtgcaacaacgtcaaaggtgatcacaccccggcggagatgggctggaagctgcggttcgtgcccgagcccccgcacggcaccatctggcagatcaaggaactcgagaagcccacgcctgcgtgggatccgttcctgctgccggagcgggctgcctga

SEQ ID NO: 370 - DNA, 501 bp, unidentified; identical to SEQ ID NO: 123
  source 1..501: mol_type = genomic DNA; note = Pseudarthrobacter phenanthrenivorans

SEQ ID NO: 371 - DNA, 501 bp, Rathayibacter tritici
  source 1..501: mol_type = genomic DNA
atgcgcacactcgtcctcaacgccggctacgagccgctcgcggtggtgtcgttcaagcgggcgctggtgctgctgatgaccgagaaggcgatgatcatcgtcgccgacgaggagcacccggtgcacggcgcgaccggcgactatgtccggccctcggtgatcgtgctgacgcgctacgtccgcatcccgagctcgcgcagcgtgccggtctcgcgtcgcggggtgctgcggcgcgacaaccaccgctgcggctactgcgggcagcacgccaacacgatcgaccacatccagccgaagtcgcggggcggccgcgactcctgggagaacctggtcgcctgctgccaccgctgcaacaacgtcaagagcgaccggacgccgctcgagatgggctggcagctgcgctccgtgccgcggatgccgcgcgcaggctcctggctcgtgcgcggcgccgagcgcacccagcccgagtgggacgactacctgacggcggcagccgcctga

SEQ ID NO: 372 - DNA, 501 bp, Arthrobacter sp.
  source 1..501: mol_type = genomic DNA
atgcgcacactcgttctgaatgctggatatgaaccgctggcggtagttaccttccgccgggcgctggtccttgtgctcaccggaaaagcaagcgtagtggccgaaggcggcgaacctgtcgtcgggccgcaggagattctgggacgcccgtccgtgatcctcctcaatcgctacatccggccgcggtacaacaggatcacggccgtcagccgtcgcggagttctccgccgcgacggccaccgctgcgcctactgcggtaaaacagcccacaccatagaccacgtccaccccaaatccaggggcggcgcggattcctgggaaaacctggttgcggcgtgcttgaaatgcaataacgccaagagcgaccacacgccggcagagatgggttggaaactccgtttcacacccggcgttccctccggaaccatgtggcagatcaaagaactcgagaagcctgcgccggactgggatccgttcctcttgccggaatccgctgcctga

SEQ ID NO: 373 - DNA, 564 bp, Azospirillum lipoferum; identical to SEQ ID NO: 103
  source 1..564: mol_type = genomic DNA

SEQ ID NO: 374 - DNA, 501 bp, Arthrobacter sp.; identical to SEQ ID NO: 106
  source 1..501: mol_type = genomic DNA

SEQ ID NO: 375 - DNA, 501 bp, Rathayibacter tritici
  source 1..501: mol_type = genomic DNA
atgcgcacactcgtcctcaacgccggctacgagccgctcgcggtggtgtcgttcaagcgggcgctggtgctgctgatgaccgagaaggcgatgatcatcgtcgccgatgaggagcacccggtgcacggcgcgaccggcgactacgtccggccctcggtcatcgtgctgacgcgctacgtccgcatcccgagctcgcgcagcgtgccggtctcgcgccgcggggtgctgcgacgcgacaaccaccgctgcggctactgcggtcagcacgccaacacgatcgaccacatccagccgaagtcgcgcggcggccgggacacctgggagaacctcgtcgcctgctgccaccgctgcaacaacctcaagagcgaccgcacgccgctcgagatgggctggcagctgcgctccgtgccgcggatgccgcgcgccggctcctggctcgtgcgcggtgccgagcgcacccagcccgagtgggacgactacctgggggccgcggccgcctga

SEQ ID NO: 376 - DNA, 501 bp, Microcella alkaliphila
  source 1..501: mol_type = genomic DNA
atgcgcactctcgttctcaacgccggttacgagcccctcgcggtcgtctcgttccgacgagccctcgtgctcgtcatgaacggcaaggccgtcgtcctcgctcgcgatgacgcgcatcccgtcatcggcatccacgacgccttcgagcgcccgagcgtcatcgtgctgcgccgctacgtccgcgtcccgcactcgcgccaggtgccgctcacccgacgcggggtgctgcgccgggacggcaaccgctgcgcctactgcggcgcttcggccacgacgatcgaccacgtcatgccgcgctcgcgcggcggcgccgactcgtgggagaacctcgtcgcctgctgcctgaagtgcaacaacatcaagagcgaccgcacgccggccgagatggggtggcgcctgcgcgtccagcccaaggccccgcacgggacggcctggctcgtgcgcggcatcgagcgcccgcagccggcgtgggacgagtacctcgcgccgctcgcggcgtag

SEQ ID NO: 377 - DNA, 537 bp, unidentified; identical to SEQ ID NO: 262
  source 1..537: mol_type = genomic DNA; note = Glutamicibacter arilaitensis

SEQ ID NO: 378 - DNA, 501 bp, Arthrobacter sp.; identical to SEQ ID NO: 131
  source 1..501: mol_type = genomic DNA

SEQ ID NO: 379 - DNA, 3279 bp, Brevibacillus laterosporus
  source 1..3279: mol_type = genomic DNA
atggcttacacaatgggaatagatgtggggattgcttcgtgtggatgggctattgtagatcttgaaagacaacgaataatagatctaggtgttcgaacttttgagaaagcagagaatccgaaaaatggagaggctcttgctgtaccaaggagagaagctagatcgagccgtaggagattacggcgaaaaaaacatcgcattgaaagattaaagcatatgtttattcggaatggactggcggttgatattcaacatcttgagcagacgttacttaaccaaaatgaaatagatgtatggcaattacgagtagatggtttggatcgaatgctaactcaaaaagagtggcttcgtgtattaattcatcttgcacaacgtcgtggttttcaatcaaatcgaaaaacagatggttcaagtgaagatggacaggttcttgtaaatgtaacggagaatgacagactaatggaagagaaagattatcgaaccatagccgaaatgatggtaaaagatgaaaaattctctgaccataagcgaaataaaaatggaaattatcatggagtagtgagcagagcttctttaatagttgaaatacatacattatttgaatcccaaagacagcaccataattttttggcatcgaaagatttcgagctgaaatatgttaatatttggtcttcacaacggcctgttgcaacaaaagatcaaatagaaaaaatgattggtacatgtactttcttaccgaaagaaaagagagctccaaaggcatcttggcattttcaatattttatgctcttgcaaacgatcaatcatatccgtataacaaatatacaaggcacgagaccattgaataaagaagaaattgaacaagtggtcaatatggcacttaccaaatcaaaggtatcttaccatgatattagaaaagtccttgatttatcagaagaatatcaatttgttggcttggattatggaaaagagaatgaaaaaaagaaagttgaaagtaaggaaacgatcatcaaattagatgattaccataagttaaataaaatttttaatgaagtggaattagctaaaggagaaacttggaaagctgttgattatgatacagtagcgtatgctctcactttctttaaagatgatgaagatattagagattatttgcaaaataaatataaagatagtaaaaatcgtcttgttaagaacgtagctaataaagaatacacaaatgaattgatagaaaaagtgagtacgctatcttttcgtaaggtggggcatttatccttgaaagccttacgaaagatcatcccgtttttggagcaaggcatgacctatgataaagcgtgccaagcggctggttttaactttcaaggtatattaaaaaagaagagatctgttgtcttgccggtcattgaccaaatttccaacccagtagtaaatcgtgctcttactcaaacgcgtaaagtaattaatgctctaatcaaaaaatatggttcacctgaaaccatacacattgaaacagcaagagagttatcaaagagatttgatgaacgaaagaatattactaaggattataaggagaatagagacaagaacgaacgtgcaaaaaaacacttatcggaattagggattattaatcctactggtcttgatatagtaaagtacaagctgtggtgtgagcaacaagggcgctgtatgtacagcaaccaacccatctcctttgaaagactgagagaatcaggttataccgaggtagatcacattattccatacagccgaagtatgaatgatagttataacaatcgcgtattggttatgactagggaaaatcgtgaaaagagtaatcaaactccttatgaatatatgggtaatgatacgcaaagttgggatgagtttgaacaaagagtcacaacaaatccacaaataaaaaaggagaaacgacagaacctcttgctaaaaggattcactaatcgtcgggaattagaaatgttagaaagaaacttaaatgatacacgttatataactaagtatctttcccattttattagtacgaatttggaattctctcctagtgataaaaagaaaaaggtagttaatacaagcggtcgtatcacttcccatttaagaagtagatggggattagaaaaaaatcgtgagcaaaatgatttacaccatgcaatggacgcggttgtaattgctgtcacctcagactcgtttattcaacaggtaacgaactactataaacggaaagaaagaagagagttgaatgggggtgataagttccctctaccttggaagttttttagagaagaagtcatagcaagattaagccctaatccaaaagaacaaattgaagcattgcccaaccatttttatagcgaagatgaacttgctgatcttcaaccgatttttgtctcaagaatgcccaagcgtagtataactggagaggcgcatcaggcacagtttcgccgagttgtgggtaaaacgaaagagggtaaaaatattactgcaaagaaaactgctttagttgatattagctatgataaaaatggtgattttaatatgtatggaagagaaacagatcctgctacttatgaagcaattaaagaaagatatctcgaatttggaggaaatgctaaaaaagcattttcaacggatttacataaaccgaaaaaagatggcaccaaagggccactgataaaatctgtgagaataatggaaaataaaacattggtacatcccgtaaataaaggaaaaggcgttgtgtataacagctctattgtaaggacagatgtatttcaaagaaaagagaaatattatttactacctgtgtacgtaacagatgtaaccaaggggaaactaccaaataaggtgattgttgctaaaaagggatacgatgattggattgaggttgatgatagcttcacatttttatacagtctatatccgaatgatttaatattcatcagacaaaatccaaaaaagaaaatatcattaaaaaaacgtattgagagtcattctatttctgatagtaaagaggttcaagaaattcttgcctattataaaggggttgatagttcaactgctgctatagagtttattactcatgatggtagttactatgcaaaaggtgttggtgtccaaaatctagattgttttgaaaaatatcaagttgatattctgggtaattattccaaagtgaaaggagaaaaacgacttgagttggagacatctgataataaccacaaaggcaaagatgtcaattcgattaaatcaactagtcgttaa

SEQ ID NO: 380 - DNA, 501 bp, Arthrobacter sp.; identical to SEQ ID NO: 149
  source 1..501: mol_type = genomic DNA

SEQ ID NO: 381 - DNA, 501 bp, Arthrobacter sp.; identical to SEQ ID NO: 134
  source 1..501: mol_type = genomic DNA

SEQ ID NO: 382 - DNA, 501 bp, Arthrobacter sp.; identical to SEQ ID NO: 149
  source 1..501: mol_type = genomic DNA

SEQ ID NO: 383 - DNA, 501 bp, Arthrobacter sp.; identical to SEQ ID NO: 134
  source 1..501: mol_type = genomic DNA

SEQ ID NO: 384 - DNA, 501 bp, Arthrobacter sp.; identical to SEQ ID NO: 278
  source 1..501: mol_type = genomic DNA

SEQ ID NO: 385 - DNA, 975 bp, Bacillus megaterium; identical to SEQ ID NO: 116
  source 1..975: mol_type = genomic DNA

SEQ ID NO: 386 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataatgttgacaatcggttagtactcacgtttgatcaagtgtatttttcctttgctaaaatatattggaatttggttattcatcatgatttaaatcaactaaatacacacaatagacaagctggaattcaaaaagagttaaaggaatttcagttaatgcatggtgtcccaaataaagtaggatttgatcgtttaccaagtaatcttcaattgcagttagtagaaagaactaaaaaagtgggggcaaggtatgtagtaggggcgttatacggagatatggaaggttccatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagaagatgtcttgttgaaagtggagaatgtttcaaaacgcgaatcgctgcatgcattctataacatactttggaaatacgaaacaaaagaatgtttttattgtgggaaagttctttctgataaacgcatcgctactcatgtagatcattttattccatggagttatatccaatcagataatatgtggaatttggtgttaagctgtcaaagttgtaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 387 - DNA, 420 bp, unidentified
  source 1..420: mol_type = genomic DNA; note = Paenibacillus yonginensis
atgattacggtactgaatacagatggtcgggaattaaccccttgtaccctggaccgagcttggcgggaggtgaatcgatcgcgtgccgtttggattaatgagcagaccattcagttgttatacaacccttttctgttccggaaatatcggaagatcgccttaaaaagagaccgttacacgtgcctatggtgcggacagcctgcaacaacggtggatcatctgatcccctccagcaagggcggttcggatctgccgcgcaaccttattgcctcctgcggcgagtgtaactcaagacgggggaatcgcccggcgtgggagtattttagagagaagatgggtttcattccccgtcccttcaagcttggttttcatattttgatagctttaggctgctcaaagatcaaaccaagatctcattaa

SEQ ID NO: 388 - DNA, 501 bp, Arthrobacter sp.
  source 1..501: mol_type = genomic DNA
atgcgcactctcgttcttaatgctggatatgaaccgctggcggttgtaaccttccgccgggcgctggttcttgtgctgaccggaaaagctagcgtagtggccgagggcgacgatcctgtcgtcgggccgcaggagattctgggccgcccgtccgtgattctcctgaaccgttacatccggccgcggtacaacacaaccaccgcggtgagccgccggggcgtcctgcgccgtgacgcccaccgctgcgcctactgcggtaaggcagcacacaccattgaccacgtccacccgaaatcgaggggcggtgcggactcctgggaaaatcttgtggccgcctgccttcggtgcaataacgtgaagggcgaccacacgccggccgagatgggctggaagctcaggttcgtgcccgcgcctccgcacggcaccatctggcagatcaaggagctggaaaagcccacccccgcgtgggaccctttccttctgccagaatccgccgcctga

SEQ ID NO: 389 - DNA, 456 bp, Paenibacillus sp.; identical to SEQ ID NO: 140
  source 1..456: mol_type = genomic DNA

SEQ ID NO: 390 - DNA, 456 bp, Paenibacillus sp.; identical to SEQ ID NO: 140
  source 1..456: mol_type = genomic DNA

SEQ ID NO: 391 - DNA, 420 bp, unidentified; identical to SEQ ID NO: 137
  source 1..420: mol_type = genomic DNA; note = Paenibacillus yonginensis

SEQ ID NO: 392 - DNA, 462 bp, Paenibacillus sp.
  source 1..462: mol_type = genomic DNA
atgaatcatagtcatgatggagaggaaccactgataacggtactcaatatggatggtcaacaactaacaccatgtttgccccatcgggcttggcgggaagttgagcgatcgcgagctgtctggattgatgagcagacgatccaactactttataatccgttcttatttcggcactatcgaaaagccgcactgaaacgagatcatcacacttgtttgtggtgcggacgatacgccacgacagtggaccatattatccctgcaagcaaaggcggctcggatcttccgcataacctgatcgcttcatgcagcgaatgcaatactaaacgaggagaccgttctgcattatcctatctcaaagaaaacgcgaaccttgttccaaatcttatgaagctctactggcgtatctttaaagctaaatacaaacatcgtctaatgaaaaaaggacacgtaagtaaagcgtag

SEQ ID NO: 393 - DNA, 4170 bp, Paenibacillus sp.
  source 1..4170: mol_type = genomic DNA
atgaaaaaaatgaattatagtatcggattggacatcggtacgaattcagttggttgggcttgcatagatgaaaacttcaatgtactaaagtatagagatcgttttgctattggtgttcatgagtttgaagctggtgaggcagcgtcggatcgtcgtatcaagcgtggcgctagaagaagatacaatcgtagaaaacgtagaattcaacttcttcaagaactttggacaccattaatgacaccaaatctgaataatcctgtacctttctttgcctatgccgatgaaacaaagcattggcgccaagataatcaattccaagatagatcccttacccacgtactaaagcttatacgagttaatcctcggaaatatccaacgatttatcatttacgaacagcattaatgaaagagaatcgtaaatttgatcttagacttgtttatttggcaatccacaatctggtaaaacatcgtggacatttcttaaatggaatagatgagtggctagatgctagtcgtggtggtaatgaagcagaaaacttatatgagctaataacagaatatcaaaaattagtgaatgactcctatgtcattgatcgtgagatcattaatgcagttattaccattattaatgataaggagaagaacaaaacagataagcatcatgcccttatggagatgcttcctagtacagttagtgaatttatgaagttgatattgaatttaacgacgaccttcgcaaaacttttcccaggctcaagccaattagaagagtataaggaattgaaagatgttcaaggtaaaattcaaatttccgctgaagacatccttgttaagttggatcagcttcttgaggatgaacgagaagttgtcgttagagctactgttgtatatcaacagcttcaacttaaggaattgttaaacggcaaactttttgtagcagaagcaaaagtggcatcatacaatcaattccacaaagaactagaacaactaaaggaaatcgttagaaactacggtactgaggaagagtacaagcaattgttcattacacctaaagcaaattggagggagtatcaaaagttacctaatatcaacttattgtgtgtacttgatcgctatactgtgcaagcaaagcctaatcaaaagatagaaaacatgctagggactagtgccgagaagcaagataatcgaaatacagagctatattttaatttgttcgtgcgtgagctcattacatcttttattccaaaagcagagacggatgatcaaagagagcagctagagcaaattattcgtaaaattgacgaacgccaatacatggtgaagcaggttaataaatcgaattctgcaattcctgttcagaatagcatgtatgaagctcgagaaattctacaacggcaagcgaagtattatcctgcaatgacgacagagtggattgaggatgtgatacaactgatccgcttcagaataccttactacgtagggccgttagtgaagtcaacgaataatcaagcttccagtccatttggctgggcgatccgtaacgataaaagcagtcgtattacaccgtggaactttgacgaagtaatcaatccatctcaaactgctgagggatttatcaagcgtatgacgaataaatgcacctaccttcatcatgaagatgtattgcccaaacactcattgctttatcaagaatttgagttgctggatgagcttaatcgcattcaaattcgtcgtccttcagaagataaacatctggatcatcgtttggatcctaagttaaagctctggcttattcgagaagtatttcaaaagaagataaaagtcagtcacgaggatgtactccaggctattctaagatctccttggaaagacagctatgctgatcatcataatggggaacgttatcaaatatttggcacatcgaaggaaaaggaattcgcgagtacacaatcaacatacattaagctgaagcaaatctttggagcggtactagtatcagcgtatccattgtacgaggaagtcatttacgcattaactgtttttaatgaggaacaaataataaagtataaactatctcagttaaaagaatcagatcctaatggatatttgcttaatgaagatgtagttaggagtttgctatattggaagccaaatggctggggtagattaagtcgaaaactattaagtggtattccagtatatgaacacgatactttaaccgctatagaggccatgcgacagcagacctatgtatttaacgaattaaagtctccaggtcaccgcatggagggtagacaggctaaggttaagcactattcacatctacaaaaacgaatagaattagagaataaactccataaacaaaacgaaaagacgacattccgttttacgtatgcagatgtaaaggatctagcaggatcaccggcaattaaacgtggaatatggaaatctctcaaaattgttgaggaactagtttccattttcggcgaacctgagcatattatgctcgaagtagcacgcgaagagggagacaaaaagcagcgtaccaaatccagagaggaacagtggaagctattaggtcaaagcttaaaatctagcgaatacgctgagttttataagcaaaacagtaagcaaattatgagtagcgagtttaagcaaacgagattatggttatatttattacaaggaggaaaatgtttatatacacagactcctctagacattaatcaactatcaagctatcaaatcgatcatataattcctcaaagaattgtgaaggatgatagtattgataatttggctttagttcttagtagcgctaatcaatccaaagaaggagtgtttgttcctctcgagattataccaaaggagaaacactattctattaaaacctattggaaaagtttattggatttgaaattaatgtctcgtaaaaaatatgagaatcttactaagaaaaaactaggtgaagctgaaattgatggtttcattcagcgtcaactagtcgagacaagacaaatcattaagcatgttcaagaactccttcaggaacgctttaaagaaacgaatattcatctcgttaaggctaatttaatctcaaaatttagacaagctaactatttacctaaagtacgtgaaatcaataataagcatcatgcaatagatggttttctcgcagcagcttttattcaatcggtaatcaagaagtatagtgaaaattatctgcaatttagtttacaacgacaaaatcttgatcagattcttgcaaaaaaggtaagtgtgaaggaagatttcttcttattccgcaatttccatgagcaacgttacccaactctcttacaagcaggagggacacaaagtgccaaggagtatttggatgaagtcatgtatgagttaccgtggcaaagcacatatgcaataggtagtgctgaagctagcttctacaatgaaacggtatattcaccgaaggcgaagaaggattttgataaggtaaaagatacacttagctatacagaattaacacacgaaaatttcaacaatttattctctattgcccttgaagtagatgaggttaagaagaaaaaaacaacgtacaagacaattattctagacgttagagtattcgattattatcaattccgtcacttgtcagaccaagaactatctgagaaacttgttgagaaatactgccctcaatacacaaatgtgaaatttattaagaaaattcataagtaccaaaaggttatatataaaggagaaccagcgtattttatctcttctggtgaactacgcaacggtaagcaacttcagcttgatagaaaattacatgctgagattgctcaattattagatccaagattcttaccgaagaaaggtgatgaatccgatcaagagccaattaatgcgctggataagtttaacgagctaagtctggtgatttttgaaaactataattatttgcctggcgtagaaaagaaaaaatttgagagtgcaagtaataagatagataacgcggaaattggtgtaaagatgattaatgaggcattgaaagtatctgctgctaatgcttctcgctcagatgttttaggtggcagatggctagtaactgttgcacctgattgtatgaaattagtagatgaatctattacgggattaaaatatagaaaataa

SEQ ID NO: 394 - DNA, 456 bp, Paenibacillus sp.; identical to SEQ ID NO: 140
  source 1..456: mol_type = genomic DNA

SEQ ID NO: 395 - DNA, 501 bp, Arthrobacter sp.
  source 1..501: mol_type = genomic DNA
atgcgcacactcgttctgaatgctggatatgaaccgctggcggtagttacattccgccgggcgctggtccttgtgctcaccggaaaagcaagcgtagtggctgaaggcgacgatccagtcgtcgggccgcgggaggttctgggacgaccgtccgtgatcctccttaaccgctacatccggccgaggtacaacaggatcaccgctgttacccgccgcggcgtgctgcgtcgggacgcacaccggtgcgcctactgcggcaaagcagcccacaccattgaccacgtcctccccaaatccaggggcggcgcggattcctgggaaaacctggtggcggcgtgcctgcgctgcaacaacgccaagagcgaccacacgcccgccgaaatgggctggacgttgcggttcaaaccggcggctcccacgggaaccatctggcagatcaaggagctcgagaagcctgctcctgcatgggatcctttcctgctgcctgaatcggcagcctga

SEQ ID NO: 396 - DNA, 3117 bp, Variovorax paradoxus
  source 1..3117: mol_type = genomic DNA
atgcgctatcgactcgccctggacctgggctcgacttcactgggctgggccatactgccactcagggacaaccggccgtacgccatcaccaaggcgggcgttcgcatcttttcggacggccgcaacccaaaggatggctcatcgctcgctgtgagccgccgagcagcgcgaggcatgcgccgacggcgcgaccgtctgctgcggcgcaaggcacgcatgatggagaagttggtcctctacggcttctttcccaaggatgcggcagcgcgcaaggcactggaagcgctgaacccttatgcgttgcgcgccaagggcttgaacgaaccactgttgccgggcgagttcgcgcgcgccctgttccacatcaaccagcggcgcggcttcaagagcaatcgcaagaccgaccggaaggacaacgacagcggcgcactgaagaaagccatcagtaccctgcgcattcaactggaggaaagcaacagccgtacggtgggcgaatggctatggcggcgaatacagggcggccagagcgtgagggcgcgctaccgtgaaacgccgttcgtcaacgacgaaggcaagaggcgcatcgacaagagctacgacctctacattgaccgcgcgatggttgagcaggagttcgatgcgctctgggcggcacaagcggccttcaacccaaccctctttcatgaaaccgcgcgcaccgatctgaaagacacgctgctgttccagcgccggctgcggccagtcaagccgggccgctgcacgcttctgcccgaggaggaacgcgcaccgctggccctgccgagtacgcagcgcttccgcatctaccaggaactcaaccatctgcggctactggacgatcagttgcaggagaggccgttgacgctggaacagcgagacctgctggcttcgacgttggaacacaaggccaggctcagcttcgagcagatgcgcaagttgctgcacctgagtggaagcacgcaattcaatctggaggatgagaagcgcaccgaactcaagggcaatgccacaggcgcattgctatcgaagaaggaattgctgtcccaggagtggaaccgactaccacccgatcttcaggatgaaatcgtctggcagctcgtgactgaagaaagcgaagaagcactcatcgcctggttgcaggcacgtgcgaatgtggatgaagcacgggcccaggcgctggccgacatcagcctgccagagggctacggcagcctcagccgcaaggcgctggcacgcatcgtgcccgcgctacgcgagcccgatgaaaacggcctgccccgcacctacgacaaggcagtgcaggcagcgggcttctcgcaccacagcgatttgggattcgacttcgagcacctccagtccgaagtggagcagactggccagcgaaccatcgcgtccacgggggaaatcaggccggtctttgccttcaggcaattgccctattacggccgggcgctgcaacggcatgtcgcattcgggagtggcaagcctgaagatgcagaagagaaacgctacggcaagattgccaaccccaccgtgcacatcggcctgaaccaagtgaaggtggtggtgaacgcgctgatccgacgctacggccgccccagcgaggtcgtgatcgaactggcgcgtgacctgaagcaaagccgcgagcagaagcaagacgcacagcgcaggcagacggagaaccagcgccgcaacgcgcgcattcgcagggaaatctcccaaatccggggaatgagcgaagagcaagtgcgccagagcgacatccagaagtggattctgtgggaggagttgagcttcgacgttgccgatcggcgctgcccctacagcggcgttcagatcagcgcggccatgctgctgagcgagcaggtagagattgagcacatccttcccttctcgcgcacattggacgacagcctgaacaatcgcacggtggccatgcgtcaggccaatcgagtcaagcgcaaccgcacgccgtgggatgcacgcgccgatttcgagaggcaaggctggagctacgaaggcatcttgctgcgcgccgagcgcatgcctctgcgcaaacgctatcgcttcgcgcccgacggttacgaacgatggctgggcgaagataaagactttctggcccgagcgctcaacgacacccggtacctctcgcgtgtggctgccagttatctgcgactggtttgccctcaaggcacccgcgttatccccgggcagatgacggcaatgctgcgcgccaagttcggcctgaacgacgttctgggggtgaatggcgagaagaaccgcaatgatcatcgccatcacgcagtggatgcctgcgtgatcggcgtcaccgaccagggattgatgcagcgcttcgcccaggccaatgccgatgcgcgcgaacacggcttgacgcggctggtcgaggacatgcctctgccctggcccacctatcgggatcatgtggcgcgcgcagtgcagcacatctgggtgagccaccgccctgaccatggctatgagggcggcatgatggaggagacctcctatggcgtccggcgcgatggcagcatcaagcaacgccgcaaggtggatggcagcgcggcgcgcgaaatcacgaatctcatccgcattgccgaagccacgcaaccgacacggcatggcatcgacgcagaaggacgaccgctaccttacaaagggtacatcggtgggagcaactactgcattgagatcactgtgagtgatggaggcaaatgggaggggaacgtgatctcaacgttccaagcctacgccatcgcccgacaaggaggaattgcgcaactacgccaccccacgcggggacagaacggccgcccgttagccatgcggctggtgatcggcgacagcgtgcggctcaaggtggacggagcggatcagacgatgcggatcgtgaagatcagcgggaacaatgggcagatattcatggcgccggtcaacgaagccaatgtggatgcgcgcaatcgcgacaagttggacgcattcgagtacatatccaaatatgctggctccctccagaaggccaaagcccgtcgggtaaccgtatccccagacggtgtattgcgtgacccgggattcgcagggtag

SEQ ID NO: 397 - DNA, 975 bp, Bacillus megaterium; identical to SEQ ID NO: 206
  source 1..975: mol_type = genomic DNA

SEQ ID NO: 398 - DNA, 498 bp, Rathayibacter tritici
  source 1..498: mol_type = genomic DNA
atgcgcactctggttctcaacgccggctacgagccgctcgcggtggtgtcgttcaagcgggccctggtcctgctgatgacggagaaggcgaccgtgatcgcggcggacgagggcaatccggtgcacagcgcgcacggcgaccacgtgcggccctcggtgatcgtgctgacccggtacgtgcgcatcccgagcggccggagcgtgccggtgtcgcgccggggcgtgctccgccgggacaaccaccgctgcggctactgcggtcagcacgcggcgacgatcgaccacatccagcccaagtcgcgcgggggccgggacacgtgggagaacctcgtcgcctgctgccagcgctgcaacaacgtcaagagcgaccgcaccccgcacgagatgggatggcgtctgcgcaccgtcccccggatgccgcgcgccggctcctggctcgtgcgcggcgcggagcggacgcagcccgactgggacgactacctggctcccgccgcctga

SEQ ID NO: 399 - DNA, 3306 bp, Shinella sp.
  source 1..3306: mol_type = genomic DNA
atgaaaacgcttggactcgatctcggcacaaactcgctcggctgggcaatcatcgaaacgcgcggtgaaccgggcgaaaacgacgagggacgcattgttgccagcggcgtgcgcatcttctctcaatccgaaatggcgggacgtgatccgcagtcgaaggcttccctggccgtggcgcggcgtcaggcgcgtgggtcaaggcgccggcgggatcgctatctgaagcggcggcagcgccttgtcgaccttctcgtcgatctcggtctgatgcccgccgacaaggccgcccgcgcggagctggtgaagaaccatggggatggaaagacgggcgacctctcgtcatccgtctatgcattgcgcgcgcgggcgctggacgaggcgctatcgccctatgagatcggccgcgcgattttccacctcaatcagcggcgcggcttcaagagcaaccgcaaggccgacagcaacgaccctgaacagggcgtgatctccgaggggatgcgcgcgctcgatgaaaagatggcggaggcgggcgcgcggaccatcggcgaatggctgcacatgcgccggctgcaaggcctgccggttcgtgcccgtatcgcgcctgacggggaaggctatgccttctatccctcgcgtggcgcgcttgagcgcgaattcgaccggatcatggcccgccagacgctcttccatcccgacctgctcaccgacgctgcaatccgggaactgcgcgaggtggtcttccaccagcgtccgctgagaccggtgaaaccggggaagtgctcctacaatccgctggaggagcggttggcgaaggcacatcccctgttccagaaatttcggctgctcaaggaactcaacgaactggaaatcgtcggggaggaccagaaacacatcaagctgacgccggagcagcgcaatgccctggcacttcagttgcgcacgagcctcaccaagcagggaaaggtgcccttctcgaagttgcggggcacgctgaagctcgggcgggaagtgcgcttcaacaaggagaccgacaaccgcagcgatctcgaaggcgacgtgatctattttcgcatgtcgcgcccggaatgcttcggtaaccgctgggcggacctgccggtggagatgcaggcggcaatcaccgagagactgcgcaccgagcctgactacagcaccctcatgaactggctgaaaacagagacagggctggatgaggttcatgcccgggcggtggccgacacaccggtgccggacggtttcggcaggctggggcaatccgcactgaccgcgctcatcgatgcgctggaaaacgaagtgggcgagggcggcttcgtcatcaccgaggccgaggcggcgaagcgggtttacggccgcaccaattccgaagccgatcccgaccgcaagggcgtggatgaactgccgaaatatcaggaggttctggcgcgccacattcctccgggaacgggcgagcccgacgatccctatgacgatttcatgggccgcatcaccaatcccacggtccacatcgcgctcaaccagttgcgccgcgtggtcaacgcgctgatccggacgtacggcaagccggaccggatcgccatcgaggtgggccgcgacctgaagatgaacgacagggagcgcgacgaggtcaaccgccggatcgggcggaacacgaaagcggcgatcgaacgaggggtgaagctgaccgagaccttccggcagttggacaccggttacaatcgcctccggctggaattgtgggaggatctgaaccagaagccggaaaaccgtgtctgcatctattgcggcaaggccatcgccggccacatgctcttcaacggcgagacggatgtggatcacatcctgccctattcgaagacgctggacgacagcaaggcgaaccggatcctgtgctgcacctcctgcaatcgcgagaagaagaaccggcctccgtcgcgcgtgccgcaatggcgcagtcgctatggcgagattctggcgcgcgccacgacgctgccgaagaacaagcaatggcgctttggcgaagacgccatgacgcgcttcggcgaggaggaaggtttcctcgcccggcaattgaccgacatgcagtatatttcccggctggcgctgacctatcttgcccatctctacgattacgaagaagtggacctcgacggtatctacaagcgccatgcccgtgtgcgcgccttgcccgggcggatgaccgagatgctgcgccgccagtggagcctcaacgagcttctgcatggccacaatctcatcggcgccgacatggccaagccgaagaaccggctggatcatcgccaccacgccatcgatgccatcgtcatcgcctgcacctcgcgcgcgctcatccagcggctggcgacggcgtccgcggcggccgaggagcgcggggccgagcgtgtcgtcgagcggatcgatcagccctggtccggcttccgcgaggatgtccgtgcggcggtgggcgccatcgtcgtcagccacaagccggatcacggcacggtgggccgcgaaggctatgcaaagggtcgcgggcagacggcgggcaagctgcacaacgataccgcctacggacacaccggcgaaaaggacgccagcggcaacgatatcgtcgtgcgccgcattgccgtcaccgatatcaggagcccggccgatatcaggaagatcaggaccaacgcgcacggccattccgagctcagggaccggctttacgaagcgacacgcgggctggagggcaaggcgttcgaacaggcggtgctggatttcgtcaagggcgatgcgaaattcaggggtatccgccacgtccgcatcaccgaagtggcgaatcccgtctggatcacgcatggcgacgggcggcacaagaagggctatctgccgggcggcaacgatcgcttcgatgtctgggaactggctgacggcagatgggatgccgaagtggtgacgaccttcgatgcgcaccggcccggtttcgtgccccgcatgcggctcgaaaaccacaatgcccgcaagatcatgagcctgaagaagggggatatgatcgcctacaacgaccctgtcagcgggcagcgtatcatcgccatcgtccgcaagttcgaccagcgcaacaagcagctttacctcgatccgcacaacgaggcgggcaatctcgacaggcgggaaaaggagaaaacctacaagccgcttcgcccgatgcccaacccattgaaaaaattcaggccgcggcaggtccgcatcgatgagatcgggcaggtgttcgatcccggtccgtggtgggaaaagcaggacggttaa

SEQ ID NO: 400 - DNA, 3117 bp, Variovorax paradoxus; identical to SEQ ID NO: 396
  source 1..3117: mol_type = genomic DNA

SEQ ID NO: 401 - DNA, 564 bp, Azospirillum brasilense
  source 1..564: mol_type = genomic DNA
ttggctccaccacccgatcactgtccggctctggtgctgaacgcagactttcgtccgctcagctactttccgctgtcgctatggtcatggcaggaggcggtcaaggccgtcttcctggaccgggtgaacatcatctccgaatatgaccgcacggttcgctctcccagtttcgagatcaagctgcccagtgtcatctcgctgaaagagttcattccggcgacgcggcggccggcctttacacgcttcaacgtcttcctgcgcgaccgcttcacctgccagtattgcggccgtcccttcccgacgcaggaactgaccttcgaccacgtcattccccggtcgcgcggcgggcgcaccacctgggacaacgtcgtcacatcctgttcggcctgcaatctgacgaaaggaaacaggctgccgcacacctgcggcatgatccccctaagtccgccctttcagccgaccgcctacgagcttcaagagaacggacgtgcattcccgccaaacttccttcacgaaagttggcgggattttctttattgggatacggaactggatcccatctga

SEQ ID NO: 402 - DNA, 336 bp, Azospirillum brasilense
  source 1..336: mol_type = genomic DNA
atgcccgtcagccctgaagacacccaactgatggacgtgcttcagggcgggcggtgtttctattgcggggaaccggtgggcgcgaaggcgaccttcgaccacctgatcccgcaggcctatggcggcgccgacgagccgtccaacgtcgttctggcgcaccgccgctgcaaccagcggaagggcgaccgcctgcccacgcccgaggagatcgagcgtttcgtctcccagcgccgccgcagccggttgggcgtctggccgcccctgctcgccttgctggacgccgaacccggcgacgagtggatgaccgtcgcacgggccatcgccgacctccgctga

SEQ ID NO: 403 - DNA, 696 bp, Paenibacillus sp.
  source 1..696: mol_type = genomic DNA
atgcaagaaatgaagaagtgcaagaaatgtggagtagagaagccggacaacaaagaacacttcgttataaaaaaggcgaagctcaaagagtatataactagccaatgcaaggaatgtcaacggttatacaataggaaataccgaactgaaaataaaaatgatatttttgagcagaggaagaattatcggaaaaataattctgtggttatttcggaacgcaaaggtaagtactacgttgcgaacaaatcgagaattatagagaaaagtaaactatattacaaagagaattcagcatctattttagtgagatcaagactctattgtaaggaatacaagtcaaaaaataaggatgtaatcgtcagacaaagacagcgcagaaaatccttgaaaagacaacttccggccacactgaacattaaacaatggagaagtgccctagaatattttgattataaatgctgctattgcggacagaaaagtccattaacccaagatcattttattcccctctctaaaggtggggaatacacaagaaataatattatacctgcatgcggaatatgtaacagttcaaaggggagtaaagattttttcgaatggtaccaggatcaaacattctatagtaaggaaagagaaattttgatccttagttgtttgaatcaaccaaatagatttcaagaactagcactcctctaa

SEQ ID NO: 404 - DNA, 1395 bp, Paenibacillus terrae
  source 1..1395: mol_type = genomic DNA
ttgctgggtacagtatccaagctattaggctccattccagtagtagggaatgtggccaatgtgatgttgaatacgtcgggtggactagcaggtaaagtggcggcaacggttttacaagcaaccgcagcgattcctgttcggagcaaaggaacgcctaccttgggagcgtctaaagaagcaggtgtccatccgttaaaacatctggcaggtttggcgcttcaaggattgattagtcagtatgagcatgagcaagcgaaacaagcctattatagcaagtggattttaggcaaagtatacacgagtgcgcgtcatgtggcgcattccggtggtcctctggagctcgttcaaaacttactgaaagaatcgaattctatggctcgtgcttatcagcaaattcccgccgagttgagacaacgctggagggcgaactacgacagttacatggcacaacaagaaaagccaacgaaagaagagaaatctcagtcttggtgggaacgagcattagagaagcaaggtgaggctgaattactgaacgcacaactacagttagaaggagcccggattagcagtgaaatggcatgggatgctactcaaggagcataccaagctggtttaagaagtgtatcacatggtaaacatcaaattgactacaacccaaaacatccgattgcaggtaaaacaggtgaagtgataggagatgtgttcagcactgtagctggagttggagagatgatgattggtacggggggagaggttggtggttttctccttgatggatcggtcgtattatctccggctggtgtgttggtgaacgttgcatctgctggtatgatatatcatggaggtgtgatgtcctaccacggggcgtcgaatgcaggaggagattctgctgaactctggaaaatgattaaagaaaaaaacaagcagggctcgacccctaagcccactgataccaagtcgagtaagggctcgccaacaccacagaaaccgaaaaagagtgttcctgaaaataaaggaagcaaggacgcagaatcaggtccttattccaatcttgaagattcaaaacatgtggggccaggcaaaaatttcacacctgcccaaaagaaaaaaattatagaggaaaatagaaaacaaaacggaggcgtggtgaaatctgatcaatctggtcaaattgctactaagccacaaaaaagccaaaaaggtgttactcccgatcagaatgaatggcaaattgatcatatttatcccaaagataaaggtggaagtaatagttatagtaatgcccaggtactaacacgtaaagaaaacagagataaatcaaatattttgccttctgattcatcgaaagccaataaagcgaatccaccttctaaacctaaaaaagggagaccataa

SEQ ID NO: 405 - DNA, 1395 bp, Paenibacillus terrae; identical to SEQ ID NO: 404
  source 1..1395: mol_type = genomic DNA

SEQ ID NO: 406 - DNA, 498 bp, unidentified
  source 1..498: mol_type = genomic DNA
ttgagaaccctggtcctgaatgcgggctatgagcccctcgccgtcgtgtcgttcaagcgcgccctcgtgctcgtgatgaaccagaaggcgtccatcatcgcggcggatgccgagcatcccgtgtgggggtccgcgggggcgtgggatcgcccctcggtgatcatcctgcgcaactacgtacggatcccgagcggtcgccacgttcccgtgtcgaggcgcggcgttctgcgccgcgacaacaaccggtgcggctactgcggggggtcggccaacacgatcgaccacgtgcttcctcgatcgaggggcggtaaggacagctgggagaatctcgtcgcctgctgcctgcgctgcaacaacatcaagggcgatcgcacgccggccgaaatgaactggaacctgcgcaccaccccgcggccgccgcacggcacgacgtggctggttcgagggatcgagcgcgcgctgccggactgggaggagtatctcgctcccgcggcctag

SEQ ID NO: 407 - DNA, 498 bp, Microbacterium aurum
  source 1..498: mol_type = genomic DNA
atgcgcactctggtgctgaacgcaggatacgaacccttggccgtcgtctcgttcaagcgagccctggtgctcgtgatgaacgacaaggccaccgtcgtcgaacacgtcgacaacgatccggtctggggtgcgagaggcgcgtacgaccgcccggccgtcatcctgctgacccgctacgtccgggtgccggggagtcgccgggtgccggtgaccagacgcggagtcctgcgccgcgacgtccaccgctgcgcctactgcggcaagggggcgacgacgatcgaccacgtgctgccgcggtcgcgcggcggggcggactcgtgggagaacctcgtggcatgctgcctgcgctgcaacaacatgaagggcgaccgcaccccgcaggagatgggctgggagctcaagtggatcccgcagccgccgcgcggggcgcagtggacggtccgcggcaccgagcgcaccgacccggtctgggagccgtatctggccctcgccgcctga

SEQ ID NO: 408 - DNA, 975 bp, Bacillus megaterium
  source 1..975: mol_type = genomic DNA
gtgagttttaatttaaaagtgggagaaataaaaaaagcttatttaacagaacaagaaatttggaagatcattaatcaattttttgctaatggtcactttaccacaacctataaatatggtttactgaaggctttaattgaaaacctatataacgttgacaatcagttagtcctcacgtttgatcaagtgtatttttcatttgctaaaatatattggaatttggttattcatcatgatttaaatcaattaaatacacccaatagacaatctggaattcaaaaagagttaaaggaatttcagttaatgcatggtgtccccaataaagtagtatttgatcgtttgccaagtaattttcaattgcagttagtagaaagaactaaaaaagtgggggcaaggtatgtagtaggggcgttatacggagatatagaagcttctatttatgaatttgacaaaagaacagaatacataaagtttaatagcagtatgtatatttttttacaaaaatatagacaaattgttattcatttaaccaattatcatttggcgaaatttttagaaaagcataatgacaaaaataagttggaagatgtcttgttgaaagtggaaaatatatcaaaacgcgaatcgctgcatgcattctataacatactttggaaatacgaaacaaaggaatgtttttattgtgggaaagttctttctgataaacgcatcgctactcatgtagatcattttattccatggagttatatccaatcagataatatgtggaatttggtgttaagctgtcaaagctgcaacacaaaaaagagcaataaacttgctaagagcttataccttgagaagatttttgttcgtaatgagaatcttaaattaacagcagttcacaatgattttaactattattcggagcgtaagttagaagctctttatgagtatgctggaatgaatggatacgtagaaaactggtctccttaa

SEQ ID NO: 409 - DNA, 3237 bp, Bacillus sp.
  source 1..3237: mol_type = genomic DNA
ttgcgatatgtattaggattggatattggaatcgcatcatgtggttgggctgtaatcaatcaagaaaaagagagaattgaagatttaggggttcgtatttttgataaagctgaaaatcccaaagacggaaaatcgttggctgcacctaggcgtgacgctcgctctactaggagaaccctacgccgtaaaaagcatagaatgcaacggataaaggtacttttagtaaaacatggcttactatccaagaccgaactcgaccatctatatgaaagtgctactgaagtcgatgtttggaacttacgtttagatgctctagagcgcaaattatatccaaaagagtttgccagaattcttattcatcttgctaaaagaagaggatttaaatctaatagaaaagaaacaactctctcagaaaacgggcaaatcttagaaagcatttcagagaaccaacagattatggagaaacaaaattatagaactgttggagaaatgattctaaaagataaaaagtttgaaaaccataaaagaaacaaaaatggtacttatgttggtactgttactcgtcaacaacttcaagatgaaatacaaatgatattccatgcacaacgtttgtacaaaaacgattatgcaacagaagaatttgaaagtagctacttagaaatctgggcatctcaacgcccctatgcttctaaagatcaaattgaaaaaatgattggttactgtaccttagagcctaaagaaaaaagagcgcctaaagcttcttggtcatttcaatattttgtagcgctacaaaccataaacaacctccgattgatcagtaaagacaggatagaagaactctcttttgaggaaaagaaacaaattatgaatctagcacttgaaaaatcaattgttaaatacattgatatccgaaaattattatctgttcctaatgaatttcaatttaatagcctatcatattcagctgacacagtagatacagcagtggaaagtagaaaatgtatagaactcaaagaatatcactctattaataaggtatataaacaaatttacggggaaaatgccctaaatttattacctaaatattacgatacaattgcatatggactaactatctttaaagatgataaagatatcctagaatatttacaaaataaatatgtaaatgcaaaaggaaaacctataagtaattttgctaacaagacctacgatgacacatttattcaggcattactgaatctgaatttcacgaaaatgggacatttatcatttaaagctttaaaaaatattattccctttttagaagaagggctatcgtacgataaggcatgtgagaaggcaggatacaactttaagggaacatcctacacagaacaaacaaagtacctacctgttatccctcagaatacaaatcctgttgtacatcgtgctctttcacaaacaagaaaagttataaatgccattataaaaaaatatggttctccaaacgcaatccatattgaaactgcaagagaattatcgaaaacattccaagaaagaaaagaaatagatagtatgtaccaggataactctaaaaagaatgaacacgccattcataagttaaaagaattggggaacattaacccctcaggaataaatattgtgaaatttaagctttggaatgagcaagatggtagatgtatgtattctggaaaatatatcgaaccacatcggttatttgaagaaggttatacggaagtagatcacatactcccctatagccgtagcttagatgatagctataacaacaaaacattaacactaggaacagaaaatcagcgtaaaggaaacaaaattccttatgaatacatggggaatacttcaatttggaatgactttgaaacaagggtacagtcaaataaaaaaatcaacaaaaagaaacaacaaaaattacttttacaacacttttcatatgcgagagaacaggaatttattgaacgcaatctgaatgatacccgttatgccacaatatatctatcatcccttattcaacaacatctcatattctctgaaagttctcgtaagaaaaaagttcatacagttagtggaattataacatctcatttacgtagtagatggggatttaacaaagatcggaaagagggacatatccatcatgctttagatgcggtaatcgttgctgtcacatccgatcacatgattcaaagagtaacaaaatattataagttaaaagagttaaatagaaaccttcaagccaaaaggatgccgtttcctgaaccttgggaaggatttcgtttagagttagaagcaaggatttctccaaatacccagcaatatttaaagggccttcgttttaaaaattacgccgatgtcaatctatccgaaatcaaatctatctttgtttcaagaatgcctaagcactctatcactggtgaattacatcaagaaacgattcgaaaatttattggctataacgaaaaagggaaagtattaaccgcaataaagacaaagttagaagatattccttttgatgccaatggtgattttcccatgtatgggaaagaaagtgatttatacacgtataatgccataaaagaacgttatttaagtcataaaaaagataaacgcaaatcattccaagagcctttatacaaacccacaaaatcaggaggaattggaccacttataaaatctattaaaataatggatacaagaaccattgttaatcctgttaatcaaggtaaaggtgtcgtgtataatagtaaaattgctcgtactgatgtattcaaaaaggaggagaagtactatttaattcccatttacacaattgatcttatgaaaaatatcttacctcaaaaagcaattacagcaggaaaaggatatgaggattggacaaccattgatcctagctttacttttttatttagtctttttcccaatgatctaattcaaattgtcccctcaaaaaataaaacaattaaagctagaacaactgtatcgaaaaaagaggtattactaccctcactcacaggatattttaaaggtgttcatagtggaactgcggggattactgttgaaacacatgatggtagtgttatagcaaatgtaggctcaaagcaattactattaattgagaaatatcaagtagatgtacttggaaattatacaaaaataaaggaggaaaaacgcattgggctgacgacatataatgataacaaaaagatcgcagcttgctaa

SEQ ID NO: 410 - DNA, 975 bp, unidentified; identical to SEQ ID NO: 361
  source 1..975: mol_type = genomic DNA

SEQ ID NO: 411 - DNA, 2982 bp, Paenibacillus polymyxa
  source 1..2982: mol_type = genomic DNA
ttggatgtactgcaaaacggaatagggtatgaaagtctacgcttatacggtccttttcaacctcagcatatggaacaattgcgaattaccagggccgttaacgatcatgcatacctgtatgtgagcggcatgctgtcggaagaacagggggtagcgtgtgttggacaggacatggagcaggaaccgattgtcatacgtcagttggatgagcaaggacagtcgattagaagattgtttcacgggctggttacacgtatgtctgtgcactgcattaggggagtatatacctttgaactggaagcggcctcgcattcctatcaaatggatatcaaaacgaaaaggcgctcctatcaggacatagatcgtacctatgatgatctggttacaacgatggttcggaaatacatgtatggagacgccattgatacggtaaccaattatgccgagttaggtacttttgttttacaatatgaagaaaccgactgggcctttttaaagcgccttgtctctcgttttggttcggtacttgtgccggagattacggctgcatcacctaagattttctttgggatgccagaaggcaagcggtacaaggtggagcgagatgtattttatagggtacgaaaaacatttcatgatttagatacaggaaaaccaggggaacgtgctggcgcgtatgtcacctatacgattgaaaatctgcaatactatgcgctgggtgacttgattacgttacccatcggacaaggcaaagagctggttgttgttcgggcagtgacagagttaaaggacggtttactccaaactcgttatgatctccaagcagaacaggatattcactatactcggtatgaaaatgatcaagccattgggatttcactcacgggaacggtgttgaaggtacgacaggatttcgtacaacttcaactggaaattgatccgaagcaagatccagctaaagcctgttggtttcccgtagccacccgatatgttgcggaagaacatagtggctggtacgacatgcctgaagttggggaacaagtggagctgtatttacctacacactgtgaacagaaagcctatgtgacagattccttgcgacaccatcgccacgccaatggacagccagatgtgaaggcatggaaacacgtacaaggtagcggcatagaaatgtctgggcaggagctgatcctgtccacgtccgatgggttttcgattacactacatgaggatacgggcatcactgtcaacagtccggggaatgtacagatccagggcggtcatgtaaagctggatgcaagtcaggaactatcactggaagctggaactgcactatatttaaaaggtggagccagcagtatggtgctagatggtgagacagacaccaaagctccagtgatttatcaggaaggtacggtaaaagctccagttttcgtagctgatcttcctcctgtacctgagcctccattgatgagtattaaagcctatgaagcggctcaaactgcagcaactgccaaagctctatccagcaatactcctaccgcgaaagtcacaagtccagcggcgcttcagcaggccaatgctttgctgggtacagtgtctaagcttttaggctccattccagcagtcgggaaagtagccaatgtgatgttgaatacatcgggtggactagcaggtaaagtggcggcaacggttatacaagtatccgcagcaatccctgttcggagcaaaggaacgcctaccctgggagcatctaaacaagcgggtgtacatccgttgaagcatttggcaggtttggcgcttcaaggattgattagtcagtatgagcatgagaaagcgaaacaagcctattatagcaagtggattttaggcaaagtatatacgagtgcacgtcatgtggcctattccggtggtaccctagagctcgttcaaaacttactgaaggaatcgaatgctatggctcatgcctatcagcaaattcccgccgagttgagacaacgctggagggcgaactacgatagttacatggcacaacaagaaaagtcaacgcaagaagagaaatctcaatcgtggtgggaacgagcattagagaagcaaggtgaggctgaattactgaacgcacaactacagttagaaggagcccggattagcagtgaaatggcatgggatgctactcaaggagcataccaagctggtttaagaagtgtatcacatggtaaacatcaaattgactacaacccaaaacatccgattgcaggtaaaacaggtgaagtgataggagatgttttcagcactgtagctggagttggagagatgatgattggtacggggggagaggttggtggttttctccttgatggatcggtcgtattgtctccggctggtgtgttggtgaacgttgcatctgctggttttatatatcatggaggtgtgatgtcctatcatggggcgtcaaatgcaggaggagattctgctgaactctggaaaatgattaaagaaaaaaacaagcagggctcgacccctcctaagcccactgataccaagtcgagtaagggctcgccaacaccacagaaaccaaaaaagagtgttcctgaaaataaaggaaacaaggatgcagaaccaggcccttattctaatcttgtagattcgaaacatgtggggccaggtaaaaacttcacacctgcccaaaagaaaaaaattatagaggaaaatagaaaacaaaacggaggcgtggtaaaatctgatcaatctggtcaaatagctactaagccacaaaaaagccaaaaaggtgttactcccgatcagaatgaatggcaaattgatcatatttaccccaaagataaaggtggaagtaacagttatagtaatgcccaggtactaacacgtaaagaaaacagagataaatcaaatattttgccttcagattcatcgaaagccaataaagcgaatccaccttctaaacctaaaaaagggagaccataa

SEQ ID NO: 412 - DNA, 588 bp, unidentified
  source 1..588: mol_type = genomic DNA; note = Paenibacillus beijingensis
ttgaattactttgctacttttcaaatgccaaagccggttaatgtaagtggaaaatcatctagtattaccaactctttcgtaaatggaataataccttgcattgttccctcagagagtgatgttaaagaatgcctggatatcttggggcttgatcccttgaatttgaagtgttcttattgtggagataactatactgaatgggatcactttagacctttaattattgataagaaaccaacaggctacatatctgatatttacaatctagtacccttatgcggtaaatgtaatcaatcaaagggaaacaaaccttggaaaacatggatattgagtaatgcgaaactagcccctaaagctaggggtattaagaacattgatgaaagaataaaaagactcgaaaatttcgagaattggaaggatgtaaaaccaattaacctcgaagagctagtagacaaagagttatggaaaaagcattgggaaaattacgagaaattaaatcagctaatgagggaaagtcaaattacttcacaaaagatacaaagtcagatcagggaaagatacaacttgatagtcaatcagggaaggtaa

SEQ ID NO: 413 - DNA, 1134 bp, Bacillus thuringiensis
  source 1..1134: mol_type = genomic DNA
atggcagatgttaaatggataaaactctctactagcatgtttgaagatgaaaagattcgattgattgaaagcttaccagatgcagacacgctactaatcatttggattaaattgttgtctcaagcgggcaggacaaatgctaatggttacattttcttgagtgaaaacattcctttcacagaagaaatgctttcaacactttttaatagaccaatagcaactgtgagacttgcattacaaacgtttaagcaatttggaatgattgatattactgatgatcagtacatatgcatctcgaattgggagaaacatcagaacatcgatgggttagataaaattcgtgaacaaaaccgattacgtaagcaaaaacaacgagagaaagaaaaacaatttctattaggaaatgacacgggaacatgcgcttattgtggagacctagctaatacactagaccatctgattcctaaatcgcatgggggattggatactcaagaaaattgcgtggcgtgttgcaaacgatgtaatgaaatgaaaacaaaccgtcctttagaagtcttccttaatgaatgtttactactaggaaccccggtgttggatttgaagttggttttaaataacaagaaaatcatgagtttagttgactatcaagacggaaaatttttacgtcccgtgacaggtcacgtgacacgtcacggtagtcacgcaacagatatagaagaagataaagaattagatatagataaagagaaagataaagataaaaagaagaaagaaaaaccatctcgtcacaagtttgaaacttgcgacaccaacggagctaagtatttgtttgaaaaaattaagggtaataatcctaaacaaaaagagcccaacttcgatacttggtctaatgactttagattgatgcgcgaaaaggataaccgtgaattacaagagattaaagatgttattgattggtgccaagctgatccattctggcaaggtaacattttatctcctaaaaagctacgtgagaagttcgaccaactaactattcaaatgaattctaaaaagggagcgaagaacaatgcagagagcggcggcagcaataccaaccgatatagccaaaaaggtgaatatgactatggattctga

SEQ ID NO: 414 - DNA, 2982 bp, Paenibacillus polymyxa; identical to SEQ ID NO: 411
  source 1..2982: mol_type = genomic DNA

SEQ ID NO: 415 - DNA, 993 bp, Bacillus simplex
  source 1..993: mol_type = genomic DNA
gtgagctttaagcttcaggttggagaaatacaaactgaatacctgactgacaaagatatatggagtcattttaactatattttttctagtaaatccaagaactctacgacttataaattcgtgttaattaagtctttgctggaaaacttatataacgtcgatgggcatctacagttatcctattctagtctttataatagttttgccaagatctattggaatttagttgttcatcatcgactaaatcagattaatatggtgaataaaaagtcagaggttcaaaaagtgcttctagttatacaagagaaatacaacattccatctaccattgtgtttgataggctatcccctgaggttcagttagaggttatgctgaaggttaaaaggaaatgtaagattaatgtgatgggtgctctatacggggacactgaggcaactatctatgagtttgataataagaaggagcaattacgatttagtccttcgtactattcgtttatgcaacgatttcagcgtgtccttcaataccttacgaattatcagttagcgttatttttagagaagtttaattctcaaggagatacaacgaaattattggttagaattgaaaatgtttcgaagcgatcttcgttggatcaattctaccgagttttatcttcattctataatgggacatgtttttattgtggtaaatcgattaaacgaaaggaagctgcacatgtagatcactttattccatggagctttgttcaggctgatcagctctggaatttggttattgcgtgttcaccctgtaatttatcaaagagcgataaattggcttcaggtatttatttggagaatctgattgaacgcaattattccttcattaaaaacgagattggtatcaaggaagacatggaagtatacactcaagaaaaattagaagacctttataaattttcaattgataatgggtatacagatatctgggtgcctaaaatagtttattaa

SEQ ID NO: 416 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaatttttcgctaatggtcactttaccacaacgtataaatatggattaatgaaagctttaattgaaaacctatataacgttgacaatcggttagtactcacgtttgatcaagtgtatttttcatttgctaaaatatattggaatttggttattcatcatgatttaaatcaattaaatacacacaatagacaagctggaattcaaaaagagctaaaggaatttcagttaatgcatggtgtccccaataaagtagtatttgatcgtttaccaagtaatcttcaattgcagttagtagaaagaactaaaaaagtgggggcgaggtatgtagtaggggtgttatacggagatatggaaggttctatttatgagtttgataaaagaacagaatacataaagtttaatagcagtatgtatatttttttacaaaaatatagacaaattgttactcatttaaccaattatcatttggcgaaatttttagaaaagcataatgacaaaaataagttagatgatgtcttattgaaagtggaaaatatttcaaaacacgaatcgctgcatgcattctataacatactttggaaatacgaaacaaaagaatgtttttattgtggaaaacttctttctgataaacgcatcgctactcatgtagatcattttattccatggagttatatccaatcagataatatgtggaatttagtgttaagctgtcaaagttgcaacacaaaaaagagcaataaacttgctaagagtgtatacctcgagaagatgtttgttcgtaacgataatcttaaattaacagcagttcacaatgattttaactattattcggagcgtaagttagaagctctttatgagtatgctggaatgaatggatacgtagaaaactggtctccttaa

SEQ ID NO: 417 - DNA, 975 bp, Bacillus megaterium
  source 1..975: mol_type = genomic DNA
gtgagttttaatttaaaagtaggagaaataaaaaaagcttatttaacagaacaagaaattcggaagatcattaatcaatttttcgctaatggtcactttaccacaacgtataaatatggattaatgaaagctttaattgaaaacctatataacgttgacaatcggttagtactcacgtttgatcaagtgtatttttcctttgctaaaatatattggaatttggttattcatcatgatttaaatcaattaaatacacacaatagacaagctggaattcaaaaagagttaaaggaatttcagttaatgcatggtgtcccaaataaagtagtatttgatcgtttaccaagtaatcttcaattgcagttaatagaaagaactaaaaaagtgggggcaaggtatgtagtaggggcgttatacggagatatggaaggttccatttatgagtttgataaaagaacagaatacataaagtttaatagaagtatgtatatttttttgcaaaagtatagacaaattgttactcatttaaccaattatcatttggcgaaatttttagaaaagcataatgacaaaaataagttagaagatgtcttgttaaaagtggagaatatttcaaaacgcgaatcgctgcatgcattctataacatactttggaaatacgaaacaaaagaatgtttttattgtgggaaagttctttctgataaacgcatcgctactcatgtagatcattttattccatggagttatatccaatcagataatatgtggaatttggtgttaagctgtcaaagctgcaacacaaaaaagagcaataaacttgctaagagtgtatacctcgagaagatgtttgttcgtaacgataatcttaaattaacagcagttcacaatgattttaactattattcggagcgtaagttagaagctctttatgagtttgctggaatgaatggatacgtagaaaactggtctccttaa

SEQ ID NO: 418 - DNA, 588 bp, Pantoea vagans
  source 1..588: mol_type = genomic DNA
atggcaaacgctatcgagcaaggccatacattctggagtgatgagtctcttggtgatcttaaaagaaaaataaaaaatcaccttagacaaaatcagcaggaatgttgttgttattgttctcgtaacattgacgatgaatttaacatggtacttgacattgagcatattattccaaaatcaaaaatcaagtcagaaatgtttgagatgatgaatttggcagtatcttgtaaaagatgcaacatgagaattaaaggtgaggatgtttcttttattgagggagattttgaagatttcaaagcttctgaaaattattatcaaaccgaatgttataagtttattcatcccaacattgataattgggatgaaaacttaacatatttagtggctcaagttaatagaagaaaaattgtttattataatgtaatgcccggaagtgcaaaaggggaatttaccaaaaaatattttgagctcgataaaattcaagctaatacatttgatgaggcacaggaagctacaggtagaaaagaaccctttgacccgaaaattgctgaacagttttcaaatctcgtcaattcaatgttaggttaa

SEQ ID NO: 419 - DNA, 975 bp, Bacillus megaterium; identical to SEQ ID NO: 408
  source 1..975: mol_type = genomic DNA

SEQ ID NO: 420 - DNA, 975 bp, Bacillus megaterium
  source 1..975: mol_type = genomic DNA
atgaatgactatattaaacaatggcataatttaattttaaattgcaactttgataacacatataaaatggtttggagcaaagcattagtagaattgtcggttaatgttagtgatgaagttgataaagatataataatttttgattttgatgatattgcaaaactttgcttgaaatattattggaatcaaactatttactttgatttaatacaaagtcctaacctaaagaaacctccagaaatacttaatcatattaaatccttaattacccatttttttgatacgaaaacttctgttcagccagttagatttgaaaagatagattttaacaaacttcaattaatagataaatacgaagaaaccgttaaaagaataattacaacgttaaagaaagacgtttgctggcgatttaaaattcttaattctaacacatacgatatatatgaattagaccgtaaaaaaggtaaggtgtattttaaaaggcaagacgtagtaacgcttaaacaaaatagtgacttgctatttacctttataaattacagatggacacaaatgttagaggggttcaattatagtccacggatttcaagaaaagtacgtgcgatagatgaaaacaatattaagcgtgtgtcgttaaaaaagtttcataagtatcttgatttaatgtttgaagacagtaaacgtaaatgcttttattgtggtgaagaaatagatgatagcaatctgtcagttgaccatgttataccttggtcttacatgttttcagatgatttatggaatttagtttactgtcataaagatgaaaactccgaaaaatctaatcgtctcccattggaagaagatatattaagattagaaaacaggaatatagctttgctagaacgtatggagaacgcaactaaagactacgaccaattacaaatagctcttgagaaaaattacgttaaacagttttggatttcatttaaagggtaa

SEQ ID NO: 421 - DNA, 975 bp, Bacillus megaterium; identical to SEQ ID NO: 9
  source 1..975: mol_type = genomic DNA

SEQ ID NO: 422 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaatttttcgctaatggtcactttaacacaacctataaatacggtttactgaaggctttaattgaaaacctatataacgttgacaatcagttagtcctcacgtttgatcaagtgtatttttcatttgctaaaatatattggaatttggttattcatcatgatttaaatcaattaaatacgtccaatagacaagctggaattcaaaaagagttaaagaaatttcagttaatgcatggtgtccccaataaagtagtatttgatcgtttgccaagtaatcttcaattgcagttagtagaaagaactaaaaaagtgggggcaaggtatgtagtaggggcgttatacggagatatggaaagttctatttatgagtttgataaaagaacagaatacataaagtttaatagcagtatgtatatttttttacaaaaatatagacaaattgttattcatttaaccaattatcatttggcgaaatttttagaaaagcataatgacaaaaataagttggaagatgtcttgttgaaagtggaaaatatatcaaaacgcgaatcgctgcatgcattctataacatactttggaaatacgaaacaaaagaatgtttttattgtgagaaagttctttctgataaacgcatcgctactcatgtagatcattttattccatggagttatatccaatcagataatatgtggaatttggtgttaagctgtcaaagttgcaacacaaaaaagagcaataaacttgctaagagcttataccttgagaagatgtttgttcgtaatgagaatcttaaattaacagcagttcacaatgattttaactattattcggagcgtaagttagaagctctttatgagtatgctggaatgaatggatacgtagaaaactggtctccttaa

SEQ ID NO: 423 - DNA, 570 bp, Bacillus sp.
  source 1..570: mol_type = genomic DNA
atgaatgatcacatagaaatgcgcacttgccgaatatgtggagaaaaaaagccacttgaaaaattcgaaattgattctcgtaaggaggacattcgaacctaccgatgctattcatgtaagcaaatgacacaagacaaagctactcgtgcttatcatcgtttaaagcaccgttcaacagctccggtcgaggtcactccagcggaaataaggcttttgttcgaagtgtttgacgggtgctgcgcctactgcggagaacggcctgaaaatgaacgcgatttacatctggatcatatcgtgccgatcagcgaattcggccgcaatactcttgcgaatcttatccctgtttgcagtcgcgataatcggtcaaagcataaccgtccgctcgcaagctatttcctagctaatcgcgaaaagtttcctaccgctaatatggcgctggtggtcgattacattgctttgctgactggctcaaagagagaggacgttgttggggatttaacagacgaacatgtggcgtatgagttgaaacaggcgcggttggctatcgagaaagaggcgagtaaaacatga

SEQ ID NO: 424 - DNA, 975 bp, Bacillus megaterium
  source 1..975: mol_type = genomic DNA
gtgagttttaatttaaaagtaggagaaataaaaaaagcttatttaacagaacaagaaatttggaagatcattaatcaattttttgctaatagtcactttaccacaacgtataaatatggattaatgaaagctttaattgaaaacctatataacgttgacaatcggttagtcctcacgtttgatcaagtatatttttcatttgctaaaatgtattggaatttggttattcatcatgatttaaatcaattaaatacacccaatagacaatctggaattcaaaaagagttaaaggaatttcagttaatgcatggtgtccccaataaagtagtatttgatcgtttgccaagtaattttcaattgcagttagtagaaagaactaaaaaagtgggggcaaggtatgtagtaggggcgttatacggagatatggaagcttctatttatgaatttgacaaaagaacagaatacataaagtttaatagcagtatgtatatttttttacaaaaatatagacaaattgttattcatttaaccaattatcatttggcgaaatttttagaaaagcataatgacaaaaataagttggaagatgtcttgttgaaagtggaaaatatatcaaaacgcgaatcgctgcatgcattctataacatactttggaaatacgaaacaaaggaatgtttttattgtgggaaagttctttctgataaacgcatcgctactcatgtagatcattttattccatggagttatatccaatcagataatatgtggaatttggtgttaagctgtcaaagctgcaacacaaaaaagagcaataaacttgctaagagcttataccttgagaagatttttgttcgtaatgagaatcttaaattaacagcagttcacaatgattttaactattattcggagcgtaagttagaagctctttatgagtatgctggaatgaatggatacgtagaaaactggtctccttaa

SEQ ID NO: 425 - DNA, 981 bp, Bacillus megaterium
  source 1..981: mol_type = genomic DNA
atggatggttatattaaacaatggaatgacctaatactaaattgtagctttaacaatacgtataaaatggtgtggagtaaggccttagttgaactatctctaagttttaatgataataatgacgaggaaactgaaactattgcttttgattttaatgagataggggagctttgtttaaagtactattggaaccaaactatttattttgatttagatcaaagtcctaacttaaagaaacctcccgagatgattacgtatactaaagagctcattaaagagttcttttttaggagaggaaatgtacagccaataagatttgaaaaggtggattttaactcacttaaaatggaacataactatgaatcgacaataaaaaacatagcaaaaacaataggacaagacgtttgctggcgctttaaaaagcttaatggtaatagctatgaaatatacgatcttgatattaatagagaaaaggtattttttagcaagcataatgtattgcaacttaaagagcatagtgattttctatttaccattataaattatagatggacgcaaatattagaggggtttaattatagtccaaggatttcaagaaaggtaagggcaatagacgaagacaatattaaacgggtatctttaaaaaagttccataagcaccttgatttaatgtttgaagatggccaacgtaaatgcttttattgcagtgaaatcatagatgatactgatgtatctattgatcatgttataccgtggtcttatatattctccgatgatctatggaatttagtttgctgtcataaaggagaaaactccaaaaaatacaaccgtcttccctcggaagaagatattgtacggcttgagaatagaaatatacttttacttcaacgtatggaaaacaagtcaagagagtacgatcaattacagattgctgttgaaaaagactacattaaaaagttttggatttcctttaagggataa

SEQ ID NO: 426 - DNA, 3846 bp, Chryseobacterium sp.
  source 1..3846: mol_type = genomic DNA
atgacaaaaaatatacttggattagatttgggaacgaactcaattggttgggcgttaatagaaattgatgataatggaactcctataagaatcgttgcaatgggttccagaattatcccaatgtcatcaaatgatagagatgaatttcaaagaggtttatccattactaagaatcaaaacagaacagtaagcaggactcaaagaaaaggttatgatcgtaaacaactaaagaaaagttatttaaaaaaaattcttaataaatataatatttatccatcagaagaattattaaaactaccgatgttggatttgtggaagttgagaagtgatgcagctaatccaaatgaaaatatttcagcagaacaattaggtcgtattttatatatgcttaatcaaaaacgcggttataaatctgcaagaagtgaagctaatgccgataaaaaagatactgattacgtacaagctgtaaaagggcggtatgctcaactaaaagataaaaatcagacaatcggacagttcttttatgaaggtttacataacgcaaaggtaaataattcttatttcagagttaaagaagaagtttatccgagagaagcatatatagaagagtttgataggatcattaatgctcaaaaagataaacacgattttcttactgaagaagttatcaatcagttaagaaatgaaattatatattatcagcgaaagctcaaatcacagaaagggttggtcagtatttgtgagtttgaaggatttgagaaaactgtttttgataaggagaagaataaaaataaagtcgtttttgcaggccccaaagttgcgccgaaaacttcacctttgtttcagctatgccgtatttgggaagttattaataatatttccttaaaaattaaaaactctgaaggaagcaaatataaatggggagagaaaattccaactttagaagaaaaggaagctttggcagattatcttttcagaaatgaaagtctgagttttacaaagcttttggaaattttatcgctgaataaaaatgatgtatatgtcaataagcaaattctgaaaggaataaaaggaaatgaaacttattcagctcttcacactgttttaggagatagtgagcttttaaattttgatgtttctattattccatccaatcatacttcgatattagttaataaaaagactggagaaattctggaagaaagagcaggtctggagctggatgcttcattggagaaagaaccattatatcaattatggcataccatatattctttgaaagacttggatgaatgcaaaaatgctttgataaaaagatttggttttgatgatgaaatttctgaaaaattgtcgcgaattgattttaataaacaggcatttggcaataaatcgaataagtcgatgagaaagattcttccttttctaatggagggttatgattattcccaagcttgtagtttagccggttataatcattctaattctttaacgaaagctgaaagagatgaattacagacctcagataaattagaacttcttccgaaaaacagtttgaggcaacctatcgttgaggagatattgaatcaaatgattaatgtggtcaatgcaattattgaacaatatgggaaaccgtctgaaataagaattgagcttgctcgtgaacttaaacaaagtaaggacgagcgaaacgataccgatttacaaaattcaagaaataaaaagcttaatgaagaaattggtaaacgcttggctgagctgggattacctgttaccaaacgatatattcagaaatataaatttatttttccatcgcttagtaagagccttaaagatgctcaggttaataatcaatgcatttattgtggagaaacttttaatctcacagaagctttaagcggtgatgcttatgatgtagaccatattgttccgaaggcattgttatttgatgattctcaaaccaataaagttttagttcatagaaaatgcaactcaacaaaaaccaatcaaactgcatatgattatattgctacaaaaggcgaagaacaagttcgtatctattctgaacgagttgatgattggttcaaaagaggaattatttcttacagtaaaatgcagaggcttaaagtttcacataaagaatatcttgaacgtaaaagtttaaaaaaggaaacagaagcggacaaaaaactctgggaaaacttcatagatagacaattgagagaaactcaatatattgcccgtaaatcaagagaaattcttcaaaaaatttgtaataatgtaacttcaacagaaggtgcagtcacggctaaacttcgtaatctttggggatgggatgatgttttgatgaatctgcaaatacctaaatataaagaattaggacaaactgttatcagagaatggattagtgaccacggtaaaaaaaaacatcaaaaagaagaaatagaaaactggacaaaaagagatgaccatcgtcatcacgctattgatgctttggtaattgcctgtacgaagcaaggttttattcagcgtatcaatactttaaattcaagcgacactaaagatgagcttagaaaggaaattgaagagtctaagaaaaagttaggacaaccttttaatgaaaatgattacggacatcttcacaattcaaaagtaactttattggaagattatttacgcacacaaagaccaccaacatttactacaaaatatgtagaacaagaagcggataaagtccttatctctttcaaagcgggtaaaaaagttgctactcttacaaaattcaaagccaccggaaaaaataaagaacaaggtgtaattgttccaagaggtgctttgcatgaacaatctgtatatggtaaaataaaagtcattgaaaaagataagcccctaaaatatttatttgaaaattcagataaaattatcaatctaaaagtcaagaatttaattcaacaacgacttgctgagaataaggatgatataaaaacagctttatcatcattaaagaaaaatccaatcttcctgaatgagcagaaaacggaaattctggaaaaatcatcttgttataatgaagcgacgattttgaaatataaattacaaagcttaaaagcaagtcaagctgatgatattgttgatagaagagtaaaatttctggtaaaagaaaggttggctcaatttggaaataaggaaaaagaagcctttaaagatacgctttggttcaatgaagaaaaacaaattcccattattaccgtacgtacgtttgctcgtcccgatgctaataatttacaggcaatcaaaaaagatgaaaatggacacgatataggctttgttcttacgggaaataatcatcatattgcgatttatgaggataaagataaaaagtttattcagcatagctgcactttttggcatgccgtggaaagaaaaaaatacaacatttctaatgtcattaaagattcatcagatgtttggagtcaactattagacaaagaacttccggaatcatttttaaacaaattacctgttgataagctaaatttaaaatttagtatgcaacaaaatgagatgtttattcttggtttatcacaagatggatttgatgaagcaataaaaaataacgataaggctttgctgagtaaacatttgtacttggtttggagtatttctgaaaataattattggttcagacatcatttagagactaaaaatactgatttaaagaaaacagagggagccaaggatagtaaaagactttataatattagaagtttgggctctttgttttccttaaacccaataaaagttagattaaatcatttaggagaaattactaaaataggagagttttaa

SEQ ID NO: 427 - DNA, 3846 bp, Chryseobacterium sp.; identical to SEQ ID NO: 426
  source 1..3846: mol_type = genomic DNA

SEQ ID NO: 428 - DNA, 999 bp, Bacillus simplex
  source 1..999: mol_type = genomic DNA
atgagccataaactgcaagtaggggaaatgaaaactgaatatttaactgatcaagatatttggagtcagtttaattttattttctctagtaaatcaaaaaattcgactacatataagtttgtgttaatcaaatccctgcttgaaaatttgtataacgtaaacgaaaagctagagctgtcttatgatatcctgtttagtagtttttcaaaaatttattggaatttagttattcatcataatctaaatcaaattaatatgctgggaaagaaagcggaagttcaaaatgtgttaggggctattcaatcaagttatcagattccaaatacgctcgtttttgataagctttcttcagaaattcaagtgcaggtcattactaaagtgaaacgaaagtgcaaaatcaatgtgatgggtgcactatacggtgataccgattctactctttatgattttgataataaaaaggaatatatacgaatcaatccttcttattattcttttatgcaacgatttcagagagtacttcactatttaacgaattatcagcttgctcttttcctggagaaattcaataacgaaggggatacaacgaatttattattgaaggtagagaatgtttccaagaggtcttccttagatcaattttatcaagtgttggaatcattctatgatgggaaatgtttttattgtgaaaaagcaattaagaaaaaagcggcagcgcacgtggatcacttcattccgtggagctttgtgcaaaacgatcagttatggaatttagtgattgcctgtagcacgtgtaatctctcgaaaaatgataagttagcctctggctatttcttggaagctttaatcgatcgaaatgaagaatttttgatgcagtccgatctttttcaaagggaagatatgcaggtatatagtgagaagaagttaacggagctttataaatattcggttgataatggctttaccgatatttggatgccgaagaaaactatattatag